(19) United States
(12) Patent Application Publication
(10) Pub. No.: US 2024/0287100 A1
(43) Pub. Date: Aug. 29, 2024

(54) ALKYLIDENE DERIVATIVES AS KRAS INHIBITORS

(71) Applicants: NIKANG THERAPEUTICS, INC., Wilmington, DE (US); SHANGHAI BLUERAY BIOPHARMA CO., LTD., Shanghai (CN)

(72) Inventors: Jiping FU, Wilmington, DE (US); Yan LOU, Wilmington, DE (US); Yongfeng SUN, Shanghai (CN)

(21) Appl. No.: 18/411,493

(22) Filed: Jan. 12, 2024

Related U.S. Application Data
(63) Continuation of application No. PCT/CN2022/105169, filed on Jul. 12, 2022.
(60) Provisional application No. 63/221,764, filed on Jul. 14, 2021.

(51) Int. Cl. C07D 519/00; A61K 31/519; A61K 45/06

(52) U.S. Cl. CPC C07D 519/00; A61K 31/519; A61K 45/06

(57) ABSTRACT

The present disclosure provides certain alkylidenyl derivatives that inhibit certain K-Ras proteins and are therefore useful for the treatment of cancers mediated by such proteins. Also provided are pharmaceutical compositions containing such compounds and processes for preparing such compounds.

### CROSS REFERENCE TO RELATED APPLICATIONS

The present application is a by-pass continuation application claiming priority to, and benefit of, PCT International Application No. PCT/CN22/105169, filed on Jul. 12, 2022, which PCT Application claims priority to and benefit of U.S. Provisional Patent Application No. 63/221,764, filed Jul. 14, 2021, both of which applications are hereby incorporated by reference herein in their entireties.

### FIELD OF THE DISCLOSURE

The present disclosure provides certain alkylidene derivatives compounds that inhibit certain K-Ras proteins and are therefore useful for the treatment of cancers mediated by such proteins. Also provided are pharmaceutical compositions containing such compounds and processes for preparing such compounds.

### BACKGROUND

Kirsten Rat Sarcoma 2 Viral Oncogene Homolog (KRAS) gene is a prevalent oncogene that encodes a small GTPase transductor protein called K-Ras. K-Ras can serve as a molecular switch by cycling between active GTP-bound and inactive GDP-bound forms (see Science 2001; 294:1299-304.). K-Ras signaling is activated by RAS guanine nucleotide exchange factors (GEFs), e.g., Son of Sevenless homologue (SOS) protein, that facilitate the GDP to GTP exchange of K-Ras (see Curr Biol 2005; 15:563-74.). The interaction between K-Ras and GTPase-activating proteins (GAPs) such as p120GAP and neurofibromin, potentiates K-Ras intrinsic GTPase activity and accelerates GTP hydrolysis and diminishing K-Ras signaling (see Curr. Biol. 2005; 15:563-74.).

K-Ras plays a crucial role in the regulation of cell proliferation, differentiation and survival by signaling through several major downstream pathways, including the MAPK, the PI3K and the Ral-GEFs pathways (see Lung Cancer 2018; 124: 53-64), among them the MAPK pathway is the best characterized (see Mol. Cell Biol. 1995; 15:6443-6453.). K-Ras-GTP binds to and activates RAF kinases, which phosphorylates MEK and subsequently phosphorylates ERK. Phospho-ERK can further activate downstream cytosolic proteins and which then translocate to the nucleus to drive the expression of diverse genes, propagating the growth signal.

PI3K pathway is also involved in RAS-mediated tumorigenesis (see Cell 2007; 129:957-968.). Upon activation by K-Ras-GTP, PI3K phosphorylates PIP2 to form PIP3, activates PDK1 and then phosphorylates AKT. pAKT yields phosphorylation of several physiological substrates, e.g., mTOR, FOXO and NF-κB that promote metabolism, cell-cycle progression, resistance to apoptosis, cell survival and migration. The Ral-GEFs signaling pathway plays a key role in RAS-mediated oncogenesis as well (see Proc. Natl. Acad. Sci. U.S.A. 1994; 91:11089-11093.). The K-Ras effector, RALGDS, stimulates the RAS family RAL-A/B small GTPases for the subsequent signaling cascades. RALGDS can also promote the JNK pathway to stimulate transcription of pro-survival and cell-cycle progression genes for cell proliferation and survival.

KRAS gene is the most frequently mutated oncogene in human cancer. KRAS mutations are associated with poor clinical outcome and found at high frequency in pancreatic cancer (˜90%), colorectal cancer (˜44%) and non-small-cell lung cancer (NSCLC) (˜29%) (see Cancer Discov. 2021; 11:1-16). KRAS mutations are also present in breast cancer, liver cancer, biliary tract malignancies, endometrial cancer, cervical cancer, bladder cancer and myeloid leukemia. The most common KRAS mutations are observed at residues G12 (77%), G13 (10%), and Q61 (6%), and the most predominant KRAS variant in human malignancies is G12D (35%), followed by G12V (29%), G12C (21%), G12A (7%), G12R (5%), and G12S (3%) (see Cancer Discov. 2021; 11:1-16). These mutations perturbate GTP hydrolysis of K-Ras by interfering with GAP binding/stimulation and/or reducing K-Ras intrinsic GTPase activity, resulting in constitutive activation of the protein and K-Ras signaling.

Targeting KRAS signaling has been a long pursuit in drug discovery. Among KRAS mutants, K-Ras G12C offers special opportunity, because it harbors a non-native cysteine residue, which can act as nucleophile and therefore can be targeted by covalent attachment. Several such covalent inhibitors, including AMG510, MRTX849, JNJ-74699157 and LY349944631, are in clinical trials for treating cancer patients with KRAS G12C mutation (see ACS Cent. Sci. 2020; 6:1753-1761). These compounds occupy a dynamic pocket in the switch II region of K-Ras thereby irreversibly locking K-Ras G12C in inactive GDP-bound state. Since KRAS mutations, including G12C, enrich predominantly active-state protein in cancer cells, sufficient residual GTPase activity and nucleotide cycling are required for effective inhibition of K-Ras by inactive state-selective drugs (see Cell 2020; 183(4):850-859). Currently, there are no molecules in clinical trial that can inhibit K-Ras G12C by binding to its active GTP form or both GTP and GDP forms. Inhibitors of active form of K-Ras should be more effective at suppressing cell growth and survival, as well as less susceptible to adaptive resistance than inhibitors binding to its inactive form. Compared to K-ras G12C mutant, other prevalent K-Ras mutants, such as G12D, do not contain non-native cysteine residue and cycle through inactive state at extremely low rate, thus making non-G12C mutant-specific drug discovery more challenging.

Given the role of K-Ras mutants in human malignancy, there is still unmet medical need for development of new treatments for cancer patients with KRAS mutations. The present disclosure fulfills this and related needs.

### SUMMARY

In a first aspect, provided is a compound of Formula (I):

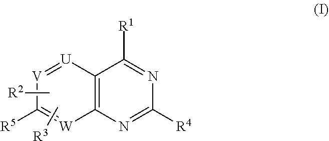

wherein:
U, V, and W are CH; or one or two of U, V, and W are N and the other of U, V, and W are CH;
R¹ is a ring of formula:

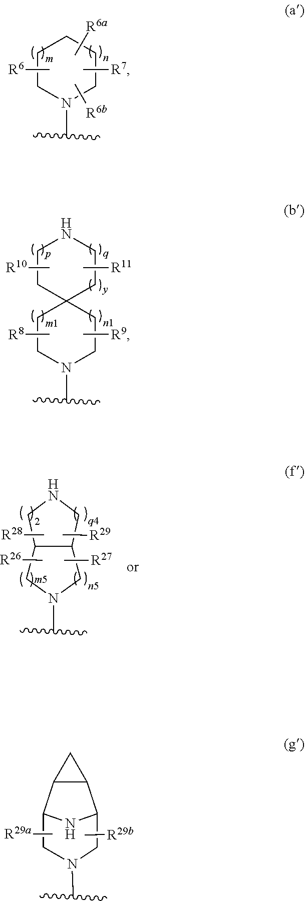

where:
one of m and n is 0, 1, or 2, and the other of m and n is 0, 1, 2, or 3;
m1, n1, m5 and n5 are independently 0, 1, or 2, provided one of m5 and n5 is at least 1;
p, q, p4 and q4 are independently 0, 1, or 2, and y is 0 or 1;
R⁶, R⁸, R¹⁰, R²⁶, and R²⁸ are independently hydrogen, deuterium, alkyl, alkoxy, halo, haloalkyl, hydroxy, hydroxylalkyl, alkoxyalkyl, cyano, or cyanomethyl, provided R⁶, R¹⁰, and R²⁸ are not attached to the ring —NH—;
R⁷, R⁹, R¹¹, R²⁷, and R²⁹ are independently hydrogen, deuterium, alkyl, alkoxy, halo, haloalkyl, hydroxy, hydroxylalkyl, or alkoxyalkyl, provided R⁷, R¹¹, and R²⁹ are not attached to the ring —NH—; or
when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;
R^{6a} is hydrogen, deuterium, alkyl, alkylidenyl, alkoxy, halo, haloalkyl, hydroxy, hydroxyalkyl, alkoxyalkyl, cyano, cyanomethyl, cyanoethyl, or 2-cyanovinyl, provided R^{6a} is not attached to the ring —NH—;
R^{6b} is hydrogen or alkyl, provided R^{6b} is not attached to the ring —NH—; or
when R^{6a} and R^{6b} are attached to the same carbon of ring (a′), they can combine to form cycloalkylene;
R^{29a} and R^{29b} are independently hydrogen, alkyl, hydroxy, cyano, or cyanomethyl provided R^{29a} and R^{29b} are not attached to the ring —NH—;
R² is hydrogen, deuterium, alkyl, halo, haloalkyl, alkoxy, hydroxy, or cyano, provided that R² is absent when two of U, V, and W are N;
R³ is hydrogen, deuterium, alkyl, halo, haloalkyl, alkoxy, cycloalkyl, cycloalkyloxy, hydroxy, or cyano;
R⁴ is —Z—R³⁰ where Z is a bond, O, NH, N(alkyl), or S; and R³⁰ is heterocyclylalkyl, fused heterocyclylalkyl, bicyclic heterocyclyl, bicyclic heterocyclylalkyl, fused bicyclic heterocyclyl, fused bicyclic heterocyclylalkyl, heterocyclyl fused bicyclic heterocyclyl, heterocyclyl fused bicyclic heterocyclylalkyl, cycloalkenyl fused bicyclic heterocyclyl, cycloalkenyl fused bicyclic heterocyclylalkyl, heterocycloalkenyl fused bicyclic heterocyclyl, heterocycloalkenyl fused bicyclic heterocyclylalkyl, bicyclic heterocycloalkenyl, bicyclic heterocycloalkenylalkyl, tricyclic heterocyclyl, tricyclic heterocyclylalkyl, fused tricyclic heterocyclyl, or fused tricyclic heterocyclylalkyl, wherein:
(1) fused heterocyclyl of fused heterocyclylalkyl is substituted with R^{a}, R^{b}, and R^{c} where R^{a} is hydrogen, alkyl, alkenyl, haloalkenyl, alkylidenyl, haloalkylidenyl, alkoxyalkylidenyl, or ═CR³¹R³²;
(2) heterocyclyl of heterocyclylalkyl and bicyclic heterocyclyl, by itself or as part of bicyclic heterocyclylalkyl, are substituted with R^{d}, R^{e}, and R^{f} where R^{d} is alkenyl, haloalkenyl, alkylidenyl, haloalkylidenyl, alkoxyalkylidenyl, or R^{d} is ═CR³³R³⁴;
(3) fused bicyclic heterocyclyl, by itself or as part of fused bicyclic heterocyclylalkyl, heterocyclyl fused bicyclic heterocyclyl, by itself or as part of heterocyclyl fused bicyclic heterocyclylalkyl, tricyclic heterocyclyl, by itself or as part of tricyclic heterocyclylalkyl, and fused tricyclic heterocyclyl, by itself or as part of fused tricyclic heterocyclylalkyl, are independently substituted with R^{g}, R^{h}, and Rⁱ where R^{g} is alkenyl, haloalkenyl, alkylidenyl, haloalkylidenyl, alkoxyalkylidenyl, or ═CR³⁵R³⁶; and
(4) bicyclic heterocycloalkenyl, by itself or as part of bicyclic heterocycloalkenylalkyl, cycloalkenyl fused bicyclic heterocyclyl, by itself or as part of cycloalkenyl fused bicyclic heterocyclylalkyl, heterocycloalkenyl fused bicyclic heterocyclyl, by itself of as part of heterocycloalkenyl fused bicyclic heterocyclylalkyl, are independently substituted with R^{j}, R^{k}, and R^{m} where R^{j} is hydrogen, alkenyl, haloalkenyl, alkylidenyl, haloalkylidenyl, or alkoxyalkylidenyl, or ═CR³⁷R³⁸—; where:
R³¹, R³³, R³⁵, and R³⁷ are independently hydrogen, alkyl, or fluoro and R³², R³⁴, R³⁶, and R³⁸ are independently cyano, alkoxyalkyloxyalkyl, cycloalkyl, cycloalkylalkyl, or cycloalkylalkyloxyalkyl (where cycloalkyl, by itself or as part of cycloalkylalkyl and cycloalkylalkyloxyalkyl is optionally substituted with one or two substituents independently selected from alkyl, halo, haloalkoxy, alkoxy, alkoxyalkyl, and hydroxy), heterocyclyl, phenyl, or heteroaryl (where heterocyclyl, phenyl, and heteroaryl are optionally substituted with one, two, or three substituents independently selected from alkyl, halo, haloalkyl, haloalkoxy, alkoxy, cyano, and hydroxy), or independently of each other, R³¹ and R³², R³³ and R³⁴, R³⁵ and R³⁶, and R³⁷ and R³⁸ together with the carbon atom to which they are attached form cycloalkylene optionally substituted with alkyl, halo, alkoxy, or hydroxy;
R^{b}, R^{c}, R^{h}, and R^{k} are independently hydrogen, alkyl, halo, haloalkyl, haloalkoxy, alkoxy, hydroxy, alkoxyalkyl, alkoxyalkoxy, alkoxyalkyloxyalkyl, cycloalkyl, cycloalkylalkyl, cycloalkoxy, cycloalkylalkyloxy, cycloalkoxyalkyl, cycloalkylalkyloxyalkyl, cycloalkylalkyloxyalkyloxy, bridged cycloalkyloxy, bridged cycloalkyloxyalkyl, bridged cycloalkylalkyloxy, bridged cycloalkylalkyloxyalkyl, heterocyclyl, heterocyclylalkyl, heterocyclyloxy, heterocyclyloxyalkyl, heterocyclylalkyloxy, heterocyclylalkyloxyalkyl (where cycloalkyl, by itself or as part of cycloalkylalkyl, cycloalkoxy, cycloalkoxyalkyl, cycloalkylalkyloxy, cycloalkylalkyloxyalkyl, and cycloalkylalkyloxyalkyloxy, bridged cycloalkyl, as part of bridged cycloalkyloxy, bridged cycloalkyloxyalkyl, bridged cycloalkylalkyloxy, and bridged cycloalkylalkyloxyalkyl, and heterocyclyl, by itself or as part of heterocyclylalkyl, heterocyclyloxy, heterocyclyloxyalkyl, heterocyclylalkyloxy, and heterocyclylalkyloxyalkyl are optionally substituted with one, two, or three substituents independently selected from alkyl, halo, haloalkyl, haloalkoxy, alkoxy, alkoxyalkyl, cyano, and hydroxy), or -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy), provided that when R³⁰ is heterocyclylalkyl, bicyclic heterocyclyl, or bicyclic heterocyclylalkyl and R^{d} is alkenyl, haloalkenyl, alkylidenyl, haloalkylidenyl, or alkoxyalkylidenyl, then R^{e} is other than hydrogen; and
R^{c}, R^{f}, Rⁱ, and R^{m} are independently hydrogen, alkyl, halo, alkoxy, alkoxyalkyl, or hydroxy; and
R⁵ is -Q-R⁴⁴ where Q is bond, alkylene, or —C(═O)—; and R⁴⁴ is cycloalkyl, fused cycloalkyl, fused spirocycloalkyl, aryl, aralkyl, heteroaryl, fused heteroaryl, or heteroaralkyl wherein aryl, aryl in aralkyl, heteroaryl, fused heteroaryl, and heteroaryl in heteroaralkyl are independently substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkenyl, cyanoalkynyl, or halo, and R^{dd} is hydrogen, alkyl, alkylthio, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or
a pharmaceutically acceptable salt thereof.

In one embodiment of the first aspect, the compound of Formula (I) is not:
4-(4-((1R,5S)-3,8-diazabicyclo[3.2.1]octan-3-yl)-8-fluoro-2-(((S)-1-methyl-4-methylenepyrrolidin-2-yl)methoxy)pyrido[4,3-d]pyrimidin-7-yl)-5-ethynyl-6-fluoronaphthalen-2-ol; or
4-(4-((1R,5S)-3,8-diazabicyclo[3.2.1]octan-3-yl)-2-((5,5-dimethyl-2-methylenetetrahydro-1H-pyrrolizin-7a(5H)-yl)methoxy)-8-fluoropyrido[4,3-d]pyrimidin-7-yl)-5-ethynyl-6-fluoronaphthalen-2-ol.

In a second aspect, provided is a pharmaceutical composition comprising a compound of Formula (I) (or any of the embodiments thereof described herein) or a pharmaceutically acceptable salt thereof and a pharmaceutically acceptable excipient.

In a third aspect, provided is a method of inhibiting K-Ras, in particular K-Ras G12D, in a cell, comprising contacting the cell with a compound of Formula (I) (or any of the embodiments thereof described herein). In one embodiment of the third aspect, the contacting is in vitro. In another embodiment of the third aspect, the contacting is in vivo.

In a fourth aspect, provided is a method of inhibiting cell proliferation in vitro or in vivo, comprising contacting a cell with a compound of Formula (I) (or any of the embodiments thereof described herein) or a pharmaceutical composition thereof as disclosed herein. In one embodiment of the fourth aspect, the contacting is in vitro. In another embodiment of the fourth aspect, the contacting is in vivo.

In a fifth aspect, provided is a method of treating cancer in a patient, preferably the patient is in need of such treatment, which method comprises administering to the patient, preferably a patient in need of such treatment, a therapeutically effective amount of a compound of Formula (I) (or any of the embodiments thereof described herein) or a pharmaceutically acceptable salt thereof or a a pharmaceutical composition thereof as disclosed herein.

In a sixth aspect, provided is a method of treating cancer associated with K-Ras, in particular K-Ras G12D, in a patient, preferably the patient is in need of such treatment, which method comprises administering to the patient, preferably a patient in need of such treatment, a therapeutically effective amount of a compound of Formula (I) (or any of the embodiments thereof described herein) or a pharmaceutically acceptable salt thereof or a a pharmaceutical composition thereof as disclosed herein.

In a seventh aspect, provided is a compound of Formula (I), (or any embodiments thereof described herein) or a pharmaceutically acceptable salt thereof or a pharmaceutical composition thereof as disclosed herein for use as a medicament. In one embodiment, the medicament is useful for the treatment of cancer.

In an eighth aspect, provided is a compound of Formula (I), (or any embodiments thereof described herein) or a pharmaceutically acceptable salt thereof or a pharmaceutical composition thereof as disclosed herein for use as a therapy.

In a ninth aspect, provided is a compound of Formula (I), (or any embodiments thereof described herein) or a pharmaceutically acceptable salt thereof or a pharmaceutical composition thereof as disclosed herein for use in the treatment of cancer.

In a tenth aspect, provided is a compound of Formula (I), (or any embodiments thereof described herein) or a pharmaceutically acceptable salt thereof or a pharmaceutical composition thereof as disclosed herein for use in the treatment of cancers associated with KRas, in particular cancers associated with K-Ras G12D.

In an eleventh aspect, provided is a compound of Formula (I), (or any embodiments thereof described herein) or a pharmaceutically acceptable salt thereof or a pharmaceutical composition thereof as disclosed herein for use in inhibiting K-Ras, in particular K-Ras G12D.

In a twelfth aspect, provided is an intermediate selected from:

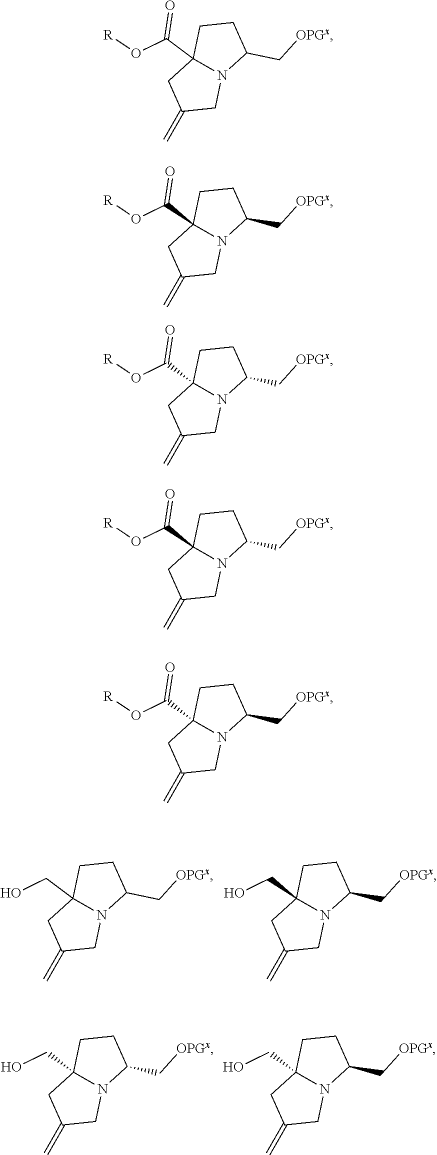

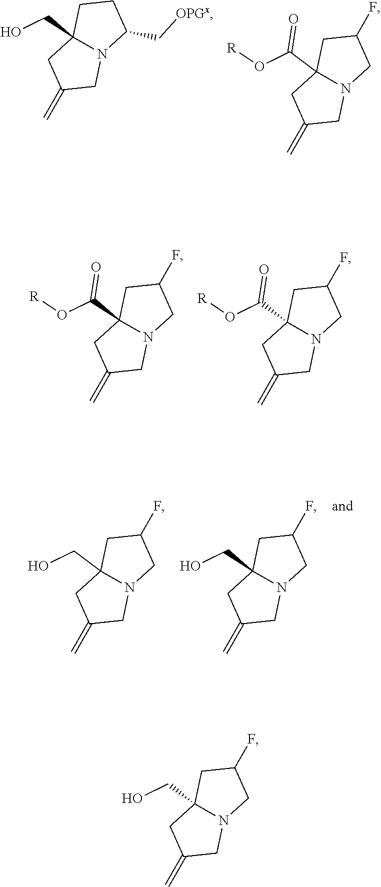

wherein each R is alkyl and each PG^{x} is a hydroxy protecting group (e.g., Boc, tert-butyldimethysilyl tert-butyldiphenylsilyl, or CBz).

In a thirteenth aspect, provided is a method for making a compound of Formula (I) where R⁴ is

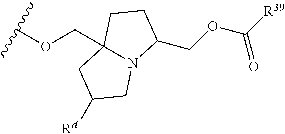

comprising the steps of:
(i) treating a compound of formula 3-a:

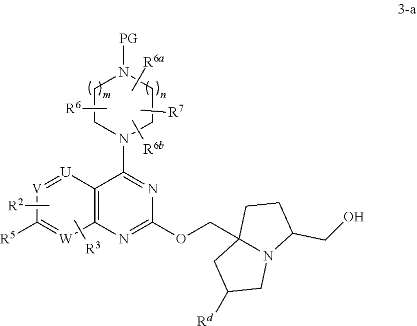

where PG is an amino protecting group and U, V, W, R², R³, R⁵, R⁶, R⁷, R^{6a}, R^{6b} are as defined in Formula (I) of the first aspect (or any one of the embodiments disclosed herein below);
with an amine of formula HR³⁹, where R³⁹ is as defined in Formula (I) of the first aspect (or any one of embodiments thereof herein below) under carbamoylation reaction conditions, to provide a compound of formula 3-b:;

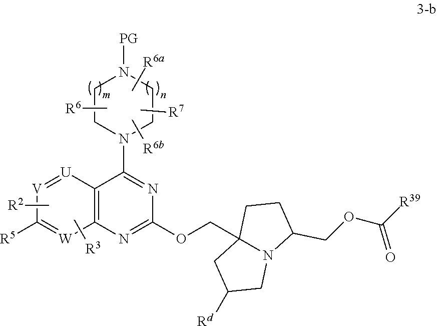

and
(ii) removing the amino protecting group.

In an embodiment the thirteenth aspect, step (1) is carried out in the present of a coupling agent (for example, N,N′-Carbonyldiimidazole or 4-nitrophenyl chloroformate).

In any of the aforementioned aspects involving the treatment of cancer, are further embodiments comprising administering the compound of Formula (I) (or any embodiments thereof disclosed herein), or a pharmaceutically acceptable salt thereof in combination with at least one additional anticancer agent. When combination therapy is used, the agents can be administered simultaneously or sequentially.

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond e.g., ethenyl, propenyl, 2-propenyl, butenyl, pentenyl, and the like.

“Alkynyl” means a linear monovalent hydrocarbon radical of two to six carbon atoms or a branched monovalent hydrocarbon radical of three to six carbon atoms containing a triple bond e.g., ethynyl, propynyl, 2-propynyl, butynyl, and the like.

“Alkylamino” means a —NHR radical where R is alkyl as defined above, e.g., methylamino, ethylamino, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, such as one or two alkoxy groups, as defined above, e.g., 2-methoxyethyl, 1-, 2-, or 3-methoxypropyl, 2-ethoxyethyl, and the like.

“Alkoxyalkyloxy” means a —OR radical where R is alkoxyalkyl as defined above. Examples include, but are not limited to, 2-methoxyethyloxy, 1-, 2-, or 3-methoxypropyloxy, 2-ethoxyethyloxy, and the like.

“Alkoxyalkyloxyalkyl” means a -(alkylene)-OR radical where R is alkyloxyalkyl as defined above. Examples include, but are not limited to, 2-methoxyethyloxymethyl, methoxymethoxymethyl, 1-, 2-, or 3-methoxypropyloxymethyl, 2-ethoxyethyloxymethyl, and the like.

“Alkylidenyl” means refers to a group of formula R═ where R is alkyl as defined above. Examples include, but are not limited to, methylidenyl (H₂C═), ethylidenyl (CH₃CH═), hexylidenyl (CH₃(CH₂)₄CH═), 2-propylidenyl (═C(CH₃)₂), and the like. For example, in the compound below:

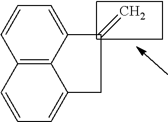

the alkylidene group, methylidenyl, is enclosed by the box which is indicated by the arrow.

“Alkoxyalkylidenyl” means refers to a group of formula=R where R is alkoxyalkyl as defined above. Examples include, but are not limited to, methoxethylidenyl (CH₃OCH₂CH═), ethoxyethylidenyl (C₂H₅OCH₂CH═), 1-methoxyethylidenyl (═C(CH₃)OCH₃), and the like. For example, in the compound below:

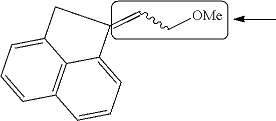 [SMILES: COCC=C1Cc2cccc3cccc1c23]

the alkoxyalkylidenyl group, methoxethylidenyl, is enclosed by the box which is indicated by the arrow.

“Alkoxycarbonyl” means a —C(O)OR radical where R is alkyl as defined above, e.g., methoxycarbonyl, ethoxycarbonyl, and the like.

“Amino” means a —NH₂ radical.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above. Examples include, but are not limited to, benzyl, phenethyl, and the like.

“Bicyclic heterocyclyl” means a saturated monovalent fused bicyclic ring of 8 to 12 ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —CO— group. More specifically the term bicyclic heterocyclyl includes, but is not limited to, hexahydro-1H-pyrrolizinyl, and the like.

“Bicyclic heterocyclylalkyl” means a -(alkylene)-R radical where R is bicyclic heterocyclyl as defined above. Examples include, but are not limited to, hexahydro-1H-pyrrolizinylmethyl, hexahydro-1H-pyrrolizinylethyl, and the like.

“Bicyclic heterocycloalkenyl” means a monovalent fused bicyclic ring of 8 to 12 ring atoms containing a double bond and in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by —CO— group. More specifically the term bicyclic heterocycloalkenyl includes, but is not limited to, 2,3-dihydro-1H-pyrrolizin-7a(5H)-yl, and the like.

“Bicyclic heterocycloalkenylalkyl” means a -(alkylene)-R radical where R is bicyclic heterocycloalkenyl as defined above. Examples include, but are not limited to, 2,3-dihydro-1H-pyrrolizin-7a(5H)-ylmethyl, 2,3-dihydro-1H-pyrrolizin-7a(5H)-ylethyl, and the like.

“Bridged cycloalkyl” means a saturated bicyclic ring having 5 to 8 ring carbon ring atoms in which two non-adjacent ring atoms are linked by a (CRR′)n group where n is 1 to 3 and R and R′ are independently H or methyl (also may be referred to herein as “bridging” group). Unless otherwise stated, bridged cycloalkyl is optionally substituted with one or two substituents independently selected from alkyl, halo, alkoxy, hydroxy, and cyano. Examples include, but are not limited to, bicyclo[1.1.1]pentyl, bicyclo[2.1.1]hexyl, and the like.

“Bridged cycloalkyloxy” means a —OR radical where R is bridged cycloalkyl as defined above. Examples include, but are not limited to, bicyclo[1.1.1]pentyloxy, bicyclo[2.1.1]hexyloxy, and the like.

“Bridged cycloalkyloxyalkyl” means a -(alkylene)-OR radical where R is bridged cycloalkyl as defined above. Examples include, but are not limited to, bicyclo[1.1.1]pentyloxy-methyl, bicyclo[1.1.1]pentyloxyethyl, bicyclo[2.1.1]hexyloxymethyl, and the like.

“Bridged cycloalkylalkyl” means a -(alkylene)-R radical where R is bridged cycloalkyl as defined above. Examples include, but are not limited to, bicyclo[1.1.1]pentylmethyl, bicyclo[2.1.1]hexylmethyl, and the like.

“Bridged cycloalkylalkyloxy” means a —O—R radical where R is bridged cycloalkylalkyl as defined above. Examples include, but are not limited to, bicyclo[1.1.1]pentylmethyloxy, bicyclo[1.1.1]pentylethyloxy, bicyclo[2.1.1]hexylmethyloxy, and the like.

“Bridged cycloalkylalkyloxyalkyl” means a -(alkylene)-OR radical where R is bridged cycloalkylalkyl as defined above. Examples include, but are not limited to, bicyclo[1.1.1]pentylmethyloxymethyl, bicyclo[1.1.1]pentylethyloxymethyl, bicyclo[1.1.1]pentylethyloxyethyl, bicyclo[2.1.1]hexylmethyloxyethyl, and the like.

“Cycloalkyl” means a monocyclic saturated monovalent hydrocarbon radical of three to ten carbon atoms. Examples include, but are not limited to, cyclopropyl, cyclobutyl, cyclopentyl, cyclohexyl, and the like.

“Cycloalkylene” means a monocyclic saturated divalent hydrocarbon radical of three to ten carbon atoms. Examples include, but are not limited to, 1,1-cyclopropylene,1,1-cyclobutylene, 1,1-cyclopentylene, and the like.

“Cycloalkylalkyl” means a -(alkylene)-R radical where R is cycloalkyl as defined above. Examples include, but are not limited to, cyclopropylmethyl cyclobutylethyl, cyclopentylmethyl, cyclohexylmethyl, and the like.

“Cycloalkyloxy” or “cycloalkoxy” means a —OR radical where R is cycloalkyl as defined above. Examples include, but are not limited to, cyclopropyloxy, cyclobutyloxy, cyclopentyloxy, cyclohexyloxy, and the like.

Cycloalkylalkyloxy” means a —OR radical where R is cycloalkylalkyl as defined above. Examples include, but are not limited to, cyclopropylmethyloxy, cyclobutylmethyloxy, cyclopentylmethyloxy, cyclohexylethyloxy, and the like.

“Cycloalkyloxyalkyl” or “cycloalkoxyalkyl” means a -(alkylene)-OR radical where R is cycloalkyl as defined above. Examples include, but are not limited to, cyclopropyloxymethyl, cyclobutyloxymethyl, and the like.

“Cycloalkylalkyloxyalkyloxy” means a —OR radical where R is cycloalkylalkyloxyalkyl as defined above. Examples include, but are not limited to, cyclopropylmethyloxyethyloxy, cyclobutylethyloxyethyloxy, and the like.

“Cycloalkylalkyloxyalkyl” means a -(alkylene)-OR radical where R is cycloalkylalkyl as defined above. Examples include, but are not limited to, cyclopropylmethyloxymethyl, cyclobutylmethoxymethyl, and the like.

“Cycloalkenyl” means a monocyclic monovalent hydrocarbon radical of three to ten carbon atoms containing a double bond. Examples include, but are not limited to, cyclobutenyl, cyclopentyl, cyclopent-2-enyl, cyclohexenyl, and the like.

“Cycloalkenyl fused bicyclic heterocyclyl” means a saturated monovalent fused bicyclic ring of 8 to 10 ring atoms in which one or two ring atoms are heteroatoms independently selected from N, O, and S(O)ₙ (where n is an integer from 0 to 2), the remaining ring atoms being C, unless stated otherwise, and where two adjacent ring atoms of the bicyclic ring are fused to two adjacent ring atoms of cycloalkenyl, as defined herein. Examples includes, but is not limited to, 2,3,3a,5,6,7,7a,8-octahydrocyclopenta[b]pyrrolizin-7a-yl, and the like.

“Cycloalkenyl fused bicyclic heterocyclylalkyl” means (alkylene)-R where R is cycloalkenyl fused bicyclic heterocyclyl as defined above. Examples includes, but is not limited to, 2,3,3a,5,6,7,7a,8-octahydrocyclopenta[b]pyrrolizin-7a-ylmethyl, 2,3,3a,5,6,7,7a,8-octahydrocyclopenta[b]pyrrolizin-7a-ylethyl, and the like.

“Cyanoalkyl” means a linear monovalent hydrocarbon radical of one to six carbon atoms or a branched monovalent hydrocarbon radical of three to six carbons substituted with cyano e.g., cyanomethyl, cyanoethyl, and the like.

“Cyanoalkenyl” means an alkenyl radical as defined above where one of the hydrogen atom in the alkynyl chain is replace by a cyano. Examples include, but are not limited to, —C═C(CN), —CH₂C═C(CN), and the like.

“Cyanoalkynyl” means an alkynyl radical as defined above where one of the hydrogen atom in the alkynyl chain is replace by a cyano. Examples include, but are not limited to, —C≡C(CN), —CH₂C≡C(CN), and the like.

“Deuterium” mean refers to ²H or D.

“Dialkylamino” means a —NRR′ radical where R and R′ are independently alkyl as defined above, e.g., dimethylamino, methylethylamino, and the like.

“Fused bicyclic heterocyclyl” means a saturated monovalent fused bicyclic ring of 8 to 10 ring atoms in which one or two ring atoms are heteroatoms independently selected from N, O, and S(O)ₙ, where n is an integer from 0 to 2, one ring atom can be —CO—, and the remaining ring atoms being C, unless stated otherwise, and where two adjacent ring atoms of the bicyclic ring are fused to two adjacent ring atoms of phenyl or a five or six membered heteroaryl, each as defined herein, unless stated otherwise. More specifically the term fused bicyclic heterocyclyl includes, but is not limited to, 2,3-dihydro-1H-pyrrolo[2,1-a]isoindol-9b(5H)-yl, 2,3-dihydro-1H-pyrrolo[1,2-a]indol-9a(9H)-yl, 1,3b,4,5,6,8-hexahydropyrrolo[3,2-a]pyrrolizin-3b-yl, and the like.

“Fused bicyclic heterocyclylalkyl” means a -(alkylene)-R radical where R is fused bicyclic heterocyclyl as defined above. Examples include, but are not limited to, hexahydro-1H-pyrrolizinylmethyl, hexahydro-1H-pyrrolizinylethyl, 2,3-dihydro-1H-pyrrolo[2,1-a]isoindol-9b(5H)-ylmethyl, 2,3-dihydro-1H-pyrrolo[1,2-a]indol-9a(9H)-ylmethyl, and the like.

“Fused cycloalkyl” as used herein, means cycloalkyl as defined above where two adjacent ring atoms of the cycloalkyl ring are fused to two adjacent ring atoms of phenyl or a five or six membered heteroaryl, each as defined herein, unless stated otherwise. The fused cycloalkyl can be attached at any atom of the ring. Non limiting examples of the fused cycloalkyl include bicyclo[4.1.0]hepta-1,3,5-triene, bicyclo[4.2.0]octa-1,3,5-triene, and the like.

“Fused spirocycloalkyl” means spiro cycloalkyl as defined herein where two adjacent ring atoms of the spiro cycloalkyl are fused to two adjacent ring atoms of a phenyl or a five or six membered heteroaryl, each as defined herein.

“Fused heterocyclyl” means a saturated monovalent monocyclic ring of 4 to 7 ring atoms having from one to three heteroatoms independently selected from N, O, and S(O) n where n is 0, one ring atoms can be —CO—, and the remaining ring atoms being carbon, and further wherein two adjacent ring atoms of the monocyclic ring are fused to two adjacent ring atoms of a cycloalkyl, phenyl or a five or six membered heteroaryl, each as defined herein, unless stated otherwise. The nitrogen atom(s) are optionally oxidized optionally quaternized and one or two carbon atoms of the fused ring atoms in the saturated monocyclic ring includes the two common ring vertices shared with the fused phenyl or five or six membered heteroaryl. The fused heterocyclyl can be attached at any atom of the ring. Non limiting examples of the fused heterocycloalkyl include 2,3-dihydrobenzo[b][1,4]-dioxinyl, 2-oxabicyclo[3.1.0]hexanyl, indolin-2-one-1-yl, indolinyl, and the like.

“Fused heterocyclylalkyl” as used herein, means a -(alkylene)-R radical where R is fused heterocyclyl, as defined herein.

“Fused tricyclic heterocyclyl” means a saturated monovalent fused tricyclic ring of 9 to 16 ring atoms, preferably 10 to 14 ring atoms, in which one or two ring atoms are heteroatoms independently selected from N, O, and S(O)ₙ, where n is an integer from 0 to 2, one ring atom can be —CO—, and the remaining ring atoms being C, unless stated otherwise, and where two adjacent ring atoms of the tricyclic ring (preferably two adjacent ring atoms of a ring other than the central ring of the tricyclic ring) are fused to two adjacent ring atoms of cycloalkyl, phenyl or a five or six membered heteroaryl, each as defined herein, unless stated otherwise. The term fused tricyclic heterocyclyl includes, but is not limited to,

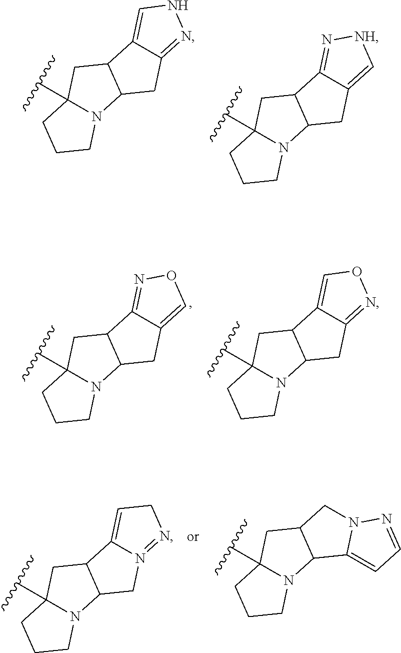

and the like.

“Fused tricyclic heterocyclylalkyl” means a -(alkylene)-R radical where R is fused tricyclic heterocyclyl as defined above. Examples include, but are not limited to,

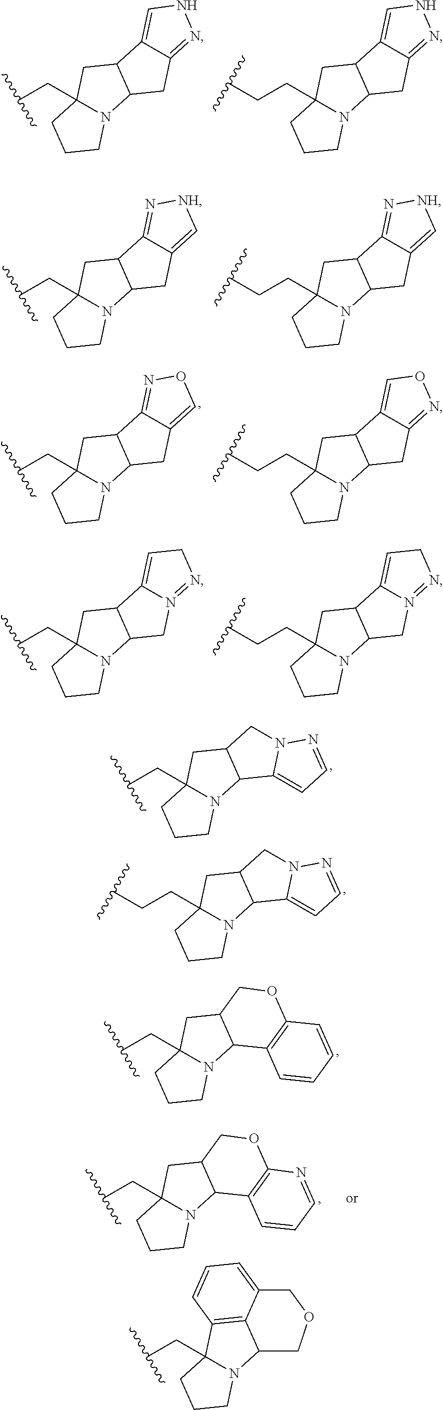

and the like.

“Fused heteroaryl” means fused bicyclic heteroaryl, as defined herein, where two adjacent ring atoms of the heteroaryl ring are fused to two adjacent ring atoms of phenyl.

“Halo” means fluoro, chloro, bromo, or iodo, preferably fluoro or chloro.

“Haloalkyl” means alkyl radical as defined above, which is substituted with one or more halogen atoms, e.g., one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkylidenyl” means refers to a group of formula ═R where R is haloalkyl as defined above. Examples include, but are not limited to, difluoromethylidenyl (═CF₂), 2,2-difluoroethylidenyl (═CHCHF₂), 1-fluoroethylidenyl=CFCH₃), and the like. For example, in the compound below:

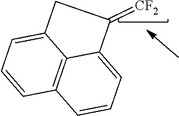

the group pointed to by the arrow is the haloalkylidenyl group, difluoromethylidenyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroalkyl” mean alkyl radical as defined above wherein one or two carbon atoms are replaced by O, NR (R is H or alkyl), or S, provided the heteroalkyl group is attached to the remainder of the molecule via a carbon atom, e.g., methoxymethyl, methylethylaminoethyl, and the like.

“Heteroaryl” means a monovalent monocyclic or fused bicyclic aromatic radical of 5 to 10 ring atoms, unless otherwise stated, where one or more, (in one embodiment, one, two, or three), ring atoms are heteroatom selected from N, O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. When the heteroaryl ring contains 5- or 6 ring atoms it is also referred to herein as 5- or 6-membered heteroaryl. When the heteroaryl ring is fused bicyclic aromatic radical 9- or 10 ring atoms it is also referred to herein as fused bicyclic heteroaryl.

“Heteroaralkyl” means a -(alkylene)-R radical where R is heteroaryl as defined above, e.g., pyridinylmethyl, and the like. When the heteroaryl ring in heteroaralkyl contains 5- or 6 ring atoms it is also referred to herein as 5- or 6-membered heteroaralkyl.

“Heterocyclyl” means a saturated or unsaturated monovalent monocyclic group of 4 to 8 ring atoms in which one or two ring atoms are heteroatom independently selected from N, O, and S(O)ₙ, where n is an integer from 0 to 2, the remaining ring atoms being C, unless stated otherwise. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic and may be referred to herein as heterocycloalkenyl. When the heterocyclyl group contains at least one nitrogen atom, it is also referred to herein as heterocycloamino and is a subset of the heterocyclyl group.

“Heterocyclylalkyl” or “heterocycloalkyl” means a -(alkylene)-R radical where R is heterocyclyl ring as defined above e.g., tetraydrofuranylmethyl, piperazinylmethyl, morpholinylethyl, and the like.

“Heterocyclyloxy” means a —OR radical where R is heterocyclyl as defined above. Examples include, but are not limited to, tetraydrofuranyloxy, pyrrolidinyloxy, piperidinyloxy, piperazinyloxy, morpholinyloxy, and the like.

“Heterocyclyloxyalkyl” means a -(alkylene)-OR radical where R is heterocyclyl as defined above. Examples include, but are not limited to, tetraydrofuranyloxymethyl, pyrrolidinyloxymethyl, pyrrolidinyloxyethyl, piperidinyloxyethyl, piperazinyloxyethyl, morpholinyloxyethyl, and the like.

“Heterocyclylalkyloxy” means a —O—R radical where R is heterocyclylalkyl as defined above. Examples include, but are not limited to, tetraydrofuranylmethyloxy, pyrrolidinylmethyloxy, pyrrolidinylethyloxy, piperidinylmethyloxy, piperazinylmethyloxy, morpholinylethyloxy, and the like.

“Heterocyclylalkyloxyalkyl” means a -(alkylene)-OR radical where R is heterocyclylalkyl as defined above. Examples include, but are not limited to, tetraydrofuranylmethyloxymethyl, pyrrolidinylmethyloxymethyl, pyrrolidinylethyloxymethyl, piperidinylmethyloxymethyl, piperazinylmethylethyl, morpholinylethyloxymethyl, and the like.

“Heterocyclyl fused bicyclic heterocyclyl” means a bicyclic heterocyclyl as defined herein (preferably a bicyclic heterocyclyl of 8 to 10 ring atoms) where two adjacent ring atoms of the bicyclic heterocyclyl are fused to two adjacent ring atoms of a hetereocyclyl ring as defined herein, provided the heterocyclyl ring contains at least two heteroatoms independently selected from N, O, and S(O)ₙ, where n is an integer from 0 to 2. The term heterocyclyl fused bicyclic heterocyclyl includes, but is not limited to,

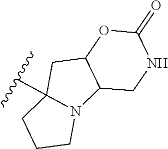 [SMILES: *C12CCCN1C1CNC(=O)OC1C2]

and the like.

“Heterocyclyl fused bicyclic heterocyclylalkyl” mean -(alkylene)-R where R is heterocyclyl fused bicyclic heterocyclyl as defined above.

“Heterocycloalkenyl fused bicyclic heterocyclyl” means a saturated monovalent fused bicyclic ring of 8 to 10 ring atoms in which one or two ring atoms are heteroatoms independently selected from N, O, and S(O)ₙ (where n is an integer from 0 to 2), the remaining ring atoms being C, unless stated otherwise, and where two adjacent ring atoms of the bicyclic ring are fused to two adjacent ring atoms of heterocycloalkenyl, as defined herein. Examples includes, but is not limited to, 1,3,5,5a,6,7,8,9a-octahydropyrano[4,3-b]pyrrolizin-5a-yl, and the like.

“Heterocycloalkenyl fused bicyclic heterocyclylalkyl” means -(alkylene)-R where R is heterocycloalkenyl fused bicyclic heterocyclyl as defined above. Examples includes, but is not limited to, 1,3,5,5a,6,7,8,9a-octahydropyrano[4,3-b]pyrrolizin-5a-ylmethyl, 1,3,5,5a,6,7,8,9a-octahydropyrano[4,3-b]pyrrolizin-5a-ylethyl, and the like.

“Oxo,” as used herein, alone or in combination, refers to ═(O).

“Optionally substituted aryl” means aryl as defined above, that is optionally substituted with one, two, or three substituents independently selected from alkyl, hydroxyl, cycloalkyl, carboxy, alkoxycarbonyl, hydroxy, alkoxy, alkylsulfonyl, amino, alkylamino, dialkylamino, halo, haloalkyl, haloalkoxy, and cyano. When aryl is phenyl, optionally substituted aryl is referred to herein as optionally substituted phenyl.

“Optionally substituted heteroaryl” means heteroaryl as defined above that is optionally substituted with one, two, or three substituents independently selected from alkyl, alkylsulfonyl, hydroxyl, cycloalkyl, carboxy, alkoxycarbonyl, hydroxy, alkoxy, halo, haloalkyl, haloalkoxy, amino, alkylamino, dialkylamino, and cyano.

“Optionally substituted heterocyclyl” means heterocyclyl as defined above that is optionally substituted with one, two, or three substituents independently selected from alkyl, alkylsulfonyl, alkylcarbonyl, hydroxyl, cycloalkyl, cycloalkylalkyl, carboxy, alkoxycarbonyl, hydroxy, hydroxyalkyl, alkoxy, alkoxyalkyl, aminoalkyl, cyanoalkyl, halo, haloalkyl, haloalkoxy, and cyano, unless stated otherwise.

“Optionally substituted heterocyclylalkyl” means -(alkylene)-R where R is optionally substituted heterocyclyl as defined above.

“Tricyclic heterocyclyl” means a saturated monovalent fused tricyclic ring of 9 to 14, preferably 12 to 14, ring atoms in which one or two ring atoms are heteroatom independently selected from N, O, and S(O)ₙ, where n is an integer from 0 to 2, one ring atom can be —CO—, and the remaining ring atoms being C, unless stated otherwise. Additionally, one or two ring carbon atoms in the heterocyclyl ring can optionally be replaced by a —CO— group. The term tricyclic heterocyclyl includes, but is not limited to,

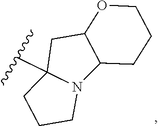 [SMILES: *C12CCCN1C1CCCOC1C2]

and the like.

“Tricyclic heterocyclylalkyl” means a -(alkylene)-R radical where R is tricyclic heterocyclyl as defined above. Examples include, but are not limited to,

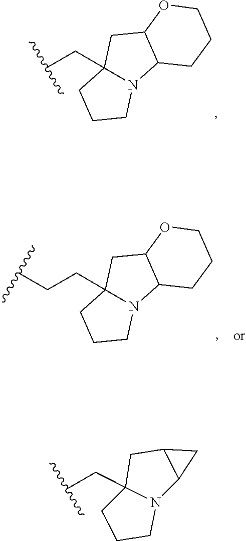 [SMILES: *CC12CCCN1C1CCCOC1C2 ; *CCC12CCCN1C1CCCOC1C2 ; *CC12CCCN1C1CC1C2]

and the like.

The present disclosure also includes protected derivatives of compounds of Formula (I). For example, when compounds of Formula (I) contain groups such as hydroxy, carboxy, or any group containing a nitrogen atom(s), these groups can be protected with suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes polymorphic forms and deuterated forms of the compound of Formula (I) or a pharmaceutically acceptable salt thereof.

The term “prodrug” refers to a compound that is made more active in vivo. Certain compounds Formula (I) may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the active compound. Prodrugs are often useful because, in some situations, they may be easier to administer than the compound, or parent drug. They may, for instance, be bioavailable by oral administration whereas the parent drug is not. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, P A, 1985, which is incorporated herein by reference in its entirety.

The compounds of Formula (I) may have asymmetric centers. Compounds of Formula (I) containing an asymmetrically substituted atom may be isolated in optically active or racemic forms. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. All chiral, diastereomeric, all mixtures of chiral or diastereomeric forms, and racemic forms are within the scope of this disclosure, unless the specific stereochemistry or isomeric form is specifically indicated. It will also be understood by a person of ordinary skill in the art that when a compound is denoted as (R) stereoisomer, it may contain the corresponding (S) stereoisomer as an impurity and vice versa.

Certain compounds of Formula (I) can exist as tautomers and/or geometric isomers. All possible tautomers and cis and trans isomers, as individual forms and mixtures thereof are within the scope of this disclosure. Additionally, as used herein the term alkyl includes all the possible isomeric forms of said alkyl group albeit only a few examples are set forth. Furthermore, when the cyclic groups such as aryl is substituted, it includes all the positional isomers albeit only a few examples are set forth. Furthermore, all hydrates of a compound of Formula (I) are within the scope of this disclosure.

The compounds of Formula (I) may also contain unnatural amounts of isotopes at one or more of the atoms that constitute such compounds. Unnatural amounts of an isotope may be defined as ranging from the amount found in nature to an amount 100% of the atom in question. that differ only in the presence of one or more isotopically enriched atoms. Exemplary isotopes that can be incorporated into compounds of the present invention, such as a compound of Formula (I) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds of Formula (I), including in Tables 1 and 2 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use.

“A pharmaceutically acceptable carrier/excipient” as used in the specification and claims includes both one and more than one such excipient.

“Spiro cycloalkyl” means a saturated bicyclic monovalent ring having 5 to 10 ring atoms in in which the rings are connected through only one atom, the connecting atom is also called the spiroatom, most often a quaternary carbon (“spiro carbon”). Unless stated otherwise, spiro cycloalkyl is optionally substituted with one or two substituents independently selected from alkyl, halo, alkoxy, hydroxy, and cyano. Examples include, but are not limited to, Representative examples include, but are not limited to, spiro[3.3]heptane, spiro[3.4]octane, spiro[3.5]-nonane, and the like.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

The phrase optionally substituted aryl in the definition of R^{dd} in Formula (I) (and similar phrases used to define other groups in Formula (I) is intended to cover aryl that is unsubstituted and aryl that is substituted with substituents denoted in the definition thereof in this Application.

Certain structures provided herein are drawn with one or more floating substituents. Unless provided otherwise or otherwise clear from the context, the substituent(s) may be present on any atom of the ring through which they are drawn, where chemically feasible and valency rules permitting. For example, in the structure of Formula (I):

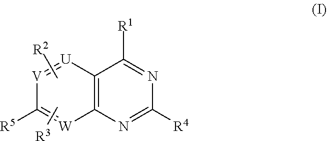

R² and R³ groups are floating substituents and can replace the hydrogen atom of any one of U, V, and W of the

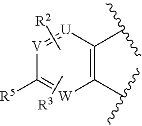

portion of the quinazoline ring ring when U, V, and W are CH.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a disease or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., delaying, arresting (i.e., stabilizing), or reducing the development or severity of the disease or its clinical symptoms; or
(3) relieving the disease, i.e., causing regression of the disease or its clinical symptoms.

In one embodiment, treating or treatment of a disease includes inhibiting the disease, i.e., delaying, arresting or reducing the development or severity of the disease or its clinical symptoms; or relieving the disease, i.e., causing regression of the disease or its clinical symptoms.

A “therapeutically effective amount” means the amount of a compound of the present disclosure or a pharmaceutically acceptable salt thereof that, when administered to a patient for treating a disease, is sufficient to affect such treatment for the disease. The “therapeutically effective amount” will vary depending on the compound, the disease and its severity and the age, weight, etc., of the mammal to be treated. The therapeutically effective amount of a K-ras inhibitor disclosed herein can be administered to the patient in a single dosage form or multiples thereof. For example, 600 mg dose of a K-ras inhibitor can be administered in a single 600 ng tablet or two 300 mg tablets.

The terms “inhibiting” and “reducing,” or any variation of these terms in relation of K-Ras G12D, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of K-Ras G12D GTPase activity; a decrease of K-Ras G12D GTP binding affinity or an increase of G12D GDP binding affinity; an increase of GTP off rate or a decrease of GDP off rate; a decrease of signaling transduction molecules levels downstream in the K-Ras pathway, e.g., a decrease in pERK level; and/or a decrease of K-Ras complex binding to downstream signaling molecules compared to normal.

Representative compounds of Formula (I) are disclosed in Compound Table 1 below:

Compound Table 1

| Cpd. | | |
|---|---|---|
| # | Compound | Name |
| 1 | 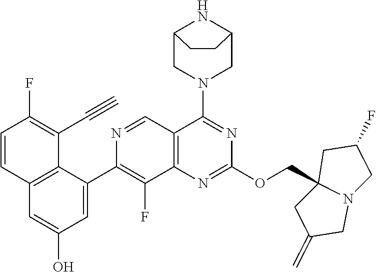 [SMILES: C#Cc1c(F)ccc2cc(O)cc(-c3ncc4c(N5CC6CCC(C5)N6)nc(OC[C@]56CC(=C)CN5C[C@@H](F)C6)nc4c3F)c12] | 4-(4-((1R,5S)-3,8-diazabicyclo[3.2.1]octan-3-yl)-8- fluoro-2-(((2S,7aR)-2-fluoro-6-methylenetetrahydro-1H- pyrrolizin-7a(5H)-yl)methoxy)pyrido[4,3-d]pyrimidin-7- yl)-5-ethynyl-6-fluoronaphthalen-2-ol |
| 2 | 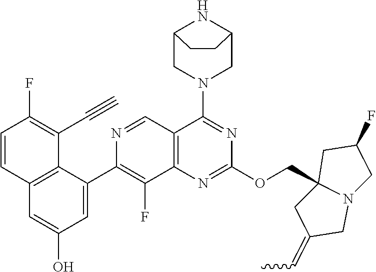 | 4-(4-((1R,5S)-3,8-diazabicyclo[3.2.1]octan-3-yl)-2- (((6R,7aR)-2-ethylidene-6-fluorotetrahydro-1H- pyrrolizin-7a(5H)-yl)methoxy)-8-fluoropyrido[4,3- d]pyrimidin-7-yl)-5-ethynyl-6-fluoronaphthalen-2-ol |
| 3 | 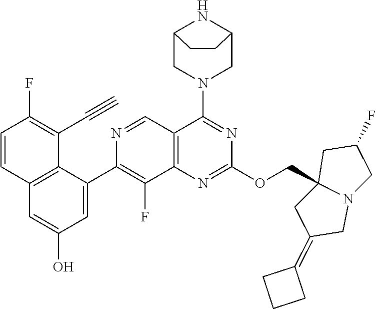 [SMILES: C#Cc1c(F)ccc2cc(O)cc(-c3ncc4c(N5CC6CCC(C5)N6)nc(OC[C@]56CC(=C7CCC7)CN5C[C@@H](F)C6)nc4c3F)c12] | 4-(4-((1R,5S)-3,8-diazabicyclo[3.2.1]octan-3-yl)-2- (((6S,7aR)-2-cyclobutylidene-6-fluorotetrahydro-1H- pyrrolizin-7a(5H)-yl)methoxy)-8-fluoropyrido[4,3- d]pyrimidin-7-yl)-5-ethynyl-6-fluoronaphthalen-2-ol |
| 4 | 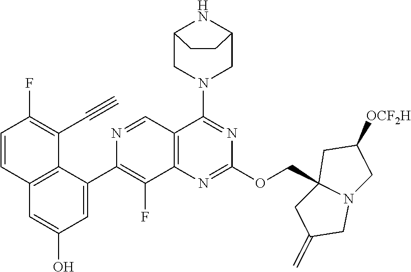 | 4-(4-((1R,5S)-3,8-diazabicyclo[3.2.1]octan-3-yl)-2- (((2R,7aR)-2-(difluoromethoxy)-6-methylenetetrahydro- 1H-pyrrolizin-7a(5H)-yl)methoxy)-8-fluoropyrido[4,3- d]pyrimidin-7-yl)-5-ethynyl-6-fluoronaphthalen-2-ol |
| 5 | 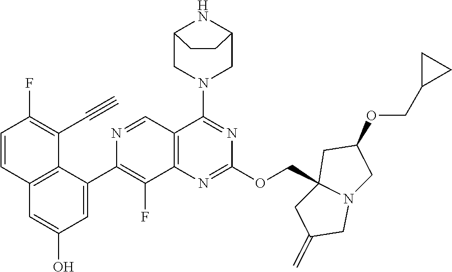 [SMILES: C#Cc1c(F)ccc2cc(O)cc(-c3ncc4c(N5CC6CCC(C5)N6)nc(OC[C@]56CC(=C)CN5C[C@H](OCC5CC5)C6)nc4c3F)c12] | 4-(4-((1R,5S)-3,8-diazabicyclo[3.2.1]octan-3-yl)-2- (((2R,7aR)-2-(cyclopropylmethoxy)-6- methylenetetrahydro-1H-pyrrolizin-7a(5H)-yl)methoxy)- 8-fluoropyrido[4,3-d]pyrimidin-7-yl)-5-ethynyl-6- fluoronaphthalen-2-ol |
| 6a | 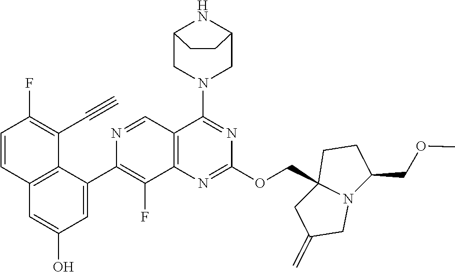 [SMILES: C#Cc1c(F)ccc2cc(O)cc(-c3ncc4c(N5CC6CCC(C5)N6)nc(OC[C@@]56CC[C@@H](COC)N5CC(=C)C6)nc4c3F)c12] | 4-(4-((1R,5S)-3,8-diazabicyclo[3.2.1]octan-3-yl)-8- fluoro-2-(((5S,7aS)-5-(methoxymethyl)-2- methylenetetrahydro-1H-pyrrolizin-7a(5H)- yl)methoxy)pyrido[4,3-d]pyrimidin-7-yl)-5-ethynyl-6- fluoronaphthalen-2-ol |
| 6b | 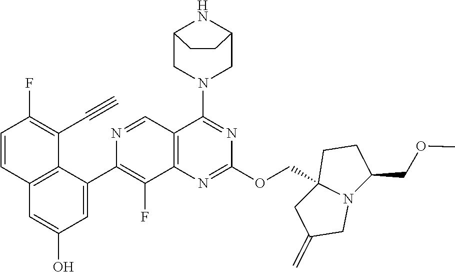 [SMILES: C#Cc1c(F)ccc2cc(O)cc(-c3ncc4c(N5CC6CCC(C5)N6)nc(OC[C@]56CC[C@@H](COC)N5CC(=C)C6)nc4c3F)c12] | 4-(4-((1R,5S)-3,8-diazabicyclo[3.2.1]octan-3-yl)-8- fluoro-2-(((5S,7aR)-5-(methoxymethyl)-2- methylenetetrahydro-1H-pyrrolizin-7a(5H)- yl)methoxy)pyrido[4,3-d]pyrimidin-7-yl)-5-ethynyl-6- fluoronaphthalen-2-ol |
| 7 | 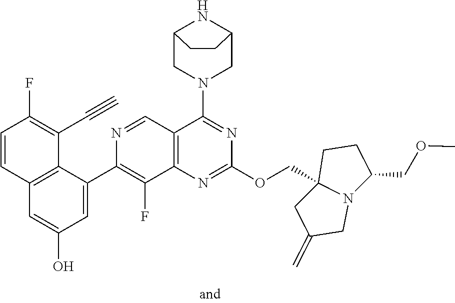 [SMILES: C#Cc1c(F)ccc2cc(O)cc(-c3ncc4c(N5CC6CCC(C5)N6)nc(OC[C@]56CC[C@H](COC)N5CC(=C)C6)nc4c3F)c12] | 4-(4-((1R,5S)-3,8-diazabicyclo[3.2.1]octan-3-yl)-8- fluoro-2-(((5R,7aR)-5-(methoxymethyl)-2- methylenetetrahydro-1H-pyrrolizin-7a(5H)- yl)methoxy)pyrido[4,3-d]pyrimidin-7-yl)-5-ethynyl-6- fluoronaphthalen-2-ol |
| | | And |
| | 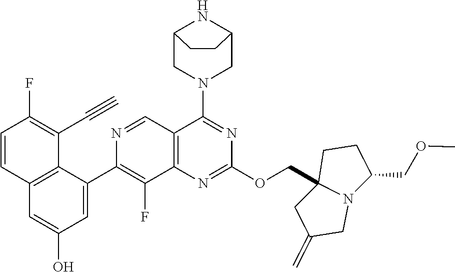 | 4-(4-((1R,5S)-3,8-diazabicyclo[3.2.1]octan-3-yl)-8- fluoro-2-(((5R,7aS)-5-(methoxymethyl)-2- methylenetetrahydro-1H-pyrrolizin-7a(5H)- yl)methoxy)pyrido[4,3-d]pyrimidin-7-yl)-5-ethynyl-6- fluoronaphthalen-2-ol |
| 8 | 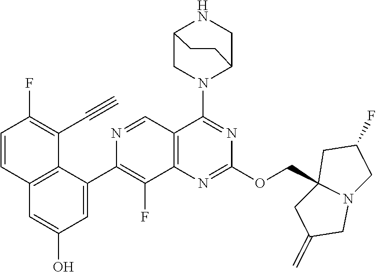 [SMILES: C#Cc1c(F)ccc2cc(O)cc(-c3ncc4c(N5CC6CCC5CN6)nc(OC[C@]56CC(=C)CN5C[C@@H](F)C6)nc4c3F)c12] | 4-(4-(2,5-diazabicyclo[2.2.2]octan-2-yl)-8-fluoro-2- (((2S,7aR)-2-fluoro-6-methylenetetrahydro-1H-pyrrolizin- 7a(5H)-yl)methoxy)pyrido[4,3-d]pyrimidin-7-yl)-5- ethynyl-6-fluoronaphthalen-2-ol |
| 9 | 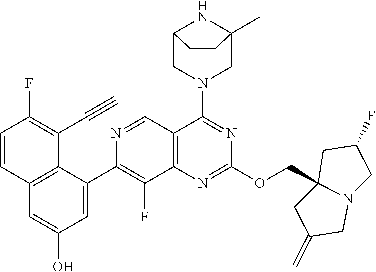 [SMILES: C#Cc1c(F)ccc2cc(O)cc(-c3ncc4c(N5CC6CCC(C)(C5)N6)nc(OC[C@]56CC(=C)CN5C[C@@H](F)C6)nc4c3F)c12] | 5-ethynyl-6-fluoro-4-(8-fluoro-2-(((2S,7aR)-2-fluoro-6- methylenetetrahydro-1H-pyrrolizin-7a(5H)-yl)methoxy)- 4-(1-methyl-3,8-diazabicyclo[3.2.1]octan-3-yl)pyrido[4,3- d]pyrimidin-7-yl)naphthalen-2-ol |
| 10 | 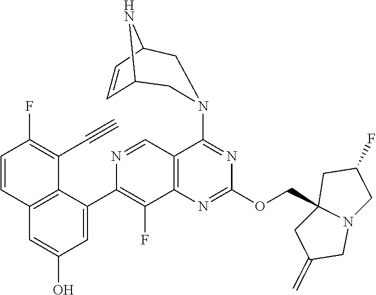 [SMILES: C#Cc1c(F)ccc2cc(O)cc(-c3ncc4c(N5CC6C=CC(C5)N6)nc(OC[C@]56CC(=C)CN5C[C@@H](F)C6)nc4c3F)c12] | 4-(4-((1R,5S)-3,8-diazabicyclo[3.2.1]oct-6-en-3-yl)-8- fluoro-2-(((2S,7aR)-2-fluoro-6-methylenetetrahydro-1H- pyrrolizin-7a(5H)-yl)methoxy)pyrido[4,3-d]pyrimidin-7- yl)-5-ethynyl-6-fluoronaphthalen-2-ol |
| 11 | 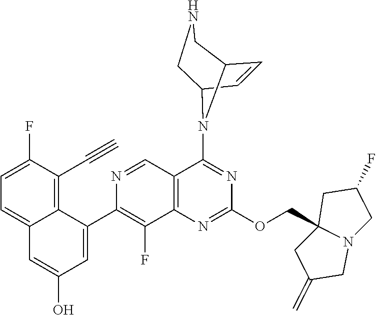 [SMILES: C#Cc1c(F)ccc2cc(O)cc(-c3ncc4c(N5C6C=CC5CNC6)nc(OC[C@]56CC(=C)CN5C[C@@H](F)C6)nc4c3F)c12] | 4-(4-(3,8-diazabicyclo[3.2.1]oct-6-en-8-yl)-8-fluoro-2- (((2S,7aR)-2-fluoro-6-methylenetetrahydro-1H-pyrrolizin- 7a(5H)-yl)methoxy)pyrido[4,3-d]pyrimidin-7-yl)-5- ethynyl-6-fluoronaphthalen-2-ol |
| 12 | 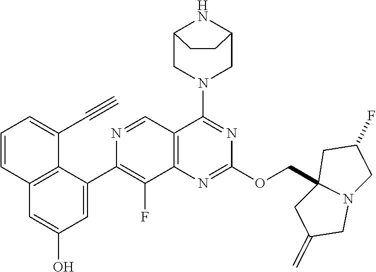 | 4-(4-((1R,5S)-3,8-diazabicyclo[3.2.1]octan-3-yl)-8- fluoro-2-(((2S,7aR)-2-fluoro-6-methylenetetrahydro-1H- pyrrolizin-7a(5H)-yl)methoxy)pyrido[4,3-d]pyrimidin-7- yl)-5-ethynylnaphthalen-2-ol |
| 13 | 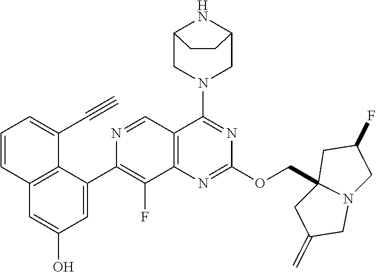 | 4-(4-((1R,5S)-3,8-diazabicyclo[3.2.1]octan-3-yl)-8- fluoro-2-(((2R,7aR)-2-fluoro-6-methylenetetrahydro-1H- pyrrolizin-7a(5H)-yl)methoxy)pyrido[4,3-d]pyrimidin-7- yl)-5-ethynylnaphthalen-2-ol |
| 14 | 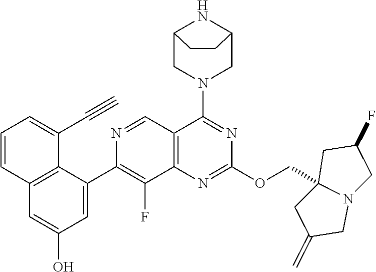 | 4-(4-((1R,5S)-3,8-diazabicyclo[3.2.1]octan-3-yl)-8- fluoro-2-(((2R,7aS)-2-fluoro-6-methylenetetrahydro-1H- pyrrolizin-7a(5H)-yl)methoxy)pyrido[4,3-d]pyrimidin-7- yl)-5-ethynylnaphthalen-2-ol |
| 15 | 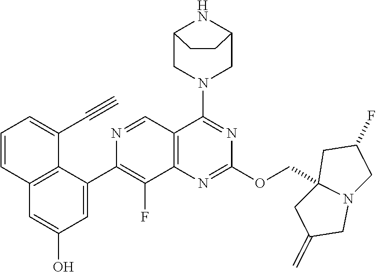 | 4-(4-((1R,5S)-3,8-diazabicyclo[3.2.1]octan-3-yl)-8- fluoro-2-(((2S,7aS)-2-fluoro-6-methylenetetrahydro-1H- pyrrolizin-7a(5H)-yl)methoxy)pyrido[4,3-d]pyrimidin-7- yl)-5-ethynylnaphthalen-2-ol |
| 16 | 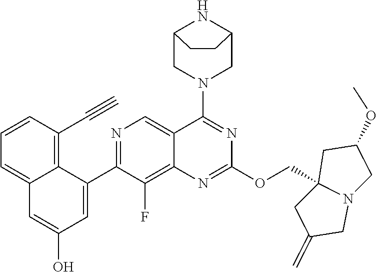 [SMILES: C#Cc1cccc2cc(O)cc(-c3ncc4c(N5CC6CCC(C5)N6)nc(OC[C@@]56CC(=C)CN5C[C@@H](OC)C6)nc4c3F)c12] | 4-(4-((1R,5S)-3,8-diazabicyclo[3.2.1]octan-3-yl)-8- fluoro-2-(((2S,7aS)-2-methoxy-6-methylenetetrahydro- 1H-pyrrolizin-7a(5H)-yl)methoxy)pyrido[4,3- d]pyrimidin-7-yl)-5-ethynylnaphthalen-2-ol |
| 17 | 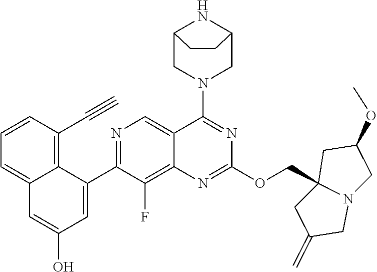 [SMILES: C#Cc1cccc2cc(O)cc(-c3ncc4c(N5CC6CCC(C5)N6)nc(OC[C@]56CC(=C)CN5C[C@H](OC)C6)nc4c3F)c12] | 4-(4-((1R,5S)-3,8-diazabicyclo[3.2.1]octan-3-yl)-8- fluoro-2-(((2R,7aR)-2-methoxy-6-methylenetetrahydro- 1H-pyrrolizin-7a(5H)-yl)methoxy)pyrido[4,3- d]pyrimidin-7-yl)-5-ethynylnaphthalen-2-ol |
| 18 | 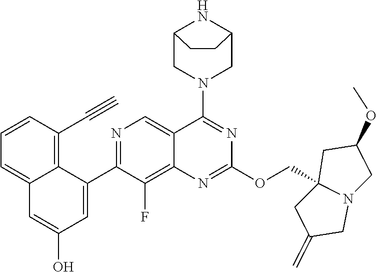 [SMILES: C#Cc1cccc2cc(O)cc(-c3ncc4c(N5CC6CCC(C5)N6)nc(OC[C@@]56CC(=C)CN5C[C@H](OC)C6)nc4c3F)c12] | 4-(4-((1R,5S)-3,8-diazabicyclo[3.2.1]octan-3-yl)-8- fluoro-2-(((2R,7aS)-2-methoxy-6-methylenetetrahydro- 1H-pyrrolizin-7a(5H)-yl)methoxy)pyrido[4,3- d]pyrimidin-7-yl)-5-ethynylnaphthalen-2-ol |
| 19 | 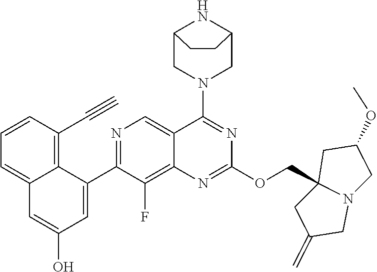 [SMILES: C#Cc1cccc2cc(O)cc(-c3ncc4c(N5CC6CCC(C5)N6)nc(OC[C@]56CC(=C)CN5C[C@@H](OC)C6)nc4c3F)c12] | 4-(4-((1R,5S)-3,8-diazabicyclo[3.2.1]octan-3-yl)-8- fluoro-2-(((2S,7aR)-2-methoxy-6-methylenetetrahydro- 1H-pyrrolizin-7a(5H)-yl)methoxy)pyrido[4,3- d]pyrimidin-7-yl)-5-ethynylnaphthalen-2-ol |
| 20a | 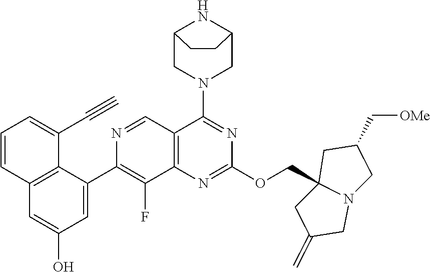 [SMILES: C#Cc1cccc2cc(O)cc(-c3ncc4c(N5CC6CCC(C5)N6)nc(OC[C@]56CC(=C)CN5C[C@@H](COC)C6)nc4c3F)c12] | 4-(4-((1R,5S)-3,8-diazabicyclo[3.2.1]octan-3-yl)-8- fluoro-2-(((2S,7aS)-2-(methoxymethyl)-6- methylenetetrahydro-1H-pyrrolizin-7a(5H)- yl)methoxy)pyrido[4,3-d]pyrimidin-7-yl)-5- ethynylnaphthalen-2-ol |
| 20b | 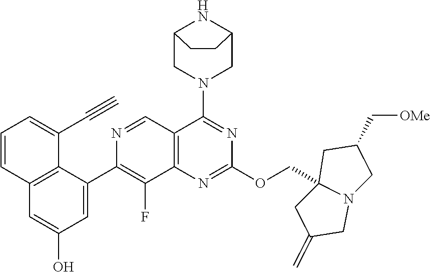 | 4-(4-((1R,5S)-3,8-diazabicyclo[3.2.1]octan-3-yl)-8- fluoro-2-(((2S,7aR)-2-(methoxymethyl)-6- methylenetetrahydro-1H-pyrrolizin-7a(5H)- yl)methoxy)pyrido[4,3-d]pyrimidin-7-yl)-5- ethynylnaphthalen-2-ol |
| 21 | 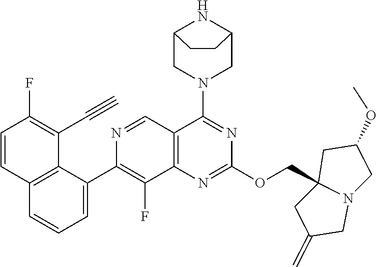 [SMILES: C#Cc1c(F)ccc2cccc(-c3ncc4c(N5CC6CCC(C5)N6)nc(OC[C@]56CC(=C)CN5C[C@@H](OC)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2S,7aR)-2- methoxy-6-methylenetetrahydro-1H-pyrrolizin-7a(5H)- yl)methoxy)pyrido[4,3-d]pyrimidine |
| 22 | 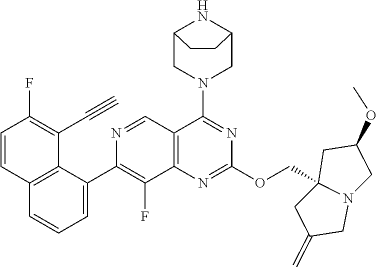 [SMILES: C#Cc1c(F)ccc2cccc(-c3ncc4c(N5CC6CCC(C5)N6)nc(OC[C@@]56CC(=C)CN5C[C@H](OC)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2R,7aS)-2- methoxy-6-methylenetetrahydro-1H-pyrrolizin-7a(5H)- yl)methoxy)pyrido[4,3-d]pyrimidine |
| 23a | 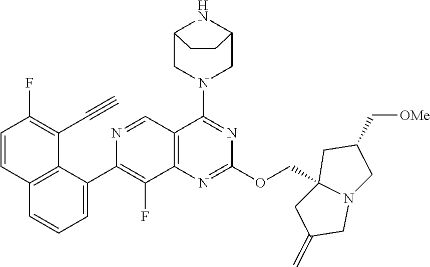 [SMILES: C#Cc1c(F)ccc2cccc(-c3ncc4c(N5CC6CCC(C5)N6)nc(OC[C@@]56CC(=C)CN5C[C@@H](COC)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2S,7aR)-2- (methoxymethyl)-6-methylenetetrahydro-1H-pyrrolizin- 7a(5H)-yl)methoxy)pyrido[4,3-d]pyrimidine |
| 23b | 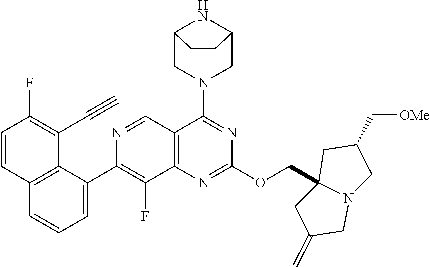 | 4-((1R,5S)-3,8-diazabicyclo[3.2.1]octan-3-yl)-7-(8- ethynyl-7-fluoronaphthalen-1-yl)-8-fluoro-2-(((2S,7aS)-2- (methoxymethyl)-6-methylenetetrahydro-1H-pyrrolizin- 7a(5H)-yl)methoxy)pyrido[4,3-d]pyrimidine |
| 24 | 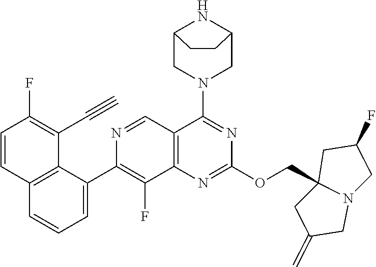 [SMILES: C#Cc1c(F)ccc2cccc(-c3ncc4c(N5CC6CCC(C5)N6)nc(OC[C@]56CC(=C)CN5C[C@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2R,7aR)- 2-fluoro-6-methylenetetrahydro-1H-pyrrolizin-7a(5H)- yl)methoxy)pyrido[4,3-d]pyrimidine |
| 25 | 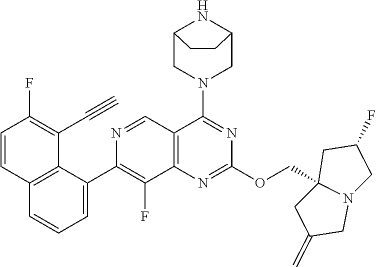 [SMILES: C#Cc1c(F)ccc2cccc(-c3ncc4c(N5CC6CCC(C5)N6)nc(OC[C@@]56CC(=C)CN5C[C@@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2S,7aS)-2- fluoro-6-methylenetetrahydro-1H-pyrrolizin-7a(5H)- yl)methoxy)pyrido[4,3-d]pyrimidine |
| 26 | 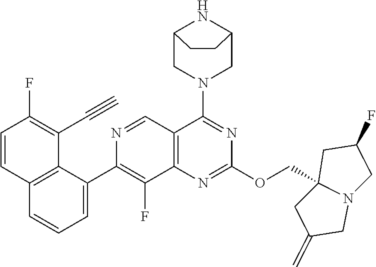 [SMILES: C#Cc1c(F)ccc2cccc(-c3ncc4c(N5CC6CCC(C5)N6)nc(OC[C@@]56CC(=C)CN5C[C@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2R,7aS)-2- fluoro-6-methylenetetrahydro-1H-pyrrolizin-7a(5H)- yl)methoxy)pyrido[4,3-d]pyrimidine |
| 27 | 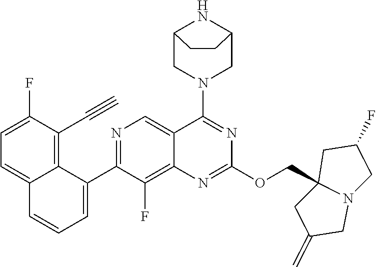 [SMILES: C#Cc1c(F)ccc2cccc(-c3ncc4c(N5CC6CCC(C5)N6)nc(OC[C@]56CC(=C)CN5C[C@@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2S,7aR)-2- fluoro-6-methylenetetrahydro-1H-pyrrolizin-7a(5H)- yl)methoxy)pyrido[4,3-d]pyrimidine |
| 28 | 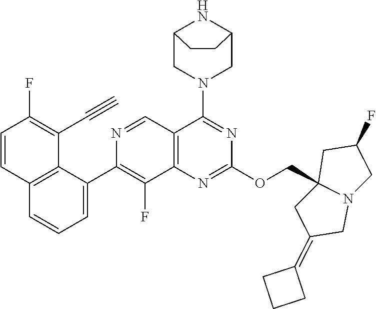 [SMILES: C#Cc1c(F)ccc2cccc(-c3ncc4c(N5CC6CCC(C5)N6)nc(OC[C@]56CC(=C7CCC7)CN5C[C@H](F)C6)nc4c3F)c12] | 4-((1R,5S)-3,8-diazabicyclo[3.2.1]octan-3-yl)-2- (((6R,7aR)-2-cyclobutylidene-6-fluorotetrahydro-1H- pyrrolizin-7a(5H)-yl)methoxy)-7-(8-ethynyl-7- fluoronaphthalen-1-yl)-8-fluoropyrido[4,3-d]pyrimidine |
| 29 | 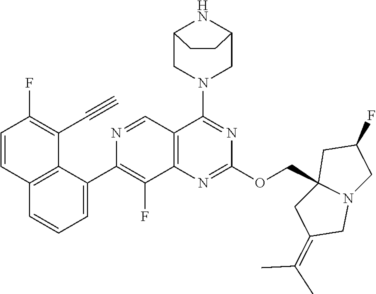 [SMILES: C#Cc1c(F)ccc2cccc(-c3ncc4c(N5CC6CCC(C5)N6)nc(OC[C@]56CC(=C(C)C)CN5C[C@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2R,7aR)- 2-fluoro-6-(propan-2-ylidene)tetrahydro-1H-pyrrolizin- 7a(5H)-yl)methoxy)pyrido[4,3-d]pyrimidine |
| 30 | 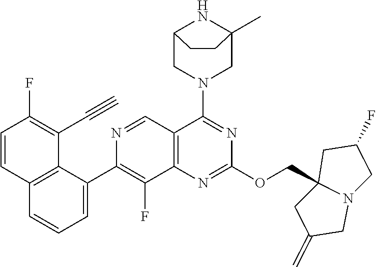 [SMILES: C#Cc1c(F)ccc2cccc(-c3ncc4c(N5CC6CCC(C)(C5)N6)nc(OC[C@]56CC(=C)CN5C[C@@H](F)C6)nc4c3F)c12] | 7-(8-ethynyl-7-fluoronaphthalen-1-yl)-8-fluoro-2- (((2S,7aR)-2-fluoro-6-methylenetetrahydro-1H-pyrrolizin- 7a(5H)-yl)methoxy)-4-(1-methyl-3,8- diazabicyclo[3.2.1]octan-3-yl)pyrido[4,3-d]pyrimidine |
| 31 | 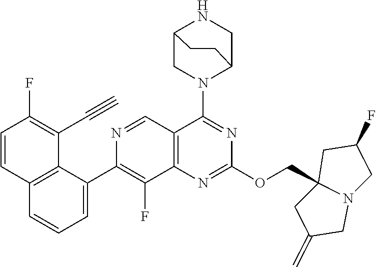 [SMILES: C#Cc1c(F)ccc2cccc(-c3ncc4c(N5CC6CCC5CN6)nc(OC[C@]56CC(=C)CN5C[C@H](F)C6)nc4c3F)c12] | 2-(7-(8-ethynyl-7-fluoronaphthalen-1-yl)-8-fluoro-2- (((2R,7aR)-2-fluoro-6-methylenetetrahydro-1H- pyrrolizin-7a(5H)-yl)methoxy)pyrido[4,3-d]pyrimidin-4- yl)-2,5-diazabicyclo[2.2.2]octane |
| 32 | 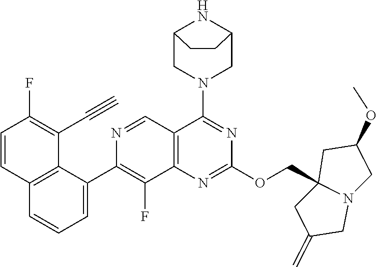 [SMILES: C#Cc1c(F)ccc2cccc(-c3ncc4c(N5CC6CCC(C5)N6)nc(OC[C@]56CC(=C)CN5C[C@H](OC)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((2R,7aR)- 2-methoxy-6-methylenetetrahydro-1H-pyrrolizin-7a(5H)- yl)methoxy)pyrido[4,3-d]pyrimidine |
| 33a | 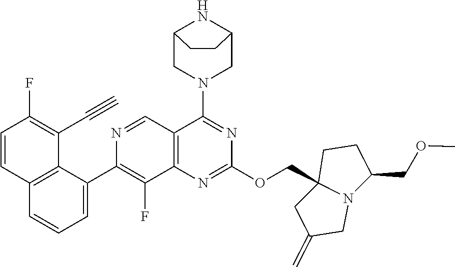 [SMILES: C#Cc1c(F)ccc2cccc(-c3ncc4c(N5CC6CCC(C5)N6)nc(OC[C@@]56CC[C@@H](COC)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- (methoxymethyl)-2-methylenetetrahydro-1H-pyrrolizin- 7a(5H)-yl)methoxy)pyrido[4,3-d]pyrimidine |
| 33b | 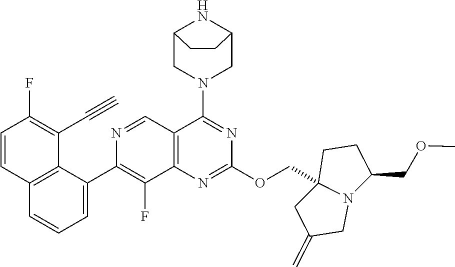 | 4-((1R,5S)-3,8-diazabicyclo[3.2.1]octan-3-yl)-7-(8- ethynyl-7-fluoronaphthalen-1-yl)-8-fluoro-2-(((5S,7aR)-5- (methoxymethyl)-2-methylenetetrahydro-1H-pyrrolizin- 7a(5H)-yl)methoxy)pyrido[4,3-d]pyrimidine |
| 34 | 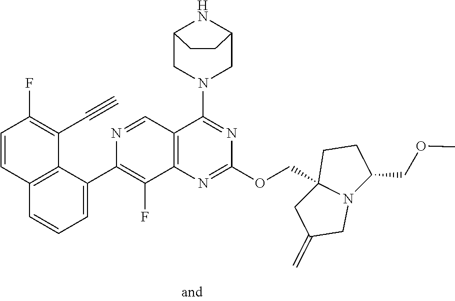 [SMILES: C#Cc1c(F)ccc2cccc(-c3ncc4c(N5CC6CCC(C5)N6)nc(OC[C@]56CC[C@H](COC)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5R,7aR)- 5-(methoxymethyl)-2-methylenetetrahydro-1H-pyrrolizin- 7a(5H)-yl)methoxy)pyrido[4,3-d]pyrimidine |
| | | And |
| | 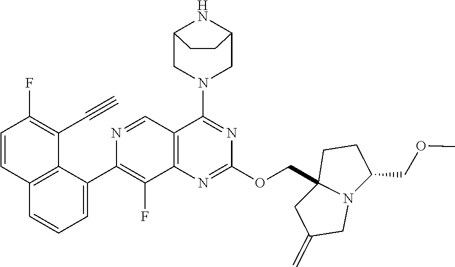 | 4-((1R,5S)-3,8-diazabicyclo[3.2.1]octan-3-yl)-7-(8- ethynyl-7-fluoronaphthalen-1-yl)-8-fluoro-2-(((5R,7aS)-5- (methoxymethyl)-2-methylenetetrahydro-1H-pyrroliziin- 7a(5H)-yl)methoxy)pyrido[4,3-d]pyrimidine |
| 35 | 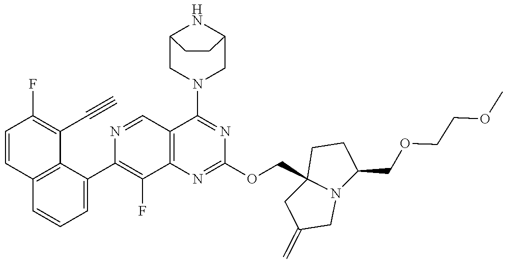 [SMILES: C#Cc1c(F)ccc2cccc(-c3ncc4c(N5CC6CCC(C5)N6)nc(OC[C@@]56CC[C@@H](COCCOC)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- ((2-methoxyethoxy)methyl)-2-methylenetetrahydro-1H- pyrrolizin-7a(5H)-yl)methoxy)pyrido[4,3-d]pyrimidine |
| 36 | 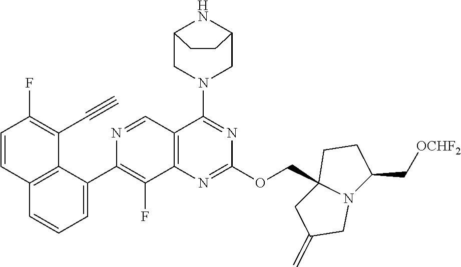 [SMILES: C#Cc1c(F)ccc2cccc(-c3ncc4c(N5CC6CCC(C5)N6)nc(OC[C@@]56CC[C@@H](COC(F)F)N5CC(=C)C6)nc4c3F)c12] | 4-((1R,5S)-3,8-diazabicyclo[3.2.1]octan-3-yl)-2- (((5S,7aS)-5-((difluoromethoxy)methyl)-2- methylenetetrahydro-1H-pyrrolizin-7a(5H)-yl)methoxy)- 7-(8-ethynyl-7-fluoronaphthalen-1-yl)-8-fluoropyrido[4,3- d]pyrimidine |
| 37 | 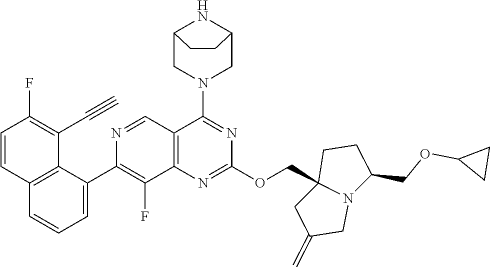 [SMILES: C#Cc1c(F)ccc2cccc(-c3ncc4c(N5CC6CCC(C5)N6)nc(OC[C@@]56CC[C@@H](COC7CC7)N5CC(=C)C6)nc4c3F)c12] | 4-((1R,5S)-3,8-diazabicyclo[3.2.1]octan-3-yl)-2- (((5S,7aS)-5-(cyclopropoxymethyl)-2- methylenetetrahydro-1H-pyrrolizin-7a(5H)-yl)methoxy)- 7-(8-ethynyl-7-fluoronaphthalen-1-yl)-8-fluoropyrido[4,3- d]pyrimidine |
| 38 | 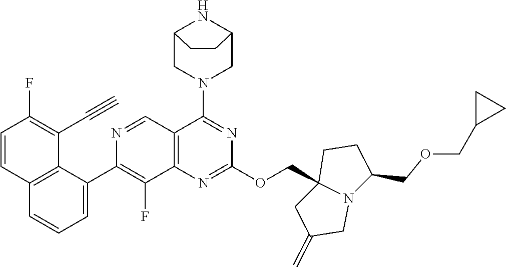 [SMILES: C#Cc1c(F)ccc2cccc(-c3ncc4c(N5CC6CCC(C5)N6)nc(OC[C@@]56CC[C@@H](COCC7CC7)N5CC(=C)C6)nc4c3F)c12] | 4-((1R,5S)-3,8-diazabicyclo[3.2.1]octan-3-yl)-2- (((5S,7aS)-5-((cyclopropylmethoxy)methyl)-2- methylenetetrahydro-1H-pyrrolizin-7a(5H)-yl)methoxy)- 7-(8-ethynyl-7-fluoronaphthalen-1-yl)-8-fluoropyrido[4,3- d]pyrimidine |
| 39 | 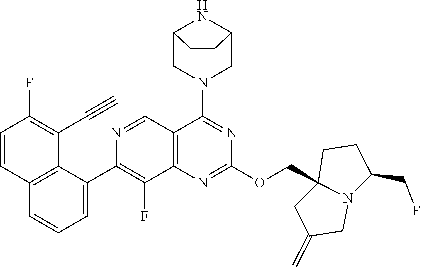 [SMILES: C#Cc1c(F)ccc2cccc(-c3ncc4c(N5CC6CCC(C5)N6)nc(OC[C@@]56CC[C@@H](CF)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- (fluoromethyl)-2-methylenetetrahydro-1H-pyrrolizin- 7a(5H)-yl)methoxy)pyrido[4,3-d]pyrimidine |
| 40 | 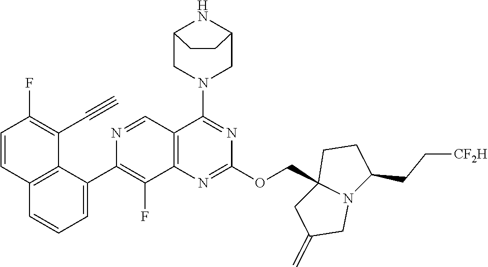 [SMILES: C#Cc1c(F)ccc2cccc(-c3ncc4c(N5CC6CCC(C5)N6)nc(OC[C@@]56CC[C@@H](CCC(F)F)N5CC(=C)C6)nc4c3F)c12] | 4-((1R,5S)-3,8-diazabicyclo[3.2.1]octan-3-yl)-2- (((5S,7aS)-5-(3,3-difluoropropyl)-2-methylenetetrahydro- 1H-pyrrolizin-7a(5H)-yl)methoxy)-7-(8-ethynyl-7- fluoronaphthalen-1-yl)-8-fluoropyrido[4,3-d]pyrimidine |
| 41 | 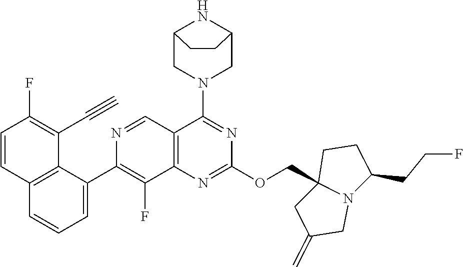 [SMILES: C#Cc1c(F)ccc2cccc(-c3ncc4c(N5CC6CCC(C5)N6)nc(OC[C@@]56CC[C@@H](CCF)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- (2-fluoroethyl)-2-methylenetetrahydro-1H-pyrrolizin- 7a(5H)-yl)methoxy)pyrido[4,3-d]pyrimidine |
| 42 | 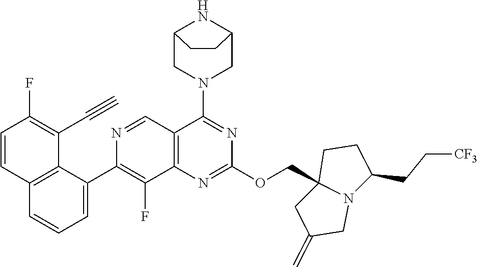 [SMILES: C#Cc1c(F)ccc2cccc(-c3ncc4c(N5CC6CCC(C5)N6)nc(OC[C@@]56CC[C@@H](CCC(F)(F)F)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-2- methylene-5-(3,3,3-trifluoropropyl)tetrahydro-1H- pyrrolizin-7a(5H)-yl)methoxy)pyrido[4,3-d]pyrimidine |
| 43 | 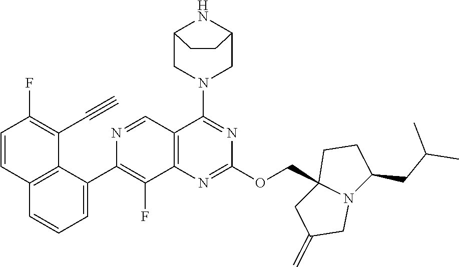 [SMILES: C#Cc1c(F)ccc2cccc(-c3ncc4c(N5CC6CCC(C5)N6)nc(OC[C@@]56CC[C@@H](CC(C)C)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- isobutyl-2-methylenetetrahydro-1H-pyrrolizin-7a(5H)- yl)methoxy)pyrido[4,3-d]pyrimidine |
| 44 | 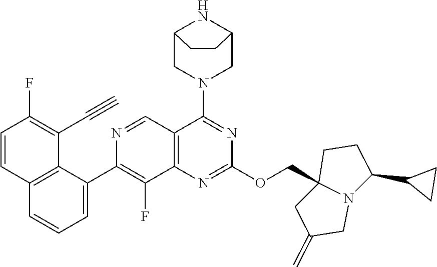 [SMILES: C#Cc1c(F)ccc2cccc(-c3ncc4c(N5CC6CCC(C5)N6)nc(OC[C@@]56CC[C@@H](C7CC7)N5CC(=C)C6)nc4c3F)c12] | 4-((1R,5S)-3,8-diazabicyclo[3.2.1]octan-3-yl)-2- (((5S,7aS)-5-cyclopropyl-2-methylenetetrahydro-1H- pyrrolizin-7a(5H)-yl)methoxy)-7-(8-ethynyl-7- fluoronaphthalen-1-yl)-8-fluoropyrido[4,3-d]pyrimidine |
| 45 | 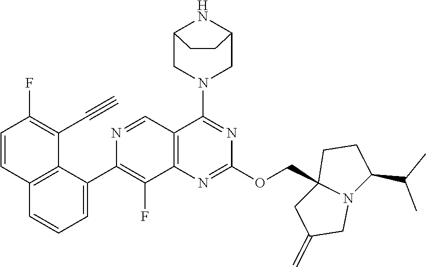 [SMILES: C#Cc1c(F)ccc2cccc(-c3ncc4c(N5CC6CCC(C5)N6)nc(OC[C@@]56CC[C@@H](C(C)C)N5CC(=C)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((5S,7aS)-5- isopropyl-2-methylenetetrahydro-1H-pyrrolizin-7a(5H)- yl)methoxy)pyrido[4,3-d]pyrimidine |
| 46 | 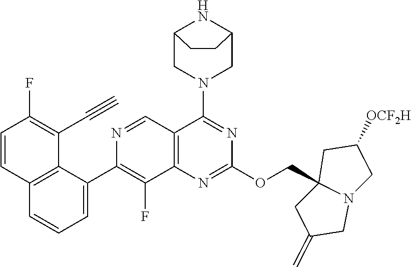 | 4-((1R,5S)-3,8-diazabicyclo[3.2.1]octan-3-yl)-2- (((2S,7aR)-2-(difluoromethoxy)-5-methylenetetrahydro- 1H-pyrrolizin-7a(5H)-yl)methoxy)-7-(8-ethynyl-7- fluoronaphthalen-1-yl)-8-fluoropyrido[4,3-d]pyrimidine |
| 47 | 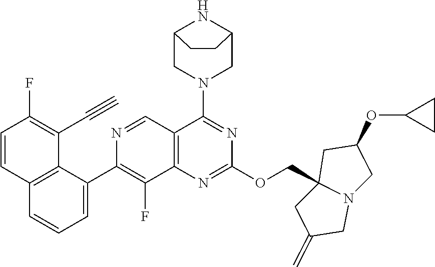 [SMILES: C#Cc1c(F)ccc2cccc(-c3ncc4c(N5CC6CCC(C5)N6)nc(OC[C@]56CC(=C)CN5C[C@H](OC5CC5)C6)nc4c3F)c12] | 4-((1R,5S)-3,8-diazabicyclo[3.2.1]octan-3-yl)-2- (((2R,7aR)-2-cyclopropoxy-5-methylenetetrahydro-1H- pyrrolizin-7a(5H)-yl)methoxy)-7-(8-ethynyl-7- fluoronaphthalen-1-yl)-8-fluoropyrido[4,3-d]pyrimidine |
| 48 | 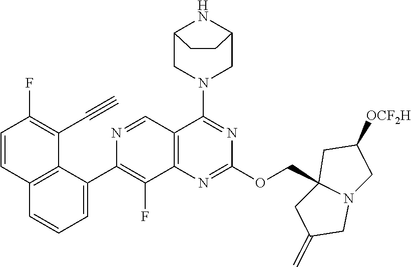 | 4-((1R,5S)-3,8-diazabicyclo[3.2.1]octan-3-yl)-2- (((2R,7aR)-2-(difluoromethoxy)-6-methylenetetrahydro- 1H-pyrrolizin-7a(5H)-yl)methoxy)-7-(8-ethynyl-7- fluoronaphthalen-1-yl)-8-fluoropyrido[4,3-d]pyrimidine |
| 49 | 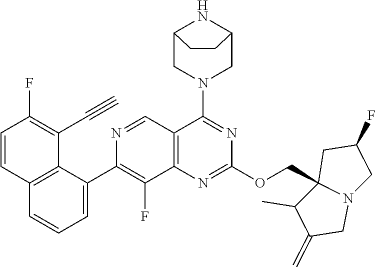 [SMILES: C#Cc1c(F)ccc2cccc(-c3ncc4c(N5CC6CCC(C5)N6)nc(OC[C@@]56C[C@@H](F)CN5CC(=C)C6C)nc4c3F)c12] | 4-((1R,5S)-3,8-diazabicyclo[3.2.1]octan-3-yl)-7-(8- ethynyl-7-fluoronaphthalen-1-yl)-8-fluoro-2-(((6R,7aS)-6- fluoro-1-methyl-2-methylenetetrahydro-1H-pyrrolizin- 7a(5H)-yl)methoxy)pyrido[4,3-d]pyrimidine |
| 50 | 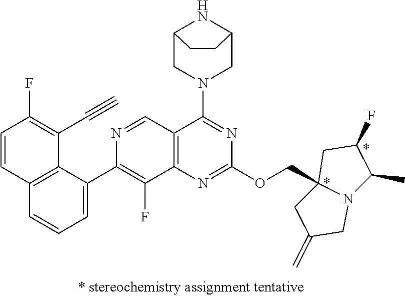 [SMILES: C#Cc1c(F)ccc2cccc(-c3ncc4c(N5CC6CCC(C5)N6)nc(OC[C@]56CC(=C)CN5[C@H](C)[C@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2- (((2R,3R,7aR)-2-fluoro-3-methyl-6-methylenetetrahydro- 1H-pyrrolizin-7a(5H)-yl)methoxy)pyrido[4,3- d]pyrimidine |
| 51 | 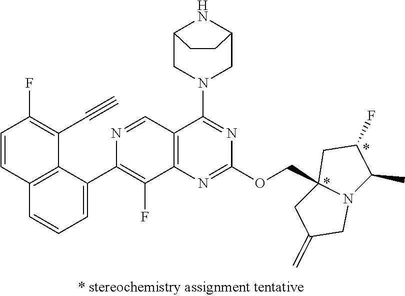 [SMILES: C#Cc1c(F)ccc2cccc(-c3ncc4c(N5CC6CCC(C5)N6)nc(OC[C@]56CC(=C)CN5[C@H](C)[C@@H](F)C6)nc4c3F)c12] | 4-((1R,5S)-3,8-diazabicyclo[3.2.1]octan-3-yl)-7-(8- ethynyl-7-fluoronaphthalen-1-yl)-8-fluoro-2- (((2S,3R,7aR)-2-fluoro-3-methyl-6-methylenetetrahydro- 1H-pyrrolizin-7a(5H)-yl)methoxy)pyrido[4,3- d]pyrimidine |
| 52 | 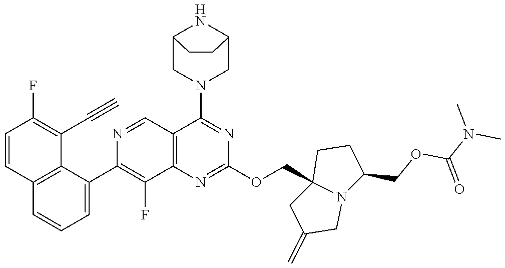 [SMILES: C#Cc1c(F)ccc2cccc(-c3ncc4c(N5CC6CCC(C5)N6)nc(OC[C@@]56CC[C@@H](COC(=O)N(C)C)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl- dimethylcarbamate |
| 53 | 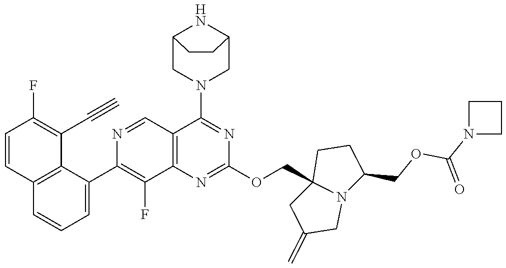 [SMILES: C#Cc1c(F)ccc2cccc(-c3ncc4c(N5CC6CCC(C5)N6)nc(OC[C@@]56CC[C@@H](COC(=O)N7CCC7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl azetidine- 1-carboxylate |
| 54 | 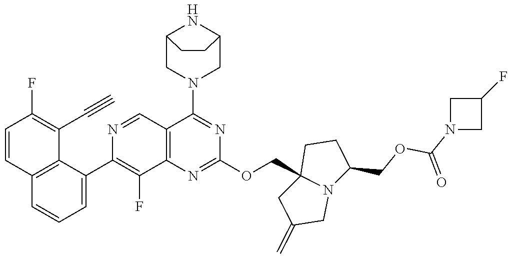 [SMILES: C#Cc1c(F)ccc2cccc(-c3ncc4c(N5CC6CCC(C5)N6)nc(OC[C@@]56CC[C@@H](COC(=O)N7CC(F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3- fluoroazetidine-1-carboxylate |
| 55 | 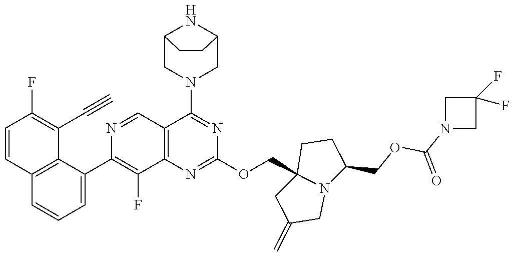 [SMILES: C#Cc1c(F)ccc2cccc(-c3ncc4c(N5CC6CCC(C5)N6)nc(OC[C@@]56CC[C@@H](COC(=O)N7CC(F)(F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3,3- difluoroazetidine-1-carboxylate |
| 56 | 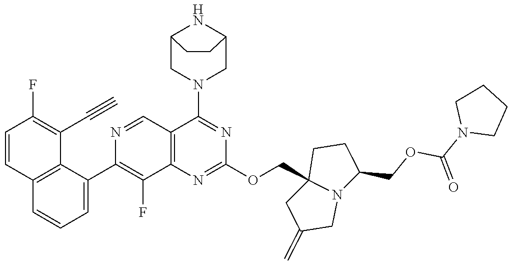 [SMILES: C#Cc1c(F)ccc2cccc(-c3ncc4c(N5CC6CCC(C5)N6)nc(OC[C@@]56CC[C@@H](COC(=O)N7CCCC7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehxahydro-1H-pyrrolizin-3-yl)methyl pyrrolidine-1-carboxylate |
| 57 | 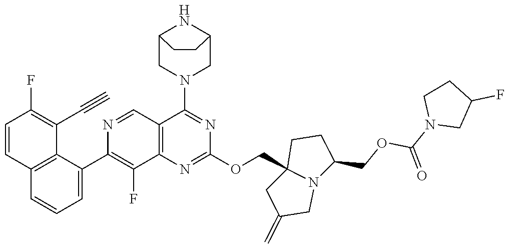 [SMILES: C#Cc1c(F)ccc2cccc(-c3ncc4c(N5CC6CCC(C5)N6)nc(OC[C@@]56CC[C@@H](COC(=O)N7CCC(F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3- fluoropyrrolidine-1-carboxylate |
| 58 | 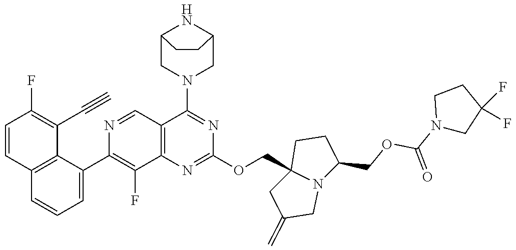 [SMILES: C#Cc1c(F)ccc2cccc(-c3ncc4c(N5CC6CCC(C5)N6)nc(OC[C@@]56CC[C@@H](COC(=O)N7CCC(F)(F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3,3- difluoropyrrolidine-1-carboxylate |
| 59 | 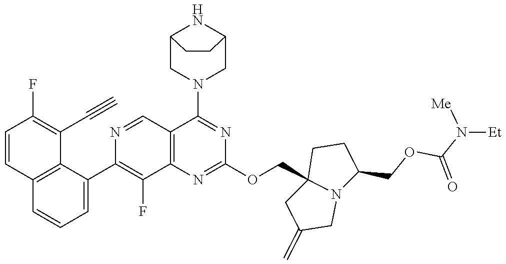 [SMILES: C#Cc1c(F)ccc2cccc(-c3ncc4c(N5CC6CCC(C5)N6)nc(OC[C@@]56CC[C@@H](COC(=O)N(C)CC)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl ethyl(methyl)carbamate |
| 60 | 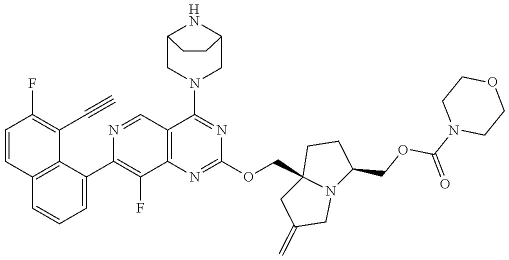 [SMILES: C#Cc1c(F)ccc2cccc(-c3ncc4c(N5CC6CCC(C5)N6)nc(OC[C@@]56CC[C@@H](COC(=O)N7CCOCC7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl morpholine-4-caboxylate |
| 61 | 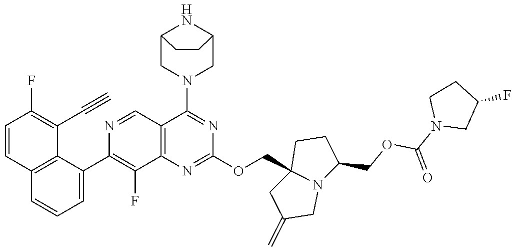 [SMILES: C#Cc1c(F)ccc2cccc(-c3ncc4c(N5CC6CCC(C5)N6)nc(OC[C@@]56CC[C@@H](COC(=O)N7CC[C@H](F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl (S)-3- fluoropyrrolidin-1-carboxylate |
| 62 | 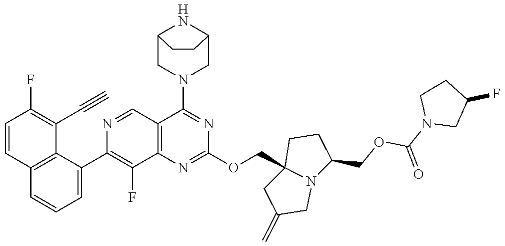 [SMILES: C#Cc1c(F)ccc2cccc(-c3ncc4c(N5CC6CCC(C5)N6)nc(OC[C@@]56CC[C@@H](COC(=O)N7CC[C@@H](F)C7)N5CC(=C)C6)nc4c3F)c12] | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehxahydro-1H-pyrrolizin-3-yl)methyl (R)-3- fluoropyrrolidine-1-carboxylate |
| 63 | 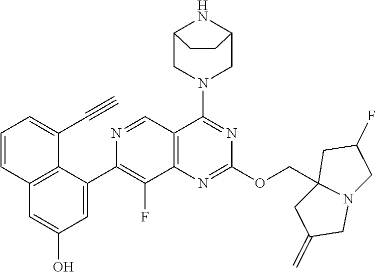 [SMILES: C#Cc1cccc2cc(O)cc(-c3ncc4c(N5CC6CCC(C5)N6)nc(OCC56CC(=C)CN5CC(F)C6)nc4c3F)c12] | 4-(4-((1R,5S)-3,8-diazabicyclo[3.2.1]octan-3-yl)-8- fluoro-2-((2-fluoro-6-methylenetetrahydro-1H-pyrrolizin- 7a(5H)-yl)methoxy)pyrido[4,3-d]pyrimidin-7-yl)-5- ethynylnaphthalen-2-ol |
| 64 | 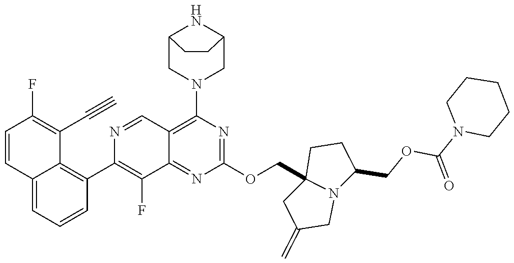 | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl piperidine-1-carboxylate |
| 65 | 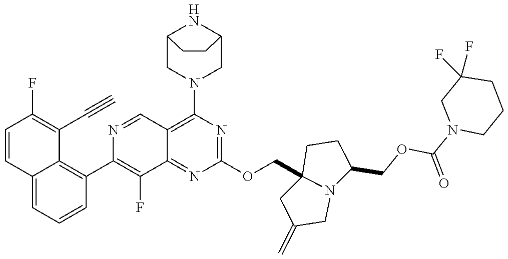 | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3,3- difluoropiperidine-1-carboxylate |
| 66 | 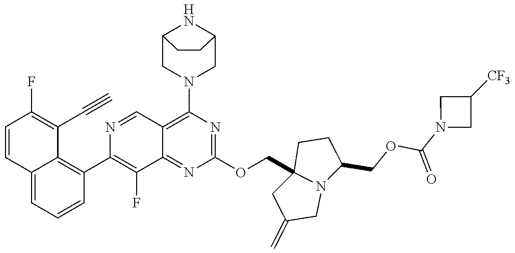 | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3- (trifluoromethyl)azetidine-1-carboxylate |
| 67 | 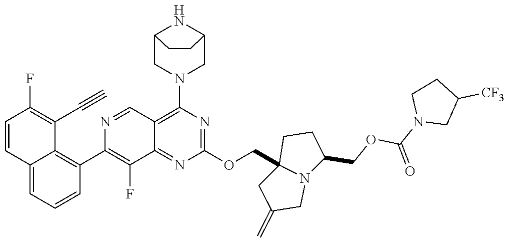 | ((3S,7aS)-7a-(((4-((1R,5S)-3,8-diazabicyclo[3.2.1]octan- 3-yl)-7-(8-ethynyl-7-fluoronaphthalen-1-yl)-8- fluoropyrido[4,3-d]pyrimidin-2-yl)oxy)methyl)-6- methylenehexahydro-1H-pyrrolizin-3-yl)methyl 3- (trifluoromethyl)pyrrolidine-1-carboxylate |

Contemplated compounds of Formula (I) are provided in Compound Table 2 below:

Compound Table 2

| Cpd | | |
|---|---|---|
| No. | Structure | Name |
| 2-1 | 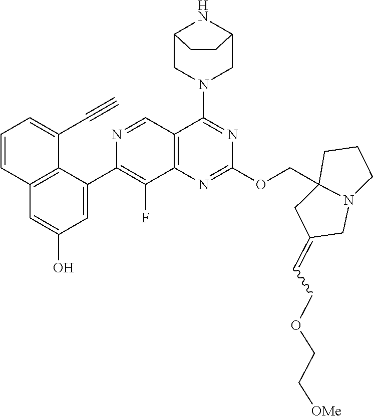 [SMILES: C#Cc1cccc2cc(O)cc(-c3ncc4c(N5CC6CCC(C5)N6)nc(OCC56CCCN5CC(=CCOCCOC)C6)nc4c3F)c12] | 4-(4-((1R,5S)-3,8-diaza- bicyclo[3.2.1]octan-3- yl)-8-fluoro-2-((2-(2-(2- methoxyethoxy)ethylidene)- tetrahydro-1H-pyrrolizin-7a(5H)- yl)methoxy)pyrido[4,3- d]pyrimidin-7-yl)-5- ethynylnaphthalen-2-ol |
| 2-2 | 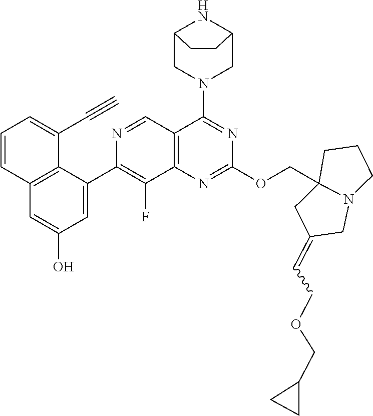 [SMILES: C#Cc1cccc2cc(O)cc(-c3ncc4c(N5CC6CCC(C5)N6)nc(OCC56CCCN5CC(=CCOCC5CC5)C6)nc4c3F)c12] | 4-(4-((1R,5S)-3,8-diaza- bicyclo[3.2.1]octan-3- yl)-2-((2-(2-(cyclopropyl- methoxy)ethylidene)tetrahydro- 1H-pyrrolizin-7a(5H)- yl)methoxy)-8- fluoropyrido[4,3- d]pyrimidin-7-yl)-5- ethynylnaphthalen-2-ol |
| 2-3 | 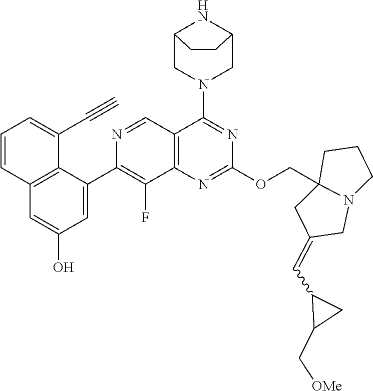 [SMILES: C#Cc1cccc2cc(O)cc(-c3ncc4c(N5CC6CCC(C5)N6)nc(OCC56CCCN5CC(=CC5CC5COC)C6)nc4c3F)c12] | 4-(4-((1R,5S)-3,8-diaza- bicyclo[3.2.1]octan-3- yl)-8-fluoro-2-((2-((2- (methoxymethyl)cyclopropyl)- methylene)tetrahydro-1H- pyrrolizin-7a(5H)- yl)methoxy)pyrido[4,3- d]pyrimidin-7-yl)-5- ethynylnaphthalen-2-ol |
| 2-4 | 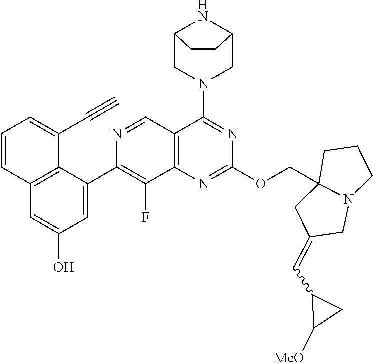 [SMILES: C#Cc1cccc2cc(O)cc(-c3ncc4c(N5CC6CCC(C5)N6)nc(OCC56CCCN5CC(=CC5CC5OC)C6)nc4c3F)c12] | 4-(4-((1R,5S)-3,8-diaza- bicyclo[3.2.1]octan-3- yl)-8-fluoro-2-((2-((2- methoxycyclopropyl)methylene)- tetrahydro-1H-pyrrolizin-7a(5H)- yl)methoxy)pyrido[4,3- d]pyrimidin-7-yl)-5- ethynylnaphthalen-2-ol |
| 2-5 | 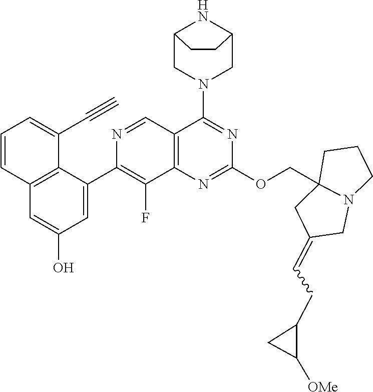 [SMILES: C#Cc1cccc2cc(O)cc(-c3ncc4c(N5CC6CCC(C5)N6)nc(OCC56CCCN5CC(=CCC5CC5OC)C6)nc4c3F)c12] | 4-(4-((1R,5S)-3,8-diaza- bicyclo[3.2.1]octan-3- yl)-8-fluoro-2-((2-(2-(2- methoxycyclopropyl)ethylidene)- tetrahydro-1H-pyrrolizin-7a(5H)- yl)methoxy)pyrido[4,3- d]pyrimidin-7-yl)-5- ethynylnaphthalen-2-ol |
| 2-6 | 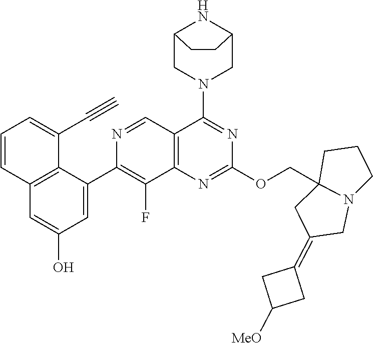 [SMILES: C#Cc1cccc2cc(O)cc(-c3ncc4c(N5CC6CCC(C5)N6)nc(OCC56CCCN5CC(=C5CC(OC)C5)C6)nc4c3F)c12] | 4-(4-((1R,5S)-3,8-diaza- bicyclo[3.2.1]octan-3- yl)-8-fluoro-2-((2-(3-methoxy- cyclobutylidene)tetrahydro- 1H-pyrrolizin-7a(5H)- yl)methoxy)pyrido[4,3- d]pyrimidin-7-yl)-5- ethynylnaphthalen-2-ol |
| 2-7 | 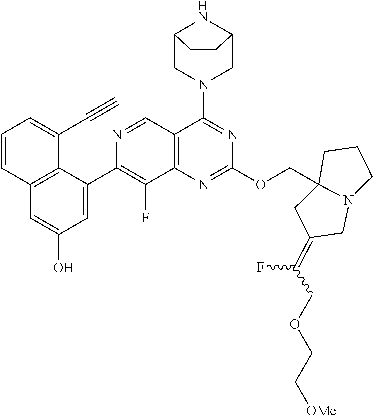 | 4-(4-((1R,5S)-3,8-diaza- bicyclo[3.2.1]octan-3- yl)-8-fluoro-2-((2-(1-fluoro-2-(2- methoxyethoxy)- ethylidene)tetrahydro-1H- pyrrolizin-7a(5H)- yl)methoxy)pyrido[4,3- d]pyrimidin-7-yl)-5- ethynylnaphthalen-2-ol |
| 2-8 | 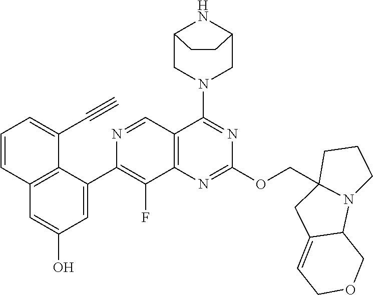 [SMILES: C#Cc1cccc2cc(O)cc(-c3ncc4c(N5CC6CCC(C5)N6)nc(OCC56CCCN5C5COCC=C5C6)nc4c3F)c12] | 4-(4-((1R,5S)-3,8-diaza- bicyclo[3.2.1]octan-3- yl)-8-fluoro-2-((1,3,5,7,8,9a- hexahydropyrano[4,3- b]pyrrolizin-5a(6H)- yl)methoxy)pyrido[4,3- d]pyrimidin-7-yl)-5- ethynylnaphthalen-2-ol |
| 2-9 | 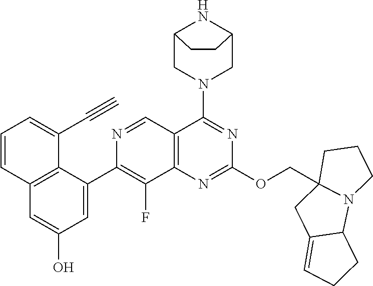 [SMILES: C#Cc1cccc2cc(O)cc(-c3ncc4c(N5CC6CCC(C5)N6)nc(OCC56CCCN5C5CCC=C5C6)nc4c3F)c12] | 4-(4-((1R,5S)-3,8-diaza- bicyclo[3.2.1]octan-3- yl)-8-fluoro-2-((2,3,3a,6,7,8- hexahydrocyclopenta- [b]pyrrolizin-7a(5H)- yl)methoxy)pyrido[4,3- d]pyrimidin-7-yl)-5- ethynylnaphthalen-2-ol |
| 2-10 | 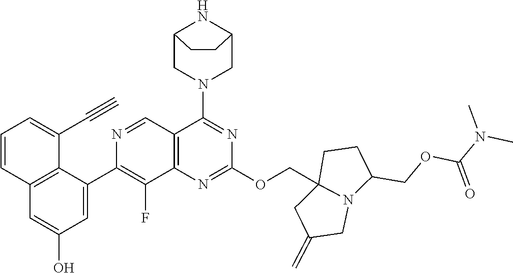 [SMILES: C#Cc1cccc2cc(O)cc(-c3ncc4c(N5CC6CCC(C5)N6)nc(OCC56CCC(COC(=O)N(C)C)N5CC(=C)C6)nc4c3F)c12] | (7a-(((4-((1R,5S)-3,8-diaza- bicyclo[3.2.1]octan- 3-yl)-7-(8-ethynyl-3- hydroxynaphthalen-1- yl)-8-fluoropyrido[4,3- d]pyrimidin-2-yl)oxy)methyl)-6- methylenehexahydro- 1H-pyrrolizin-3-yl)methyl dimethylcarbamate |
| 2-11 | 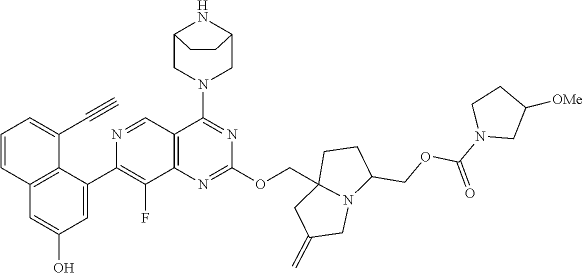 [SMILES: C#Cc1cccc2cc(O)cc(-c3ncc4c(N5CC6CCC(C5)N6)nc(OCC56CCC(COC(=O)N7CCC(OC)C7)N5CC(=C)C6)nc4c3F)c12] | (7a-(((4-((1R,5S)-3,8-diaza- bicyclo[3.2.1]octan- 3-yl)-7-(8-ethynyl-3- hydroxynaphthalen-1- yl)-8-fluoropyrido[4,3- d]pyrimidin-2-yl)oxy)methyl)-6- methylenehexahydro- 1H-pyrrolizin-3-yl)methyl 3- methoxypyrrolidine-1-carboxylate |
| 2-12 | 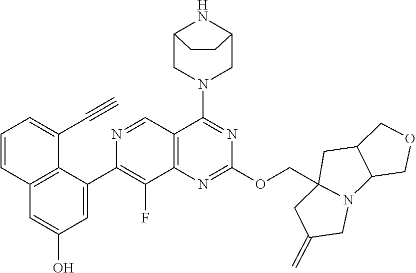 [SMILES: C#Cc1cccc2cc(O)cc(-c3ncc4c(N5CC6CCC(C5)N6)nc(OCC56CC(=C)CN5C5COCC5C6)nc4c3F)c12] | 4-(4-((1R,5S)-3,8-diaza- bicyclo[3.2.1]octan-3- yl)-8-fluoro-2-((6- methylenehexahydro-1H- furo[3,4-b]- pyrrolizin-7a(5H)- yl)methoxy)pyrido[4,3- d]pyrimidin-7-yl)-5- ethynylnaphthalen-2-ol |
| 2-13 | 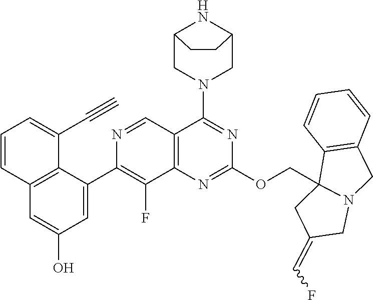 [SMILES: C#Cc1cccc2cc(O)cc(-c3ncc4c(N5CC6CCC(C5)N6)nc(OCC56CC(=CF)CN5Cc5ccccc56)nc4c3F)c12] | 4-(4-((1R,5S)-3,8-diaza- bicyclo[3.2.1]octan-3- yl)-8-fluoro-2-((2- (fluoromethylene)-2,3- dihydro-1H- pyrrolo[2,1-a]isoindol-9b(5H)- yl)methoxy)pyrido[4,3- d]pyrimidin-7-yl)-5- ethynylnaphthalen-2-ol |
| 2-14 | 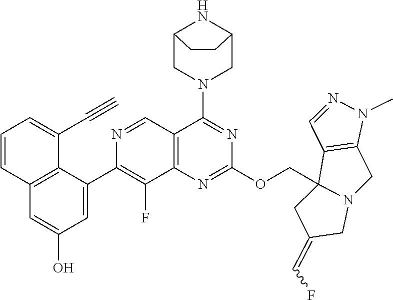 [SMILES: C#Cc1cccc2cc(O)cc(-c3ncc4c(N5CC6CCC(C5)N6)nc(OCC56CC(=CF)CN5Cc5c6cnn5C)nc4c3F)c12] | 4-(4-((1R,5S)-3,8-diaza- bicyclo[3.2.1]octan-3- yl)-8-fluoro-2-((5- (fluoromethylene)-1- methyl-1,5,6,8- tetrahydropyrazolo[4,3- a]pyrrolizin-3b(4H)- yl)methoxy)pyrido[4,3- d]pyrimidin-7-yl)-5-ethynyl- naphthalen-2-ol |
| 2-15 | 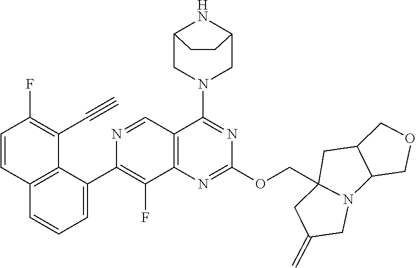 [SMILES: C#Cc1c(F)ccc2cccc(-c3ncc4c(N5CC6CCC(C5)N6)nc(OCC56CC(=C)CN5C5COCC5C6)nc4c3F)c12] | 7a-(((4-((1R,5S)-3,8-diaza- bicyclo[3.2.1]octan- 3-yl)-7-(8-ethynyl-7- fluoronaphthalen-1- yl)-8-fluoropyrido[4,3- d]pyrimidin-2-yl)oxy)methyl)-6- methyleneoctahydro-1H- furo[3,4-b]pyrrolizine |

### Embodiment A

In further embodiments 1-109 below, the present disclosure includes:

In further embodiments 1-109 below, the present disclosure includes:
1. In embodiment 1, provided is a compound of Formula (I) as defined in the first aspect of the Summary, or a pharmaceutically acceptable salt thereof and/or the first embodiment thereof in the Summary.
2. In embodiment 2, the compound of embodiment 1, or a pharmaceutically acceptable salt thereof, is wherein R³⁰ is fused heterocyclylalkyl where fused heterocyclyl of fused heterocyclylalkyl is substituted with R^{a}, R^{b}, and R^{c} as defined in the Summary.
3. In embodiment 3, the compound of any one of embodiments 1 and 2, or a pharmaceutically acceptable salt thereof, is wherein the fused heterocyclyl of fused heterocyclylalkyl is isoindolinyl substituted with R^{a}, R^{b}, and R^{c} as defined therein.
4. In embodiment 4, the compound of embodiment 1, 2 or 3, or a pharmaceutically acceptable salt thereof, is wherein R^{a} is hydrogen.
5. In embodiment 5, the compound of any one of embodiments 1 to 3, or a pharmaceutically acceptable salt thereof, is wherein R^{a} is alkyl.
6. In embodiment 6, the compound of any one of embodiments 1 to 3 and 5, or a pharmaceutically acceptable salt thereof, is wherein R^{a} is methyl, ethyl, n-propyl, isopropyl, n-, iso-, sec-, or tert-butyl.
7. In embodiment 7, the compound of any one of embodiments 1 to 3, or a pharmaceutically acceptable salt thereof, is wherein R^{a} is alkenyl or haloalkenyl.
8. In embodiment 8, the compound of any one of embodiments 1 to 3 and 7, or a pharmaceutically acceptable salt thereof, is wherein R^{a} is vinyl, 2-fluorovinyl, 2-propen-1-yl, or 2-buten-1-yl.
9. In embodiment 9, the compound of embodiment 1, or a pharmaceutically acceptable salt thereof, is wherein R³⁰ is heterocyclylalkyl, bicyclic heterocyclyl, or bicyclic heterocylalkylalkyl, where heterocyclyl of heterocyclylalkyl and bicyclic heterocyclyl, by itself or as part of bicyclic heterocyclylalkyl are substituted with R^{d}, R^{e}, and R^{f} as defined therein.
10. In embodiment 10, the compound of any one of embodiments 1 and 9, or a pharmaceutically acceptable salt thereof, is wherein R³⁰ is heterocyclylalkyl where heterocyclyl of heterocyclylalkyl is substituted with R^{d}, R^{e}, and R^{f} as defined therein.
11. In embodiment 11, the compound of any one of embodiments 1, 9 and 10, or a pharmaceutically acceptable salt thereof, is wherein the heterocyclylalkyl is pyrrolidin-2-ylmethyl, piperidin-2-ylmethyl, or piperidin-3-ylmethyl, preferably pyrrolidin-2-ylmethyl substituted with R^{d}, R^{e}, and R^{f} as defined therein.
12. In embodiment 12, the compound of any one of embodiments 1 and 9, or a pharmaceutically acceptable salt thereof, is wherein R³⁰ is bicyclic heterocyclalkylalkyl substituted with R^{d}, R^{e}, and R^{f} as defined therein.
13. In embodiment 13, the compound of any one of embodiments 1, 9, and 12, or a pharmaceutically acceptable salt thereof, is wherein the bicyclic heterocyclylalkyl is hexahydro-1H-pyrrolizin-7a-ylalkyl, preferably, hexahydro-1H-pyrrolizin-7a-ylmethyl, where hexahydro-1H-pyrrolizin-7a-yl is substituted with R^{d}, R^{e}, and R^{f} as defined therein.
14. In embodiment 14, the compound of any one of embodiments 1, 9, and 12, or a pharmaceutically acceptable salt thereof, is wherein the bicyclic heterocyclylalkyl is a ring of formula:

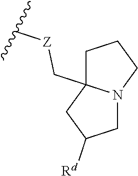

and is substituted with R^{e} and R^{f} as defined therein, preferably

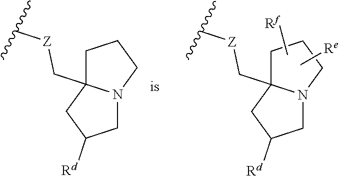

where R^{d}, R^{e} and R^{f} are as defined therein.
15. In embodiment 15, the compound of embodiment 1, or a pharmaceutically acceptable salt thereof, is wherein R³⁰ is fused bicyclic heterocyclyl, fused bicyclic heterocyclylalkyl, heterocyclyl fused bicyclic heterocyclyl, heterocyclyl fused bicyclic heterocyclylalkyl, tricyclic heterocyclyl, tricyclic heterocyclylalkyl, fused tricyclic heterocyclyl, or fused tricyclic heterocyclylalkyl, wherein fused bicyclic heterocyclyl, by itself or as part of fused bicyclic heterocyclylalkyl, heterocyclyl fused bicyclic heterocyclyl, by itself or as part of heterocyclyl fused bicyclic heterocyclylalkyl, tricyclic heterocyclyl, by itself or as part of tricyclic heterocyclylalkyl, and fused tricyclic heterocyclyl, by itself or as part of fused tricyclic heterocyclylalkyl, are independently substituted with R^{g}, R^{h}, and Rⁱ as defined therein.
15a. In embodiment 15a, the compound of embodiment 1 or 15, or a pharmaceutically acceptable salt thereof, is wherein R³⁰ is fused bicyclic heterocyclyl substituted with R^{g}, R^{h}, and Rⁱ as defined therein.
16. In embodiment 16, the compound of embodiment 1, 15, or 15a, or a pharmaceutically acceptable salt thereof, is wherein the fused bicyclic heterocyclyl is a ring of formula:

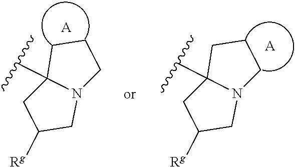

where ring A is phenyl or 5- or 6-membered heteroaryl and the fused bicyclic heterocyclyl is additionally substituted with R^{h} and Rⁱ where R^{g}, R^{h} and Rⁱ are as defined as defined therein, preferably ring A is phenyl or 5- or 6-membered heteroaryl substituted with R^{h} and Rⁱ as defined therein.
17. In embodiment 17, the compound of any one of embodiments 1 and 15, or a pharmaceutically acceptable salt thereof, is wherein R³⁰ is fused bicyclic heterocylalkylalkyl where fused bicyclic heterocyclyl of fused bicyclic heterocyclylalkyl is substituted with R^{g}, R^{h}, and Rⁱ as defined therein.
18. In embodiment 18, the compound of embodiment 1, 15, or 17, or a pharmaceutically acceptable salt thereof, is wherein the fused bicyclic heterocyclyl of fused bicyclic heterocyclylalkyl is 2,3-dihydro-1H-pyrrolo[2,1-a]isoindol-9b(5H)-yl, 2,3-dihydro-1H-pyrrolo[1,2-a]indol-9a(9H)-yl, 1,3b,4,5,6,8-hexahydropyrrolo[3,2-a]pyrrolizin-3b-yl, 1-methyl-1,3b,4,5,6,8-hexahydro-pyrrolo[4,3-a]pyrrolizin-3b-yl, 4b,6,7,9-tetrahydro-5H-pyrido[3,2-a]pyrrolizin-4b-yl, 3,3a,4,5-tetrahydro-2H-pyrano[4,3,2-cd]isoindol-5-yl, or 11,2,3,5,10,10a-hexahydropyrrolo[1,2-b]isoquinolin-10a-yl substituted with R^{g}, R^{h}, and Rⁱ as defined therein.
19. In embodiment 19, the compound of embodiment 1, 15, or 17, or a pharmaceutically acceptable salt thereof, is wherein fused bicyclic heterocyclylalkyl is a ring of formula:

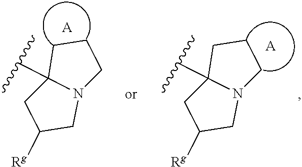

where ring A is phenyl or 5- or 6-membered heteroaryl and the fused bicyclic heterocyclyl is additionally substituted with R^{h} and Rⁱ where R^{g}, R^{h} and Rⁱ are as defined as defined therein, preferably ring A is phenyl or 5- or 6-membered heteroaryl substituted with R^{h} and Rⁱ as defined therein.
20. In embodiment 20, the compound of any one of embodiments 1 and 15, or a pharmaceutically acceptable salt thereof, is wherein R³⁰ is tricyclic heterocyclyl substituted with R^{g}, R^{h}, and Rⁱ as defined therein.
21. In embodiment 21, the compound of any one of embodiments 1, 15, and 20, or a pharmaceutically acceptable salt thereof, is wherein the tricyclic heterocyclyl is a ring of formula:

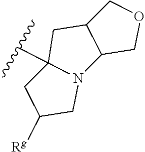

substituted with R^{h}, and Rⁱ as defined therein.
22. In embodiment 22, the compound of any one of embodiments 1 and 15, or a pharmaceutically acceptable salt thereof, is wherein R³⁰ is tricyclic heterocyclylalkyl where tricyclic heterocyclyl of tricyclic heterocyclylalkyl is substituted with R^{g}, R^{h}, and Rⁱ as defined therein.
23. In embodiment 21, the compound of any one of embodiments 1, 15, and 22, or a pharmaceutically acceptable salt thereof, is wherein the tricyclic heterocyclylalkyl is a ring of formula:

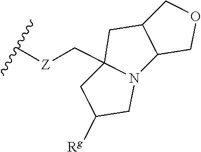

substituted with R^{h}, and Rⁱ as defined therein.
24. In embodiment 24, the compound of any one of embodiments 1 and 15, or a pharmaceutically acceptable salt thereof, is wherein R³⁰ is fused tricyclic heterocyclyl substituted with R^{g}, R^{h}, and Rⁱ as defined therein.
25. In embodiment 25, the compound of embodiment 1, 15, or 24, or a pharmaceutically acceptable salt thereof, is wherein the fused tricyclic heterocyclyl is a ring of formula:

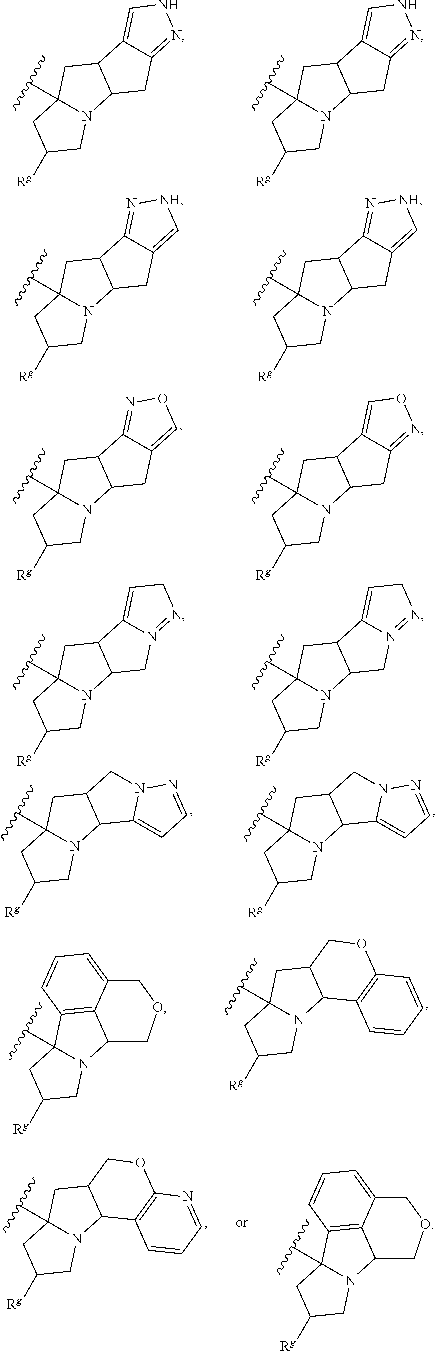

26. In embodiment 26, the compound of any one of embodiments 1 and 15, or a pharmaceutically acceptable salt thereof, is wherein R³⁰ is fused tricyclic heterocyclylalkyl where fused tricyclic heterocyclyl of fused tricyclic heterocyclylalkyl is substituted with R^{g}, R^{h}, and Rⁱ as defined therein.
27. In embodiment 27, the compound of embodiment 1, 15, or 26, or a pharmaceutically acceptable salt thereof, is wherein the fused tricyclic heterocyclylalkyl is a ring of formula:

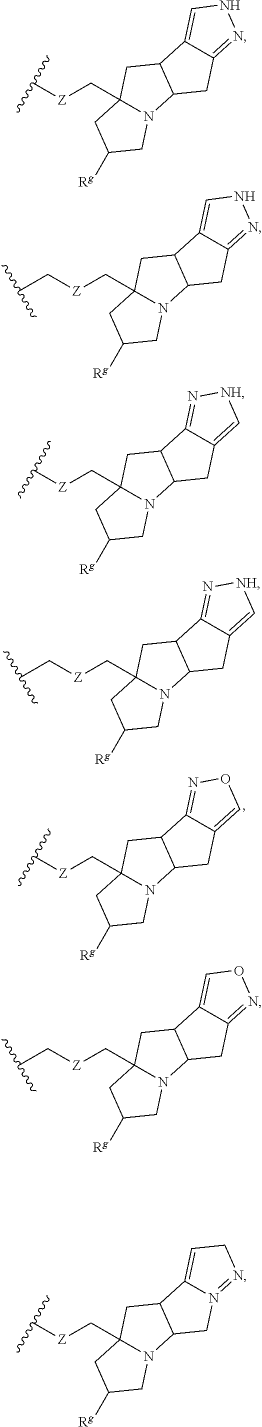

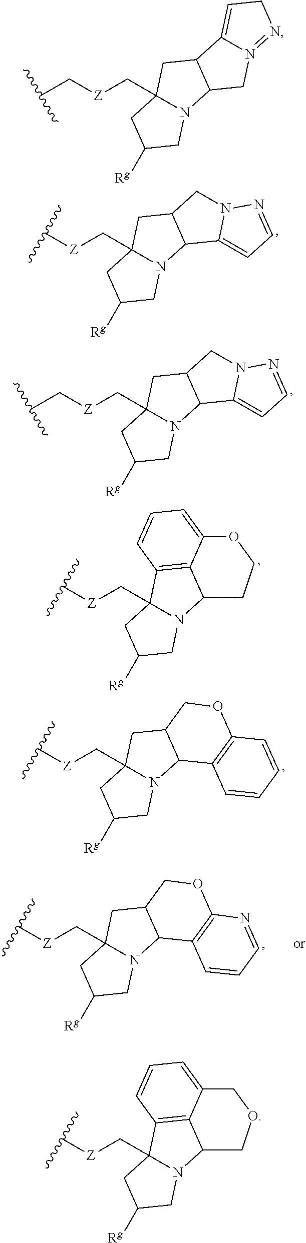

substituted with R^{h} and Rⁱ as defined therein.
28. In embodiment 28, the compound of embodiment 1, or a pharmaceutically acceptable salt thereof, is wherein R³⁰ is bicyclic heterocycloalkenyl, bicyclic heterocycloalkenylalkyl, cycloalkenyl fused bicyclic heterocyclyl, cycloalkenyl fused heterocyclylalkyl, heterocycloalkenyl fused heterocyclyl, and heterocycloalkenyl fused heterocyclylalkyl, wherein bicyclic heterocycloalkenyl, by itself or as part of bicyclic heterocycloalkenylalkyl, cycloalkenyl fused bicyclic heterocyclyl, by itself or as part of cycloalkenyl fused bicyclic heterocyclylalkyl and heterocycloalkenyl fused bicyclic heterocyclyl, by itself of as part of heterocycloalkenyl fused bicyclic heterocyclylalkyl, are independently substituted with R^{j}, R^{k}, and R^{m} as defined therein.
29. In embodiment 29, the compound of any one of embodiments 1 and 28, or a pharmaceutically acceptable salt thereof, is wherein R³⁰ is bicyclic heterocycloalkenyl or bicyclic heterocycloalkenylalkyl substituted with R^{j}, R^{k}, and R^{m} as defined therein.
30. In embodiment 30, the compound of any one of embodiments 1, 28, and 29, or a pharmaceutically acceptable salt thereof, is wherein R³⁰ is bicyclic heterocycloalkenyl substituted with R^{j}, R^{k}, and R^{m} as defined therein.
30a. In embodiment 30a, the compound of embodiment 30, or a pharmaceutically acceptable salt thereof, is wherein bicyclic heterocycloalkenyl is a ring of formula:

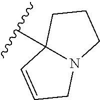 [SMILES: *C12C=CCN1CCC2]

substituted with Rⁱ, R^{k}, and R^{m} as defined therein.
31. In embodiment 31, the compound of any one of embodiments 1, 28, and 29, or a pharmaceutically acceptable salt thereof, is wherein R³⁰ is bicyclic heterocycloalkenylalkyl substituted with R^{j}, R^{k}, and R^{m} as defined therein.
31a. In embodiment 31a, the compound of embodiment 30, or a pharmaceutically acceptable salt thereof, is wherein cycloalkenyl fused bicyclic heterocylalkylalkyl is a ring of formula:

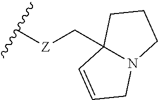

substituted with R^{j}, R^{k}, and R^{m} as defined therein.
32. In embodiment 32, the compound of any one of embodiments 1 and 28, or a pharmaceutically acceptable salt thereof, is wherein R³⁰ is cycloalkenyl fused bicyclic heterocyclyl substituted with R^{j}, R^{k}, and R^{m} as defined therein.
33. In embodiment 33, the compound of any one of embodiments 1 and 28, or a pharmaceutically acceptable salt thereof, is wherein R³⁰ is cycloalkenyl fused bicyclic heterocylalkylalkyl substituted with R^{j}, R^{k}, and R^{m} as defined therein.
34. In embodiment 34, the compound of embodiment 1, 28, or 33, or a pharmaceutically acceptable salt thereof, is wherein cycloalkenyl fused bicyclic heterocylalkylalkyl is a ring of formula:

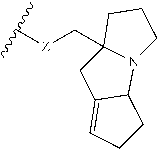

substituted with R^{j}, R^{k}, and R^{m} as defined therein.
35. In embodiment 35, the compound of any one of embodiments 1 and 28, or a pharmaceutically acceptable salt thereof, is wherein R³⁰ is heterocyclcloalkenyl fused bicyclic heterocyclyl substituted with R^{j}, R^{k}, and R^{m} as defined therein.
36. In embodiment 36, the compound of any one of embodiments 1 and 28, or a pharmaceutically acceptable salt thereof, is wherein R³⁰ is heterocycloalkenyl fused bicyclic heterocylalkylalkyl substituted with R^{j}, R^{k}, and R^{m} as defined therein.
37. In embodiment 37, the compound of embodiment 1, 28, or 36, or a pharmaceutically acceptable salt thereof, is wherein the heterocycloalkenyl fused bicyclic heterocylalkylalkyl is a ring of formula:

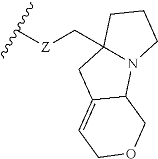

substituted with R^{j}, R^{k}, and R^{m} as defined therein.
37a. In embodiment 37a, the compound of embodiment 1, or a pharmaceutically acceptable salt thereof, is wherein R⁴ is a group selected from:

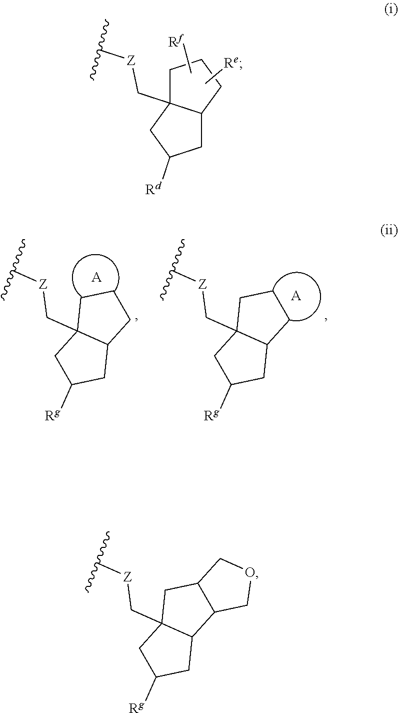

(where ring A is phenyl or 5- or 6-membered heteroaryl, each ring substituted with R^{h} and Rⁱ) (iii)

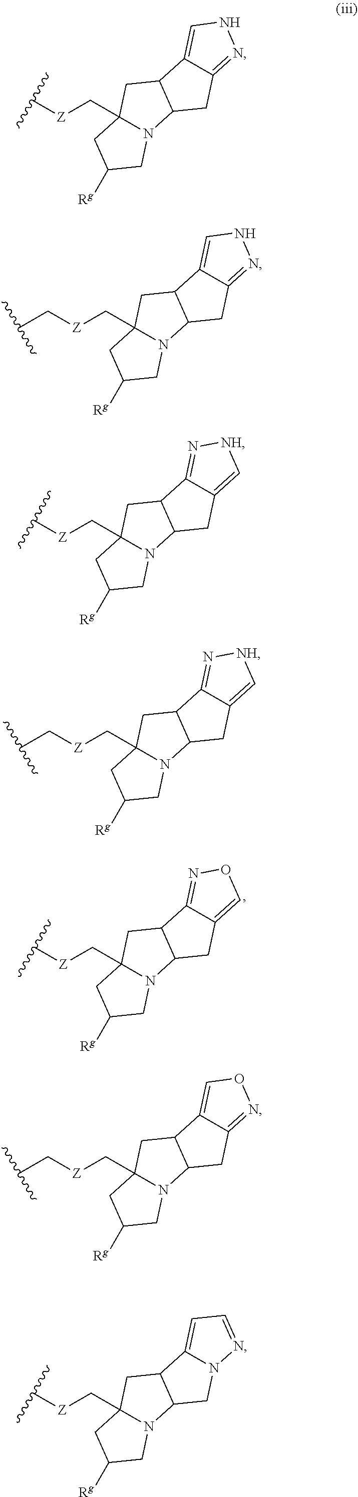

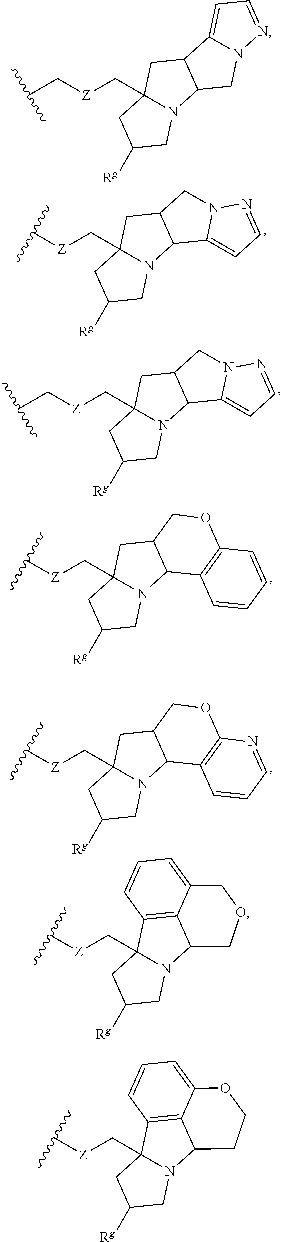

(where each ring is substituted with R^{h} and Rⁱ); and

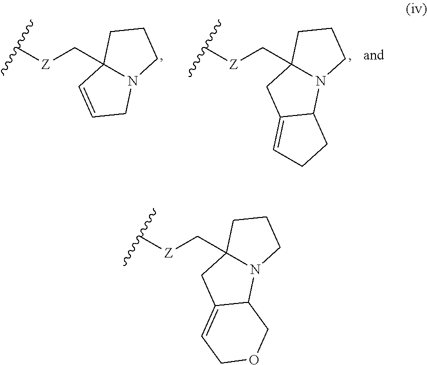

(where each ring is substituted with R^{k} and R^{m}).
38. In embodiment 38, the compound of any one of embodiments 1 to 3 and 9 to 37a, or a pharmaceutically acceptable salt thereof, is wherein R^{a}, R^{d}, R^{g}, and R^{j} are independently haloalkenyl, alkylidenyl, haloalkylidenyl, or alkoxyalkylidenyl.
39. In embodiment 39, the compound of any one of embodiments 1 to 3 and 9 to 38, or a pharmaceutically acceptable salt thereof, is wherein R^{a}, R^{d}, R^{g}, and R^{j} are alkylidenyl.
40. In embodiment 39, the compound of any one of embodiments 1 to 3, and 9 to 39, or a pharmaceutically acceptable salt thereof, is wherein R^{a}, R^{d}, R^{g}, and R^{j} are methylidenyl.
41. In embodiment 41, the compound of any one of embodiments 1 to 3 and 9 to 38, or a pharmaceutically acceptable salt thereof, is wherein R^{a}, R^{d}, R^{g}, and R^{j} are haloalkylidenyl.
42. In embodiment 42, the compound of any one of embodiments 1 to 3, 9 to 38 and 41, or a pharmaceutically acceptable salt thereof, is wherein R^{a}, R^{d}, R^{g}, and R^{j} are independently ═CHF, ═CF₂, ═CHCH₂F, ═C(CH₃)F, or =CHCHF₂.
43. In embodiment 43, the compound of any one of embodiments 1 to 3 and 9 to 38, or a pharmaceutically acceptable salt thereof, is wherein R^{a}, R^{d}, R^{g}, and R^{j} are alkoxyalkylidenyl.
44. In embodiment 44, the compound of any one of embodiments 1 to 3, 9 to 38, and 43, or a pharmaceutically acceptable salt thereof, is wherein R^{a}, R^{d}, R^{g}, and R^{j} are independently ═CH₂O(CH₂)₂OCH₃ or ═CH₂O(CH₂)₂OC₂H₅.
45. In embodiment 45, the compound of any one of embodiments 1 to 3 and 9 to 37a, or a pharmaceutically acceptable salt thereof, is wherein R^{a}, R^{d}, R^{g}, and R^{j} are ═CR³¹R³² ═CR³³R⁴, ═CR³⁵R³⁶, and ═CR³⁷R³⁸ respectively.
46. In embodiment 46, the compound of any one of embodiments 1 to 3, 9 to 37a, and 45, or a pharmaceutically acceptable salt thereof, is wherein R³¹, R³³, R³⁵, and R³⁷ are hydrogen.
47. In embodiment 47, the compound of any one of embodiments 1 to 3, 9 to 37a, and 45, or a pharmaceutically acceptable salt thereof, is wherein R³¹, R³³, R³⁵, and R³⁷ are each fluoro.
48. In embodiment 48, the compound of any one of embodiments 1 to 3, 9 to 37a, and 45, or a pharmaceutically acceptable salt thereof, is wherein R³¹, R³³, R³⁵, and R³⁷ are each alkyl.
49. In embodiment 48, the compound of any one of embodiments 1 to 3, 9 to 37a, 45, and 48, or a pharmaceutically acceptable salt thereof, is wherein R³¹, R³³, R³⁵, and R³⁷ are independently methyl, ethyl, or propyl.
50. In embodiment 50, the compound of any one of embodiments 1 to 3, 9 to 37a, and 45 to 49, or a pharmaceutically acceptable salt thereof, is wherein R³², R³⁴, R³⁶, and R³⁸ are independently cyano, alkoxyalkyloxyalkyl, cycloalkyl, cycloalkylalkyl, or cycloalkylalkyloxyalkyl (where cycloalkyl, by itself or as part of cycloalkylalkyl, and cycloalkylalkyloxyalkyl is optionally substituted with one or two substituents independently selected from alkyl, halo, haloalkoxy, alkoxy, alkoxyalkyl, and hydroxy).
51. In embodiment 51, the compound of any one of embodiments 1 to 3, 9 to 37a, and 45 to 50, or a pharmaceutically acceptable salt thereof, is wherein R³², R³⁴, R³⁶, and R³⁸ are independently cyano, methoxymethyloxymethyl, 2-methoxyethyloxymethyl, cyclopropyl, cyclobutyl, cyclopentyl, cyclopropylmethyl, cyclobutylmethyl, cyclopentylmethyl, cyclopropylethyl, cyclobutylethyl, cyclopentylethyl, cyclopropylmethyloxymethyl, cyclobutylmethyloxymethyl, or cyclopentylmethyloxymethyl, wherein cyclopropyl, cyclobutyl or cyclopentyl, by itself or as part of cyclopropylmethyl, cyclobutylmethyl, cyclopentylmethyl, cyclopropylethyl, cyclobutylethyl, cyclopentylethyl, cyclopropylmethyloxymethyl, cyclobutylmethyloxymethyl, and cyclopentylmethyloxymethyl, respectively, are optionally substituted with methoxy, fluoro, or methoxymethyl.
52. In embodiment 52, the compound of any one of embodiments 1 to 3, 9 to 37a, and 45 to 50, or a pharmaceutically acceptable salt thereof, is wherein R³², R³⁴, R³⁶, and R³⁸ are cyano.
52a. In embodiment 52a, the compound of any one of embodiments 1 to 3, 9 to 37a, and 45 to 50, or a pharmaceutically acceptable salt thereof, is wherein R³², R³⁴, R³⁶, and R³⁸ are independently methoxymethyloxymethyl or 2-methoxyethyloxymethyl.
52b. In embodiment 52b, the compound of any one of embodiments 1 to 3, 9 to 37a, and 45 to 50, or a pharmaceutically acceptable salt thereof, is wherein R³², R³⁴, R³⁶, and R³⁸ are independently cyclopropyl, cyclobutyl, cyclopentyl, cyclopropylmethyl, cyclobutylmethyl, cyclopentylmethyl, cyclopropylethyl, cyclobutylethyl, cyclopentylethyl, cyclopropylmethyloxymethyl, cyclobutylmethyloxymethyl, or cyclopentylmethyloxymethyl, wherein cyclopropyl, cyclobutyl or cyclopentyl, by itself or as part of cyclopropylmethyl, cyclobutylmethyl, cyclopentylmethyl, cyclopropylethyl, cyclobutylethyl, cyclopentylethyl, cyclopropylmethyloxymethyl, cyclobutylmethyloxymethyl, and cyclopentylmethyloxymethyl, respectively, are optionally substituted with methoxy, fluoro, or methoxymethyl.
53. In embodiment 53, the compound of any one of embodiments 1 to 3, 9 to 37a, and 45 to 49, or a pharmaceutically acceptable salt thereof, is wherein R³², R³⁴, R³⁶, and R³⁸ are independently heterocyclyl, phenyl, or heteroaryl (where heterocyclyl, phenyl, and heteroaryl are optionally substituted with one, two, or three substituents independently selected from alkyl, halo, haloalkyl, haloalkoxy, alkoxy, cyano, and hydroxy).
54. In embodiment 54, the compound of any one of embodiments 1 to 3, 9 to 37a, 45 to 49, and 53 or a pharmaceutically acceptable salt thereof, is wherein R³², R³⁴, R³⁶, and R³⁸ are independently phenyl, pyrrolidinyl, furanyl, pyranyl, piperidinyl, morpholinyl, or 5- or 6-membereing heteroaryl, each ring optionally substituted with one, two, or three substituents independently selected from alkyl, halo, haloalkyl, haloalkoxy, alkoxy, cyano, and hydroxy.
55. In embodiment 55, the compound of any one of embodiments 1 to 3, 9 to 37a, 45 to 49, 53 and 54, or a pharmaceutically acceptable salt thereof, is wherein R³², R³⁴, R³⁶, and R³⁸ are independently phenyl, pyrrolidinyl, furanyl, pyranyl, piperidinyl, morpholinyl, 5- or 6-membereing heteroaryl, each ring optionally substituted with one, two, or three substituents independently selected from methyl, ethyl, propyl, fluoro, chloro, difluoromethyl, trifluoromethyl, difluoromethoxy, trifluoromethoxy, methoxy, cyano, or hydroxy.
56. In embodiment 56, the compound of any one of embodiments 1 and 28 to 37a, or a pharmaceutically acceptable salt thereof, is wherein R^{j} is hydrogen.
57. In embodiment 57, the compound of any one of embodiments 1 to 3, 9 to 37a, and 45, or a pharmaceutically acceptable salt thereof, is wherein R³¹ and R³², R³³ and R⁴, R³⁵ and R³⁶ and R³⁷ and R³⁸ together with the carbon atom to which they are attached form cycloalkylene optionally substituted with alkyl, halo, alkoxy, or hydroxy.
58. In embodiment 58, the compound of any one of embodiments 1 to 3, 9 to 37a, 45 and 57, or a pharmaceutically acceptable salt thereof, is wherein R³¹ and R³², R³³ and R³⁴, R³⁵ and R³⁶, and R³⁷ and R³⁸ together with the carbon atom to which they are attached form cyclopropyl, cyclobutylene, or cyclopentylene, each ring optionally substituted with methyl, fluoro, or methoxy.
59. In embodiment 59, the compound of any one of embodiments 1 to 58, or a pharmaceutically acceptable salt thereof, wherein R^{b}, R^{e}, R^{h}, and R^{k} are independently hydrogen, alkyl, halo, haloalkyl, haloalkoxy, alkoxy, hydroxy, cycloalkyl, cycloalkylalkyl, heterocyclyl, or heterocyclylalkyl, (where cycloalkyl, by itself or as part of cycloalkylalkyl, and heterocyclyl, by itself or as part of heterocyclylalkyl, are optionally substituted with one, two, or three substituents independently selected from alkyl, halo, haloalkyl, haloalkoxy, alkoxy, alkoxyalkyl, cyano, and hydroxy).
60. In embodiment 60, the compound of any one of embodiments 1 to 59, or a pharmaceutically acceptable salt thereof, is wherein R^{b}, R^{e}, R^{h}, and R^{k} are independently hydrogen, methyl, fluoro, chloro, difluoromethyl, trifluoromethyl, difluoromethoxy, trifluoromethoxy, methoxy, ethoxy, hydroxy, cyclopropyl, cyclopropylmethyl, pyrrolidinyl, pyrrolidinylmethyl, pyrrolidinylethyl, piperidinyl, piperidinylmethyl, piperidinylethyl, piperizinyl, piperizinylmethyl, or piperizinylethyl (wherein cyclopropyl, by itself or as part of cyclopropylmethyl, pyrrolidinyl, by itself or as part of pyrrolidinylmethyl and pyrrolidinylethyl, piperidinyl, by itself or as part of piperidinylmethyl and piperidinylethyl, and piperazinyl, by itself or as part of piperazinylmethyl and piperazinylethyl are optionally substituted with one or two substituents independently selected from methyl, chloro, fluoro, trifluoromethyl, trifluoromethoxy, methoxy, methoxymethyl, and cyano).
61. In embodiment 61, the compound of any one of embodiments 1 to 58, or a pharmaceutically acceptable salt thereof, wherein R^{b}, R^{e}, R^{h}, and R^{k} are independently alkoxyalkyl, alkoxyalkoxy, alkoxyalkyloxyalkyl, cycloalkoxy, cycloalkoxyalkyl, cycloalkylalkyloxy, cycloalkylalkyloxyalkyl, cycloalkylalkyloxyalkyloxy, bridged cycloalkyloxy, bridged cycloalkyloxyalkyl, bridged cycloalkylalkyloxy, bridged cycloalkylalkyloxyalkyl, heterocyclyloxy, heterocyclyloxyalkyl, heterocyclylalkyloxy, heterocyclylalkyloxyalkyl (where cycloalkyl as part of cycloalkoxy, cycloalkylalkyloxy, cycloalkoxyalkyl, cycloalkylalkyloxyalkyl, and cycloalkylalkyloxyalkyloxy, bridged cycloalkyl, as part of bridged cycloalkyloxy, bridged cycloalkyloxyalkyl, bridged cycloalkylalkyloxy, and bridged cycloalkylalkyloxyalkyl, and heterocyclyl as part of heterocyclyloxy, heterocyclyloxyalkyl, heterocyclylalkyloxy, and heterocyclylalkyloxyalkyl, are optionally substituted with one, two, or three substituents independently selected from alkyl, halo, haloalkyl, haloalkoxy, alkoxy, alkoxyalkyl, cyano, and hydroxy).
62. In embodiment 62, the compound of any one of embodiments 1 to 58, or a pharmaceutically acceptable salt thereof, is wherein R^{b}, R^{e}, R^{h}, and R^{k} are independently methoxymethyl, methoxyethyl, methoxymethyloxy, methoxyethyloxy, methoxymethyloxymethyl, methoxyethyloxymethyl, cyclopropyloxy, cyclopropylmethyloxy, cyclopropyloxymethyl, cycloalkyloxyethyloxy, cyclopropylmethyloxymethyl, bicyclo[1.1.1]pentyloxy, bicyclo[1.1.1]pentyloxymethyl, bicyclo[1.1.1]pentylmethyloxy, bicyclo[1.1.1]pentyl-methyloxymethyl, tetrahydrofuranyloxy, pyrrolidinyloxy, pyrrolidinyloxymethyl, pyrrolidinyloxyethyl, pyrrolidinylmethyloxy, pyrrolidinylethyloxy, pyrrolidinylmethyloxymethyl, pyrrolidinylethyloxymethyl, piperidinyloxy, piperidinyloxymethyl, piperidinyloxyethyl, piperidinylmethyloxy, piperidinylethyloxy, piperidinylmethyloxymethyl, piperidinylethyloxymethyl, piperazinyloxy, piperazinyloxymethyl, piperazinyloxyethyl, piperazinylmethyloxy, piperazinylethyloxy, piperazinylmethyloxymethyl, piperazinylethyloxymethyl, morpholinyloxy, morpholinyloxymethyl, morpholinyloxyethyl, morpholinylmethyloxy, morpholinylethyloxy, morpholinylmethyloxymethyl, morpholinylethyloxymethyl (wherein cyclopropyl as part of cyclopropyloxy, cyclopropylmethyloxy, cycloalkyloxyethyloxy, cyclopropyloxymethyl, and cyclopropylmethyloxymethyl, bicyclo[1.1.1]pentyl as part of bicyclo[1.1.1]pentyloxy, bicyclo[1.1.1]pentyloxymethyl, bicyclo[1.1.1]pentylmethyloxy, and bicycl[1.1.1]pentylmethyloxymethyl, pyrrolidinyl as part of pyrrolidinyloxy, pyrrolidinyloxymethyl, pyrrolidinyloxyethyl, pyrrolidinylmethyloxy, pyrrolidinylethyloxy, pyrrolidinylmethyl-oxymethyl, and pyrrolidinylethyloxymethyl, piperidinyl as part of piperidinyloxy, piperidinyloxymethyl, piperidinyloxyethyl, piperidinylmethyloxy, piperidinylethyloxy, piperidinylmethyloxymethyl, and piperidinylethyloxymethyl, piperazinyl as part of piperazinyloxy, piperazinyloxymethyl, piperazinyloxyethyl, piperazinylmethyloxy, piperazinylethyloxy, piperazinylmethyloxymethyl, and piperazinylethyloxymethyl, and morpholinyl as part of morpholinyloxy, morpholinyloxymethyl, morpholinyloxyethyl, morpholinylmethyloxy, morpholinylethyloxy, morpholinylmethyl-oxymethyl, and morpholinylethyloxymethyl are optionally substituted with one or two substituents independently selected from methyl, chloro, fluoro, trifluoromethyl, trifluoromethoxy, methoxy, methoxymethyl, and cyano).
63. In embodiment 63, the compound of any one of embodiments 1 to 58, or a pharmaceutically acceptable salt thereof, is wherein R^{b}, R^{e}, R^{h}, and R^{k} are independently methoxymethyl, methoxyethyl, methoxymethyloxy, methoxyethyloxy, methoxymethyloxymethyl, or methoxyethyloxymethyl.
64. In embodiment 64, the compound of any one of embodiments 1 to 58, or a pharmaceutically acceptable salt thereof, is wherein R^{b}, R^{e}, R^{h}, and R^{k} are independently cyclopropyloxy, cyclopropylmethyloxy, cyclopropyloxymethyl, cycloalkyloxyethyloxy, or cyclopropylmethyloxymethyl (wherein cyclopropyl as part of cyclopropyloxy, cyclopropyloxymethyl, cyclopropylmethyloxy, cycloalkyloxyethyloxy, and cyclopropylmethyloxymethyl are optionally substituted with one or two substituents independently selected from methyl, chloro, fluoro, trifluoromethyl, trifluoromethoxy, methoxy, methoxymethyl, and cyano).
65. In embodiment 65, the compound of any one of embodiments 1 to 58, or a pharmaceutically acceptable salt thereof, is wherein R^{b}, R^{e}, R^{h}, and R^{k} are independently bicyclo[1.1.1]pentyloxy, bicyclo[1.1.1]pentyloxymethyl, bicyclo[1.1.1]pentylmethyloxy, or bicyclo[1.1.1]pentyl-methyloxymethyl (wherein bicyclo[1.1.1]pentyl as part of bicyclo[1.1.1]pentyloxy, bicyclo[1.1.1]pentyloxymethyl, bicyclo[1.1.1]pentylmethyloxy, and bicycl[1.1.1]pentylmethyloxymethyl are optionally substituted with one or two substituents independently selected from methyl, chloro, fluoro, trifluoromethyl, trifluoromethoxy, methoxy and cyano).
66. In embodiment 66, the compound of any one of embodiments 1 to 58, or a pharmaceutically acceptable salt thereof, is wherein R^{b}, R^{e}, R^{h}, and R^{k} are independently tetrahydrofuranyloxy, pyrrolidinyloxy, pyrrolidinyloxymethyl, pyrrolidinyloxyethyl, pyrrolidinylmethyloxy, pyrrolidinylethyloxy, pyrrolidinylmethyloxymethyl, pyrrolidinylethyloxymethyl, piperidinyloxy, piperidinyloxymethyl, piperidinyloxyethyl, piperidinylmethyloxy, piperidinylethyloxy, piperidinylmethyloxymethyl, or piperidinylethyloxymethyl (wherein pyrrolidinyl as part of pyrrolidinyloxy, pyrrolidinyloxymethyl, pyrrolidinyloxyethyl, pyrrolidinylmethyloxy, pyrrolidinylethyloxy, pyrrolidinylmethyl-oxymethyl, and pyrrolidinylethyloxymethyl, piperidinyl as part of piperidinyloxy, piperidinyloxymethyl, piperidinyloxyethyl, piperidinylmethyloxy, piperidinylethyloxy, piperidinylmethyloxymethyl, and piperidinylethyloxymethyl are optionally substituted with one or two substituents independently selected from methyl, chloro, fluoro, trifluoromethyl, trifluoromethoxy, methoxy, methoxymethyl, and cyano).
67. In embodiment 67, the compound of any one of embodiments 1 to 58, or a pharmaceutically acceptable salt thereof, is wherein R^{b}, R^{e}, R^{h}, and R^{k} are -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy).
68. In embodiment 68, the compound of any one of embodiments 1 to 58 and 67, or a pharmaceutically acceptable salt thereof, is wherein R^{b}, R^{e}, R^{h}, and R^{k} are -(alkylene)-OC(O)R³⁹ where alkylene is methylene or ethylene and R³⁹ is methylamino, dimethylamino, or diethylamino.
69. In embodiment 69, the compound of any one of embodiments 1 to 58 and 67, or a pharmaceutically acceptable salt thereof, is wherein R^{b}, R^{e}, R^{h}, and R^{k} are -(alkylene)-OC(O)R³⁹ where alkylene is methylene or ethylene and R³⁹ is pyrrolidin-1-yl, piperidin-1-yl, or morpholin-4-yl, each ring optionally substituted with one or two substituents independently selected from methyl, fluoro, trifluoromethyl, trifluoromethoxy, and methoxy.
70. In embodiment 70, the compound of any one of embodiments 1 to 58 and 67, or a pharmaceutically acceptable salt thereof, is wherein R^{b}, R^{e}, R^{h}, and R^{k} are R^{b} is 3-methoxypyrrolidin-1-ylcarbonyloxymethyl.
71. In embodiment 71, the compound of any one of embodiments 1 to 70, or a pharmaceutically acceptable salt thereof, is wherein R^{c}, R^{f}, Rⁱ, and R^{m} are independently hydrogen, methyl, ethyl, methoxy, ethoxy, methyloxy, ethyloxy, chloro, or fluoro.
72. In embodiment 72, the compound of any one of embodiments 1 to 70, or a pharmaceutically acceptable salt thereof, is wherein R^{c}, R^{f}, Rⁱ, and R^{m} are hydrogen.
73. In embodiment 73, the compound of any one of embodiments 1 to 70, or a pharmaceutically acceptable salt thereof, is wherein R^{c}, R^{f}, Rⁱ, and R^{m} are independently methyl, methoxy, methyloxy, chloro, or fluoro.
74. In embodiment 74, the compound of embodiment 1, 2, or 3, or a pharmaceutically acceptable salt thereof, is wherein fused heterocyclylalkyl is:

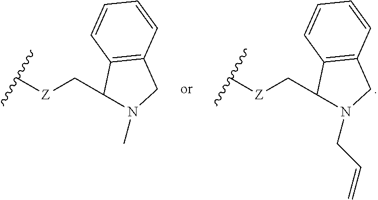

75. In embodiment 75, the compound of any one of embodiments 1, 9, 12, and 14, or a pharmaceutically acceptable salt thereof, is wherein the bicyclic heterocyclylalkyl is:

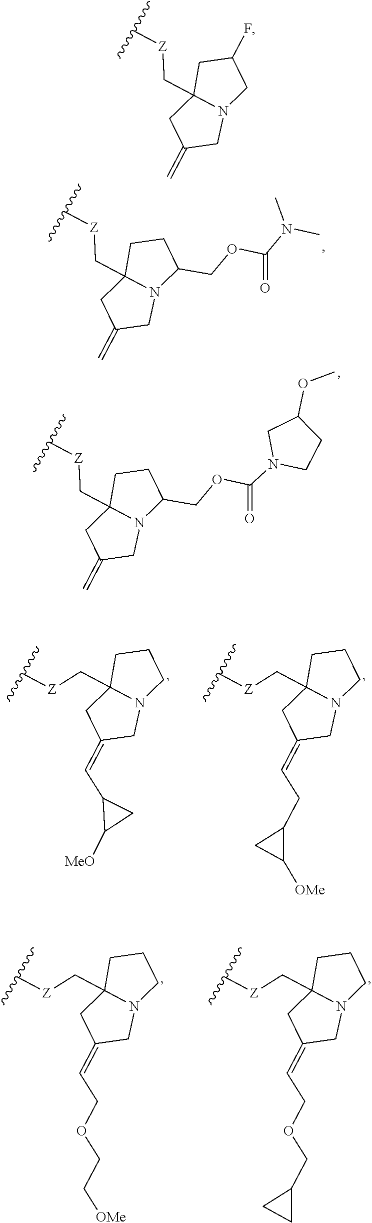

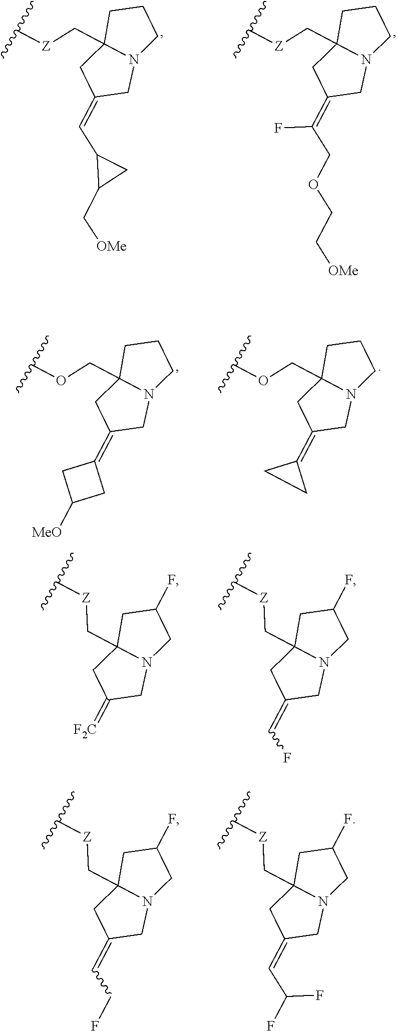

76. In embodiment 76, the compound of any one of embodiments 1, 9, 12, and 14, or a pharmaceutically acceptable salt thereof, is wherein the bicyclic heterocyclylalkyl is:

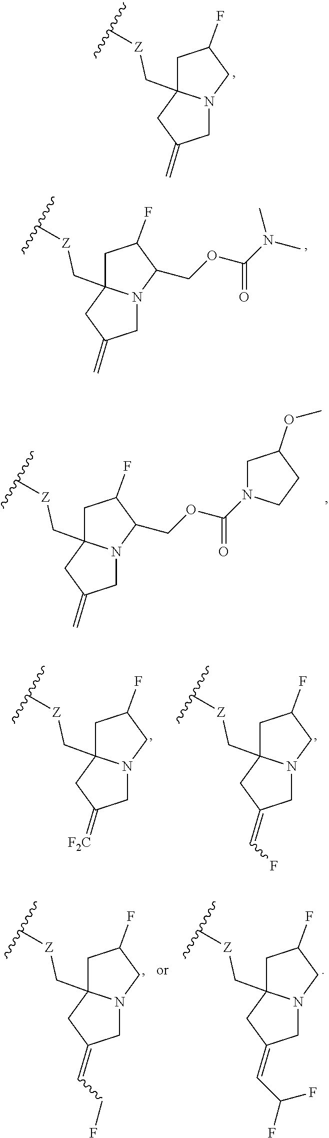

77. In embodiment 77, the compound of any one of embodiments 1, 9, 12, and 14, or a pharmaceutically acceptable salt thereof, is wherein the bicyclic heterocyclylalkyl is:

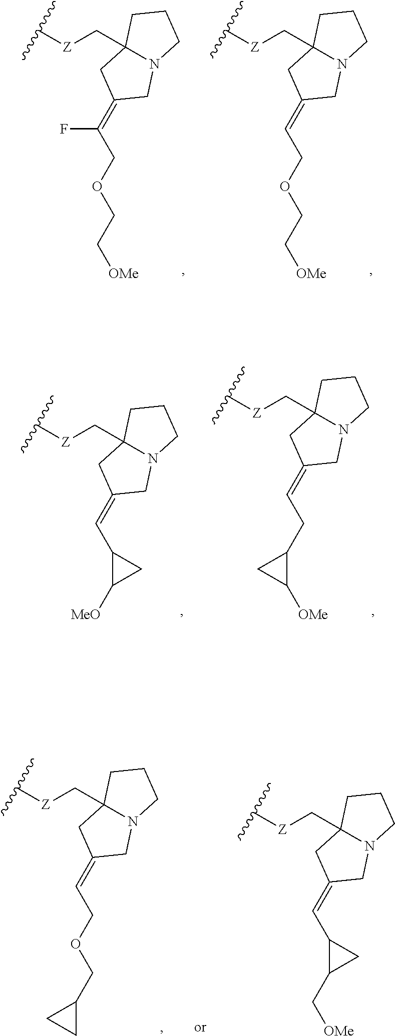

78. In embodiment 78, the compound of any one of embodiments 1, 15, and 19, or a pharmaceutically acceptable salt thereof, is wherein fused bicyclic heterocyclylalkyl is a ring of formula:

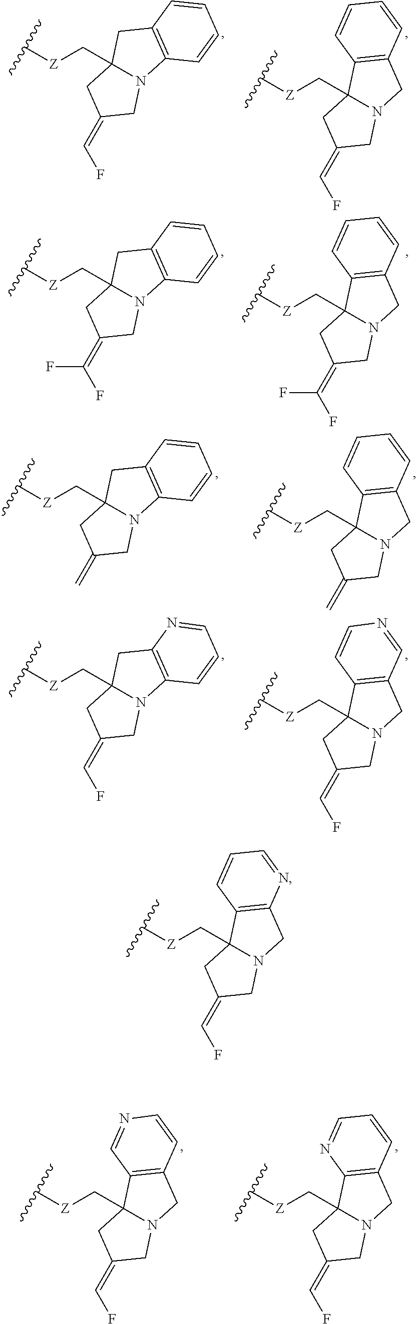

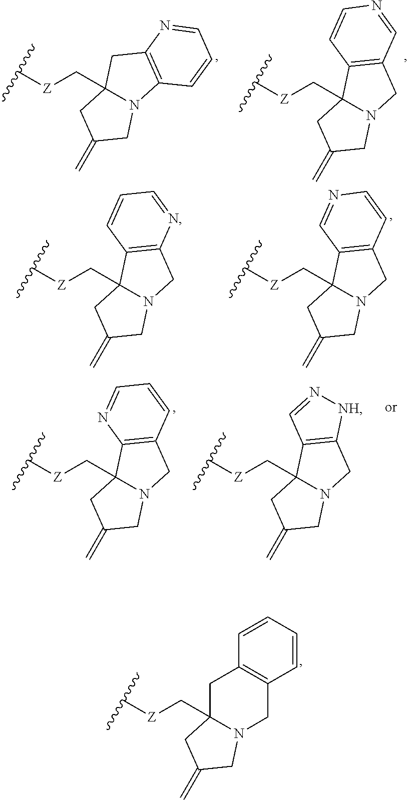

where phenyl and heteroaryl rings are substituted with R^{h} and Rⁱ as defined therein.
79. In embodiment 79, the compound of embodiment 1, 15, and 22, or a pharmaceutically acceptable salt thereof, is wherein tricyclic heterocyclylalkyl is

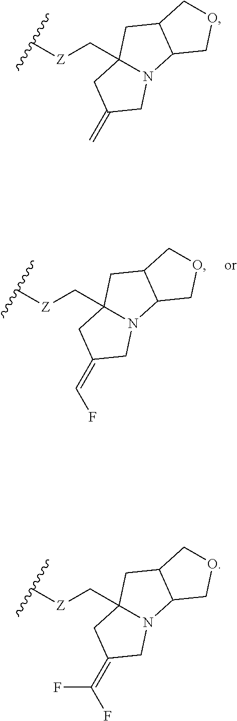

80. In embodiment 80, the compound of any one of embodiments 1 to 79, or a pharmaceutically acceptable salt thereof, is wherein Z is O.
81. In embodiment 81, the compound of embodiment 1, or a pharmaceutically acceptable salt thereof, is wherein R⁴ is a group selected from:

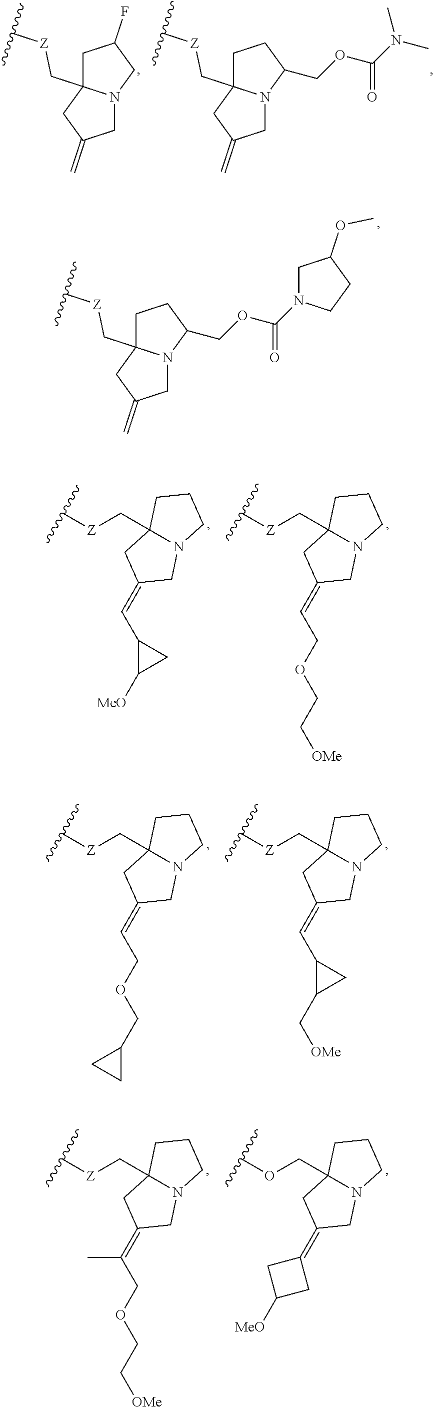

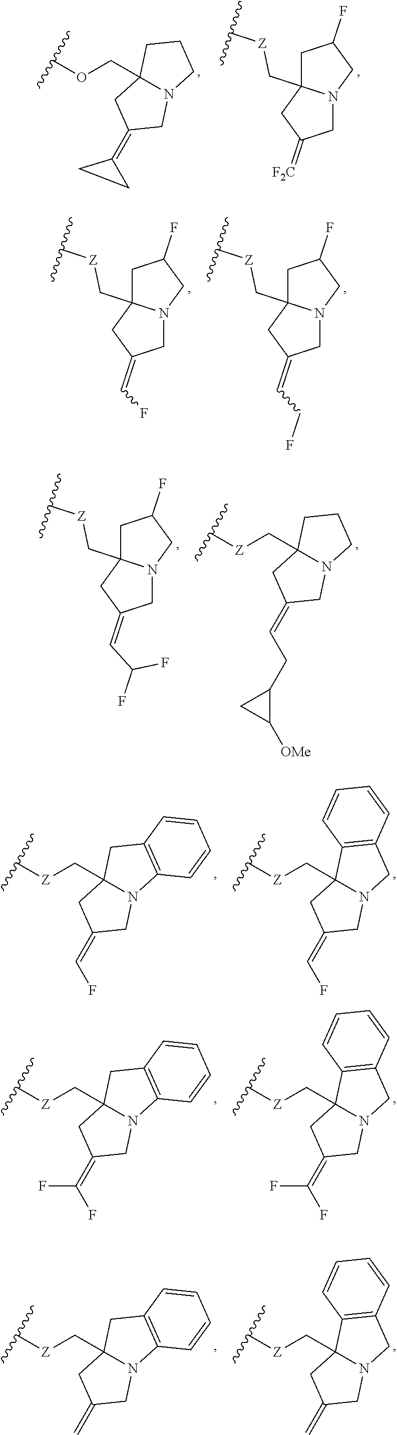

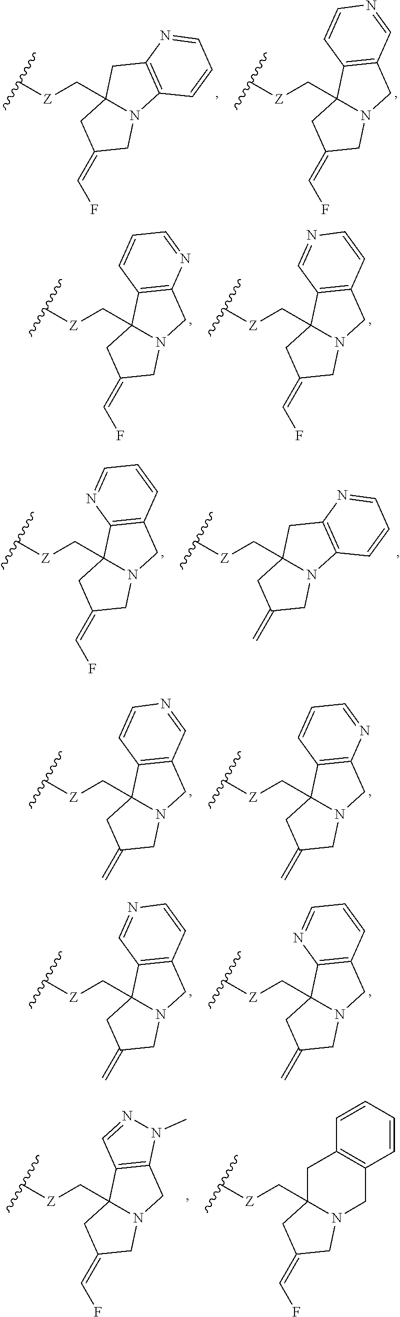

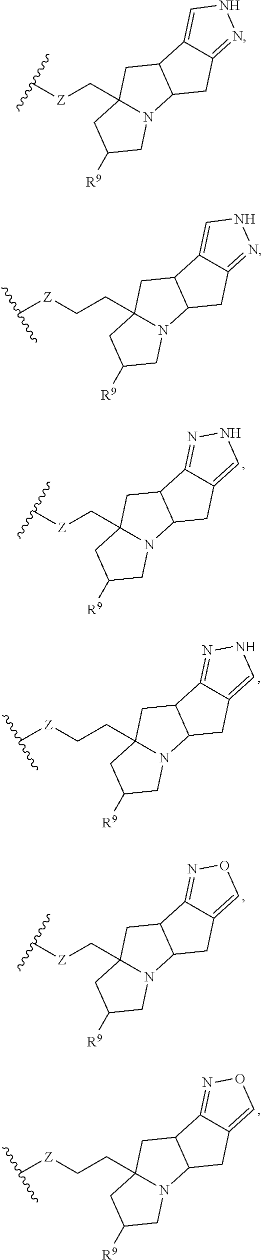

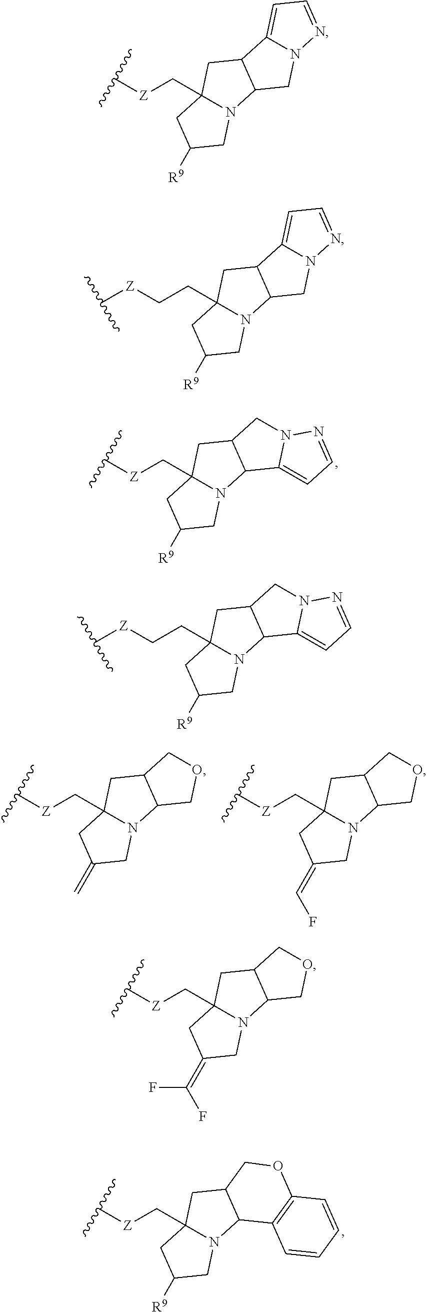

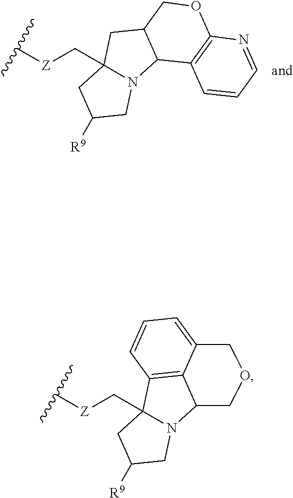

where each R^{g} is ═CH₂, ═CHF, or ═CF₂; preferably, Z is O.
82. In embodiment 82, the compound of any one of embodiments 1 to 81, or a pharmaceutically acceptable salt thereof, is wherein:
R¹ is a ring of formula

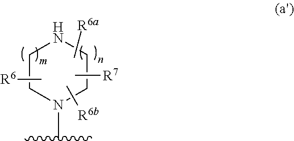

one of m and n is 0, 1, or 2, and the other of m and n is 0, 1, 2, or 3.
82a. In embodiment 82a, the compound of embodiment 1, is wherein the compound is a compound of Formula (I′c):

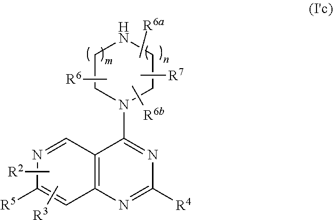

wherein:
one of m and n is 0, 1, or 2, and the other of m and n is 0, 1, 2, or 3;
R⁶ is hydrogen, deuterium, alkyl, alkoxy, halo, haloalkyl, hydroxy, hydroxylalkyl, alkoxyalkyl, cyano, or cyanomethyl, provided R⁶ is not attached to the ring —NH—;
R⁷ is hydrogen, deuterium, alkyl, alkoxy, halo, haloalkyl, hydroxy, hydroxylalkyl, or alkoxyalkyl, provided R⁷ is not attached to the ring —NH—; or
when R⁶ and R⁷ are attached to the carbon atoms of the ring that are opposite or diagonal to each other, R⁶ and R⁷ can combine to form —(CH₂)_{z}— where (z is 1, 2, or 3), or —CH═CH—;
R^{6a} is hydrogen, deuterium, alkyl, alkylidenyl, alkoxy, halo, haloalkyl, hydroxy, hydroxyalkyl, alkoxyalkyl, cyano, cyanomethyl, cyanoethyl, or 2-cyanovinyl, provided R^{6a} is not attached to the ring —NH—;
R^{6b} is hydrogen or alkyl, provided R^{6b} is not attached to the ring —NH—; or
when R^{6a} and R^{6b} are attached to the same carbon of ring (a′), R^{6a} and R^{6b} can combine to form cycloalkylene;
R² is hydrogen, deuterium, alkyl, halo, haloalkyl, alkoxy, hydroxy, or cyano,;
R³ is hydrogen, deuterium, alkyl, halo, haloalkyl, alkoxy, cycloalkyl, cycloalkyloxy, hydroxy, or cyano;
R⁴ is —Z—R³⁰ where Z is a bond, O, NH, N(alkyl), or S; and R³⁰ is bicyclic heterocyclylalkyl wherein bicyclic heterocyclyl part of bicyclic heterocyclylalkyl, is substituted with R^{d}, R^{e}, and R^{f} where R^{d} is alkenyl, haloalkenyl, alkylidenyl, haloalkylidenyl, alkoxyalkylidenyl, or R^{d} is ═CR³³R³⁴;
where R³³ is hydrogen, alkyl, or fluoro and R³⁴ is cyano, alkoxyalkyloxyalkyl, cycloalkyl, cycloalkylalkyl, or cycloalkylalkyloxyalkyl (where cycloalkyl, by itself or as part of cycloalkylalkyl and cycloalkylalkyloxyalkyl is optionally substituted with one or two substituents independently selected from alkyl, halo, haloalkoxy, alkoxy, alkoxyalkyl, and hydroxy), heterocyclyl, phenyl, or heteroaryl (where heterocyclyl, phenyl, and heteroaryl are optionally substituted with one, two, or three substituents independently selected from alkyl, halo, haloalkyl, haloalkoxy, alkoxy, cyano, and hydroxy), or R³³ and R³⁴ together with the carbon atom to which they are attached form cycloalkylene optionally substituted with alkyl, halo, alkoxy, or hydroxy;
R^{e} is hydrogen, alkyl, halo, haloalkyl, haloalkoxy, alkoxy, hydroxy, alkoxyalkyl, alkoxyalkoxy, alkoxyalkyloxyalkyl, cycloalkyl, cycloalkylalkyl, cycloalkoxy, cycloalkylalkyloxy, cycloalkoxyalkyl, cycloalkylalkyloxyalkyl, cycloalkylalkyloxyalkyloxy, bridged cycloalkyloxy, bridged cycloalkyloxyalkyl, bridged cycloalkylalkyloxy, bridged cycloalkylalkyloxyalkyl, heterocyclyl, heterocyclylalkyl, heterocyclyloxy, heterocyclyloxyalkyl, heterocyclylalkyloxy, heterocyclylalkyloxyalkyl (where cycloalkyl, by itself or as part of cycloalkylalkyl, cycloalkoxy, cycloalkoxyalkyl, cycloalkylalkyloxy, cycloalkylalkyloxyalkyl, and cycloalkylalkyloxyalkyloxy, bridged cycloalkyl, as part of bridged cycloalkyloxy, bridged cycloalkyloxyalkyl, bridged cycloalkylalkyloxy, and bridged cycloalkylalkyloxyalkyl, and heterocyclyl, by itself or as part of heterocyclylalkyl, heterocyclyloxy, heterocyclyloxyalkyl, heterocyclylalkyloxy, and heterocyclylalkyloxyalkyl are optionally substituted with one, two, or three substituents independently selected from alkyl, halo, haloalkyl, haloalkoxy, alkoxy, alkoxyalkyl, cyano, and hydroxy), or -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy), provided that when R^{d} is alkenyl, haloalkenyl, alkylidenyl, haloalkylidenyl, or alkoxyalkylidenyl, then R^{e} is other than hydrogen; and
R^{f} is hydrogen, alkyl, halo, alkoxy, alkoxyalkyl, or hydroxy; and
R⁵ is -Q-R⁴⁴ where Q is bond, alkylene, or —C(═O)—; and R⁴⁴ is cycloalkyl, fused cycloalkyl, fused spirocycloalkyl, aryl, aralkyl, heteroaryl, fused heteroaryl, or heteroaralkyl wherein aryl, aryl in aralkyl, heteroaryl, fused heteroaryl, and heteroaryl in heteroaralkyl are independently substituted with R^{aa}, R^{bb}, R^{cc} and R^{dd} wherein R^{aa} and R^{bb} are each independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkenyl, cyanoalkynyl, or halo, and R^{dd} is hydrogen, alkyl, alkylthio, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or
a pharmaceutically acceptable salt thereof.
82b. In embodiment 82b, the compound of embodiment 82a, or a pharmaceutically acceptable salt thereof, is wherein

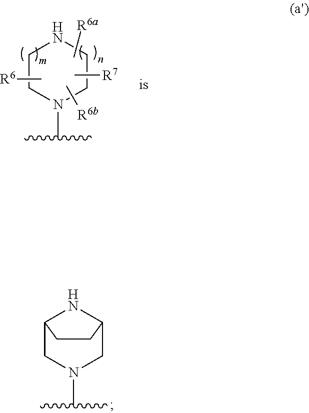

and the bicyclic heterocyclylalkyl is a ring of formula:

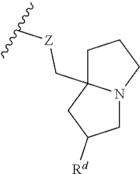

substituted with R^{e} and R^{f} and where R^{d} is alkylidenyl.
82c. In embodiment 82c, the compound of embodiment 82a or 82b, or a pharmaceutically acceptable salt thereof, is wherein the bicyclic heterocyclylalkyl is a ring of formula:

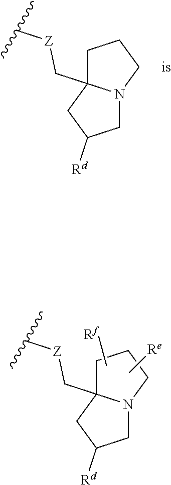

wherein R^{d} is methylidenyl and Z is —O—.
82d. In embodiment 82d, the compound of embodiment 82a to 82c, or a pharmaceutically acceptable salt thereof, is wherein R^{e} is -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy).
82e. In embodiment 1e, the compound of embodiment 82a to 82c, or a pharmaceutically acceptable salt thereof, is wherein R^{e} is -(alkylene)-OC(O)R³⁹ where alkylene is methylene or ethylene and R³⁹ is methylamino, dimethylamino, or diethylamino.
82f. In embodiment 1f, the compound of embodiment 82a to 1c, or a pharmaceutically acceptable salt thereof, is wherein R^{e} is -(alkylene)-OC(O)R³⁹ where alkylene is methylene or ethylene and R³⁹ is pyrrolidin-1-yl, piperidin-1-yl, or morpholin-4-yl, each ring optionally substituted with one or two substituents independently selected from methyl, fluoro, trifluoromethyl, trifluoromethoxy, and methoxy.
82g. In embodiment 1g, the compound of embodiment 82a to 82c, or a pharmaceutically acceptable salt thereof, is wherein R^{e} is hydrogen, alkyl, halo, haloalkyl, haloalkoxy, alkoxy, hydroxy, cycloalkyl, cycloalkylalkyl, heterocyclyl, or heterocyclylalkyl, (where cycloalkyl, by itself or as part of cycloalkylalkyl, and heterocyclyl, by itself or as part of heterocyclylalkyl, are optionally substituted with one, two, or three substituents independently selected from alkyl, halo, haloalkyl, haloalkoxy, alkoxy, alkoxyalkyl, cyano, and hydroxy).
82h. In embodiment 1h, the compound of embodiment 82a to 82c, or a pharmaceutically acceptable salt thereof, is wherein R^{e} is hydrogen, methyl, fluoro, chloro, difluoromethyl, trifluoromethyl, difluoromethoxy, trifluoromethoxy, methoxy, ethoxy, hydroxy, cyclopropyl, cyclopropylmethyl, pyrrolidinyl, pyrrolidinylmethyl, pyrrolidinylethyl, piperidinyl, piperidinylmethyl, piperidinylethyl, piperizinyl, piperizinylmethyl, or piperizinylethyl (wherein cyclopropyl, by itself or as part of cyclopropylmethyl, pyrrolidinyl, by itself or as part of pyrrolidinylmethyl and pyrrolidinylethyl, piperidinyl, by itself or as part of piperidinylmethyl and piperidinylethyl, and piperazinyl, by itself or as part of piperazinylmethyl and piperazinylethyl are optionally substituted with one or two substituents independently selected from methyl, chloro, fluoro, trifluoromethyl, trifluoromethoxy, methoxy, methoxymethyl, and cyano).
82i. In embodiment 82i, the compound of embodiment 82a to 82c, or a pharmaceutically acceptable salt thereof, is wherein R^{e} is alkoxyalkyl, alkoxyalkoxy, alkoxyalkyloxyalkyl, cycloalkoxy, cycloalkoxyalkyl, cycloalkylalkyloxy, cycloalkylalkyloxyalkyl, cycloalkylalkyloxyalkyloxy, bridged cycloalkyloxy, bridged cycloalkyloxyalkyl, bridged cycloalkylalkyloxy, bridged cycloalkylalkyloxyalkyl, heterocyclyloxy, heterocyclyloxyalkyl, heterocyclylalkyloxy, heterocyclylalkyloxyalkyl (where cycloalkyl as part of cycloalkoxy, cycloalkylalkyloxy, cycloalkoxyalkyl, cycloalkylalkyloxyalkyl, and cycloalkylalkyloxyalkyloxy, bridged cycloalkyl, as part of bridged cycloalkyloxy, bridged cycloalkyloxyalkyl, bridged cycloalkylalkyloxy, and bridged cycloalkylalkyloxyalkyl, and heterocyclyl as part of heterocyclyloxy, heterocyclyloxyalkyl, heterocyclylalkyloxy, and heterocyclylalkyloxyalkyl, are optionally substituted with one, two, or three substituents independently selected from alkyl, halo, haloalkyl, haloalkoxy, alkoxy, alkoxyalkyl, cyano, and hydroxy).
82j. In embodiment 82j, the compound of embodiment 82a to 82c, or a pharmaceutically acceptable salt thereof, is wherein R^{e} is methoxymethyl, methoxyethyl, methoxymethyloxy, methoxyethyloxy, methoxymethyloxymethyl, or methoxyethyloxymethyl.
82k. In embodiment 82k, the compound of embodiment 82a to 82c, or a pharmaceutically acceptable salt thereof, is wherein R^{e} is cyclopropyloxy, cyclopropylmethyloxy, cyclopropyloxymethyl, cycloalkyloxyethyloxy, or cyclopropylmethyloxymethyl (wherein cyclopropyl as part of cyclopropyloxy, cyclopropyloxymethyl, cyclopropylmethyloxy, cycloalkyloxyethyloxy, and cyclopropylmethyloxymethyl are optionally substituted with one or two substituents independently selected from methyl, chloro, fluoro, trifluoromethyl, trifluoromethoxy, methoxy, methoxymethyl, and cyano).
82l. In embodiment 82l, the compound of embodiment 82a to 82k, or a pharmaceutically acceptable salt thereof, is wherein R^{f} is hydrogen, methyl, ethyl, methoxy, ethoxy, methyloxy, ethyloxy, chloro, or fluoro.
82m. In embodiment 82m, the compound of embodiment 82a to 82k, or a pharmaceutically acceptable salt thereof, is wherein R^{f} is hydrogen.
82n. In embodiment 82n, the compound of embodiment 82a to 82c, or a pharmaceutically acceptable salt thereof, is wherein

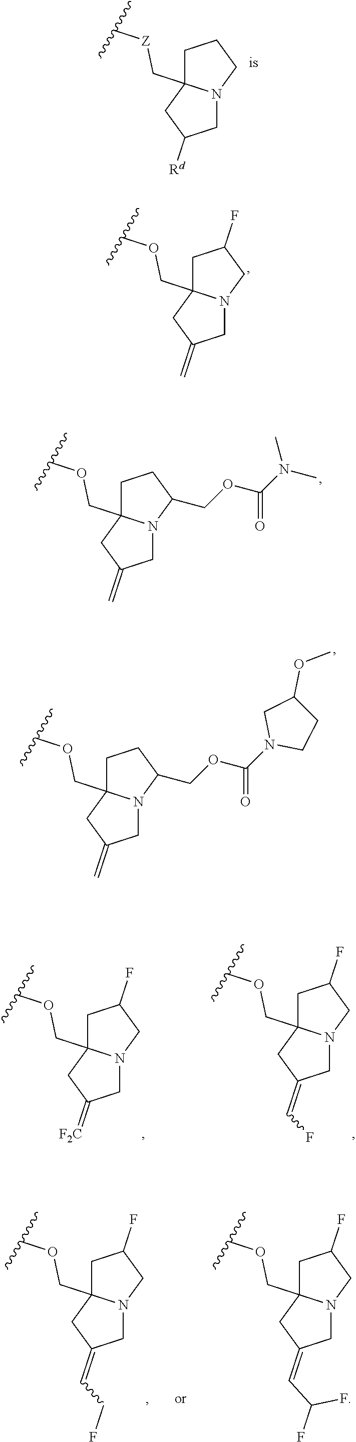

82o. In embodiment 82o, the compound of embodiment 82a to 82n, or a pharmaceutically acceptable salt thereof, is a compound having a structure of formula (I′d):

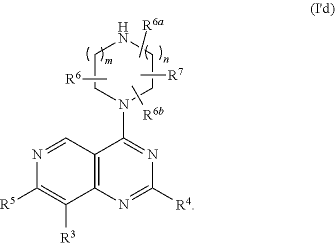

82a1. In embodiment 82a1, the compound of any one of embodiments 1 to 82a and 82c to 82o, or a pharmaceutically acceptable salt thereof, is wherein m and n are each 1, or one of m and n is 1 and the other of m and n is 2.
82b1. In embodiment 82b1, the compound of any one of embodiments 1 to 82a and 82c to 82o, or a pharmaceutically acceptable salt thereof, is wherein m and n are each 1, or one of m and n is 1.
82c1. In embodiment 82c1, the compound of any one of embodiments 1 to 82a and 82c to 82o, or a pharmaceutically acceptable salt thereof, is wherein one of m and n is 1 and the other of m and n is 2.
82d1. In embodiment 82d1, the compound of any one of embodiments 1 to 82a and 82c to 82o, or a pharmaceutically acceptable salt thereof, is wherein m and n are each 1.
82e1. In embodiment 82e1, the compound of any one of embodiments 1 to 82a and 82c to 82o, or a pharmaceutically acceptable salt thereof, is wherein m is 1 and n is 3.
82f1. In embodiment 82f1, the compound of any one of embodiments 1 to 82a and 82c to 82e1, or a pharmaceutically acceptable salt thereof, is wherein R⁶ and R⁷ are independently selected from hydrogen, methyl, and ethyl, and R^{6a} and R^{6b} are hydrogen.
82g1. In embodiment 82g1, the compound of any one of embodiments 1 to 82a and 82c to 82e1, or a pharmaceutically acceptable salt thereof, is wherein R⁶ is cyanomethyl and R⁷ is hydrogen, methyl, or ethyl, preferably R⁷ is hydrogen, and R^{6a} and R^{6b} are hydrogen.
82h1. In embodiment 82h1, the compound of any one of embodiments 1 to 82a and 82c to 82e1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably z is 2, and R^{6a} and R^{6b} are hydrogen.
82i1. In embodiment 82i1, the compound of any one of embodiments 1 to 82a and 82c to 82e1, or a pharmaceutically acceptable salt thereof, is wherein R⁶ and R⁷ are attached to the carbon atoms of the ring that are opposite or diagonal to each other and combine to form —CH═CH— and R^{6a} and R^{6b} are hydrogen.
82j 1. In embodiment 82j 1, the compound of any one of embodiments 1 to 82a and 82c to 82e1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, R^{6b} is hydrogen and R^{6a} is attached to a carbon of the —(CH₂)_{z}— group and is alkylidenyl, preferably =CH₂.
82k1. In embodiment 82k, the compound of any one of embodiments 1 to 82a and 82c to 82e1, or a pharmaceutically acceptable salt thereof, is wherein R⁶ and R⁷ are attached to the carbon atoms of the ring that are opposite or diagonal to each other and combine to form —(CH₂)_{z}— where z is 1, 2, or 3, preferably 2, and R^{6a} and R^{6b} are attached to the same carbon of the —(CH₂)_{z}— group and are combined to form cycloalkylene, preferably 1,1-cyclopropylene.
82l1. In embodiment 82l1, the compound of any one of embodiments 1 to 82a and 82c to 82o, or a pharmaceutically acceptable salt thereof, is wherein R¹ is:

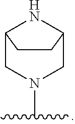 [SMILES: *N1CC2CCC(C1)N2]

82m1. In embodiment 82m1, the compound of any one of embodiments 1 to 82a and 82c to 82o, or a pharmaceutically acceptable salt thereof, is wherein R¹ is:

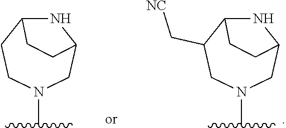 [SMILES: *N1CCC2CCC(C1)N2 ; *N1CC2CCC(N2)C(CC#N)C1]

82n1. In embodiment 82n1, the compound of any one of embodiments 1 to 82a and 82c to 82o, or a pharmaceutically acceptable salt thereof, is wherein R¹ is:

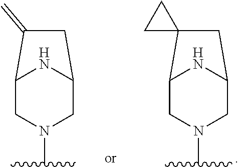

82o1. In embodiment 82o1, the compound of any one of embodiments 1 to 82a and 82c to 82o, or a pharmaceutically acceptable salt thereof, is wherein R¹ is:

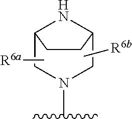

where R^{6a} and R^{6b} are other than hydrogen.
82p1. In embodiment 82p1, the compound of any one of embodiments 1 to 82a and 82c to 82o, or a pharmaceutically acceptable salt thereof, is wherein R¹ is:

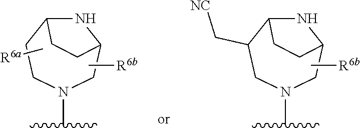

where R^{6b} is other than hydrogen.
82q1. In embodiment 82q1, the compound of any one of embodiments 1 to 82a and 82c to 82o, or a pharmaceutically acceptable salt thereof, is wherein R¹ is:

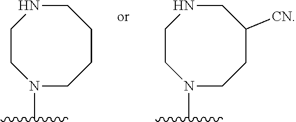 [SMILES: *N1CCCCNCC1 ; *N1CCNCC(C#N)CC1]

82r1. In embodiment 82r1, the compound of any one of embodiments 1 to 82a and 82c to 82o, or a pharmaceutically acceptable salt thereof, is wherein R¹ is:

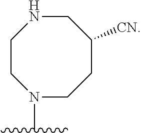 [SMILES: *N1CCNC[C@H](C#N)CC1]

83. In embodiment 83, the compound of any one of embodiments 1 to 81, or a pharmaceutically acceptable salt thereof, is wherein R¹ is a ring of formula

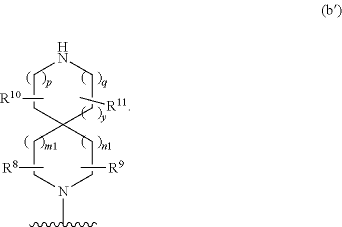

83a. In embodiment 83a, the compound of any one of embodiments 1 to 81 and 83, or a pharmaceutically acceptable salt thereof, is wherein each of m1, n1, p, q, and y is 1.
83b. In embodiment 83b, the compound of any one of embodiments 1 to 81 and 83, or a pharmaceutically acceptable salt thereof, is wherein each of m1, n1, p, and q is 0 and y is 1.
83c. In embodiment 83c, the compound of any one of embodiments 1 to 81 and 83, or a pharmaceutically acceptable salt thereof, is wherein each of m1 and n1 are 1, p is 0, 1, or 2, q is 0, and y is 0 or 1.
83d. In embodiment 83d, the compound of any one of embodiments 1 to 81 and 83, or a pharmaceutically acceptable salt thereof, is wherein m1 is 0 or 1, n1 is 0, p is 0, 1, or 2, q is 0 and y is 0.
83e. In embodiment 83e, the compound of any one of embodiments 1 to 81 and 83 to 83d, or a pharmaceutically acceptable salt thereof, is wherein R⁸, R⁹, R¹⁰ and R¹¹ are hydrogen.
83f. In embodiment 83f, the compound of any one of embodiments 1 to 81 and 83 to 83d, or a pharmaceutically acceptable salt thereof, is wherein one or two of R⁸, R⁹, R¹⁰ and R¹¹ are independently methyl, or ethyl and the other of R⁸, R⁹, R¹⁰ and R¹¹ are hydrogen.
83g. In embodiment 83g, the compound of any one of embodiments 1 to 81 and 83 to 83d, or a pharmaceutically acceptable salt thereof, is wherein one of R⁸ and R¹⁰ is cyanomethyl and the other is hydrogen and R⁹ and R¹¹ are hydrogen.
84. In embodiment 84, the compound of any one of embodiments 1 to 81, or a pharmaceutically acceptable salt thereof, is wherein R¹ is a ring of formula

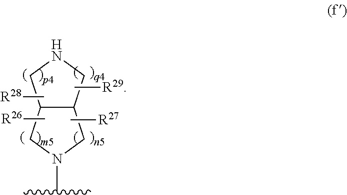

84a. In embodiment 84a, the compound of any one of embodiments 1 to 81 and 84, or a pharmaceutically acceptable salt thereof, is wherein each m5, n5, p4 and q4 is 1.
84b. In embodiment 84b, the compound of any one of embodiments 1 to 81 and 84, or a pharmaceutically acceptable salt thereof, is wherein m5 is 1, n5 is 0, p4 is 0, and q4 is 2.
84c. In embodiment 84c, the compound of any one of embodiments 1 to 81 and 84, or a pharmaceutically acceptable salt thereof, is wherein one of m5 and n5 is 1 or 2 and the other of m5 and n5 is 0, 1, or 2; and one of p4 and q4 is 1 or 2 and the other of p4 and q4 is 0, 1, or 2.
84d. In embodiment 84d, the compound of any one of embodiments 1 to 81 and 84 to 84c, or a pharmaceutically acceptable salt thereof, is wherein R²⁶ to R²⁹ are each hydrogen.
84e. In embodiment 84e, the compound of any one of embodiments 1 to 81 and 84 to 84c, or a pharmaceutically acceptable salt thereof, is wherein one or two of of R²⁶ to R²⁹ are methyl and the other of R²⁶ to R²⁹ are hydrogen.
84f. In embodiment 84f, the compound of any one of embodiments 1 to 81 and 84 to 84c, or a pharmaceutically acceptable salt thereof, is wherein R²⁸ is cyano and is attached to the bridgehead carbon that is adjacent to ring N attaching ring (f′) to the remainder of the compound of Formula (IIA′) or (II′), and R²⁶, R²⁷, and R²⁹ are hydrogen.
84g. In embodiment 84g, the compound of any one of embodiments 1 to 81 and 84, or a pharmaceutically acceptable salt thereof, is wherein R¹ is:

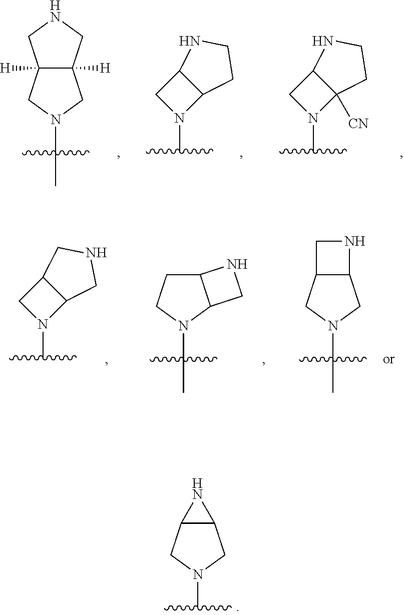

85. In embodiment 85, the compound of any one of embodiments 1 to 81, or a pharmaceutically acceptable salt thereof, is wherein R¹ is a ring of formula:

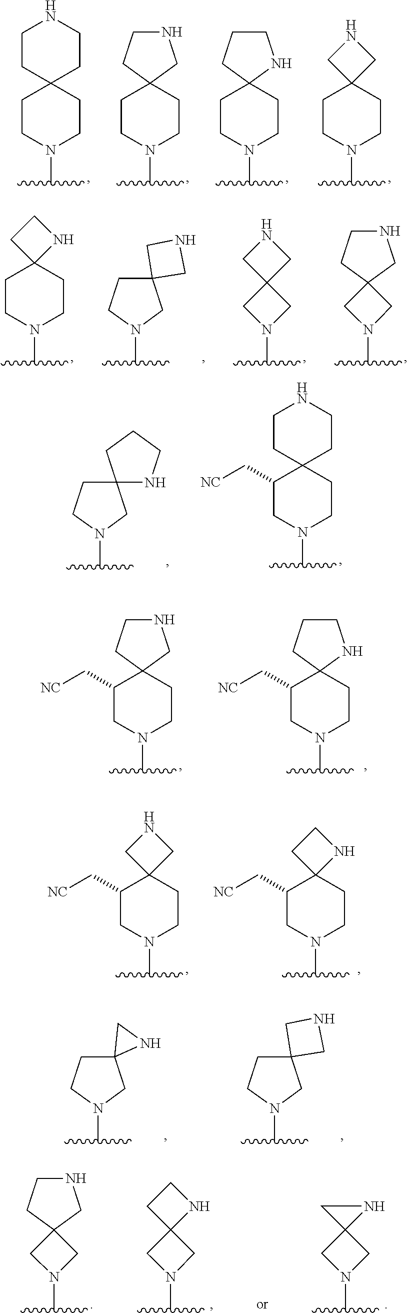

86. In embodiment 86, the compound of any one of embodiments 1 to 81, or a pharmaceutically acceptable salt thereof, is wherein R¹ is a ring of formula:

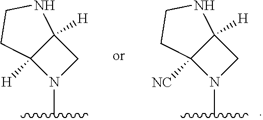 [SMILES: *N1C[C@@H]2NCC[C@@H]21 ; *N1C[C@@H]2NCC[C@@]21C#N]

87. In embodiment 87, the compound of any one of embodiments 1 to 81, or a pharmaceutically acceptable salt thereof, is wherein R¹ is a ring of formula

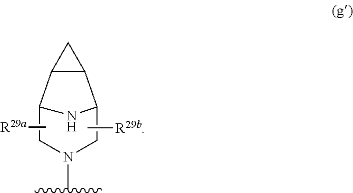

In a subembodiment, R^{29a} and R^{29b} are hydrogen.
88. In embodiment 88, the compound of Formula (I) of any one of embodiments 1 to 82, and 82a1 to 87, or a pharmaceutically acceptable salt thereof, has a structure of formula (I′a) as follows:

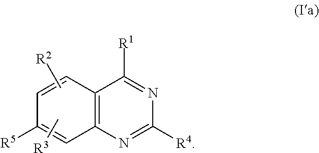

89. In embodiment 89, the compound of Formula (I) of any one of embodiments 1 to 82, and 82a1 to 87, or a pharmaceutically acceptable salt thereof, has a structure of formula (Ib′) or (IIb′) as follows:

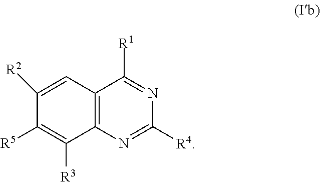

90. In embodiment 90, the compound of Formula (I) of any one of embodiments 1 to 82, and 82a1 to 87, or a pharmaceutically acceptable salt thereof, has a structure of formula (I′c) as follows:

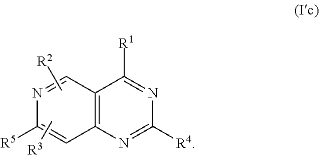

91. In embodiment 91, the compound of Formula (I) of any one of embodiments 1 to 82, and 82a1 to 87, or a pharmaceutically acceptable salt thereof, has a structure of formula (I′d) as follows:

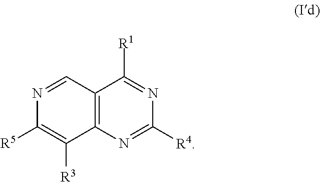

92. In embodiment 92, the compound of Formula (I) of any one of embodiments 1 to 82, and 82a1 to 87, or a pharmaceutically acceptable salt thereof, has a structure of formula (I′e) as follows:

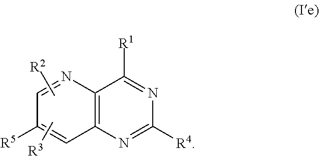

93. In embodiment 93, the compound of Formula (I) of any one of embodiments 1 to 82, and 82a1 to 87, or a pharmaceutically acceptable salt thereof, has a structure of formula (I′f) as follows:

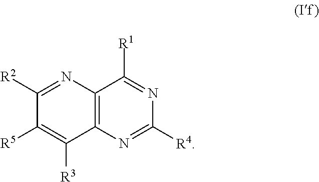

94. In embodiment 94, the compound of any one of embodiments 1 to 93, or a pharmaceutically acceptable salt thereof, is wherein R⁵ is -Q-R⁴⁴ where Q is bond and R⁴⁴ is cycloalkyl, fused cycloalkyl, fused spirocycloalkyl, aryl, heteroaryl, or fused heteroaryl, wherein aryl, heteroaryl, and fused heteroaryl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkenyl, cyanoalkynyl, or halo, and R^{dd} is hydrogen, alkyl, cycloalkyl, halo, haloalkyl, haloalkoxy, alkoxy, alkylthio, heteroalkyl, hydroxyalkyl, amino, cyano, optionally substituted aryl, optionally substituted heteroaryl, optionally substituted heterocyclyl, or optionally substituted heterocyclylalkyl.
95. In embodiment 95, the compound of any one of embodiments 1 to 93, or a pharmaceutically acceptable salt thereof, is wherein R⁵ is -Q-R⁴⁴ where Q is alkylene and R⁴⁴ is cycloalkyl, aryl, or fused heteroaryl, wherein aryl, fused heteroaryl, and heteroaryl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.
96. In embodiment 96, the compound of any one of embodiments 1 to 93, or a pharmaceutically acceptable salt thereof, is wherein R⁵ is -Q-R⁴⁴ where Q is —C(O)— and R⁴ is cycloalkyl, aryl, fused heteroaryl, or heteroaryl, wherein aryl, fused heteroaryl and heteroaryl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.
97. In embodiment 97, the compound of any one of embodiments 1 to 93, or a pharmaceutically acceptable salt thereof, is wherein R⁴⁴ is cycloalkyl, fused cycloalkyl, aryl, or heteroaryl wherein aryl, and heteroaryl are substituted with R^{aa}, R^{bb}, R^{cc} and R^{dd} wherein R^{aa} and R^{bb} are independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkenyl, cyanoalkynyl, or halo, and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.
98. In embodiment 98, the compound of any one of embodiments 1 to 93, or a pharmaceutically acceptable salt thereof, is wherein R⁵ is -Q-R⁴⁴ where Q is bond and R⁴ is phenyl or naphthyl substituted with R^{aa}, R^{bb}, R^{cc} and R^{dd}.
99. In embodiment 99, the compound of any one of embodiments 1 to 93, or a pharmaceutically acceptable salt thereof, is wherein R⁵ is -Q-R⁴⁴ where Q is bond and R⁴ is phenyl or naphthyl substituted with R^{aa}, R^{bb}, and R^{dd} where R^{aa} and R^{bb} are independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.
100. In embodiment 100, the compound of any one of embodiments 1 to 99, or a pharmaceutically acceptable salt thereof, is wherein R^{aa} and R^{bb} independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanovinyl, 2-cyanoethyn-1-yl, or fluoro, and R^{dd} is hydrogen, methyl, fluoro, amino, or cyclopropyl.
101. In embodiment 101, the compound of any one of embodiments 1 to 93, or a pharmaceutically acceptable salt thereof, is wherein R⁵ is -Q-R⁴⁴ where Q is bond and R⁴ is heteroaryl or fused heteroaryl substituted with R^{aa}, R^{bb}, R^{cc} and R^{dd}.
102. In embodiment 102, the compound of any one of embodiments 1 to 93 and 101, or a pharmaceutically acceptable salt thereof, is wherein R⁵ is -Q-R⁴⁴ where Q is bond and R³⁶ is a monocyclic heteroaryl (e.g., pyridyl, pyrimidinyl) substituted with R^{aa}, R^{bb}, R^{cc} and R^{dd}.
103. In embodiment 103, the compound of any one of embodiments 1 to 93 and 101, or a pharmaceutically acceptable salt thereof, is wherein R⁵ is -Q-R³⁶ where Q is bond and R³⁶ is bicyclic heteroaryl (e,g, quinolinyl, isoquinolinyl, or indazolyl), substituted with R^{aa}, R^{bb}, R^{cc} and R^{dd}.
104. In embodiment 104, the compound of any one of embodiments 1 to 93 and 101 to 103, or a pharmaceutically acceptable salt thereof, is wherein the heteroaryl is substituted with R^{aa}, R^{bb}, and R^{dd} where R^{aa} and R^{bb} independently selected from hydrogen, alkyl, halo, haloalkyl, hydroxy, alkoxy, haloalkoxy, cycloalkyl, amino, cyano, and hydroxyalkyl and R^{dd} is hydrogen, alkyl, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl.
105. In embodiment 105, the compound of any one of embodiments 1 to 93 and 101 to 103, or a pharmaceutically acceptable salt thereof, is wherein R^{aa} and R^{bb} are independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen or fluoro, and R^{dd} is hydrogen, methyl, fluoro, amino, or cyclopropyl.
106. In embodiment 106, the compound of any one of embodiments 1 to 93, or a pharmaceutically acceptable salt thereof, is wherein R⁵ is -Q-R⁴⁴ where Q is bond and R⁴ is:

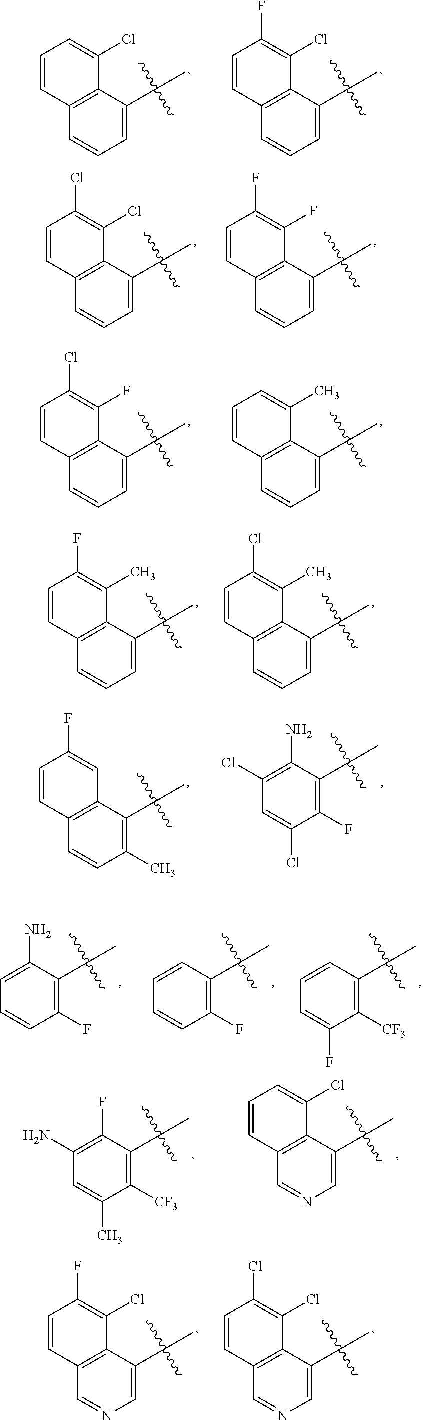

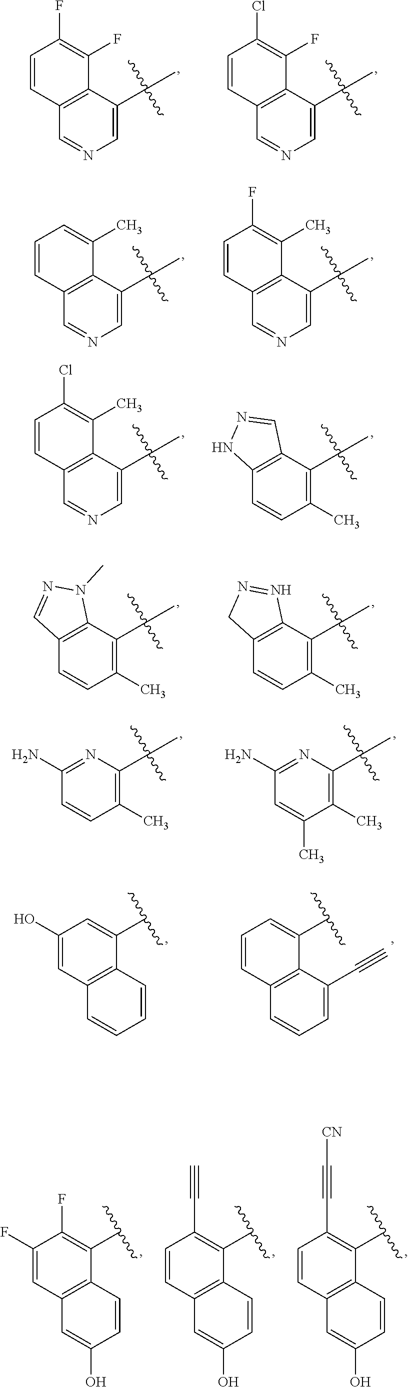

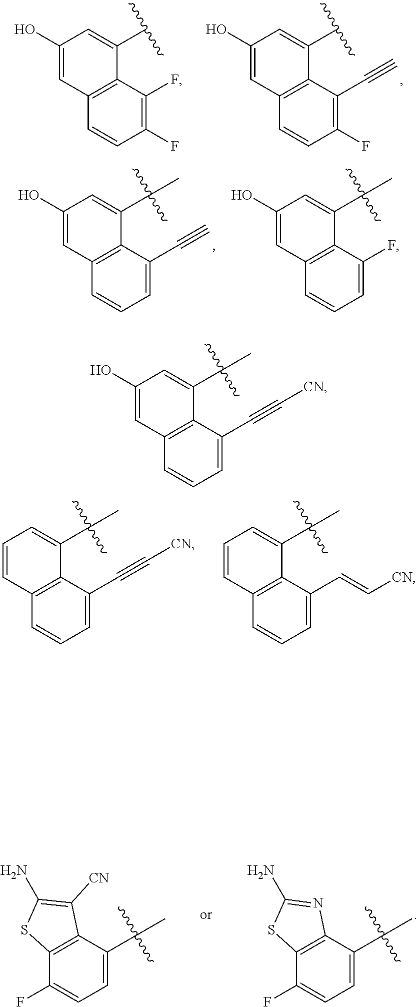

106a. In embodiment 106a, the compound of any one of embodiments 1 to 93, or a pharmaceutically acceptable salt thereof, is wherein R⁵ is -Q-R⁴⁴ where Q is bond and R⁴ is:

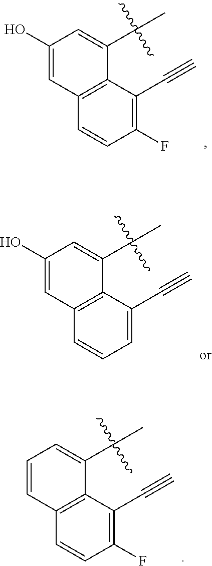 [SMILES: *c1cc(O)cc2ccc(F)c(C#C)c12 ; *c1cc(O)cc2cccc(C#C)c12 ; *c1cccc2ccc(F)c(C#C)c12]

106b. In embodiment 106b, the compound of any one of embodiments 1 to 93 and 106a, or a pharmaceutically acceptable salt thereof, is wherein R⁵ is -Q-R⁴⁴ where Q is bond and R⁴ is:

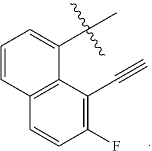 [SMILES: *c1cccc2ccc(F)c(C#C)c12]

107. In embodiment 107, the compound of any one of embodiments 1 to 106b, or a pharmaceutically acceptable salt thereof, is wherein R² is hydrogen, halo, or alkyl, and R³ is hydrogen, halo, cycloalkyloxy, or alkyl.
108. In embodiment 108, the compound of any one of embodiments 1 to 106b, or a pharmaceutically acceptable salt thereof, is wherein R² and R³ are each hydrogen.
109. In embodiment 109, the compound of any one of embodiments 1 to 106b, or a pharmaceutically acceptable salt thereof, is wherein R² is hydrogen or chloro and R³ is hydrogen, fluoro, or cyclopropyloxy, preferably R³ is fluoro.

### General Synthetic Scheme

Compounds Formula (I) can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds Formula (I) can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) where R¹ is, for example, a ring of formula (a′) where R^{6a} and R^{6b} are hydrogen, respectively, R⁴ is —O—R³⁰, and other groups are as defined in the Summary can be prepared as illustrated and described in Scheme 1 below.

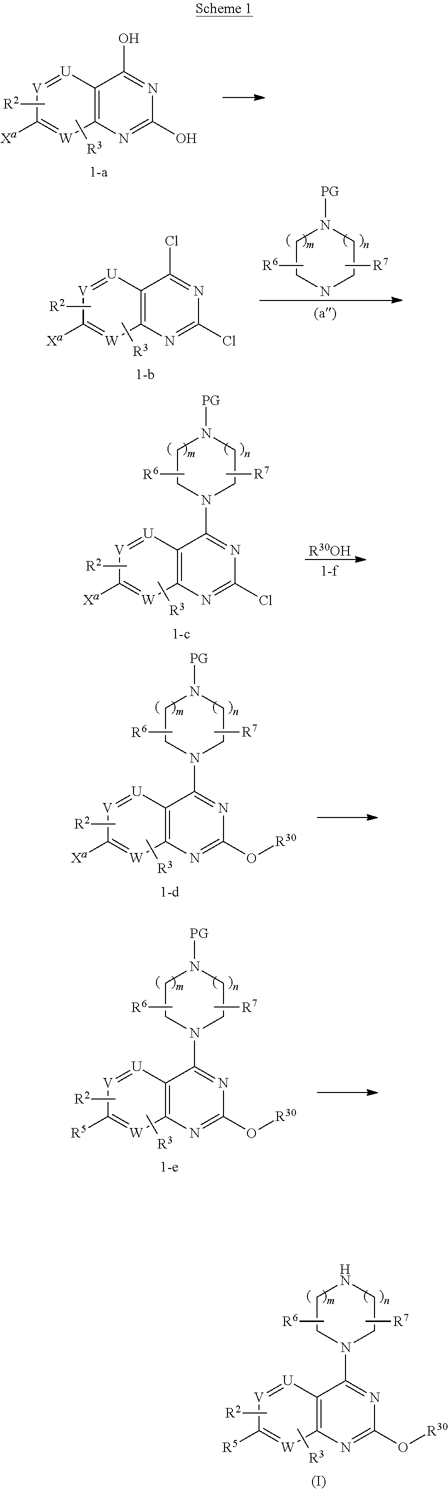

Chlorination of a compound of formula 1-a where X^{a} is a halogen, and other groups as defined in the Summary or a precursor group thereof (a precursor group as used herein is one that can be converted to a group covered by Formula (I). A representative example of conversion of a precursor group to a group covered by Formula (I) is illustrated and described in Scheme 3 below) with a suitable chlorination reagent such as POCl₃ optionally in presence of a base such as DIPEA provides a 2,4-dichloro compound of formula 1-b. Compounds of formula 1-a is either commercially available or they can be prepared by method well known in the art. Once such method is illustrated and described in Method A (i) and (ii) below.

Treatment of compound 1-b with an amine of formula (a″) where m, n, R⁶ and R⁷ are as defined in the Summary or a precursor group thereof and PG is a suitable amino protecting group such as Boc, CBz, and the like, in the presence of a base such as DEA or DBU and the like, provides a 2-chloro compound of formula 1-c. Displacement of chloro group at C-2 position in compound 1-c with an alcohol of formula 1-f where R³⁰ is as defined in the Summary or a precursor group thereof, provides a compound of formula 1-d. Alcohols of formula 1-f are either commercially available or can be made by methods known in the art. Representative methods for preparing compound of formula 1-f are described in methods (c) to (e) below. Compounds of formula 1-d where R⁴ is other than —O—R³⁰ can be prepared by methods well known in the art such as PCT application publication No. WO2019099524.

Amines of formula (a″) are either commercially available or can be made by methods known in the art. For example, benzyl 2-(cyanomethyl)piperazine-1-carboxylate, tert-butyl 2-(cyanomethyl)piperazine-1-carboxylate, benzyl 2,5-dimethylpiperazine-1-carboxylate, tert-butyl 2-methylpiperazine-1-carboxylate, tert-butyl piperazine-1-carboxylate, benzyl piperazine-1-carboxylate are commercially available. Others can be prepared by methods well known in the art.

Various R⁵ group, other than hydrogen, can be installed in compound 1-d by reacting compound 1-d and a suitable organometallic reagent of formula R⁵-M where R⁵ is cycloalkyl, aryl or heteroaryl as defined in the Summary and M is boronic acid, boronic ester, or stannane, under Suzuki, Negeshi, and Stille reaction conditions to provide a compound of formula 1-e.

Removal of amino protecting group PG in 1-e under standard reaction condition provides a compound of Formula (I). It will be apparent to a person of ordinary skilled in the art, that compounds of Formula (I) where R¹ is a group of formula (b′), (f′) or (g′) can be similarly prepared by using appropriate mono-protected amines. For example, compound of Formula (I) where R¹ is a ring of formula (b′) can be prepared using tert-butyl 2,7-diazaspiro[3.5]nonane-2-carboxylate, and tert-butyl 2,6-diazaspiro[3.4]octane-2-carboxylate. Other mono-protected amines can be prepared by methods disclosed in PCT application publication No. WO2019099524 or by methods well known in the art. Some such methods are described in Methods (a) and (b) below.

Compounds of formula 1-a can be prepared by methods well known in the art. For example,

1. Compounds of Formula 1-a where X^{a} is halogen, U is CH, V is N, W is CH, R² and R³ are as defined in the Summary (or any embodiments thereof) can be prepared as illustrated and described below.

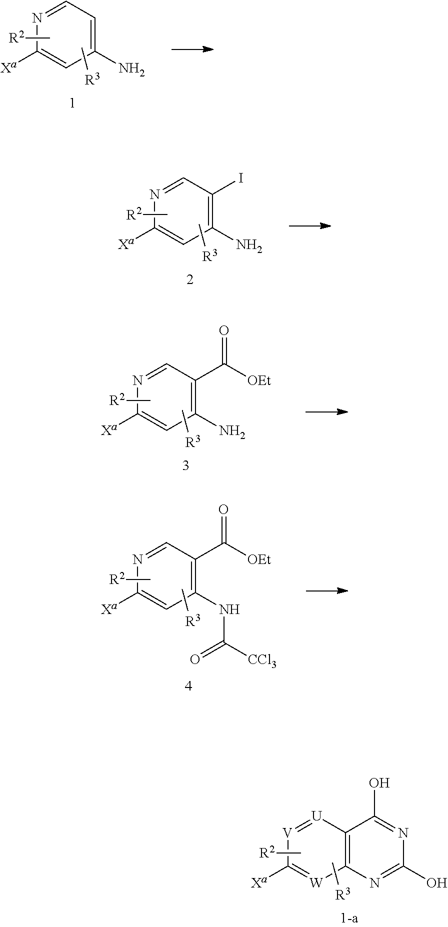

Iodination of a compound of formula 1 where X^{a} is a halo and R² and R³ are as defined in the Summary, with NIS and a suitable acid such as TsOH provides a compound of formula 2. The iodine in 2 can be converted to ethyl carboxylate under carbonylation condition including Pd catalyst such as Pd(PPh₃)₄ in carbon monoxide atmosphere and ethanol solvent to provide a compound of formula 3. Compound 3 can react with triphosgene to provide trichloroacetamido compound of formula 4, which upon treatment with ammonia in an organic solvent such as methanol, undergoes cyclization to provide compound of formula 1-a. Compounds of formula 1 are either commercially available or can be made by methods known in the art. For example, 2-chloro-3-fluoropyridin-4-amine and 2-chloropyridin-4-amine are commercially available.

2. Compounds of Formula 1-a where X^{a} is halogen, U, V and W are CH, R² and R³ are as defined in the Summary (or any embodiments thereof) can be by reacting a compound of formula

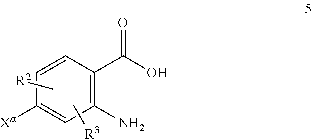

with urea at elevated temperature. Compounds of formula 5 are either commercially available or can be made by methods known in the art. For example, 2-amino-4-bromo-5-chloro-3-fluorobenzoic acid, 2-amino-4-bromo-3-fluorobenzoic acid and 2-amino-4-bromobenzoic acid are commercially available.

### Method (a):

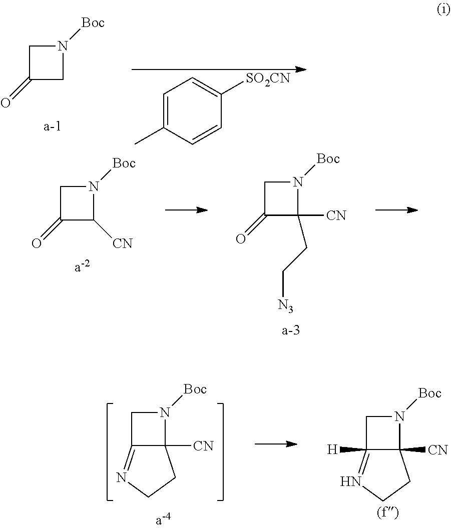

Deprotonation of Boc protected oxoazetidine a-1 with a strong base such as LDA followed by treatment with 4-methylbenzenesulfonyl cyanide affords CN substituted oxoazetidine a-2, which then is deprotonated with a base such as NaH, followed by alkylation with 1-azido-2-bromoethane provides bi-substituted oxoazetidine a-3. The azide group of a-3 can be reduced by catalytic hydrogenation to form NH₂ group which reacts with ketone group intramolecularly to form the imine intermediate a-4. Further reduction of imine group on a-4 by catalytical hydrogenation or sodium triacetoxyborohydride provides bicyclic amine a-5.

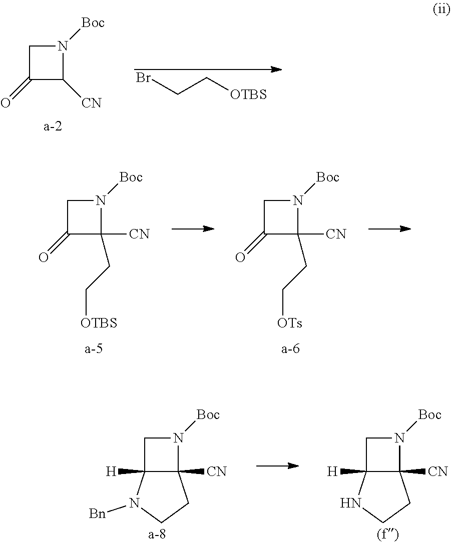 [SMILES: CC(C)(C)OC(=O)N1CC(=O)C1C#N ; CC(C)(C)[Si](C)(C)OCCBr ; CC(C)(C)OC(=O)N1CC(=O)C1(C#N)CCO[Si](C)(C)C(C)(C)C ; Cc1ccc(S(=O)(=O)OCCC2(C#N)C(=O)CN2C(=O)OC(C)(C)C)cc1 ; CC(C)(C)OC(=O)N1C[C@H]2N(Cc3ccccc3)CC[C@]21C#N ; CC(C)(C)OC(=O)N1C[C@H]2NCC[C@]21C#N]

Alternatively, compound a-2 can be deprotonated an then alkylated with (2-bromoethoxy)-(tert-butyl)dimethylsilane to afford the bi-substituted oxoazetidine a-5. Removal of the TBS group of a-5 under conditions well known the art, followed by conversion of the hydroxy into a suitable leaving group such as tosylate provides compound a-6. Reaction of a-6 with benzylamine under reductive amination conditions, followed by intramolecular cyclization of the resulting amine compound provides bicyclic amine a-8, which upon removal of the benzyl protecting group provides amine compound (f″).

### Method (b):

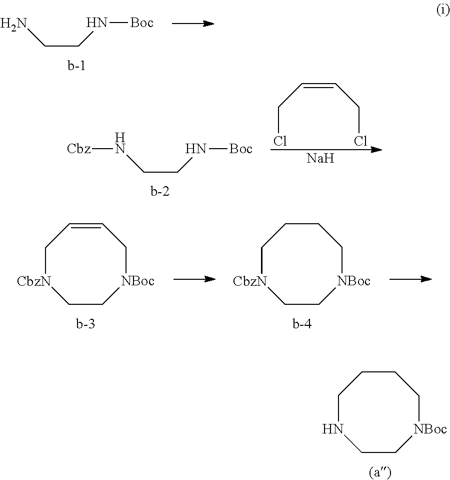

Protection of the free amine group of compound b-1 with Cbz group provides compound b-2. Deprotonation of b-2 with a strong base such as NaH, followed by cyclization with (Z)-1,4-dichlorobut-2-ene provides alkene b-3. The double bond of b-3 is then hydrogenated to produce 1,4-diazocane b-4, which upon removal of the Cbz group under conditions well known in the art, provides an amine of formula (a″).

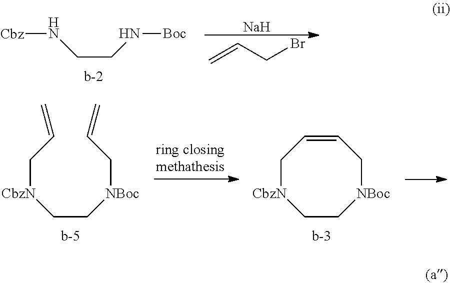

Alternatively, alkene b-3 can be made from diene b-5 by intramolecular ring closing metathesis. Compound b-5 can be prepared by treating compound b-2 with a deprotonating agent such as sodium hydride and treating the resulting deprotonated intermediate with allyl bromide. Compound b-3 can then be converted to compound (a″) as described above.

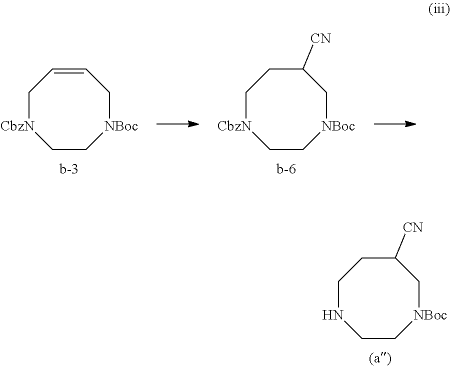 [SMILES: CC(C)(C)OC(=O)N1C/C=C\CN(C(=O)OCc2ccccc2)CC1 ; CC(C)(C)OC(=O)N1CCN(C(=O)OCc2ccccc2)CCC(C#N)C1 ; CC(C)(C)OC(=O)N1CCNCCC(C#N)C1]

Ni-catalyzed hydrocyanation of compound b-3 in the presence of zinc powder and formamide provides nitrile b-6, which upon removal of the Cbz group provides compound (a″).

Compounds of formula R³⁰—OH can be prepared by methods well know in the art. Some such methods are described below in Methods (c) to (e) below.

### Method (c):

R³⁰OH of formula

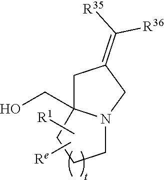

where t is 1 to 3, R^{e} is hydrogen, alkyl, fluoro, alkoxy, or hydroxy R^{f}, R³⁵, and R³⁶ are as defined in the Summary can be prepared as illustrated and described below.

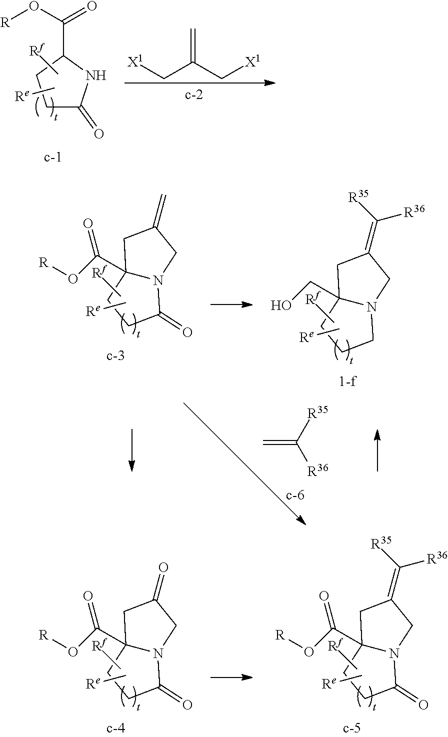

Compound of formula c-1 where t is 1 to 3, R is alkyl, R^{e} is hydrogen, alkyl, fluoro, alkoxy, or hydroxy and R^{f} is as defined in the Summary can undergo cyclization with a compound of formula c-2 where each X¹ is halo, e.g., 3-chloro-2-(chloromethyl)prop-1-ene, in the presence of a base such as LHMDS to provide a compound of formula c-3, which can be reduced with a suitable reducing reagent such as LiAlH₄ to afford a compound of formula 1-f where R³⁵ and R³⁶ are H. Compounds of formula c-2 are commercially available, or they can be prepared by methods well known in the art.

Compounds of formula 1-f where R³⁵ and R³⁶ are as defined in the Summary can be prepared by converting compound c-3 to corresponding ketone of formula c-4 under oxidative cleavage condition such as NaIO₄ and catalytic amount of RuCl₃. Treatment of ketone c-4 under standard Wittig olefination condition can provide olefin of formula c-5. Alternatively, compound c-3 can undergo cross metathesis with compound of formula c-6 in the presence of an olefin metathesis catalyst such as Hoveyda-Grubbs Catalyst® [CAS No. 301224-40-8] or Zhan catalyst [CAS No. 918870-76-5] to provide compound c-5. Reduction of the ester group of c-5 with a suitable reducing reagent such as LiAlH₄ provides a compound of formula 1-f.

### Method (d):

R³⁰OH of formula c-5,

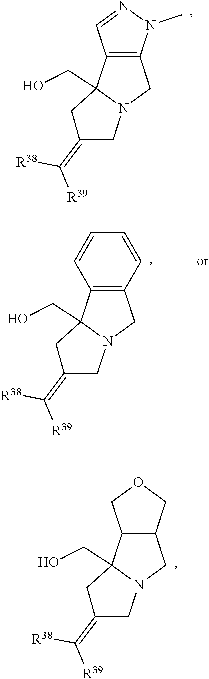

where phenyl and tetrahydrofuran moiety can be substituted with R^{h} and Rⁱ as defined in the Summary and R³⁸ and R³⁹ are as defined in the Summary and can be prepared as illustrated and described below.

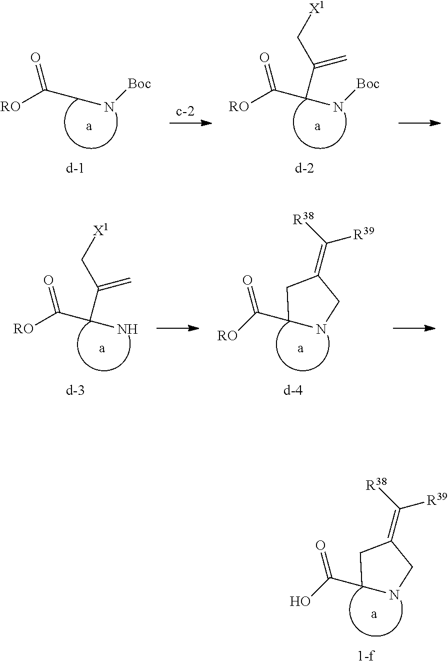

Treatment of a compound of formula d-1 where ring a is:

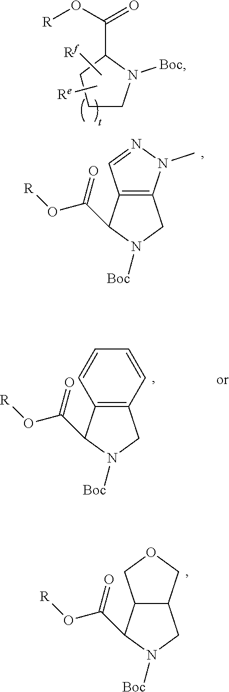

phenyl and tetrahydrofuran moiety can be substituted with R^{h} and Rⁱ as defined in the Summary and R is alkyl, with a base such as LHMDS, followed by treatment with a reagent of formula c-2 can provide a compound of formula d-2.

Removal of the Boc group in compound d-2 under acid condition such as HCl or TFA provides compound d-3 which can be converted to compound of formula 1-f where ring a is as defined above, as described in Method (c) above.

It will be readily apparent to a person skilled in the art that based on the nature of the R^{e} and R^{f} groups in a compound of formula d-4, the process illustrated above may include additional optional steps such as addition and removal of protecting groups and/or modification of R^{e} and/or R^{f} to other R^{e} and R^{f} groups as defined in the Summary. For example, compounds of formula d-4 where with R^{e} is hydroxyalkyl can be alkylated, followed by reduction ester group to provide a compound of formula 1-f.

Compounds of formula d-1 either commercially available or they can be prepared by methods well known in the art. For examples, ethyl 2,3-dihydro-1H-isoindole-1-carboxylate, ethyl 4,7dihydro-5,6-dimethyl-2H-isoindole-1-carboxylate, 2-(tert-butyl) 1-methyl isoindoline-1,2-dicarboxylate, 1H-furo[3,4-c]pyrrole-4,5(3H)-dicarboxylic acid, tetrahydro-, 5-(1,1-dimethylethyl) ester are commercially available.

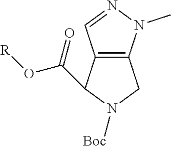

can be prepared as illustrated and described below.

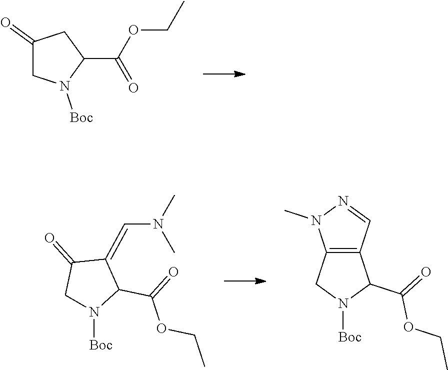 [SMILES: CCOC(=O)C1CC(=O)CN1C(=O)OC(C)(C)C ; CCOC(=O)C1/C(=C\N(C)C)C(=O)CN1C(=O)OC(C)(C)C ; CCOC(=O)C1c2cnn(C)c2CN1C(=O)OC(C)(C)C]

Reaction between 1-(tert-butyl) 2-ethyl 4-oxopyrrolidine-1,2-dicarboxylate and DMA in DMF solvent can provide 1-(tert-butyl) 2-ethyl (E)-3-((dimethylamino)methylene)-4-oxo-pyrrolidine-1,2-dicarboxylate, which can react with methylhydrazine to provide 5-(tert-butyl) 4-methyl 1-ethyl-4,6-dihydropyrrolo[3,4-c]pyrazole-4,5(1H)-dicarboxylate.

Alternatively, R³⁰OH of formula

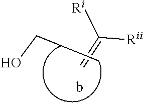

where ring b is an R³⁰ ring described where ═CRⁱRⁱⁱ are alkylidene, haloalkylidene, alkoxyalkylidene, ═CR³¹R³², ═CR³⁵R³⁶, ═CR³⁸R³⁹ or ═CR⁴¹R⁴² as defined in the Summary can be prepared as described below:

### Method (e)

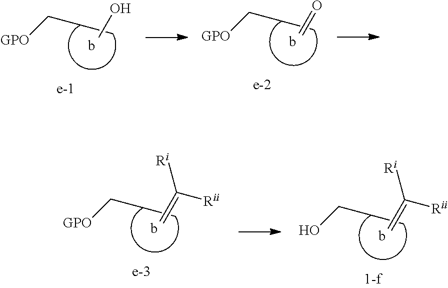

Treatment of a hydroxy compound of formula e-1 where PG is a suitable hydroxy protecting group such as alkylcarbonyl with a suitable oxidizing agent such as Dess-martin reagent or under Swern oxidations provides a keto compound of formula e-2, which can be coverted to alkylidene compound of formula e-3 as described in Method c above. Removal of the hydroxy protecting group then provides a compound of formula 1-f.

Compounds of Formula (I) where R¹ is, for example, a ring of formula (a′), R⁴ is —O—R³⁰, and other groups are as defined in the Summary can be prepared as illustrated and described in Scheme 2 below.

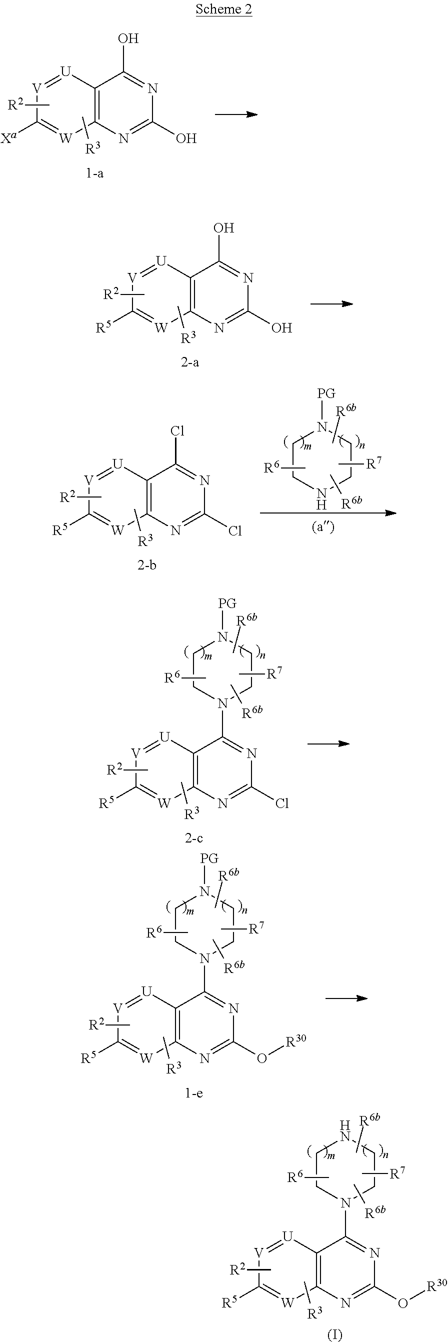

Coupling reaction between compound 1-a and a suitable organometallic reagent of formula R⁵-M where R⁵ is cycloalkyl, aryl or heteroaryl as defined in the Summary or a precursor group thereof and M is boronic acid, boronic ester, or stannane, under Suzuki, Negeshi, and Stille reaction conditions respectively, to provide a compound of formula 2-a. Chlorination of a compound of formula 2-a with a suitable chlorination reagent such as POCl₃ optionally in presence of a base such as DIPEA provides compound of formula 2-b. Compound 2-b is converted to a compound of Formula (I) as described in Scheme 1 above.

Compounds of Formula (I) where R⁴ is

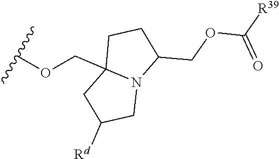

can be prepared by a precursor group of formula

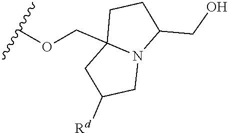

as illustrated and described in Scheme 3 below

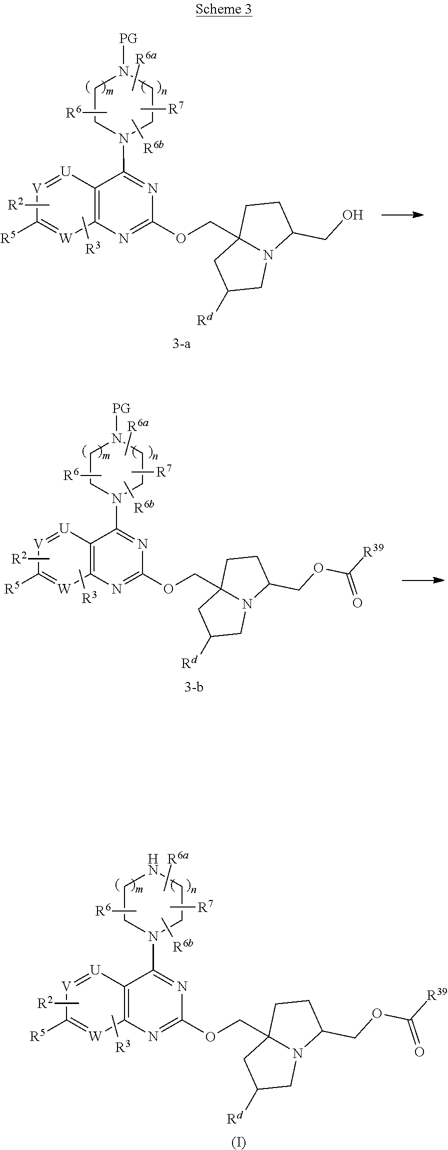

Treatment of a compound of formula 3-a with a coupling agent such as CDI, and the like, treatment of the resulting intermediate with an amine of formula HR³⁹, where R³⁹ is as defined in the Summary provides a compound of formula 3-b which can the converted to compound of Formula (I) as described above.

### Utility

The present disclosure provides treatment of cancer mediated by K-ras, in particular with K-ras G12D mutants. In some embodiments, the cancer is pancreatic cancer, colorectal cancer, lung cancer, gall bladder cancer, thyroid cancer, and bile duct cancer. In certain embodiments the lung cancer is a non-small cell lung carcinoma (NSCLC), for example adenocarcinoma, squamous-cell lung carcinoma or large-cell lung carcinoma. In some embodiments, the lung cancer is a small cell lung carcinoma. Other lung cancers treatable with the disclosed compounds include, but are not limited to, glandular tumors, carcinoid tumors and undifferentiated carcinomas.

K-ras G12D mutations are observed in hematological malignancies that affect blood, bone marrow, and/or lymph nodes. As such the compounds of Formula (I) or a pharmaceutically acceptable salt thereof can be used for the treatment of acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias, lymphomas such as all subtypes of Hodgkins lymphoma or non-Hodgkins lymphoma, plasma cell malignancies such as multiple myeloma, mantle cell lymphoma, and Waldenstrom's macroglubunemia.

The compounds of Formula (I), or a pharmaceutically acceptable salt thereof can be used for the treatment of a hyperproliferative disorder or metastasis in human who suffers from a cancer such as acute myeloid leukemia, cancer in adolescents, adrenocortical carcinoma childhood, AIDS related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. The compounds of Formula (I), or a pharmaceutically acceptable salt thereof can also be used for the treatment of a non-cancerous hyperproliferative disorder such as benign hyperplasia of the skin (e.g., psoriasis), restenosis, or prostate (e.g., benign prostatic hypertrophy (BPH)).

### Testing

The K-Ras G12D activity of the compounds of Formula (I), or a pharmaceutically acceptable salt thereof can be tested using the in vitro assay described in Biological Examples 1 below.

### Pharmaceutical Compositions

In general, the compounds Formula (I) (unless stated otherwise, reference to compound/compounds of Formula (I) herein includes any embodiments thereof described herein or a pharmaceutically acceptable salt thereof) will be administered in a therapeutically effective amount by any of the accepted modes of administration for agents that serve similar utilities. Therapeutically effective amounts of compounds Formula (I) may range from about 0.01 to about 500 mg per kg patient body weight per day, which can be administered in single or multiple doses. A suitable dosage level may be from about 0.1 to about 250 mg/kg per day; about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions can be provided in the form of tablets containing about 1.0 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount of the compound Formula (I), i.e., the active ingredient, will depend upon numerous factors such as the severity of the disease to be treated, the age and relative health of the patient, the potency of the compound being utilized, the route and form of administration, and other factors.

In general, compounds Formula (I) will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance.

The compositions are comprised of in general, a compound of Formula (I) in combination with at least one pharmaceutically acceptable excipient. Acceptable excipients are generally non-toxic, aid administration, and do not adversely affect the therapeutic benefit of the compound of Formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

The compounds of Formula (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds of Formula (I) may also be formulated as a depot preparation. Such long -acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds of Formula (I) may also be formulated in rectal compositions such as suppositories or retention enemas, e.g., containing conventional suppository bases such as cocoa butter, polyethylene glycol, or other glycerides.

Certain compounds of Formula (I) may be administered topically, that is by non-systemic administration. This includes the application of a compound of Formula (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.10% to T % w/w of the formulation.

For administration by inhalation, compounds of Formula (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds of Formula (I) may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator. Other suitable pharmaceutical excipients and their formulations are described in Remington's Pharmaceutical Sciences, edited by E. W. Martin (Mack Publishing Company, 20th ed., 2000).

The level of the compound of Formula (I) in a formulation can vary within the full range employed by those skilled in the art. Typically, the formulation will contain, on a weight percent (wt. %) basis, from about 0.01-99.99 wt. % of a compound of Formula (I) based on the total formulation, with the balance being one or more suitable pharmaceutical excipients. For example, the compound is present at a level of about 1-80 wt. %.

### Combinations and Combination Therapies

The compounds of Formula (I) or a pharmaceutically acceptable salt thereof may be used in combination with one or more other drugs in the treatment of diseases or conditions for which compounds of Formula (I) or the other drugs may have utility. Such other drug(s) may be administered, by a route and in an amount commonly used therefore, contemporaneously or sequentially with a compound of Formula (I) or a pharmaceutically acceptable salt thereof. When a compound of Formula (I) or a pharmaceutically acceptable salt thereof is used contemporaneously with one or more other drugs, a pharmaceutical composition in unit dosage form containing such other drugs and the compound of Formula (I) or a pharmaceutically acceptable salt thereof can be used. Accordingly, the pharmaceutical compositions of the present disclosure also include those that contain one or more other drugs, in addition to a compound of Formula (I) or a pharmaceutically acceptable salt thereof. The combination therapy may also include therapies in which the compound of Formula (I) or a pharmaceutically acceptable salt thereof and one or more other drugs are administered on different overlapping schedules. It is also contemplated that when used in combination with one or more other active ingredients, the compounds of Formula (I) and the other active ingredients may be used in lower doses than when each is used singly. The weight ratio of the compound of this disclosure to the second active ingredient may be varied and will depend upon the effective dose of each ingredient. Generally, an effective dose of each will be used.

Where the subject in need is suffering from or at risk of suffering from cancer, the patient can be treated with a compound of Formula (I) or a pharmaceutically acceptable salt thereof in any combination with one or more other anti-cancer agents including but not limited to:
MAP kinase pathway (RAS/RAF/MEK/ERK) inhibitors including but not limited to: Vemurafanib (PLX4032, CAS No. 918504-65-1), Dabrafenib (CAS No. 1195765-45-7), Encorafenib (LGX818 CAS No. 1269440-17-6), TQ-B3233, XL-518 (Cas No. 1029872-29-4, available from ACC Corp); trametinib (CAS No. 871700-17-3), selumetinib (AZD6244 CAS No. 606143-52-6), TQ-B3234, PD184352 (CAS No. 212631-79-3), PD325901 (CAS No. 391210-10-9), TAK-733 (CAS No. 1035555-63-5), pimasertinib (CAS No. 1236699-92-5), binimetinib (CAS No. 606143-89-9), refametinib (CAS No. 923032-37-5), cobimetinib (GDC-0973 CAS No. 934660-93-2), AZD8330 (CAS No. 869357-68-6), BVD-523 (CAS No. 869886-67-9), LTT462 (CAS No. 869886-67-9), AMG510 (CAS No. 2296729-00-3), ARS853 (CAS No. 1629268-00-3), and any RAS inhibitors disclosed in patents WO2016049565, WO2016164675, WO2016168540, WO2017015562, WO2017058728, WO2017058768, WO2017058792, WO2017058805, WO2017058807, WO2017058902, WO2017058915, WO2017070256, WO2017087528, WO2017100546, WO2017172979, WO2017201161, WO2018064510, WO2018068017, and WO2018119183;
SHP2 inhibitors including but not limited to: SHP099 (CAS No. 2200214-93-1), TNO155 (CAS No. 1801765-04-7), RMC4630, JAB-3312, JAB-3068 and ERAS-601;
SOS1 inhibitors including but not limited to BI1701963 and BAY-293;
CSF1R inhibitors (PLX3397, LY3022855,) and CSF1R antibodies (IMC-054, RG7155);
TGF beta receptor kinase inhibitor such as LY2157299;
BTK inhibitor such as ibrutinib; BCR-ABL inhibitors: Imatinib (CAS No. 152459-95-5); Inilotinib hydrochloride; Nilotinib (CAS No. 923288-95-3); Dasatinib (BMS-345825 CAS No. 302962-49-8); Bosutinib (SKI-606 CAS No. 380843-75-4); Ponatinib (AP24534 CAS No. 943319-70-8); Bafetinib (INNO406 CAS No. 859212-16-1); Danusertib (PHA-739358 CAS No. 827318-97-8), AT9283 (CAS No. 896466-04-9); Saracatinib (AZD0530 CAS No. 379231-04-6); and PF-03814735 (CAS 942487-16-3);
ALK inhibitors: PF-2341066 (XALKOPJ®; crizotinib); 5-chloro-N4-(2-(isopropyl-sulfonyl)phenyl)-N2-(2-methoxy-4-(4-(4-methylpiperazin-1-yl)piperidin-1-yl)phenyl)pyrimidine-2,4-diamine; GSK1838705A (CAS No. 1116235-97-2); CH5424802 (CAS No. 1256580-46-7); Ceritinib (ZYKADIA CAS No. 1032900-25-6); TQ-B3139, and TQ-B3101;
PI3K inhibitors: 4-[2-(1H-indazol-4-yl)-6-[[4-(methylsulfonyl)-piperazin-1-yl]methyl]thieno[3,2-d]pyrimidin-4-yl]morpholine (also known as GDC 0941 and described in PCT Publication Nos. WO 09/036082 and WO 09/055730), BEZ235 or NVP-BEZ235 (CAS No. 915019-65-7), disclosed in PCT Publication No. WO 06/122806);
Vascular Endothelial Growth Factor (VEGF) receptor inhibitors: Bevacizumab (sold under the trademark Avastin® by Genentech/Roche), axitinib, (N-methyl-2-[[3-[(E)-2-pyridin-2-ylethenyl]-1H-indazol-6-yl]sulfanyl]benzamide, also known as AG013736, and described in PCT Publication No. WO 01/002369), Brivanib Alaninate ((S)-((R)-1-(4-(4-fluoro-2-methyl-4H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-TH-indol-6-yl)-2-[(4-pyridinylmethyl)amino]-3-pyridinecarboxamide, and described in PCT Publication No. WO 02/066470), pasireotide (also known as SOM230, and described in PCT Publication No. WO 02/010192), sorafenib (sold under the tradename Nexavar®, CAS No. 284461-73-0); or AL-2846;
MET inhibitor such as foretinib (CAS No. 849217-64-7), cabozantinib (CAS No. 1140909-48-3), capmatinib (CAS No. 1029712-80-8), tepotinib (CAS No. 1100598-32-0), savolitinib (CAS No. 1313725-88-0, or crizotinib (CAS No. 877399-52-5);
FLT3 inhibitors—sunitinib malate (CAS No. 341031-54-7, sold under the tradename Sutent® by Pfizer); PKC412 (CAS No. 120685-11-2, midostaurin); tandutinib (CAS No. 387867-13-2), sorafenib (CAS No. 284461-73-0), lestaurtinib (CAS No.:111358-88-4), KW-2449 (CAS No. 1000669-72-6), quizartinib (AC220, CAS No. 950769-58-1), or crenolanib (CAS No. 670220-88-9);
Epidermal growth factor receptor (EGFR) inhibitors: Gefitnib (sold under the tradename Iressa®), N-[4-[(3-chloro-4-fluorophenyl)amino]-7-[[(3S)-tetrahydro-3-furanyl]oxy]-6-quinazolinyl]-4(dimethylamino)-2-butenamide, sold under the tradename Tovok® by Boehringer Ingelheim), cetuximab (sold under the tradename Erbitux® by Bristol-Myers Squibb), or panitumumab (sold under the tradename Vectibix® by Amgen);
HER2 receptor inhibitors: Trastuzumab (sold under the trademark Herceptin® by Genentech/Roche), neratinib (also known as HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443), lapatinib (CAS No. 231277-92-2) or lapatinib ditosylate (CAS No: 388082-77-7) (sold under the trademark Tykerb® by GlaxoSmithKline); or Trastuzumab emtansine (in the United States, ado-trastuzumab emtansine, trade name Kadcyla)—an antibody-drug conjugate consisting of the monoclonal antibody trastuzumab (Herceptin) linked to the cytotoxic agent mertansine (DM1);
HER dimerization inhibitors: Pertuzumab (sold under the trademark Omnitarg®, by Genentech);
FGFR inhibitors: Erdafitinib (CAS No. 1346242-81-6), Pemigatinib (CAS No. 1513857-77-6) or Infigratinib (CAS No. 872511-34-7)
Aurora kinase inhibitors: TAS-119 (CAS No. 1453099-83-6), LY3295668 (CAS No. 1919888-06-4), or alisertib (CAS No. 1028486-01-2);
CD20 antibodies: Rituximab (sold under the trademarks Riuxan® and MabThera® by Genentech/Roche), tositumomab (sold under the trademarks Bexxar® by GlaxoSmithKline), or ofatumumab (sold under the trademark Arzerra® by GlaxoSmithKline);
Tyrosine kinase inhibitors: Erlotinib hydrochloride (CAS No. 183319-69-9, sold under the trademark Tarceva® by Genentech/Roche), Linifanib (N-[4-(3-amino-1H-indazol-4-yl)phenyl]-N′-(2-fluoro-5-methylphenyl)urea, also known as ABT 869, available from Genentech), sunitinib malate (CAS No. 341031-54-7, sold under the tradename Sutent® by Pfizer), bosutinib (4-[(2,4-dichloro-5-methoxyphenyl)amino]-6-methoxy-7-[3-(4-methylpiperazin4-yl)propoxy]quinoline-3-carbonitrile, also known as SKI-606, and described in U.S. Pat. No. 6,780,996), dasatinib (CAS No. 302962-49-8, sold under the tradename Sprycel® by Bristol-Myers Squibb), armala (CAS No. 444731-52-6, also known as pazopanib, sold under the tradename Votrient® by GlaxoSmithKline), imatinib (CAS No. 152459-95-5) and imatinib mesylate (CAS No. 220127-57-1) (sold under the tradenames Gilvec® and Gleevec® by Novartis);
DNA Synthesis inhibitors: Capecitabine (CAS No. 154361-50-9) (sold under the trademark Xeloda® by Roche), gemcitabine hydrochloride (CAS No. 122111-03-9) (sold under the trademark Gemzar® by Eli Lilly and Company), or nelarabine ((2R3S,4R,5R)-2-(2-amino-6-methoxypurin-9-yl)-5-(hydroxymethyl)oxolane-3,4-diol, sold under the tradenames Arranon® and Atriance® by GlaxoSmithKline);
Antineoplastic agents: oxaliplatin (CAS No. 61825-94-3) (sold under the tradename Eloxatin® ay Sanofi-Aventis and described in U.S. Pat. No. 4,169,846);
Human Granulocyte colony-stimulating factor (G-CSF) modulators: Filgrastim (sold under the tradename Neupogen® by Amgen);
Immunomodulators: Afutuzumab (available from Roche®), pegfilgrastim (sold under the tradename Neulasta® by Amgen), lenalidomide (CAS No. 191732-72-6, also known as CC-5013, sold under the tradename Revlimid®), or thalidomide (CAS No. 50-35-1, sold under the tradename Thalomid®);
CD40 inhibitors: Dacetuzumab (also known as SGN-40 or huS2C6, available from Seattle Genetics, Inc);
Pro-apoptotic receptor agonists (PARAs): Dulanermin (also known as AMG-951, available from Amgen/Genentech);
Hedgehog antagonists: 2-chloro-N-[4-chloro-3-(2-pyridinyl)phenyl]-4-(methylsulfonyl)-benzamide (also known as GDC-0449, and described in PCT Publication No. WO 06/028958);
Phospholipase A2 inhibitors: Anagrelide (CAS No. 58579-51-4, sold under the tradename Agrylin®);
BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386);
MCL-1 inhibitors: MIK665 (CAS No. 1799631-75-6, S64315), AMG 397, and AZD5991 (CAS No. 2143010-83-5); Aromatase inhibitors: Exemestane (CAS No. 107868-30-4, sold under the trademark Aromasin® by Pfizer), letrozole (CAS No. 112809-51-5, sold under the tradename Femara® by Novartis), or anastrozole (CAS No. 120511-73-1, sold under the tradename Arimidex®);
Topoisomerase I inhibitors: Irinotecan (CAS No. 97682-44-5, sold under the trademark Camptosar® by Pfizer), topotecan hydrochloride (CAS No. 119413-54-6, sold under the tradename Hycamtin® by GlaxoSmithKline);
Topoisomerase II inhibitors: etoposide (CAS No. 33419-42-0, also known as VP-16 and Etoposide phosphate, sold under the tradenames Toposar®, VePesid® and Etopophos®), or teniposide (CAS No. 29767-20-2, also known as VM-26, sold under the tradename Vumon®);
mTOR inhibitors: Temsirolimus (CAS No. 162635-04-3, sold under the tradename Torisel® by Pfizer), ridaforolimus (CAS No. 572924-54-0, formally known as deferolimus, AP23573 and MK8669, and described in PCT Publication No. WO 03/064383), or everolimus (CAS No. 159351-69-6, sold under the tradename Afmitor® by Novartis);
Proteasome inhibitor such as carfilzomib (CAS No. 868540-17-4), MLN9708 (CAS No. 1201902-80-8), delanzomib (CAS No. 847499-27-8), or bortezomib (CAS No. 179324-69-7);
BET inhibitors such as INCB054329 (CAS No. 1628607-64-6), OTX015 (CAS No. 202590-98-5), or CPI-0610 (CAS No. 1380087-89-7);
LSD1 inhibitors such as GSK2979552, or INCB059872;
HIF-2α inhibitors such as PT2977 (1672668-24-4), NKT2152, or PT2385 (CAS No. 1672665-49-4);
Osteoclastic bone resorption inhibitors: 1-hydroxy-2-imidazol-1-yl-phosphonoethyl) phosphonic acid monohydrate (sold under the tradename Zometa® by Novartis);
CD33 Antibody Drug Conjugates: Gemtuzumab ozogamicin (sold under the tradename Mylotarg® by Pfizer/Wyeth);
CD22 Antibody Drug Conjugates: Inotuzumab ozogamicin (also referred to as CMC-544 and WAY-207294, available from Hangzhou Sage Chemical Co., Ltd.);
CD20 Antibody Drug Conjugates: Ibritumomab tiuxetan (sold under the tradename Zevalin®);
Somatostain analogs: octreotide (also known as octreotide acetate, sold under the tradenames Sandostatin® and Sandostatin LAR®);
Synthetic Interleukin-11 (IL-l 1): oprelvekin (sold under the tradename Neumega® by Pfizer/Wyeth);
Synthetic erythropoietin: Darbepoetin alfa (sold under the tradename Aranesp® by Amgen);
Receptor Activator for Nuclear Factor k B (RANK) inhibitors: Denosumab (sold under the tradename Prolia® by Amgen);
Thrombopoietin mimetic peptibodies: Romiplostim (sold under the tradename Nplate® by Amgen;
Cell growth stimulators: Palifermin (sold under the tradename Kepivance® by Amgen);
Anti-insulin-like Growth Factor-1 receptor (IGF-1R) antibodies: Figitumumab (also known as CP-751,871, available from ACC Corp), robatumumab (CAS No. 934235-44-6);
Anti-CSl antibodies: Elotuzumab (HuLuc63, CAS No. 915296-00-3);
CD52 antibodies: Alemtuzumab (sold under the tradename Campath®);
Histone deacetylase inhibitors: Voninostat (sold under the tradename Zolinza® by Merck);
Alkylating agents: Temozolomide (sold under the tradenames Temodar® and Temodal® by Schering-Plough/Merck), dactinomycin (also known as actinomycin-D and sold under the tradename Cosmegen®), melphalan (also known as L-PAM, L-sarcolysin, and phenylalanine mustard, sold under the tradename Alkeran®), altretamine (also known as hexamethylmelamine (HMM), sold under the tradename Hexalen®), carmustine (sold under the tradename BiCNU®), bendamustine (sold under the tradename Treanda®), busulfan (sold under the tradenames Busulfex® and Myleran®), carboplatin (sold under the tradename Paraplatin®), lomustine (also known as CCNU, sold under the tradename CeeNU®), cisplatin (also known as CDDP, sold under the tradenames Platinol® and Platinol®-AQ), chlorambucil (sold under the tradename Leukeran®), cyclophosphamide (sold under the tradenames Cytoxan® and Neosar®), dacarbazine (also known as DTIC, DIC and imidazole carboxamide, sold under the tradename DTIC -Dome®), altretamine (also known as hexamethylmelamine (HMM) sold under the tradename Hexalen®), ifosfamide (sold under the tradename Ifex®), procarbazine (sold under the tradename Matulane®), mechlorethamine (also known as nitrogen mustard, mustine and mechloroethamine hydrochloride, sold under the tradename Mustargen®), streptozocin (sold under the tradename Zanosar®), thiotepa (also known as thiophosphoamide, TESPA and TSPA, sold under the tradename Thioplex®;
Biologic response modifiers: bacillus calmette-guerin (sold under the tradenames theraCys® and TICE® BCG), or Denileukin diftitox (sold under the tradename Ontak®);
Anti-tumor antibiotics: doxorubicin (sold under the tradenames Adriamycin® and Rubex®), bleomycin (sold under the tradename Lenoxane®), daunorubicin (also known as dauorubicin hydrochloride, daunomycin, and rubidomycin hydrochloride, sold under the tradename Cerubidine®), daunorubicin liposomal (daunorubicin citrate liposome, sold under the tradename DaunoXome®), mitoxantrone (also known as DHAD, sold under the tradename Novantrone®), epirubicin (sold under the tradename Ellence™), idarubicin (sold under the tradenames Idamycin®, Idamycin PFS®), or mitomycin C (sold under the tradename Mutamycin®);
Anti -microtubule agents: Estramustine (CAS No. 52205-73-9, sold under the tradename Emcyl®);
Cathepsin K inhibitors: Odanacatib (CAS No. 603139-19-1, also know as MK-0822 available from Lanzhou Chon Chemicals, ACC Corp., and ChemieTek, and described in PCT Publication no. WO 03/075836);
Epothilone B analogs: Ixabepilone (CAS No. 219989-84-1, sold under the tradename Lxempra® by Bristol-Myers Squibb);
Heat Shock Protein (HSP) inhibitors: Tanespimycin (17-allylamino-17-demethoxygeldanamycin, also known as KOS-953 and 17-AAG, available from SIGMA, and described in U.S. Pat. No. 4,261,989), NVP-HSP990 (CAS No. 934343-74-5), AUY922 (CAS No. 747412-49-3), AT13387 (CAS No. 912999-49-6), STA-9090 (CAS No. 888216-25-9), Debio 0932, KW-2478 (CAS No. 819812-04-9), XL888 (CAS No. 1149705-71-4), CNF2024 (CAS No. 848695-25-0), and TAS-116 (CAS No. 1260533-36-5);
TpoR agonists: Eltrombopag (sold under the tradenames Promacta® and Revolade® by GlaxoSmithKline);
Anti-mitotic agents: Docetaxel (CAS No. 114977-28-5, sold under the tradename Taxotere® by Sanofi-Aventis); Adrenal steroid inhibitors: aminoglutethimide (CAS No. 125-84-8, sold under the tradename Cytadren®);
Anti-androgens: Nilutamide (CAS No. 63612-50-0, sold under the tradenames Nilandron® and Anandron®), bicalutamide (CAS No. 90357-06-5, sold under tradename Casodex®), or flutamide (CAS No. 13311-84-7, sold under the tradename Fulexin™;
Androgens: Fluoxymesterone (CAS No. 76-43-7, sold under the tradename Halotestin®);
CDK (CDK1, CDK2, CDK3, CDK5, CDK7, CDK8, or CDK9) inhibitors including but not limited to: Alvocidib (CAS No. 146426-40-6, pan-CDK inhibitor, also known as flovopirdol or HMR-1275, 2-(2-chlorophenyl)-5,7-dihydroxy-8-[(3S,4R)-3-hydroxy-1-methyl -4-piperidinyl]-4-chromenone, and described in U.S. Pat. No. 5,621,002);
CDK2 inhibitor PF-07104091;
CDK4/6 inhibitors: pabociclib (CAS No. 827022-33-3), ribociclib (CAS No. 1211441-98-3), abemaciclib (CAS No. 1231929-97-7), PF-06873600 (CAS No. 2185857-97-8), NUV-422 and Trilaciclib (CAS No. 1374743-00-6);
CDK7 inhibitors CT7001 (CAS No. 1805789-54-1) and SY-1365 (CAS No. 1816989-16-8);
CDK9 inhibitors AZD 4573 (CAS No. 2057509-72-3), P276-00 (CAS No. 920113-03-7), AT7519 (CAS No. 844442-38-2), CYC065 (CAS No. 1070790-89-4) or TP-1287;
Gonadotropin-releasing hormone (GnRH) receptor agonists: Leuprolide or leuprolide acetate (sold under the tradenames Viadure® by Bayer AG, Eligard® by Sanofi-Aventis and Lupron® by Abbott Lab);
Taxane anti-neoplastic agents: Cabazitaxel (1-hydroxy-7,10-dimethoxy-9-oxo-5,20-epoxytax-11-ene-2a,4,13a-triyl-4-acetate-2-benzoate-13-[(2R,3S)-3-{[(tert-butoxy)carbonyl]-amino}-2-hydroxy-3-phenylpropanoate), or larotaxel ((2a,3x,4a,5b,7a,10b,13a)-4,10-bis(acetyloxy)-13-({(2R,3S)-3-[(tert-butoxycarbonyl)amino]-2-hydroxy-3-phenylpropanoyl} oxy)-1-hydroxy-9-oxo-5,20-epoxy-7,19-cyclotax-11-en-2-ylbenzoate);
5HT1a receptor agonists: Xaliproden (also known as SR57746, 1-[2-(2-naphthyl)ethyl]-4-[3-(trifluoromethyl)phenyl]-1,2,3,6-tetrahydropyridine, and described in U.S. Pat. No. 5,266,573);
HPC vaccines: Cervarix® sold by GlaxoSmithKline, Gardasil® sold by Merck;
Iron Chelating agents: Deferasinox (CAS No. 201530-41-8, sold under the tradename Exjade® by Novartis);
Anti-metabolites: Claribine (2-chlorodeoxyadenosine, sold under the tradename Leustatin®), 5-fluorouracil (sold under the tradename Adrucil®), 6-thioguanine (sold under the tradename Purinethol®), pemetrexed (sold under the tradename Alimta®), cytarabine (also known as arabinosylcytosine (Ara-C), sold under the tradename Cytosar-U®), cytarabine liposomal (also known as Liposomal Ara-C, sold under the tradename DepoCyt™), decitabine (sold under the tradename Dacogen®), hydroxyurea (sold under the tradenames Hydrea®, Droxia™ and Mylocel™), fludarabine (sold under the tradename Fludara®), floxuridine (sold under the tradename FUDR®), cladribine (also known as 2-chlorodeoxyadenosine (2-CdA) sold under the tradename Leustatin™), methotrexate (also known as amethopterin, methotrexate sodium (MTX), sold under the tradenames Rheumatrex® and Trexall™), or pentostatin (sold under the tradename Nipent®);
Bisphosphonates: Pamidronate (CAS No. 57248-88-1, sold under the tradename Aredia®), zoledronic acid CAS No. 118072-93-8 (sold under the tradename Zometa®);
Demethylating agents: 5-azacitidine (CAS No. 320-67-2, sold under the tradename Vidaza®), decitabine (CAS No. 2353-33-5, sold under the tradename Dacogen®);
Plant Alkaloids: Paclitaxel protein-bound (sold under the tradename Abraxane®), vinblastine (also known as vinblastine sulfate, vincaleukoblastine and VLB, sold under the tradenames Alkaban-AQ® and Velban®), vincristine (also known as vincristine sulfate, LCR, and VCR, sold under the tradenames Oncovin® and Vincasar Pfs®), vinorelbine (sold under the tradename Navelbine®), or paclitaxel (sold under the tradenames Taxol and Onxal™);
Retinoids: Ali tretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Clarus®, Decutan®, Isotane®, Izotech®, Oratane®, Isotret®, and Sotret®), or bexarotene (sold under the tradename Targretin®);
Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,11S,13S,14S,16R,17R)-9-fluoro-11,17-dihydroxy-17-(2-hydroxyacetyl)-10,13,16-trimethyl-6,7,8,9, 10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), or methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®);
Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®);
Estrogen receptor downregulators: Fulvestrant (CAS No. 129453-61-8, sold under the tradename Faslodex®);
Anti-estrogens: tamoxifen (CAS No. 10540-29-1, sold under the tradename Novaldex®); or Toremifene (CAS No. 89778-27-8, sold under the tradename Fareston®);
Selective estrogen receptor modulators (SERMs): Raloxifene (CAS No. 84449-90-1, sold under the tradename Evista®);
Leutinizing hormone releasing hormone (LFfRH) agonists: Goserelin (CAS No. 145781-92-6, sold under the tradename Zoladex®); Progesterones: megestrol (also known as megestrol acetate, CAS No. 595-33-5, sold under the tradename Megace®);
Miscellaneous cytotoxic agents: Arsenic trioxide (sold under the tradename Trisenox®), or asparaginase (also known as L-asparaginase, Erwinia L-asparaginase, sold under the tradenames Elspar® and Kidrolase®);

Exemplary immune checkpoint inhibitors include inhibitors (smack molecules or biologies) against immune checkpoint molecules such as CD27, CD28, CD40, CD 122, CD96, CD73, CD39, CD47, 0X40, GITR, CSF1R, JAK, PI3K delta, PI3K gamma, TAM kinase, arginase, CD137 (also known as 4-1BB), ICOS, A2AR, A2BR, HIF-2a, B7-H3, B7-H4, BTLA, CTLA-4, LAG3, TIM3, VISTA, CD96, TIGIT, PD-1, PD-L1 and PD-L2. In some embodiments, the immune checkpoint molecule is a stimulatory checkpoint molecule selected from CD27, CD28, CD40, ICOS, 0X40, GITR, CD137 and STING. In some embodiments, the immune checkpoint molecule is an inhibitory checkpoint molecule selected from B7-H3, B7-H4, BTLA, CTLA-4, IDO, TDO, Arginase, KIR, LAG3, PD-1, TIM3, CD96, TIGIT and VISTA. In some embodiments, the compounds provided herein can be used in combination with one or more agents selected from KIR inhibitors, TIGIT inhibitors, LAIR1 inhibitors, CD 160 inhibitors, 2B4 inhibitors and TGFR beta inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP -224. In some embodiments, the anti-PD-1 monoclonal antibody is nivolumab, or pembrolizumab or PDR001. In some embodiments, the anti -PD 1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A (atezolizumab) or MEDI4736 (durvalumab). In some embodiments, the anti-PD-L1 small molecule inhibitor is INCB86550.

In some embodiments, the inhibitor of an immune checkpoint molecule is an inhibitor of CTLA-4, e.g., an anti-CTLA-4 antibody. In some embodiments, the anti-CTLA-4 antibody is ipilimumab or tremelimumab. In some embodiments, the inhibitor of an immune checkpoint molecule is an inhibitor of LAG3, e.g., an anti-LAG3 antibody. In some embodiments, the anti-LAG3 antibody is BMS-986016 or LAG525. In some embodiments, the inhibitor of an immune checkpoint molecule is an inhibitor of GITR, e.g., an anti-GITR antibody. In some embodiments, the anti-GITR antibody is TRX518 or, MK-4166, INCAGN01876 or MK-1248. In some embodiments, the inhibitor of an immune checkpoint molecule is an inhibitor of 0X40, e.g., an anti-OX40 antibody or OX40L fusion protein. In some embodiments, the anti-OX40 antibody is MED 10562 or, INCAGN01949, GSK2831781, GSK-3174998, MOXR-0916, PF-04518600 or LAG525. In some embodiments, the OX40L fusion protein is MEDI6383.

Compounds of the invention can also be used to increase or enhance an immune response, including increasing the immune response to an antigen; to improve immunization, including increasing vaccine efficacy; and to increase inflammation. In some embodiments, the compounds of the invention can be sued to enhance the immune response to vaccines including, but not limited, Listeria vaccines, oncolytic viral vaccines, and cancer vaccines such as GVAX® (granulocyte-macrophage colony-stimulating factor (GM-CF) gene-transfected tumor cell vaccine). Anti-cancer vaccines include dendritic cells, synthetic peptides, DNA vaccines and recombinant viruses. Other immune-modulatory agents also include those that block immune cell migration such as antagonists to chemokine receptors, including CCR2 and CCR4; Sting agonists and Toll receptor agonists. Other anti-cancer agents also include those that augment the immune system such as adjuvants or adoptive T cell transfer. Compounds of this application may be effective in combination with CAR (Chimeric antigen receptor) T cell treatment as a booster for T cell activation.

A compound of the invention can also be used in combination with the following adjunct therapies: Anti-nausea drugs: NK-1 receptor antagonists: Casopitant (sold under the tradenames Rezonic® and Zunrisa® by GlaxoSmithKline); and Cytoprotective agents: Amifostine (sold under the tradename Ethyol®), leucovorin (also known as calcium leucovorin, citrovorum factor and folinic acid). The disclosure of the PCT applications referred to herein above are incorporated herein by reference in their entirety.

### EXAMPLES

The following preparations of intermediates and compounds of of the disclosure (Examples) are given to enable those skilled in the art to more clearly understand and to practice the present disclosure. They should not be considered as limiting the scope of the disclosure, but merely as being illustrative and representative thereof.

### SYNTHETIC EXAMPLES

### Intermediate 1

### Synthesis of (2-(fluoromethylene)tetrahydro-1H-pyrrolizin-7a(5H)-yl)methanol

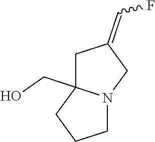 [SMILES: OCC12CCCN1CC(=CF)C2]

### Step 1: ethyl-2-(fluoromethylene)-5-oxotetrahydro-1H-pyrrolizine-7a(5H)-carboxylate

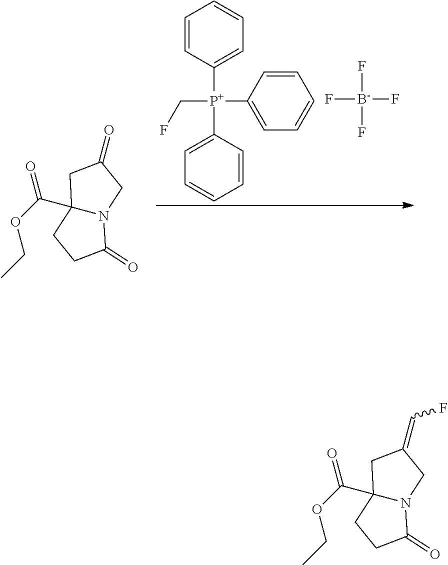 [SMILES: CCOC(=O)C12CCC(=O)N1CC(=O)C2 ; FC[P+](c1ccccc1)(c1ccccc1)c1ccccc1 ; F[B-](F)(F)F ; CCOC(=O)C12CCC(=O)N1CC(=CF)C2]

To a stirred mixture of (fluoromethyl)triphenylphosphanium tetrafluoroborate (3.6 g, 9.42 mmol, 2.49 eq.) in THF (26 mL) was added n-BuLi (3.8 mL, 2.5 M, 9.50 mmol, 2.51 eq.) dropwise at −78° C. under nitrogen atmosphere. After stirring for 1 h at −78° C., a solution of ethyl 2,5-dioxo-tetrahydropyrrolizine-7a-carboxylate (800 mg, 3.79 mmol, 1.00 eq.) in THF (30 mL) was added dropwise over 10 min at −78° C. The resulting mixture was warmed and stirred overnight at RT. The reaction mixture was quenched with saturated NH₄Cl (aq.) at RT, diluted with water, and then extracted with EtOAc. The combined organic layer was concentrated and then purified by silica gel column chromatography, eluted with PE/EA (5:2), to afford the title compound (100 mg, 11.6%) as a colorless oil.

### Step 2: (2-(fluoromethylene)tetrahydro-1H-pyrrolizin-7a(5H)-yl)methanol

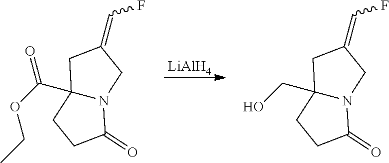 [SMILES: CCOC(=O)C12CCC(=O)N1CC(=CF)C2 ; "LiAlH4" ; O=C1CCC2(CO)CC(=CF)CN12]

To a stirred solution of ethyl-2-(fluoromethylene)-5-oxotetrahydro-1H-pyrrolizine-7a(5H)-carboxylate (80 mg, 0.35 mmol, 1.00 eq.) in THF (0.80 mL) was added a solution of LiAlH₄ in THF (0.70 mL, 2.0 M, 1.40 mmol, 4.00 eq.) slowly at 0° C. under nitrogen atmosphere. The resulting mixture was refluxed for 3h at 70° C. under nitrogen atmosphere, cooled and then was added Na₂SO₄. The resulting mixture was quenched by addition of water, filtered and the solid cake was washed with THF. The filtrate was concentrated to afford the title compound (70 mg) as a colorless oil, which was used for next step without further purification.

### Intermediate 2

### Synthesis of (2-(2,2-difluoroethylidene)tetrahydro-1H-pyrrolizin-7a(5H)-yl)methanol

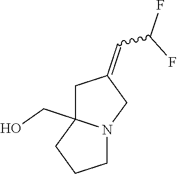 [SMILES: OCC12CCCN1CC(=CC(F)F)C2]

### Step 1: potassium 2-bromo-2,2-difluoroacetate

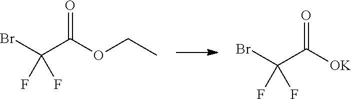

To a stirred mixture of KOH (4.15 g, 73.90 mmol, 1.00 eq.) in MeOH (70 mL) was added ethyl 2-bromo-2,2-difluoroacetate (15.0 g, 73.90 mmol, 1.00 eq.) dropwise at 0° C. The resulting mixture was stirred for 16h at RT and then concentrated. The residue was triturated with DCM to give the title compound (14.0 g, 88.9%) as a white solid.

### Step 2: 2,2-difluoro-2-(triphenylphosphonio)acetate

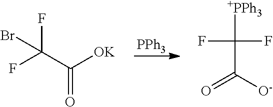

To a stirred mixture of potassium 2-bromo-2,2-difluoroacetate (14.0 g, 65.72 mmol, 1.00 eq.) in DMF (140 mL) was added PPh₃ (34.0 g, 129.63 mmol, 1.97 eq.) at RT under N₂ atmosphere. After stirring at RT for 16 h, the reaction mixture was filtered and the solid cake wash washed with cool DMF, water and then Et₂O to give the title compound (12.0 g, 51.2%) as a white solid.

### Step 3: (difluoromethyl)triphenylphosphonium bromide

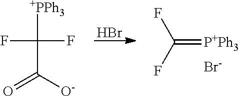

To a stirred mixture of 2,2-difluoro-2-(triphenylphosphonio)acetate (11.0 g, 30.87 mmol, 1.00 eq.) in THF (30 mL) was added 40% HBr aqueous solution (5.0 mL) at RT under N₂ atmosphere. The resulting mixture was refluxed for 1h, cooled, concentrated to remove most THF, and then extracted with DCM. The combined organic layers were washed with water, brine, dried over anhydrous Na₂SO₄, filtered and then concentrated. The residue was purified by trituration with Et₂O to give the title compound (8.0 g, 66.1%) as a white solid.

### Step 4: ethyl 2-(2,2-difluoroethylidene)-5-oxotetrahydro-1H-pyrrolizine-7a(5H)-carboxylate

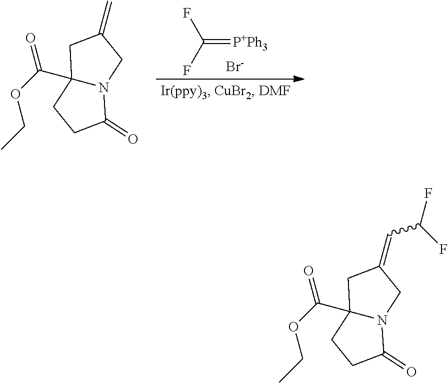

A mixture of Ir(ppy)₃ (75 mg, 0.115 mmol, 0.03 eq.), CuBr₂ (171 mg, 0.765 mmol, 0.2 equiv), (difluoromethyl)triphenylphosphonium bromide (4.51 g, 11.499 mmol, 3.00 eq.) and ethyl 2-methylidene-5-oxo-tetrahydropyrrolizine-7a-carboxylate (800 mg, 3.823 mmol, 1.00 eq.) in DMF (5 mL) was degassed for three times by the freeze-pump-thaw procedure. The reaction mixture was irradiated with blue LEDs for 10 h, and then was added DBU (3.49 g, 22.924 mmol, 6.00 eq.). After stirring for 10h, the reaction mixture was poured into brine, extracted with EtOAc. The combined organic layers were washed with brine, dried with Na₂SO₄ and then concentrated. The residue was purified by column chromatography on silica gel to give the title compound (380 mg, 38.3%) as a light-yellow solid.

### Step 5: (2-(2,2-difluoroethylidene)tetrahydro-1H-pyrrolizin-7a(5H)-yl)methanol

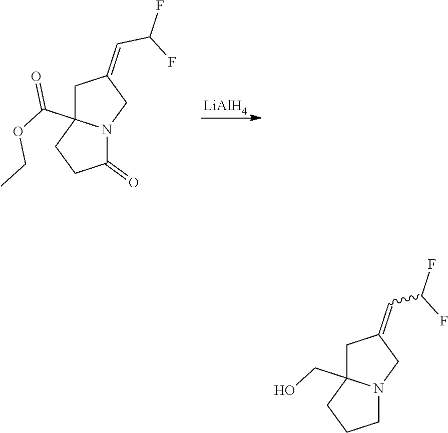 [SMILES: CCOC(=O)C12CCC(=O)N1C/C(=C\C(F)F)C2 ; "LiAlH4" ; OCC12CCCN1CC(=CC(F)F)C2]

To a stirred solution of ethyl 2-(2,2-difluoroethylidene)-5-oxotetrahydro-TH-pyrrolizine-7a(5H)-carboxylate (300 mg, 1.157 mmol, 1.00 eq.) in THF (3.0 mL) was charged with 2.0 M LiAlH₄ in THF (2.3 mL, 4.60 mmol, 3.98 eq.) over 10 min at 0° C. under nitrogen atmosphere. The resulting mixture was stirred for 10 min at 0° C., and then refluxed for 3h. To the cooled reaction mixture was added Na₂SO₄, followed by water slowly at RT. The resulting mixture was filtered and the solid cake was washed with THF. The filtrate was concentrated to afford the title compound (230 mg, 97.8%) as a colorless oil. MS (ES, m/z): [M+1]⁺=228.2.

### Intermediate 3

### Synthesis of ((3aR,7aR,8aS)-hexahydro-TH-furo[3,4-b]pyrrolizin-7a(5H)-yl)methanol and ((3aS,7aS,8aR)-hexahydro-TH-furo[3,4-b]pyrrolizin-7a(5H)-yl)methanol

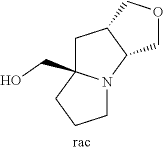 [SMILES: OC[C@]12CCCN1[C@H]1COC[C@H]1C2]

### Step 1: 2-(tert-butyl) 7a-methyl (2S,3R,7aR)-3-((benzyloxy)methyl)tetrahydro-1H-pyrrolizine-2,7a(5H)-dicarboxylate and 2-(tert-butyl) 7a-methyl (2R,3S,7aS)-3-((benzyloxy)methyl)tetrahydro-1H-pyrrolizine-2,7a(5H)-dicarboxylate

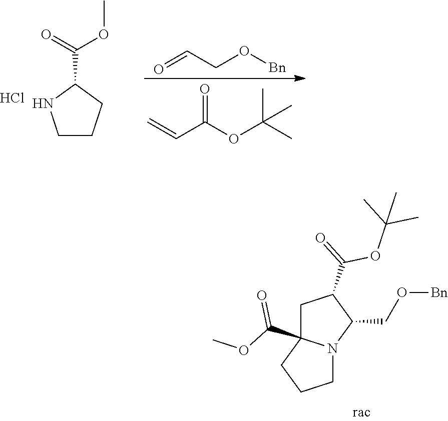

To a stirred solution of methyl L-prolinate hydrochloride (22.0 g, 132.83 mmol, 1.00 eq.) in toluene (800 mL) were added AgOAc (1.11 g, 6.65 mmol, 0.050 eq.), 2-(benzyloxy) acetaldehyde (19.95 g, 132.84 mmol, 1.00 eq.), tert-butyl acrylate (17.00 g, 132.64 mmol, 1.00 eq.) and triethylamine (14.79 g, 146.16 mmol, 1.10 eq.) at RT. After stirring at RT for 16 h under dark condition, the reaction mixture was concentrated and the residue was purified by silica gel column chromatography, eluted with EA/PE (0-50%), to afford the title compound (2.50 g, 4.8%) as a light -yellow oil.

### Step 2: 2-(tert-butyl) 7a-methyl (2S,3R,7aR)-3-(hydroxymethyl)tetrahydro-1H-pyrrolizine-2,7a(5H)-dicarboxylate and 2-(tert-butyl) 7a-methyl (2R,3S,7aS)-3-(hydroxymethyl)tetrahydro-1H-pyrrolizine-2,7a(5H)-dicarboxylate

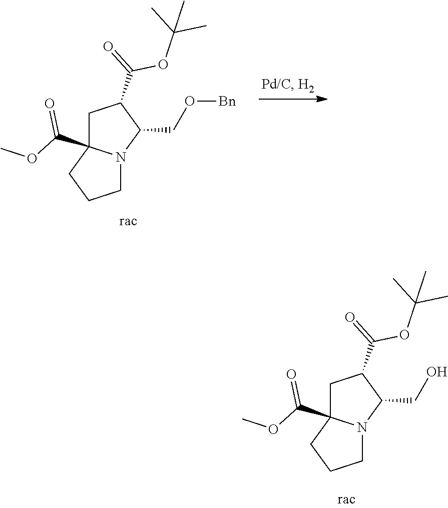 [SMILES: COC(=O)[C@]12CCCN1[C@@H](COCc1ccccc1)[C@@H](C(=O)OC(C)(C)C)C2 ; COC(=O)[C@]12CCCN1[C@@H](CO)[C@@H](C(=O)OC(C)(C)C)C2]

A mixture of 2-(tert-butyl) 7a-methyl (2S,3R,7aR)-3-((benzyloxy)methyl)tetrahydro-1H-pyrrolizine-2,7a(5H)-dicarboxylate and 2-(tert-butyl) 7a-methyl (2R,3S,7aS)-3-((benzyloxy)-methyl)tetrahydro-1H-pyrrolizine-2,7a(5H)-dicarboxylate (2.40 g, 6.16 mmol, 1.00 eq.), HCl (g) in MeOH (1 drop) and 10% Pd/C (7.20 g) in MeOH (240 mL) was stirred for 16 h at 50° C. under hydrogen atmosphere (20 atm). The reaction mixture was basified to pH=9 with ammonia aqueous solution. The resulting mixture was filtered, and the filter cake was washed with MeOH. The filtrate was concentrated, and the residue was purified by silica gel column chromatography, eluted with MeOH/CH₂Cl₂ (0-10%), to afford the title compound (900 mg, 48.9%) as a yellow oil.

### Step 3: 2-(tert-butyl) 7a-methyl (2S,3R,7aR)-3-((tosyloxy)methyl)tetrahydro-1H-pyrrolizine-2,7a(5H)-dicarboxylate and 2-(tert-butyl) 7a-methyl (2R,3S,7aS)-3-((tosyloxy)methyl)tetrahydro-1H-pyrrolizine-2,7a(5H)-dicarboxylate

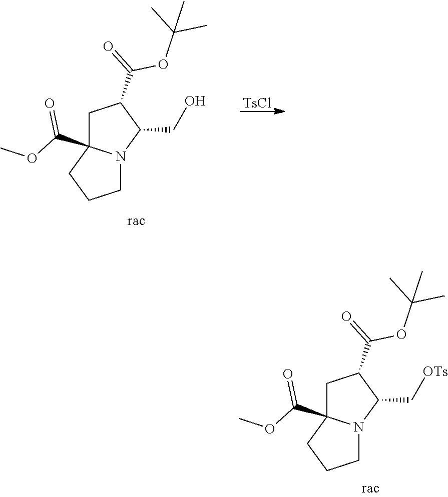

To a stirred solution of 2-(tert-butyl) 7a-methyl (2S,3R,7aR)-3-(hydroxymethyl) tetrahydro-1H-pyrrolizine-2,7a(5H)-dicarboxylate and 2-(tert-butyl) 7a-methyl (2R,3S,7aS)-3-(hydroxymethyl)tetrahydro-1H-pyrrolizine-2,7a(5H)-dicarboxylate (900 mg, 3.01 mmol, 1.00 eq.), DMAP (73 mg, 0.60 mmol, 0.20 eq.) and triethylamine (608 mg, 6.01 mmol, 2.00 eq.) in DCM (18 mL) was added a solution of p-toluenesulfonyl chloride (745 mg, 3.91 mmol, 1.30 eq.) in DCM (3.6 mL) dropwise at 5° C. The resulting mixture was stirred at RT for 2 h, concentrated and then purified by silica gel column chromatography, eluted with EA/PE (0-50%), to afford the title compound (900 mg, 65.8%) as a colorless oil.

### Step 4: ((2S,3R,7aR)-2,7a-bis(hydroxymethyl)hexahydro-1H-pyrrolizin-3-yl)methyl 4-methylbenzenesulfonate and ((2R,3S,7aS)-2,7a-bis(hydroxymethyl)hexahydro-1H-pyrrolizin-3-yl)methyl 4-methylbenzenesulfonate

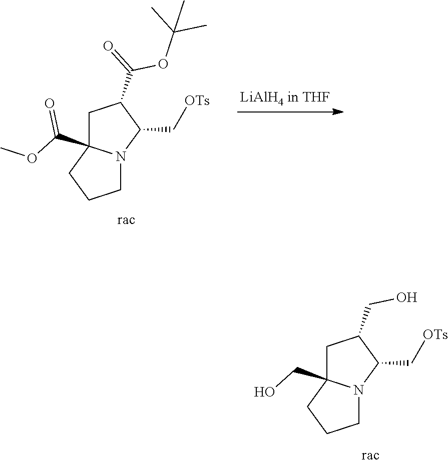 [SMILES: COC(=O)[C@]12CCCN1[C@@H](COS(=O)(=O)c1ccc(C)cc1)[C@@H](C(=O)OC(C)(C)C)C2 ; Cc1ccc(S(=O)(=O)OC[C@H]2[C@@H](CO)C[C@@]3(CO)CCCN23)cc1]

To a stirred solution of 2-(tert-butyl) 7a-methyl (2S,3R,7aR)-3-((tosyloxy)methyl) tetrahydro-1H-pyrrolizine-2,7a(5H)-dicarboxylate and 2-(tert-butyl) 7a-methyl (2R,3S,7aS)-3-((tosyloxy)methyl)tetrahydro-1H-pyrrolizine-2,7a(5H)-dicarboxylate (760 mg, 1.68 mmol, 1.00 eq.) in THF (5.0 mL) was added 1.0 M LiAlH₄ in THF (5.0 mL, 5.00 mmol, 2.98 eq.) dropwise at 5° C. After stirring at 5° C. for additional 1h, the reaction mixture was diluted with THF, added Na₂SO₄, quenched with water at 5° C. The resulting mixture was filtered, and the filter cake was washed with THF. The filtrate was concentrated to afford the crude title compound (450 mg, 75.6%) as a colorless oil, which was used for next step without further purification.

### Step 5: ((3aR,7aR,8aS)-hexahydro-1H-furo[3,4-b]pyrrolizin-7a(5H)-yl)methanol and ((3aS,7aS,8aR)-hexahydro-1H-furo[3,4-b]pyrrolizin-7a(5H)-yl)methanol

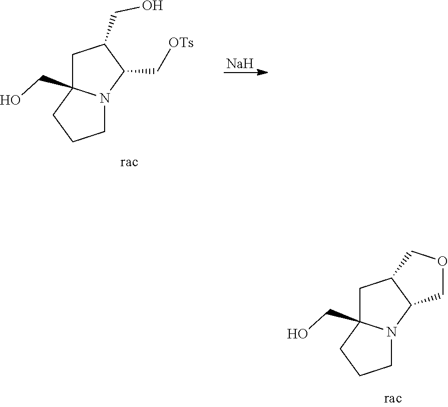

To a stirred solution of ((2S,3R,7aR)-2,7a-bis(hydroxymethyl)hexahydro-1H-pyrrolizin-3-yl)methyl 4-methylbenzenesulfonate and ((2R,3S,7aR)-2,7a-bis(hydroxymethyl)hexahydro-1H-pyrrolizin-3-yl)methyl 4-methylbenzenesulfonate (450 mg, 1.27 mmol, 1.00 eq.) in THF (12 mL) was added 60% sodium hydride (152 mg, 3.80 mmol, 3.00 eq.) in portions at 5° C. The resulting mixture was stirred at RT for 40 h. This reaction mixture was used for next step directly without further purification.

### Intermediate 4

### Synthesis of (2,3-dihydro-1H-pyrrolo[1,2-a]indol-9a(9H)-yl)methanol

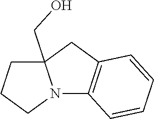 [SMILES: OCC12CCCN1c1ccccc1C2]

### Step 1: 1-(tert-butyl) 2-ethyl 1H-indole-1,2-dicarboxylate

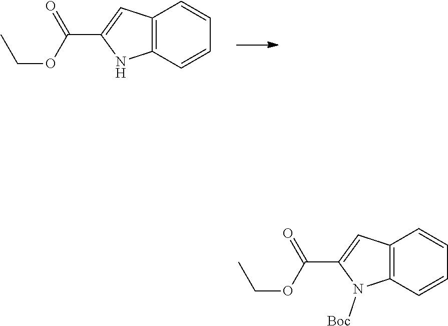 [SMILES: CCOC(=O)c1cc2ccccc2[nH]1 ; CCOC(=O)c1cc2ccccc2n1C(=O)OC(C)(C)C]

To a stirred solution of ethyl 1H-indole-2-carboxylate (30.0 g, 158.66 mmol, 1.00 eq.) and DMAP (3.87 g, 31.68 mmol, 0.20 eq.) in CH₃CN (400 mL) was added a solution of Boc₂O (69.2 g, 317.07 mmol, 2.00 eq.) in CH₃CN (200 mL) dropwise at 0° C. under nitrogen atmosphere. The reaction mixture was stirred for 3h at RT, concentrated and then purified by silica gel column chromatography, eluted with PE/EA (7:1), to afford the title compound (38.1 g, 83.0%) as a white solid.

### Step 2: 1-(tert-butyl) 2-ethyl indoline-1,2-dicarboxylate

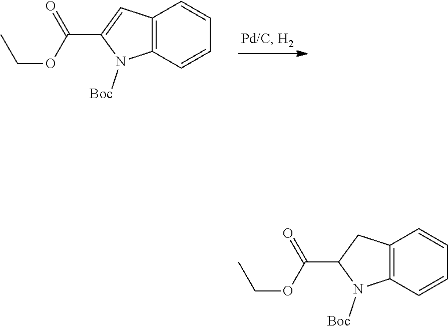 [SMILES: CCOC(=O)c1cc2ccccc2n1C(=O)OC(C)(C)C ; CCOC(=O)C1Cc2ccccc2N1C(=O)OC(C)(C)C]

A mixture of 1-(tert-butyl) 2-ethyl 1H-indole-1,2-dicarboxylate (38.1 g, 131.68 mmol, 1.00 eq.) and 10% Pd/C (3.8 g) in EtOH (380 mL) was stirred for 6 h at RT under hydrogen atmosphere (1 atm). The reaction mixture was filtered and concentrated to give the title compound (37.0 g, 96.4%) as an off-white solid.

### Step 3: 1-(tert-butyl) 2-ethyl 2-(3-chloropropyl)indoline-1,2-dicarboxylate

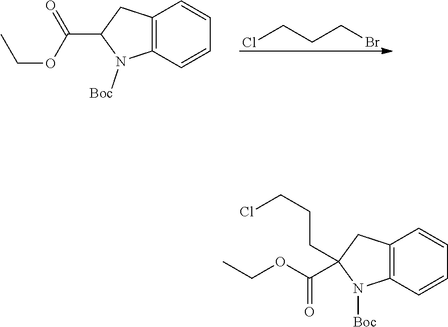

To a stirred solution of 1-(tert-butyl) 2-ethyl indoline-1,2-dicarboxylate (15.0 g, 51.48 mmol, 1.00 eq.) in THF (300 mL) was added 1.0 M LiHMDS in THF (77 mL, 77 mmol, 1.50 eq.) dropwise at −78° C. under nitrogen atmosphere. After stirring for 30 min, 1-chloro-3-bromopropane (40.5 g, 257.24 mmol, 5.00 eq.) was added dropwise at −78° C. The resulting mixture was warm to RT and then stirred overnight. The reaction mixture was quenched with saturated NH₄Cl (aq.) and then extracted with EtOAc. The combined organic layers were washed with water, brine, dried over anhydrous Na₂SO₄, filtered and then concentrated. The residue was purified by silica gel column chromatography, eluted with EtOAc/PE (0-30%), to afford the title compound (10.0 g, 52.8%) as a yellow oil.

### Step 4: ethyl 2-(3-chloropropyl)indoline-2-carboxylate

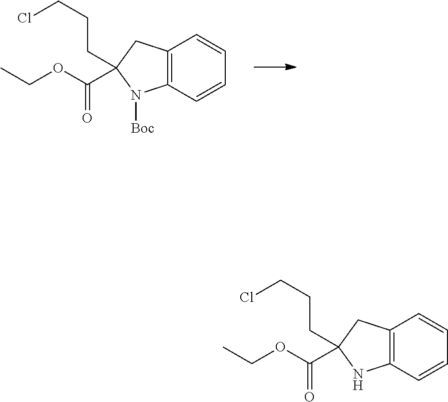 [SMILES: CCOC(=O)C1(CCCCl)Cc2ccccc2N1C(=O)OC(C)(C)C ; CCOC(=O)C1(CCCCl)Cc2ccccc2N1]

A mixture of 1-(tert-butyl) 2-ethyl 2-(3-chloropropyl)indoline-1,2-dicarboxylate (10.0 g, 27.18 mmol, 1.00 eq.) in TFA (100 mL) was stirred for 30 min at RT. The reaction mixture was concentrated and the residue was purified by silica gel column chromatography, eluted with EA/PE (0-70%), to afford the title compound (7.0 g, 96.2%) as a yellow oil.

### Step 5: ethyl 2,3-dihydro-1H-pyrrolo[1,2-a]indole-9a(9H)-carboxylate

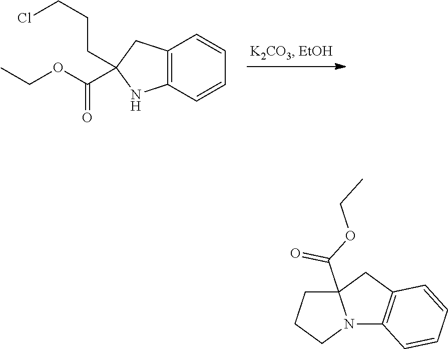 [SMILES: CCOC(=O)C1(CCCCl)Cc2ccccc2N1 ; CCOC(=O)C12CCCN1c1ccccc1C2]

A mixture of ethyl ethyl 2-(3-chloropropyl)indoline-2-carboxylate (1.50 g, 5.60 mmol, 1.00 eq.) and K₂CO₃ (3.87 g, 28.00 mmol, 5.00 eq.) in EtOH (30 mL) was stirred for 2 h at RT. The resulting mixture was diluted with water and then extracted with EtOAc. The combined organic layers were washed with water, brine, dried over anhydrous Na₂SO₄, filtered and then concentrated to give the title compound (1.02 g, 78.8%) as a yellow oil.

### Step 6: (2,3-dihydro-TH-pyrrolo[1,2-a]indol-9a(9H)-yl)methanol

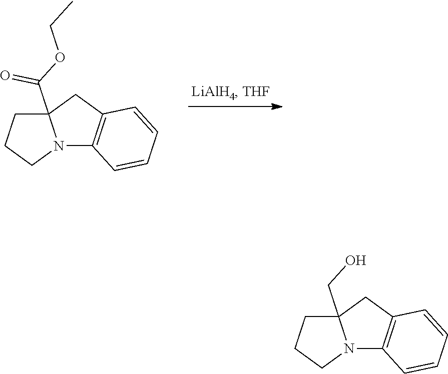 [SMILES: CCOC(=O)C12CCCN1c1ccccc1C2 ; OCC12CCCN1c1ccccc1C2]

To a stirred solution of ethyl 2,3-dihydro-TH-pyrrolo[1,2-a]indole-9a(9H)-carboxylate (1.0 g, 4.32 mmol, 1.00 eq.) in THF (10 mL) was added 1.0 M LiAlH₄ in THF (5.2 mL, 5.2 mmol, 1.20 eq.) dropwise at RT. The resulting mixture was stirred for 2 h at RT, quenched by addition of MeOH and Na₂SO₄·10H₂O. The resulting mixture was filtered and then concentrated. The residue was purified by silica gel column chromatography, eluted with CH₂Cl₂/MeOH (10:1), to afford the title compound (450 mg, 55.1%) as a yellow oil.

### Intermediate 5

### Synthesis of (2,3-dihydro-1H-pyrrolo[2,1-a]isoindol-9b(5H)-yl)methanol

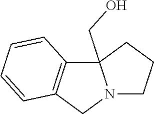 [SMILES: OCC12CCCN1Cc1ccccc12]

### Step 1: methyl (2-bromobenzyl)prolinate

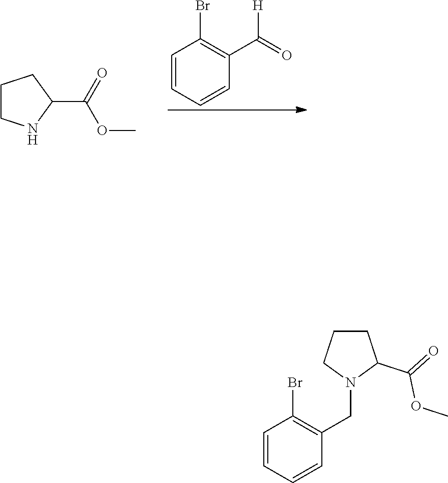

A solution of methyl prolinate (5.0 g, 38.71 mmol, 1.00 eq.) and 2-bromobenzaldehyde (14.32 g, 77.40 mmol, 2.00 eq.) in DCM (50 mL) was stirred at 25° C. for 1 h. To this solution was added NaBH(OAc)₃ (16.41 g, 77.43 mmol, 2.00 eq.) in portions at 5° C., and the resulting mixture was stirred at 25° C. for 5h. The reaction mixture was diluted with water and then extracted with EtOAc. The combined organic layers were washed with brine, dried over anhydrous Na₂SO₄, filtered and then concentrated. The residue was purified by silica gel column chromatography, eluted with PE/EA (9/1), to afford the title compound (4.5 g, 39.0%) as a colorless oil.

### Step 2: tert-butyl 2,3-dihydro-1H-pyrrolo[2,1-a]isoindole-9b(5H)-carboxylate

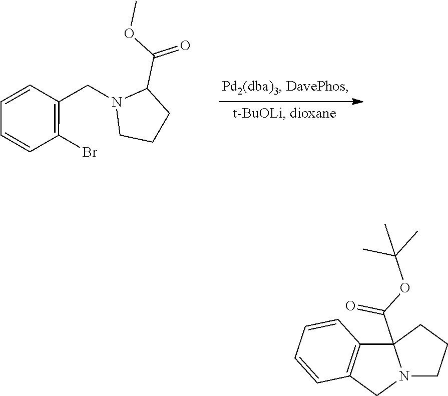 [SMILES: COC(=O)C1CCCN1Cc1ccccc1Br ; CC(C)(C)OC(=O)C12CCCN1Cc1ccccc12]

A mixture of t-BuOLi (2.35 g, 29.34 mmol, 3.50 eq.), DavePhos (0.16 g, 0.42 mmol, 0.05 eq.) and Pd₂(dba)₃ (0.15 g, 0.17 mmol, 0.02 eq.) in 1,4-dioxane (25 mL) was stirred at 25° C. for 5 min under N₂ atmosphere. To this mixture were added dodecane (0.43 g, 2.52 mmol, 0.30 eq.) and methyl (2-bromobenzyl)prolinate (2.5 g, 8.38 mmol, 1.00 eq.) at 25° C. The resulting mixture was stirred at 85° C. for 12 h under N₂ atmosphere, diluted with water and then extracted with DCM. The combined organic layers were washed with brine, dried over anhydrous Na₂SO₄, filtered and then concentrated. The residue was purified by silica gel column chromatography, eluted with PE/EA (1/5), to afford the title compound (1.1 g, 50.6%) as a light-yellow solid.

### Step 3: (2,3-dihydro-1H-pyrrolo[2,1-a]isoindol-9b(5H)-yl)methanol

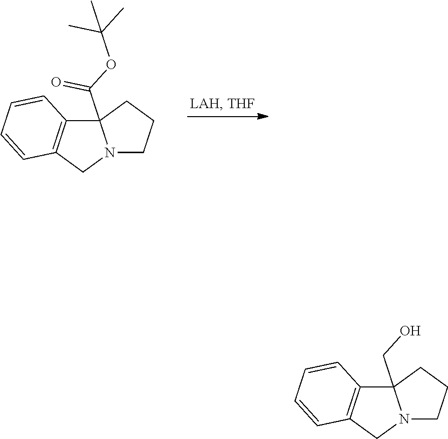 [SMILES: CC(C)(C)OC(=O)C12CCCN1Cc1ccccc12 ; OCC12CCCN1Cc1ccccc12]

To a stirred solution of tert-butyl 2,3-dihydro-1H-pyrrolo[2,1-a]isoindole-9b(5H)-carboxylate (1.0 g, 3.86 mmol, 1.00 eq.) in THF (1.0 mL) was added 1.0 M LiAlH₄ in THF (5.8 mL, 5.8 mmol, 1.50 eq.) dropwise at 0° C. under N₂ atmosphere. The resulting mixture was stirred at 0° C. for 1h, quenched with water, 15% aqueous NaOH and then water in sequence at 0° C. The resulting mixture was filtered, and the filter cake was washed with EtOAc. The filtrate was concentrated to give the title compound (369.7 mg, 50.6%) as a light-yellow solid.

### Intermediate 6

### Synthesis of 3,8-diazabicyclo[3.2.1]oct-6-ene-6-carbonitrile bis(2,2,2-trifluoroacetate)

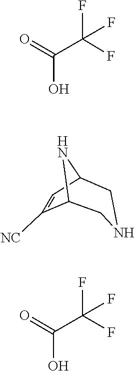 [SMILES: O=C(O)C(F)(F)F ; N#CC1=CC2CNCC1N2 ; O=C(O)C(F)(F)F]

### Step 1: tert-butyl 3-bromo-9-(2-phenylpropan-2-yl)-3a,4,5,7,8,8a-hexahydro-6H-4,8-epiminoisoxazolo[4,5-d]azepine-6-carboxylate

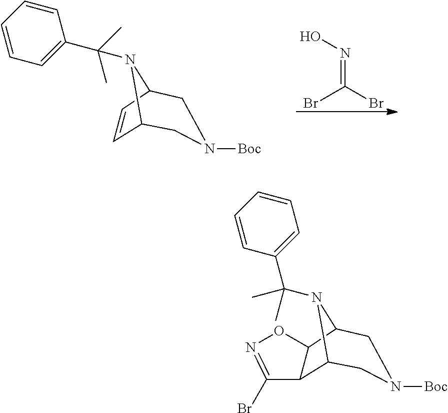

A mixture of tert-butyl (1R,5S)-8-(2-phenylpropan-2-yl)-3,8-diazabicyclo[3.2.1]oct-6-ene-3-carboxylate (1.00 g, 3.04 mmol, 1.00 eq.), hydroxycarbonimidic dibromide (1.24 g, 6.11 mmol, 2.00 eq.) and NaHCO₃ (1.28 g, 15.24 mmol, 5.01 eq.) in EtOAc (10 mL) was stirred for 16 h at 75° C. under nitrogen atmosphere. The reaction mixture was cooled, quenched with water, and then extracted with EtOAc. The combined organic layers were washed with brine, dried with Na₂SO₄, filtered and then concentrated. The residue was purified by silica gel column chromatography, eluted with PE/EA (12:1), to afford the title compound (0.95 g, 69.4%) as a white solid.

### Step 2: tert-butyl 6-cyano-7-hydroxy-8-(2-phenylpropan-2-yl)-3,8-diazabicyclo[3.2.1]octane-3-carboxylate

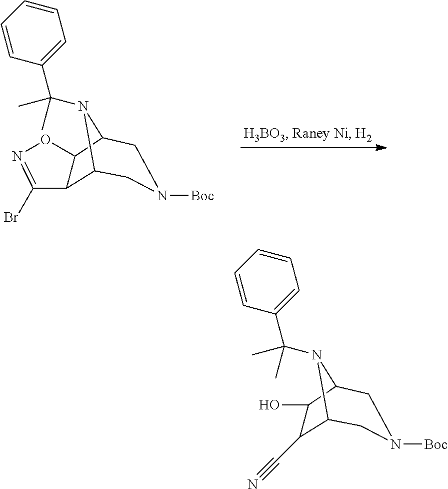 [SMILES: CC(C)(C)OC(=O)N1CC2C3ON=C(Br)C3C(C1)N2C(C)(C)c1ccccc1 ; CC(C)(C)OC(=O)N1CC2C(O)C(C#N)C(C1)N2C(C)(C)c1ccccc1]

A mixture of tert-butyl (3aR,4S,8S,8aR)-3-bromo-9-(2-phenylpropan-2-yl)-3a,4,5,7,8,8a-hexahydro-6H-4,8-epiminoisoxazolo[4,5-d]azepine-6-carboxylate (900 mg, 2.00 mmol, 1.00 eq.), boric acid (371 mg, 6.00 mmol, 3.00 eq.) and Raney Ni (8.56 mg) in MeOH (5 mL) and H₂O (1 mL) was stirred for 2 h at RT under hydrogen atmosphere (2 atm). The resulting mixture was filtered, and the filter cake was washed with MeOH. The filtrate was concentrated under reduced pressure to give the title compound (380 mg, 51.0%) as an off-white oil.

### Step 3: tert-butyl 6-cyano-8-(2-phenylpropan-2-yl)-3,8-diazabicyclo[3.2.1]oct-6-ene-3-carboxylate

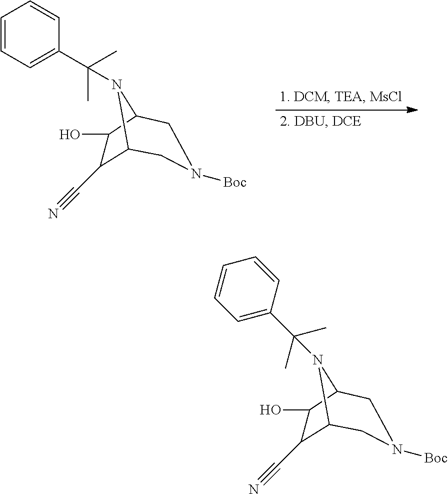 [SMILES: CC(C)(C)OC(=O)N1CC2C(O)C(C#N)C(C1)N2C(C)(C)c1ccccc1 ; CC(C)(C)OC(=O)N1CC2C(O)C(C#N)C(C1)N2C(C)(C)c1ccccc1]

To a stirred solution of tert-butyl (1S,5S,7R)-6-cyano-7-hydroxy-8-(2-phenylpropan-2-yl)-3,8-diazabicyclo[3.2.1]octane-3-carboxylate (380 mg, 1.02 mmol, 1.00 eq.) and TEA (207.02 mg, 2.05 mmol, 2.00 eq.) in DCM (10 mL) was added MsCl (140.61 mg, 1.23 mmol, 1.20 eq.) dropwise at 0° C. under nitrogen atmosphere. After stirring at RT for 16 h, the reaction mixture was quenched with water and then extracted with DCM. The combined organic layers were concentrated. The residue was dissolved in DCE (10 mL), and to the resulting solution was added DBU (467.18 mg, 3.07 mmol, 3.00 eq.) dropwise at RT. After stirring further at RT for 1h, the reaction mixture was quenched with water and then extracted with EtOAc. The organic layer was dried over Na₂SO₄, filtered and then concentrated. The residue was purified by silica gel column chromatography, eluted with PE/EA (10:1) to afford the title compound (295 mg, 81.4%) as a white solid.

### Step 4: 3,8-diazabicyclo[3.2.1]oct-6-ene-6-carbonitrile bis(2,2,2-trifluoroacetate)

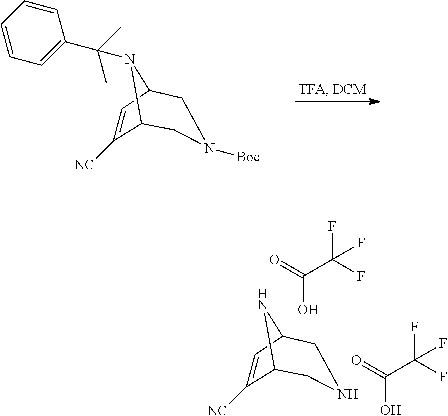

A mixture of tert-butyl (1R,5S)-6-cyano-8-(2-phenylpropan-2-yl)-3,8-diazabicyclo [3.2.1]oct-6-ene-3-carboxylate (290 mg, 0.82 mmol, 1.00 eq.) in TFA (3 mL) was stirred for 2 h at 80° C. under nitrogen atmosphere. The reaction mixture was cooled and then concentrated. The residue was triturated with Et₂O to afford the title compound (215 mg, 72.0%) as a white oil.

### Intermediate 7

### Synthesis of (1R,5S)-3,8-diazabicyclo[3.2.1]oct-6-ene bis(2,2,2-trifluoroacetate)

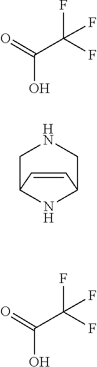 [SMILES: O=C(O)C(F)(F)F ; C1=CC2CNCC1N2 ; O=C(O)C(F)(F)F]

### Step 1: tert-butyl (3S,4R)-3,4-dihydroxypyrrolidine-1-carboxylate

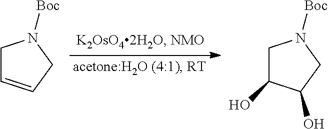 [SMILES: CC(C)(C)OC(=O)N1CC=CC1 ; CC(C)(C)OC(=O)N1C[C@@H](O)[C@@H](O)C1]

To a stirred solution of tert-butyl 2,5-dihydro-1H-pyrrole-1-carboxylate (2.5 g, 14.78 mmol, 1.00 eq.) in acetone (24 mL) and H₂O (6 mL) was added NMO (2.6 g, 22.16 mmol, 1.50 eq.) and K₂OsO₄·2H₂O (163 mg, 0.44 mmol, 0.030 eq.) in portions at RT under nitrogen atmosphere. The resulting mixture was stirred for 3 h at RT, quenched with aqueous Na₂S₂O₃ and then extracted with EtOAc. The combined organic layers were washed with brine, dried over anhydrous Na₂SO₄, filtered and then concentrated. The residue was purified by silica gel column chromatography, eluted with DCM/EA (10:1 to 1:10), to afford the title compound (2.8 g, 93.2%) as a yellow oil.

### Step 2: tert-butyl bis(2-hydroxybut-3-en-1-yl)carbamate

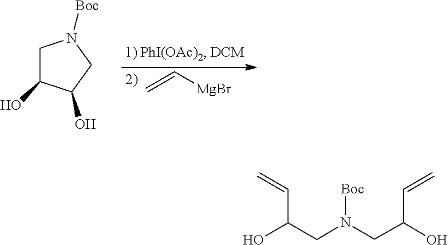

To stirred solution tert-butyl (3R,4S)-3,4-dihydroxypyrrolidine-1-carboxylate (2.50 g, 12.30 mmol, 1.00 eq.) in DCM (25 mL) was added (diacetoxyiodo)benzene (5.94 g, 18.44 mmol, 1.50 eq.) slowly at 0° C. under nitrogen atmosphere. The resulting mixture was stirred for 1 h at RT, cooled to −78° C. and then added 1.0 M vinylmagnesium bromide in THF (74 mL, 74.0 mmol, 6.02 eq.) slowly over 20 minutes. After stirring for 16 h at RT, the reaction mixture was quenched with 1.0 M aqueous HCl and then extract with DCM. The combined organic layers were washed with water, dried over Na₂SO₄, filtered and then concentrated. The residue was purified by silica gel column chromatography. eluted with PE/EA (1/1), to afford the title compound (2.6 g, 82.1%) as a yellow oil.

### Step 3: ((tert-butoxycarbonyl)azanediyl)bis(but-3-ene-1,2-diyl) bis(2,2,2-trichloroacetimidate)

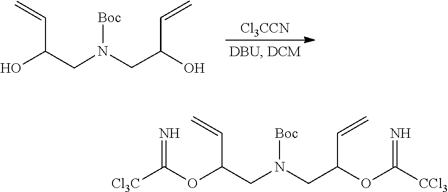

To a stirred solution of tert-butyl bis(2-hydroxybut-3-en-1-yl)carbamate (1.2 g, 4.66 mmol, 1.00 eq.) in DCM (16 mL) were add 2,2,2-trichloroacetonitrile (4.04 g, 27.98 mmol, 6.00 eq.), DBU (0.35 g, 2.30 mmol, 0.49 eq.) dropwise at 0° C. After stirring for 18h at RT, the reaction mixture was concentrated and the residue was purified by silica gel column chromatography, eluted with PE/EA (10:1), to afford title compound (0.8 g, 31.3%) as a yellow oil.

### Step 4: tert-butyl (3S,5R)-4-(2-phenylpropan-2-yl)-3,5-divinylpiperazine-1-carboxylate

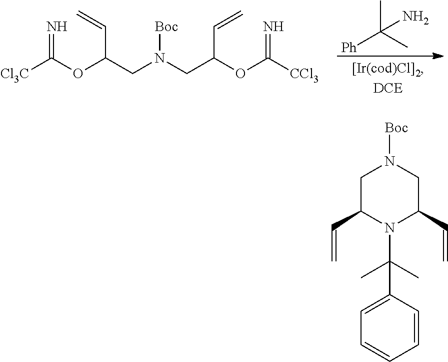

To a stirred solution of 2-phenylpropan-2-amine (238 mg, 1.76 mmol, 1.21 eq.) in DCE (5 mL) was added [Ir(cod)Cl]₂ (49 mg, 0.074 mmol, 0.051 eq.), followed by a solution of ((tert-butoxycarbonyl)azanediyl)bis(but-3-ene-1,2-diyl) bis(2,2,2-trichloroacetimidate) (800 mg, 1.46 mmol, 1.00 eq.) in DCE (4.4 mL) dropwise at 0° C. under nitrogen atmosphere. After stirring overnight at RT, the reaction mixture was concentrated and the residue was purified by silica gel column chromatography, eluted with PE/EA (5:1), to afford the title compound (315 mg, 60.3%) as a yellow oil.

### Step 5: (1R,5S)-8-(2-phenylpropan-2-yl)-3,8-diazabicyclo[3.2.1]oct-6-ene-3-carboxylate

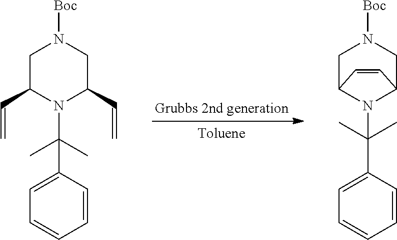 [SMILES: C=C[C@@H]1CN(C(=O)OC(C)(C)C)C[C@H](C=C)N1C(C)(C)c1ccccc1 ; CC(C)(C)OC(=O)N1CC2C=CC(C1)N2C(C)(C)c1ccccc1]

To a stirred solution of tert-butyl (3S,5R)-4-(2-phenylpropan-2-yl)-3,5-divinylpiperazine-1-carboxylate (315 mg, 0.88 mmol, 1.00 eq.) in toluene (10 mL) was added Grubbs 2nd generation catalyst (37.51 mg, 0.044 mmol, 0.050 eq.) in one portion at RT under nitrogen atmosphere. The reaction mixture was stirred for 12 h at 120° C., cooled and then concentrated. The residue was purified by silica gel column chromatography, eluted with PE/EA (10:1), to afford the title compound (70 mg, 23.9%) as a light-yellow oil.

### Step 6: (1R,5S)-3,8-diazabicyclo[3.2.1]oct-6-ene bis(2,2,2-trifluoroacetate)

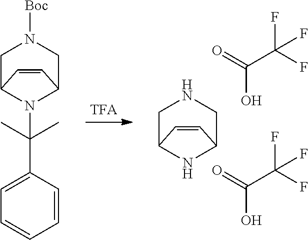

A mixture of tert-butyl (1R,5S)-8-(2-phenylpropan-2-yl)-3,8-diazabicyclo[3.2.1]oct-6-ene-3-carboxylate (200 mg, 0.61 mmol, 1.00 eq.) in TFA (3 mL) was refluxed for 2 h under nitrogen atmosphere. The reaction mixture was concentrated and the residue was triturated with Et₂O to afford the title compound (172.3 mg, 83.6%) as a white oil.

### Intermediate 8

### Synthesis of 7,9-diazatricyclo[3.3.1.0^{2,4}]nonane bis(2,2,2-trifluoroacetate)

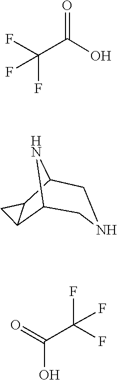 [SMILES: O=C(O)C(F)(F)F ; C1NCC2NC1C1CC21 ; O=C(O)C(F)(F)F]

### Step 1: tert-butyl 9-(2-phenylpropan-2-yl)-7,9-diazatricyclo[3.3.1.02,4]nonane-7-carboxylate

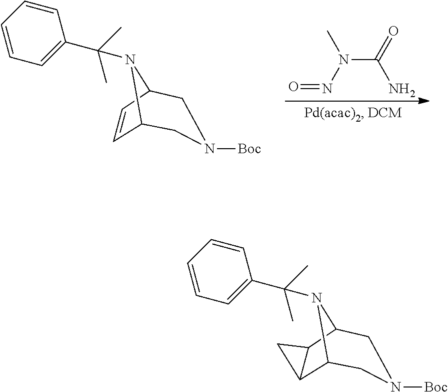

To a stirred mixture of KOH (3.0 g, 21.39 mmol, 14.07 eq., 40% aqueous solution) and ether (10 mL) was added 1-methyl-1-nitrosourea (1.0 g, 9.70 mmol, 6.38 eq.) in portions at 0-5° C. The resulting mixture was stirred for 5 min at the same temperature and the ether layer was separated. To a stirred solution of tert-butyl (1R,5S)-8-(2-phenylpropan-2-yl)-3,8-diazabicyclo[3.2.1]oct-6-ene-3-carboxylate (500 mg, 1.52 mmol, 1.00 eq.) and Pd(acac)₂ (46 mg, 0.15 mmol, 0.10 eq.) in DCM (10 mL) was added the above ether solution dropwise at RT. The resulting mixture was stirred for 30 min at RT, diluted with water and then extracted with DCM. The combined organic layers were washed with water, brine, dried over anhydrous Na₂SO₄, filtered and then concentrated. The residue was purified by silica gel column chromatography, eluted with EA/PE (0-20%), to afford the title compound (350 mg, 67.1%) as a yellow oil.

### Step 2: 7,9-diazatricyclo[3.3.1.0^{2,4}]nonane bis(2,2,2-trifluoroacetate)

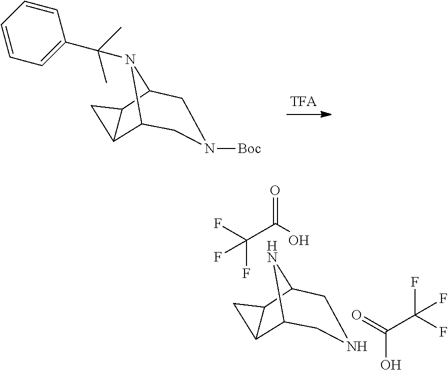

Proceeding analogously as described in Intermediate 6, Step 4 above, but using tert-butyl (1R,2R,4S,5S)-9-(2-phenylpropan-2-yl)-7,9-diazatricyclo[3.3.1.02,4]nonane-7-carboxylate and TFA provided the title compound.

### Intermediate 9

### Synthesis of 6-methylene-3,8-diazabicyclo[3.2.1]octane bis(2,2,2-trifluoroacetate)

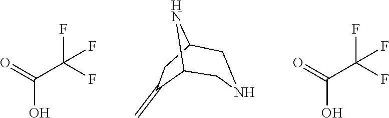

### Step 1: tert-butyl 6-hydroxy-8-(2-phenylpropan-2-yl)-3,8-diazabicyclo[3.2.1]octane-3-carboxylate

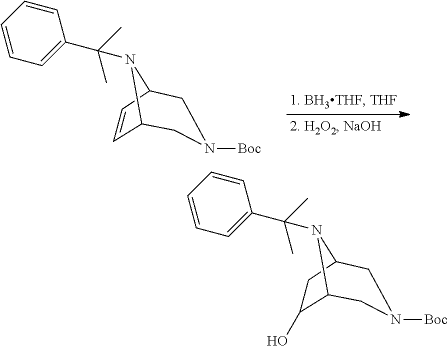

To a stirred solution of tert-butyl 8-(2-phenylpropan-2-yl)-3,8-diazabicyclo[3.2.1]oct-6-ene-3-carboxylate (374 mg, 1.14 mmol, 1.00 eq.) in THF (12 mL) was added 1.0 M BH₃·THF (4.5 mL, 4.5 mmol, 3.95 eq.) dropwise at 0° C. under nitrogen atmosphere. After stirring overnight at RT, 5% NaOH (aq.) (4.5 mL) was added over 10 min, followed by 30% H₂O₂ (390 mg, 3.44 mmol, 3.02 eq.) at 0° C. The resulting mixture was stirred for 1h at RT and then extracted with DCM. The organic layer was concentrated and the residue was purified by trituration with Et₂O to afford the title compound (300 mg, 76.3%) as a white oil.

### Step 2: tert-butyl 6-oxo-8-(2-phenylpropan-2-yl)-3,8-diazabicyclo[3.2.1]octane-3-carboxylate

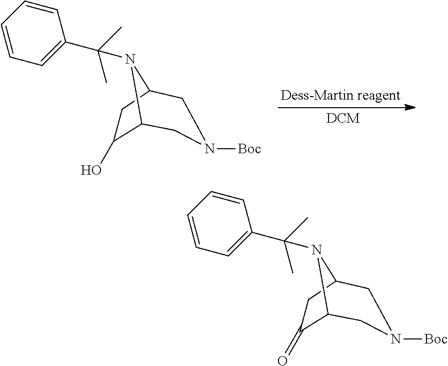

To a stirred solution of tert-butyl 6-hydroxy-8-(2-phenylpropan-2-yl)-3,8-diazabicyclo[3.2.1] octane-3-carboxylate (300 mg, 0.87 mmol, 1.00 eq.) in DCM (10 mL) was added Dess-Martin reagent (551 mg, 1.30 mmol, 1.49 eq.) in portions at 0° C. After stirring at 0° C. for 1h, the reaction mixture was diluted with water and then extracted with EtOAc. The organic layer was washed with water, NaHCO₃ (aq.), dried over Na₂SO₄, filtered and then concentrated. The residue was purified by silica gel column chromatography, eluted with PE/EA (10:1), to afford the title compound (210 mg, 70.1%) as a yellow oil.

### Step 3: tert-butyl 6-methylene-8-(2-phenylpropan-2-yl)-3,8-diazabicyclo[3.2.1]octane-3-carboxylate

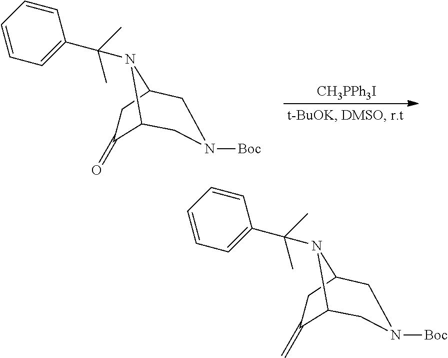

A solution of methyltriphenylphosphanium iodide (528 mg, 1.31 mmol, 3.00 eq.) and t-BuOK (147 mg, 1.31 mmol, 3.00 eq.) in DMSO (3 mL) was stirred for 10 min at 10° C. under nitrogen atmosphere. To this stirred mixture was added a solution of tert-butyl (1R,5R)-6-oxo-8-(2-phenylpropan-2-yl)-3,8-diazabicyclo[3.2.1]octane-3-carboxylate (150 mg, 0.44 mmol, 1.00 eq.) in DMSO (1 mL) dropwise at 10° C. After stirring at RT for 3 h, the reaction mixture was quenched with water and then extracted with EtOAc. The combined organic layers were washed with brine, dried over anhydrous Na₂SO₄, filtered and then concentrated. The residue was purified by silica gel column chromatography, eluted with PE/EA (10:1), to afford the title compound (99 mg, 65.9%) as a yellow oil.

### Step 4: 6-methylene-3,8-diazabicyclo[3.2.1]octane bis(2,2,2-trifluoroacetate)

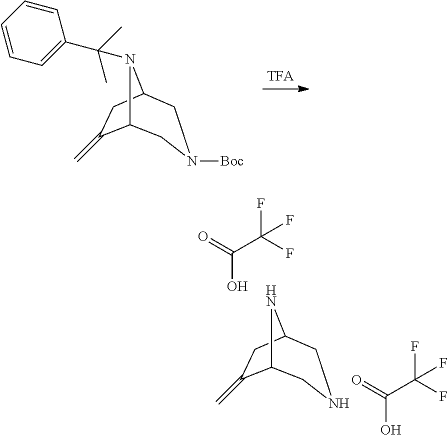

Proceeding analogously as described in Intermediate 6, Step 4 above, but using tert-butyl 6-methylene-8-(2-phenylpropan-2-yl)-3,8-diazabicyclo[3.2.1]octane-3-carboxylate and TFA provided the title compound.

### Intermediate 10

### Synthesis of ((2R,3R,7aR)-2-fluoro-3-methyl-6-methylenetetrahydro-1H-pyrrolizin-7a(5H)-yl)methanol

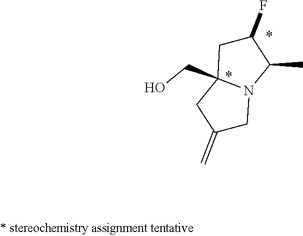

### Step 1: 1-(tert-butyl) 2-methyl (2R,4S,5R)-2-(2-(chloromethyl)allyl)-5-methyl-4-((4-nitrobenzoyl)oxy)pyrrolidine-1,2-dicarboxylate

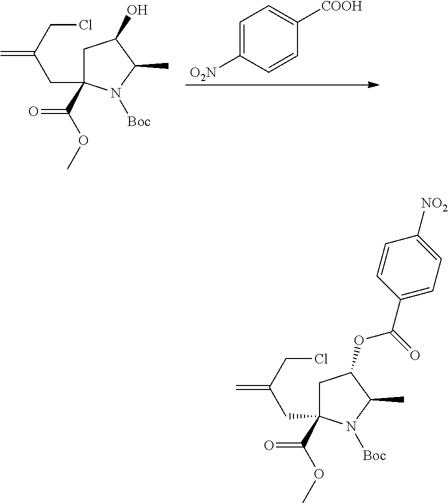

To a solution of 1-(tert-butyl) 2-methyl (2R,4R,5R)-2-(2-(chloromethyl)allyl)-4-hydroxy-5-methylpyrrolidine-1,2-dicarboxylate (6.00 g, 17.2 mmol, 1 eq), 4-nitrobenzoic acid (3.17 g, 18.9 mmol, 1.1 eq) and PPh₃ (6.79 g, 25.8 mmol, 1.5 eq) in THF (60 mL) was added DIAD (5.23 g, 25.8 mmol, 5.03 mL, 1.5 eq) at 0° C. and the mixture was stirred at 20° C. for 12 h. The mixture was quenched with water and extracted with EtOAc. The combined organic layers were concentrated and the residue was purified by column chromatography, eluted with EA/PE (0-20%) to afford the title compound (5.00 g) as a yellow oil.

### Step 2: methyl (2S,3R,8R)-3-methyl-6-methylene-2-(4-nitrobenzoyl)oxy-2,3,5,7-tetrahydro-1H-pyrrolizine-8-carboxylate

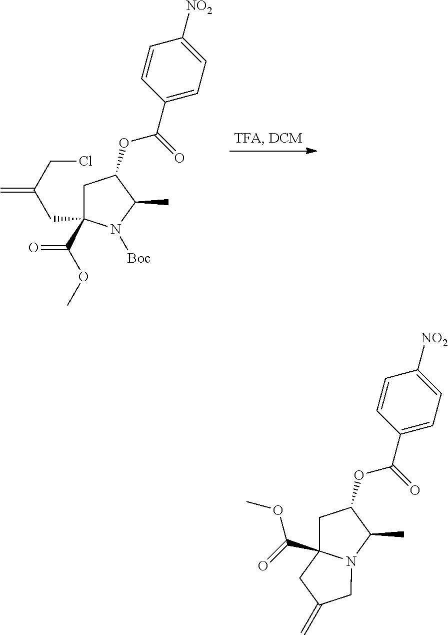 [SMILES: C=C(CCl)C[C@]1(C(=O)OC)C[C@H](OC(=O)c2ccc([N+](=O)[O-])cc2)[C@@H](C)N1C(=O)OC(C)(C)C ; C=C1CN2[C@H](C)[C@@H](OC(=O)c3ccc([N+](=O)[O-])cc3)C[C@@]2(C(=O)OC)C1]

To a solution of 1-tert-butyl 2-methyl (2R,4S,5R)-2-[2-(chloromethyl)allyl]-5-methyl-4-(4-nitrobenzoyl)oxy-pyrrolidine-1,2-dicarboxylate (1.00 g, 2.01 mmol, 1 eq) in DCM (12 mL) was added TFA (6.16 g, 54.0 mmol, 4 mL, 26.8 eq) and the mixture was stirred at 20° C. for 2 h. The mixture was concentrated and the residue was dissolved in THF. K₂CO₃ (834 mg, 6.04 mmol, 3 eq) was added and the resulting mixture was stirred at 20° C. for 2 h. The mixture was then filtered and concentrated to afford the title compound (2.9 g, crude).

### Step 3: methyl (2S,3R,8R)-2-hydroxy-3-methyl-6-methylene-2,3,5,7-tetrahydro-1H-pyrrolizine-8-carboxylate

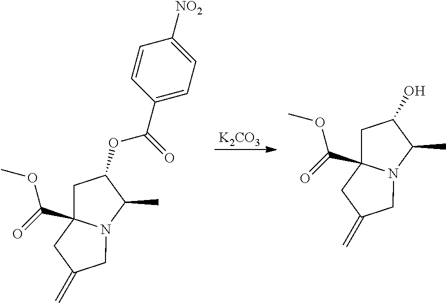

To a solution of methyl (2S,3R,8R)-3-methyl-6-methylene-2-(4-nitrobenzoyl)oxy-2,3,5,7-tetrahydro-1H-pyrrolizine-8-carboxylate (2.90 g, 8.05 mmol, 1 eq) in MeOH (5 mL) was added K₂CO₃ (2.78 g, 20.1 mmol, 2.5 eq) and the mixture was stirred at 20° C. for 2 h. The mixture was filtered and the filtrate was concentrated. The residue was purified by column chromatography, eluted with EA/PE (0-50%) to afford the title compound (700 mg).

### Step 4. methyl (2R,3R,8R)-2-fluoro-3-methyl-6-methylene-2,3,5,7-tetrahydro-1H-pyrrolizine-8-carboxylate

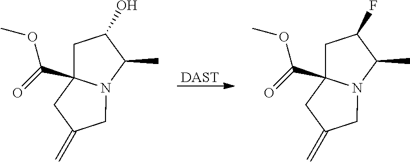

To a solution of methyl (2S,3R,8R)-2-hydroxy-3-methyl-6-methylene-2,3,5,7-tetrahydro-1H-pyrrolizine-8-carboxylate (0.25 g, 1.18 mmol, 1 eq) in DCM (4 mL) was added DAST (572 mg, 3.55 mmol, 469 uL, 3 eq) dropwise at −60° C. The mixture was warmed to rt and stirred for 12 h. The reaction mixture was cooled in ice water bath and quenched with water. The mixture was extracted with EtOAc and the combined organic layers were concentrated. The residue was purified by column chromatography, eluted with EA/PE (0-30%), to afford the title compound (170 mg).

### Step 5: ((2R,3R,7aR)-2-fluoro-3-methyl-6-methylenetetrahydro-1H-pyrrolizin-7a(5H)-yl)methanol

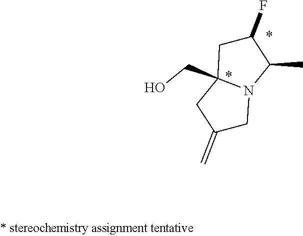

The title compound was prepared by proceeding analogously as described in Intermediate 11, Step 5, by reducing the ester group with LiAlH₄. MS (ES, m/z): [M+H]⁺=186.2.

### Intermediate 11

### Synthesis of ((2S,7aR)-2-fluoro-6-methylenetetrahydro-1H-pyrrolizin-7a(5H)-yl)methanol

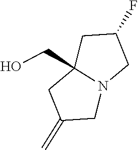

### Step 1: 1-(tert-butyl) 2-methyl (2R,4R)-4-((tert-butyldimethylsilyl)oxy)-2-(2-(chloromethyl)allyl)-pyrrolidine-1,2-dicarboxylate

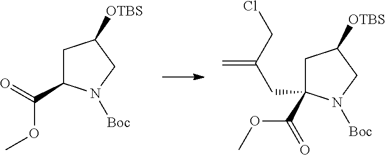 [SMILES: COC(=O)[C@H]1C[C@@H](O[Si](C)(C)C(C)(C)C)CN1C(=O)OC(C)(C)C ; C=C(CCl)C[C@]1(C(=O)OC)C[C@@H](O[Si](C)(C)C(C)(C)C)CN1C(=O)OC(C)(C)C]

A solution of HMDS (52 g, 320 mmol, 1.15 equiv) in THF (1000 mL) was treated with n-BuLi (128 mL, 320 mmol, 1.15 equiv) for 30 min at −78° C. under nitrogen atmosphere, followed by the addition of 1-(tert-butyl) 2-methyl (2R,4R)-4-((tert-butyldimethylsilyl)oxy)pyrrolidine-1,2-dicarboxylate (100 g, 278 mmol, 1.0 equiv) dropwise at −78° C. 3-Chloro-2-(chloromethyl)prop-1-ene (87 g, 695 mmol, 2.5 equiv) was added dropwise over 20 min. at −78° C. and the resulting mixture was stirred for additional 12 h at room temperature. The reaction was quenched with NH₄Cl (aq.) at 5° C. and the mixture was extracted with EtOAc. The combined organic layers were washed with brine, dried over anhydrous Na₂SO₄ and concentrated. The residue was purified by silica gel column chromatography, eluted with EA/PE (0-30%) to afford to afford the title compound (87 g).

### Step 2: 1-(tert-butyl) 2-methyl (2R,4R)-2-(2-(chloromethyl)allyl)-4-hydroxypyrrolidine-1,2-dicarboxylate

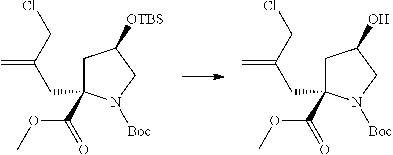 [SMILES: C=C(CCl)C[C@]1(C(=O)OC)C[C@@H](O[Si](C)(C)C(C)(C)C)CN1C(=O)OC(C)(C)C ; C=C(CCl)C[C@]1(C(=O)OC)C[C@@H](O)CN1C(=O)OC(C)(C)C]

To a solution of 1-(tert-butyl) 2-methyl (2R,4R)-4-((tert-butyldimethylsilyl)oxy)-2-(2-(chloromethyl)allyl)pyrrolidine-1,2-dicarboxylate (100 g, 223 mmol, 1.0 equiv) in THF (1000 mL) was added TBAF (267 mL, 267 mmol, 1.2 equiv, 1 M in THF) dropwise at room temperature and the resulting mixture was stirred for 16 h at room temperature. The reaction mixture was then concentrated under reduced pressure and the residue was purified by silica gel column chromatography, eluted with EA/PE (0-10%) to afford the title compound (65 g).

### Step 3: 1-(tert-butyl) 2-methyl (2R,4S)-2-(2-(chloromethyl)allyl)-4-fluoropyrrolidine-1,2-dicarboxylate

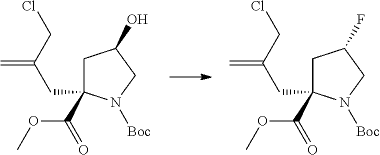 [SMILES: C=C(CCl)C[C@]1(C(=O)OC)C[C@@H](O)CN1C(=O)OC(C)(C)C ; C=C(CCl)C[C@]1(C(=O)OC)C[C@H](F)CN1C(=O)OC(C)(C)C]

To a stirred solution of 1-(tert-butyl) 2-methyl (2R,4R)-2-(2-(chloromethyl)allyl)-4-hydroxypyrrolidine-1,2-dicarboxylate (60 g, 180 mmol, 1.0 equiv) in DCM (600 mL) was added BAST (59.6 g, 270 mmol, 1.5 equiv) dropwise at −78° C. The resulting mixture was stirred for 16 h at room temperature. The reaction was quenched by adding water and the mixture was extracted with DCM. The combined organic layers were washed with water and brine, dried over anhydrous Na₂SO₄ and concentrated. The residue was purified by silica gel column chromatography, eluted with EA/PE (0-40%) to afford the title compound (30 g).

### Step 4: methyl (2S,7aR)-2-fluoro-6-methylenetetrahydro-1H-pyrrolizine-7a(5H)-carboxylate

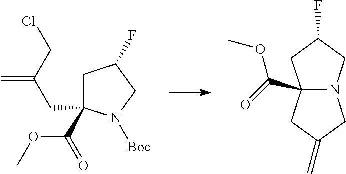 [SMILES: C=C(CCl)C[C@]1(C(=O)OC)C[C@H](F)CN1C(=O)OC(C)(C)C ; C=C1CN2C[C@@H](F)C[C@@]2(C(=O)OC)C1]

To a solution of 1-tert-butyl 2-methyl (2R,4S)-2-[2-(chloromethyl)prop-2-en-1-yl]-4-fluoropyrrolidine-1,2-dicarboxylate (30 g, 89.3 mmol, 1 equiv.) in DCM (225 mL) was added TFA (75 mL) dropwise at 0° C. and the resulting mixture was stirred for 4 h at room temperature. The reaction mixture was then concentrated under reduced pressure and the residue was basified to pH 8 by adding NH₃ solution in MeOH at 0-5° C. The mixture was concentrated under reduced pressure and the residue was purified by silica gel column chromatography, eluted with EA/PE (0-50%) to afford the title compound (16 g).

### Step 5: ((2S,7aR)-2-fluoro-6-methylenetetrahydro-1H-pyrrolizin-7a(5H)-yl)methanol

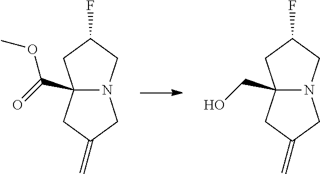

To a stirred solution of methyl (2S,7aR)-2-fluoro-6-methylenetetrahydro-1H-pyrrolizine-7a(5H)-carboxylate (2 g, 10 mmol, 1 equiv) in THF (20 mL) was added LiAlH₄ (10 mL, 10 mmol, 1.0 equiv, 1.0 M in THF) dropwise at 0° C. and the resulting mixture was stirred for 1 h at room temperature. The reaction was quenched with 15% NaOH(aq) and water at 0° C. The resulting mixture was filtered, the filter cake was washed with EtOAc. The filtrate was concentrated under reduced pressure to afford the title compound (1.73 g). MS (ES, m/z): [M+H]⁺=172.2.

### Intermediate 12

### Synthesis of ((2R,7aR)-2-fluoro-6-methylenetetrahydro-1H-pyrrolizin-7a(5H)-yl)methanol

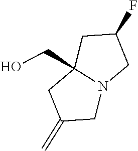

### Step 1: 1-(tert-butyl) 2-methyl (2R,4R)-2-(2-(chloromethyl)allyl)-4-fluoropyrrolidine-1,2-dicarboxylate

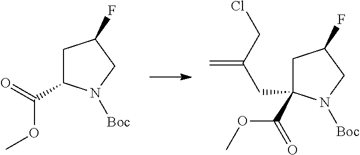 [SMILES: COC(=O)[C@@H]1C[C@@H](F)CN1C(=O)OC(C)(C)C ; C=C(CCl)C[C@]1(C(=O)OC)C[C@@H](F)CN1C(=O)OC(C)(C)C]

To a stirred solution of 1-tert-butyl 2-methyl (2S,4R)-4-fluoropyrrolidine-1,2-dicarboxylate (40 g, 162 mmol, 1.0 equiv) in THF (400 mL) added LiHMDS (324 mL, 324 mmol, 2.0 equiv, 1.0 M in THF) dropwise at −78° C. under nitrogen atmosphere. The resulting mixture was stirred for 15 min. 3-Chloro-2-(chloromethyl)prop-1-ene (30.4 g, 243 mmol, 1.5 equiv) was added dropwise at -78° C. under nitrogen atmosphere. The resulting mixture was stirred overnight room temperature under nitrogen atmosphere. The reaction was acidified with 1.0 M HCl aq. solution at 0° C. to pH=7 and the resulting mixture was extracted with EtOAc. The combined organic layers were washed with water and brine, dried over anhydrous Na₂SO₄ and concentrated. The residue was purified by silica gel column chromatography, eluted with EA/PE (0-10%) to afford the title compound (20 g).

### Step 2: methyl (2R,7aR)-2-fluoro-6-methylenetetrahydro-1H-pyrrolizine-7a(5H)-carboxylate

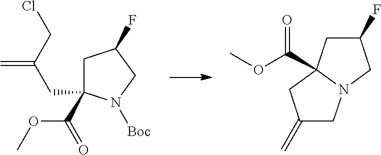 [SMILES: C=C(CCl)C[C@]1(C(=O)OC)C[C@@H](F)CN1C(=O)OC(C)(C)C ; C=C1CN2C[C@H](F)C[C@@]2(C(=O)OC)C1]

To a stirred solution of 1-(tert-butyl) 2-methyl (2R,4R)-2-(2-(chloromethyl)allyl)-4-fluoropyrrolidine-1,2-dicarboxylate (20 g, 59.5 mmol, 1 equiv.) in DCM (150 mL) was added TFA (50 mL) dropwise at 0° C. and the resulting mixture was stirred for 4 h at room temperature. The mixture was then concentrated under reduced pressure and the residue was basified to pH 8 with NH₃ solution in MeOH at 0-5° C. The mixture was then concentrated under reduced pressure and the residue was purified by silica gel column chromatography, eluted with EA/PE (0-50%) to afford the title compound (10.7 g).

### Step 3: ((2R,7aR)-2-fluoro-6-methylenetetrahydro-1H-pyrrolizin-7a(5H)-yl)methanol

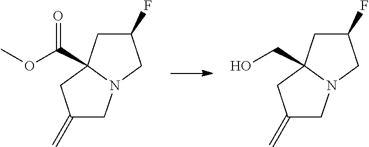

The title compound was prepared by proceeding analogously as described in Intermediate 11, Step 5, by reducing the ester group with LiAlH₄. MS (ES, m/z): [M+H]⁺=172.2.

### Intermediate 13

### Synthesis of ((2R,7aS)-2-fluoro-6-methylenetetrahydro-1H-pyrrolizin-7a(5H)-yl)methanol

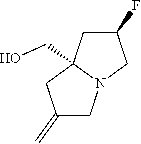

### Step 1: 1-(tert-butyl) 2-methyl (2S,4S)-4-((tert-butyldimethylsilyl)oxy)-2-(2-(chloromethyl)allyl)-pyrrolidine-1,2-dicarboxylate

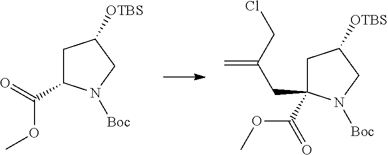 [SMILES: COC(=O)[C@@H]1C[C@H](O[Si](C)(C)C(C)(C)C)CN1C(=O)OC(C)(C)C ; C=C(CCl)C[C@@]1(C(=O)OC)C[C@H](O[Si](C)(C)C(C)(C)C)CN1C(=O)OC(C)(C)C]

To a stirred solution of HMDS (52 g, 320 mmol, 1.15 equiv) in THF (1000 mL) was added n-BuLi (20 g, 320 mmol, 1.15 equiv) dropwise at −78° C. under nitrogen atmosphere and the resulting mixture was stirred for 30 min at −78° C. under nitrogen atmosphere. 1-(tert-butyl) 2-Methyl (2S,4S)-4-((tert-butyldimethylsilyl)oxy)-pyrrolidine-1,2-dicarboxylate (100 g, 278 mmol, 1 equiv.) was added and the resulting mixture was stirred for 15 min at −78° C. under nitrogen atmosphere. 3-Chloro-2-(chloromethyl)prop-1-ene (87 g, 695 mmol, 2.5 equiv) was added to the above solution and the resulting mixture was stirred for 2 h at room temperature under nitrogen atmosphere. The reaction was quenched with sat. NH₄Cl (aq.) at 0° C. and the mixture was extracted with EtOAc. The combined organic layers were washed with brine, dried over anhydrous Na₂SO₄ and concentrated. The residue was purified by silica gel column chromatography, eluted with PE/EA (10:1) to afford the title compound (75 g).

### Step 2: methyl (2S,7aS)-2-hydroxy-6-methylenetetrahydro-1H-pyrrolizine-7a(5H)-carboxylate

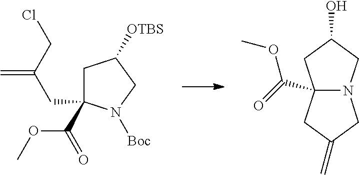 [SMILES: C=C(CCl)C[C@]1(C(=O)OC)C[C@H](O[Si](C)(C)C(C)(C)C)CN1C(=O)OC(C)(C)C ; C=C1CN2C[C@@H](O)C[C@]2(C(=O)OC)C1]

To a solution of 1-(tert-butyl) 2-methyl (2R,4S)-4-((tert-butyldimethylsilyl)oxy)-2-(2-(chloromethyl)allyl)pyrrolidine-1,2-dicarboxylate (60 g, 134 mmol, 1 equiv.) in DCM (450 mL) was added TFA (150 mL) dropwise at 0° C. and the resulting mixture was stirred for 16 h at room temperature. The mixture was concentrated under vacuum and the residue was basified to pH 8 with NH₃ solution in MeOH. The mixture was concentrated under reduced pressure and the residue was purified by silica gel column chromatography, eluted with MeOH/DCM (0-10%) to afford the title compound (18.8 g).

### Step 3: methyl (2R,7aS)-2-fluoro-6-methylenetetrahydro-1H-pyrrolizine-7a(5H)-carboxylate

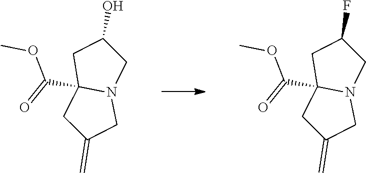

To a stirred solution of methyl (2S,7aS)-2-hydroxy-6-methylenetetrahydro-1H-pyrrolizine-7a(5H)-carboxylate (1.0 g, 5.1 mmol, 1 equiv.) in DCM (10 mL) was added DAST (1.23 g, 7.6 mmol, 1.5 equiv) dropwise at −78° C. and the resulting mixture was stirred for 2 h at room temperature. The mixture was then extracted with DCM and the combined organic layers were washed with brine, dried over anhydrous Na₂SO₄ and concentrated. The residue was purified by silica gel column chromatography, eluted with EA/PE (0-50%) to afford the title compound (300 mg).

### Step 4: ((2R,7aS)-2-fluoro-6-methylenetetrahydro-1H-pyrrolizin-7a(5H)-yl)methanol

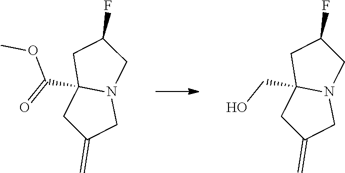

The title compound was prepared by proceeding analogously as described in Intermediate 11, Step 5, by reducing the ester group with LiAlH₄. MS (ES, m/z): [M+H]⁺=172.1

### Intermediate 14

### Synthesis of: ((2S,7aS)-2-fluoro-6-methylenetetrahydro-1H-pyrrolizin-7a(5H)-yl)methanol

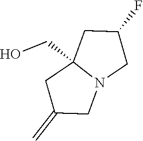

### Step 1: 1-(tert-butyl) 2-methyl (2S,4R)-2-(2-(chloromethyl)allyl)-4-((4-nitrobenzoyl)oxy)-pyrrolidine-1,2-dicarboxylate

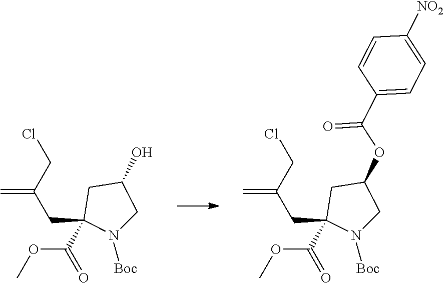 [SMILES: C=C(CCl)C[C@@]1(C(=O)OC)C[C@H](O)CN1C(=O)OC(C)(C)C ; C=C(CCl)C[C@@]1(C(=O)OC)C[C@@H](OC(=O)c2ccc([N+](=O)[O-])cc2)CN1C(=O)OC(C)(C)C]

To a stirred solution of 1-(tert-butyl) 2-methyl (2S,4S)-2-(2-(chloromethyl)allyl)-4-hydroxypyrrolidine-1,2-dicarboxylate (7.5 g, 22.5 mmol, 1 equiv.), 4-nitrobenzoic acid (4.5 g, 27.0 mmol, 1.2 equiv.) and PPh₃ (7.1 g, 27.0 mmol, 1.2 equiv) in THF (75 mL) was slowly added DEAD (4.7 g, 27 mmol, 1.2 equiv) at 0° C. The resulting mixture was allowed to warm to room temperature and then stirred for 1 h at 25° C. under N₂ atmosphere. The reaction was quenched with aq. NaHCO₃ solution at 0° C. and the resulting mixture was extracted with EtOAc. The combined organic layers were washed with water and brine, dried over anhydrous Na₂SO₄ and concentrated. The residue was purified by silica gel column chromatography, eluted with EA/PE (0˜20%) to afford the title compound (5.0 g).

### Step 2: methyl (6R,7aS)-2-methylene-6-((4-nitrobenzoyl)oxy)tetrahydro-1H-pyrrolizine-7a(5H)-carboxylate

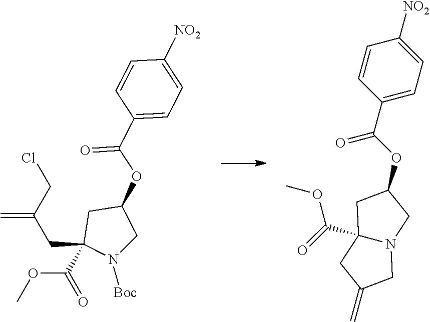 [SMILES: C=C(CCl)C[C@@]1(C(=O)OC)C[C@@H](OC(=O)c2ccc([N+](=O)[O-])cc2)CN1C(=O)OC(C)(C)C ; C=C1CN2C[C@H](OC(=O)c3ccc([N+](=O)[O-])cc3)C[C@]2(C(=O)OC)C1]

A solution of 1-(tert-butyl) 2-methyl (2S,4R)-2-(2-(chloromethyl)allyl)-4-((4-nitrobenzoyl)oxy)pyrrolidine-1,2-dicarboxylate (11 g, 22.78 mmol, 1.0 equiv.) in DCM (100 mL) was added TFA (50 mL) dropwise at 0° C. and the resulting mixture was stirred for 1 h at room temperature. The reaction mixture was then concentrated under vacuum and the residue was neutralized to pH 7 with NH₃ solution in MeOH. The mixture was concentrated under vacuum and the residue was purified by silica gel column chromatography, eluted with PE/EA (0˜20%) to afford the title compound (6.3 g).

### Step 3: methyl (2R,7aS)-2-hydroxy-6-methylenetetrahydro-1H-pyrrolizine-7a(5H)-carboxylate

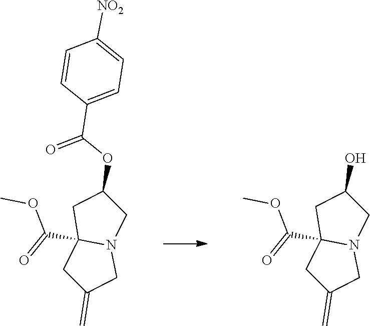 [SMILES: C=C1CN2C[C@H](OC(=O)c3ccc([N+](=O)[O-])cc3)C[C@]2(C(=O)OC)C1 ; C=C1CN2C[C@H](O)C[C@]2(C(=O)OC)C1]

A mixture of methyl (6R,7aS)-2-methylene-6-((4-nitrobenzoyl)oxy)tetrahydro-1H-pyrrolizine-7a(5H)-carboxylate (5.8 g, 16.7 mmol, 1.0 equiv) and K₂CO₃ (4.63 g, 33.5 mmol, 2.0 equiv) in MeOH (58 mL) was stirred for 1 h at room temperature. The reaction mixture was concentrated under vacuum and the residue was purified by silica gel column chromatography, eluted with MeOH/DCM (0˜10%) to afford the title compound (2.8 g).

### Step 4: methyl (2S,7aS)-2-fluoro-6-methylenetetrahydro-1H-pyrrolizine-7a(5H)-carboxylate

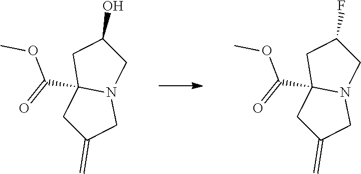

To a solution of methyl (2R,7aS)-2-hydroxy-6-methylenetetrahydro-1H-pyrrolizine-7a(5H)-carboxylate (2.0 g, 10 mmol, 1 equiv.) in DCM (20 mL) was added BAST (3.37 g, 15 mmol, 1.5 equiv) dropwise at 0° C. under N₂ atmosphere and the resulting mixture was stirred for 16 h at room temperature. The mixture was basified to pH=7 with NH₃ solution in MeOH and then concentrated under vacuum. The residue was purified by silica gel column chromatography, eluted with PE/EA (0˜50%) to afford the title compound (900 mg).

### Step 5: ((2S,7aS)-2-fluoro-6-methylenetetrahydro-1H-pyrrolizin-7a(5H)-yl)methanol

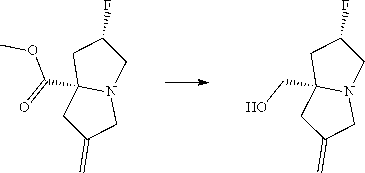

The title compound was prepared by proceeding analogously as described in Intermediate 11, Step 5, by reducing the ester group with LiAlH₄. MS (ES, m/z): [M+H]⁺=172.2

### Intermediate 15

### Synthesis of ((6R,7aR)-2-ethylidene-6-fluorotetrahydro-1H-pyrrolizin-7a(5H)-yl)methanol

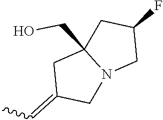

### Step 1: methyl (6R,7aR)-2-ethylidene-6-fluorotetrahydro-1H-pyrrolizine-7a(5H)-carboxylate

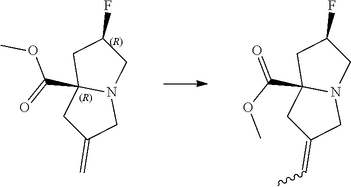

A tube was charged with of methyl (2R,7aR)-2-fluoro-6-methylidene-tetrahydro-1H-pyrrolizine-7a-carboxylate (2.5 g, 12.55 mmol, 1.0 equiv), Grubbs 2^{nd} generation catalyst (3.2 g, 3.76 mmol, 0.3 equiv) and DCM (50 mL). Propylene gas was bubbled into the mixture at 0° C. for 10 min. The tube was then sealed and stirred overnight at 40° C. After cooling to rt, the mixture was concentrated under reduced pressure. The residue was purified by silica gel column chromatography, eluted with EA/PE (0-50%). The product was further purified by prep-HPLC to afford the title compound (1 g) as a brown oil.

### Step 2: ((6R,7aR)-2-ethylidene-6-fluorotetrahydro-1H-pyrrolizin-7a(5H)-yl)methanol

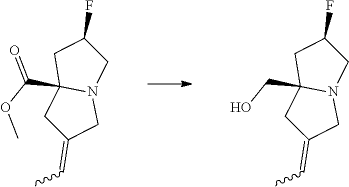

The title compound was prepared by proceeding analogously as described in Intermediate 11, Step 5, by reducing the ester group with LiAlH₄. MS (ES, m/z): [M+H]⁺=186.2

### Intermediate 16

### Synthesis of ((6S,7aR)-2-cyclobutylidene-6-fluorotetrahydro-1H-pyrrolizin-7a(5H)-yl)methanol

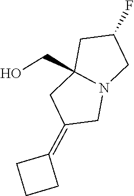 [SMILES: OC[C@]12CC(=C3CCC3)CN1C[C@@H](F)C2]

The title compound was prepared by proceeding analogously as described in Intermediate 15, Steps 1-2, using methyl (2S,7aR)-2-fluoro-6-methylenetetrahydro-1H-pyrrolizine-7a(5H)-carboxylate and methylenecyclobutane instead of methyl (2R,7aR)-2-fluoro-6-methylidene-tetrahydro-1H-pyrrolizine-7a-carboxylate and propylene in Step 1. MS (ES, m/z): [M+H]⁺=212.2.

### Intermediate 17

### Synthesis of ((6R,7aR)-2-cyclobutylidene-6-fluorotetrahydro-1H-pyrrolizin-7a(5H)-yl)methanol

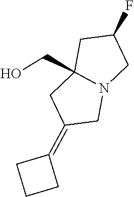 [SMILES: OC[C@]12CC(=C3CCC3)CN1C[C@H](F)C2]

The title compound was prepared by proceeding analogously as described in Intermediate 15, Steps 1-2, using methylenecyclobutane instead of propylene in Step 1. MS (ES, m/z): [M+H]⁺=212.2.

### Intermediate 18

### Synthesis of ((2R,7aR)-2-fluoro-6-(propan-2-ylidene)tetrahydro-1H-pyrrolizin-7a(5H)-yl)-methanol

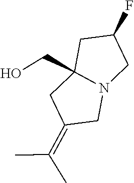

The title compound was prepared by proceeding analogously as described in Intermediate 15, Steps 1-2, using 2-methylprop-1-ene instead of propylene in Step 1. MS (ES, m/z): [M+H]⁺=200.2.

### Intermediate 19

### Synthesis of ((2S,7aS)-2-methoxy-6-methylenetetrahydro-1H-pyrrolizin-7a(5H)-yl)methanol

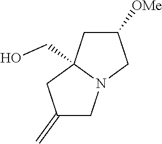

### Step 1: 1-(tert-butyl) 2-methyl (2S,4S)-2-(2-(chloromethyl)allyl)-4-hydroxypyrrolidine-1,2-dicarboxylate

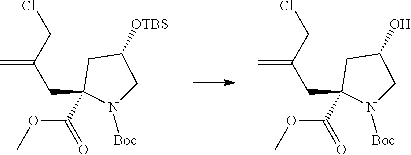 [SMILES: C=C(CCl)C[C@@]1(C(=O)OC)C[C@H](O[Si](C)(C)C(C)(C)C)CN1C(=O)OC(C)(C)C ; C=C(CCl)C[C@@]1(C(=O)OC)C[C@H](O)CN1C(=O)OC(C)(C)C]

To a solution of 1-tert-butyl 2-methyl (2S,4S)-4-[(tert-butyldimethylsilyl)oxy]-2-[2-(chloromethyl)prop-2-en-1-yl]pyrrolidine-1,2-dicarboxylate (25 g, 56 mmol, 1 equiv) in THF (250 mL) was added TBAF (18 g, 67 mmol, 1.2 equiv) at room temperature and the resulting mixture was stirred for 2 h at room condition. The mixture was then concentrated under reduced pressure and the residue was purified by silica gel column chromatography, eluted with PE/EA (5:1) to afford the title compound (15 g).

### Step 2: 1-(tert-butyl) 2-methyl (2S,4S)-2-(2-(chloromethyl)allyl)-4-methoxypyrrolidine-1,2-dicarboxylate

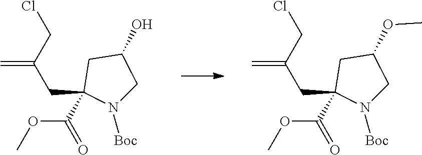 [SMILES: C=C(CCl)C[C@@]1(C(=O)OC)C[C@H](O)CN1C(=O)OC(C)(C)C ; C=C(CCl)C[C@@]1(C(=O)OC)C[C@H](OC)CN1C(=O)OC(C)(C)C]

A mixture of 1-(tert-butyl) 2-methyl (2S,4S)-2-(2-(chloromethyl)allyl)-4-hydroxy-pyrrolidine-1,2-dicarboxylate (2.6 g, 7.8 mmol, 1.0 equiv), CH₃I (2.2 g, 15.6 mmol, 2.0 equiv) and Ag₂O (9.0 g, 39 mmol, 5.0 equiv) in acetone (30 mL) was stirred overnight at room temperature. The reaction mixture was filtered and the filter cake was washed with acetone. The filtrate was concentrated under reduced pressure to give the title compound (2.3 g).

### Step 5: methyl (2S,7aS)-2-methoxy-6-methylenetetrahydro-1H-pyrrolizine-7a(5H)-carboxylate

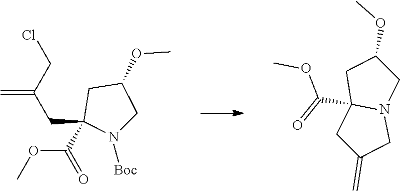 [SMILES: C=C(CCl)C[C@@]1(C(=O)OC)C[C@H](OC)CN1C(=O)OC(C)(C)C ; C=C1CN2C[C@@H](OC)C[C@]2(C(=O)OC)C1]

To a solution of 1-(tert-butyl) 2-methyl (2S,4S)-2-(2-(chloromethyl)allyl)-4-methoxy-pyrrolidine-1,2-dicarboxylate (2.3 g, 6.6 mmol, 1.0 equiv) in DCM (25 mL) was added TFA (8 mL) dropwise at 0-5° C. and the resulting mixture was stirred overnight at room temperature. The mixture was concentrated under reduced pressure and the residue was basified to pH=8 with NH₃ solution in MeOH. The volatile solvents were removed under vacuum and the residue was purified by silica gel column chromatography, eluted with DCM/MeOH (10:1) to afford the title compound (880 mg).

### Step 6: ((2S,7aS)-2-methoxy-6-methylenetetrahydro-1H-pyrrolizin-7a(5H)-yl)methanol

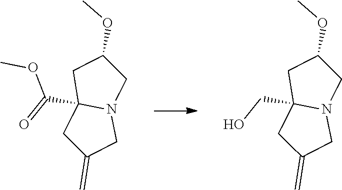

The title compound was prepared by proceeding analogously as described in Intermediate 11 Step 5, by reducing the ester group with LiAlH₄. MS (ES, m/z): [M+Na]⁺=206.2

### Intermediate 20

### Synthesis of ((2R,7aR)-2-methoxy-6-methylenetetrahydro-1H-pyrrolizin-7a(5H)-yl)methanol

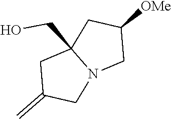

The title compound was prepared by proceeding analogously as described in Intermediate 19, Steps 2-6, using 1-(tert-butyl) 2-methyl (2R,4R)-2-(2-(chloromethyl)allyl)-4-hydroxypyrrolidine-1,2-dicarboxylate instead of 1-(tert-butyl) 2-methyl (2S,4S)-2-(2-(chloromethyl)allyl)-4-hydroxypyrrolidine-1,2-dicarboxylate in Step 2. MS (ES, m/z): [M+H]⁺=184.2

### Intermediate 21

### Synthesis of ((2R,7aS)-2-methoxy-6-methylenetetrahydro-1H-pyrrolizin-7a(5H)-yl)methanol

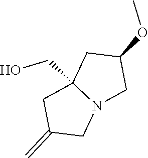

### Step 1: 1-(tert-butyl) 2-methyl (2S,4R)-2-(2-(chloromethyl)allyl)-4-hydroxypyrrolidine-1,2-dicarboxylate

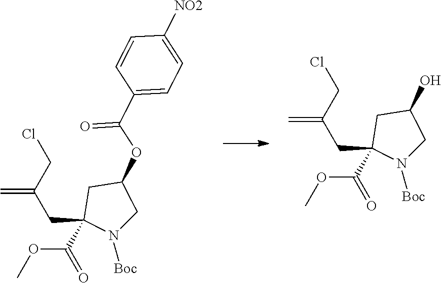 [SMILES: C=C(CCl)C[C@@]1(C(=O)OC)C[C@@H](OC(=O)c2ccc([N+](=O)[O-])cc2)CN1C(=O)OC(C)(C)C ; C=C(CCl)C[C@@]1(C(=O)OC)C[C@@H](O)CN1C(=O)OC(C)(C)C]

A mixture of 1-(tert-butyl) 2-methyl (2S,4R)-2-(2-(chloromethyl)allyl)-4-((4-nitro-benzoyl)oxy)pyrrolidine-1,2-dicarboxylate (4.8 g, 10 mmol, 1 equiv) and LiOH·H₂O (1.2 g, 50 mmol, 5 equiv.) in THF (50 mL) was stirred for 1 h at 50° C. After cooling to room temperature, the mixture was extracted with EtOAc. The combined organic layers were washed with water and brine, dried over anhydrous Na₂SO₄ and concentrated. The crude product (2.9 g) was used in the next step immediately without further purification.

### Step 2: ((2R,7aS)-2-methoxy-6-methylenetetrahydro-1H-pyrrolizin-7a(5H)-yl)methanol

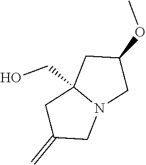

The title compound was prepared by proceeding analogously as described in Intermediate 19. Steps 2-6, using 1-(tert-butyl) 2-methyl (2S,4R)-2-(2-(chloromethyl)allyl)-4-hydroxypyrrolidine-1,2-dicarboxylate instead of 1-(tert-butyl) 2-methyl (2S,4S)-2-(2-(chloromethyl)allyl)-4-hydroxypyrrolidine-1,2-dicarboxylate in Step 2. MS (ES, m/z): [M+H]⁺=184.2.

### Intermediate 22

### Synthesis of ((2S,7aR)-2-methoxy-6-methylenetetrahydro-1H-pyrrolizin-7a(5H)-yl)methanol

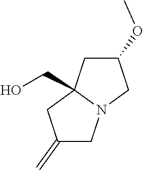

### Step 1: 1-(tert-butyl) 2-methyl (2R,4S)-2-(2-(chloromethyl)allyl)-4-((4-nitrobenzoyl)oxy)-pyrrolidine-1,2-dicarboxylate

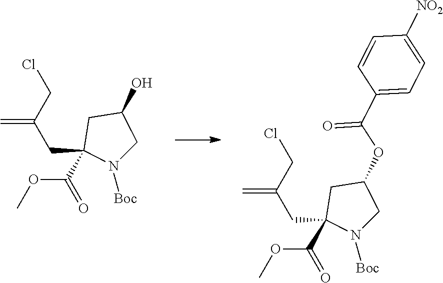 [SMILES: C=C(CCl)C[C@@]1(C(=O)OC)C[C@@H](O)CN1C(=O)OC(C)(C)C ; C=C(CCl)C[C@]1(C(=O)OC)C[C@H](OC(=O)c2ccc([N+](=O)[O-])cc2)CN1C(=O)OC(C)(C)C]

A solution of 1-(tert-butyl) 2-methyl (2R,4R)-2-(2-(chloromethyl)allyl)-4-hydroxy-pyrrolidine-1,2-dicarboxylate (13 g, 39 mmol, 1.0 equiv.) in THF (140 mL) was added 4-nitrobenzoic acid (7.8 g, 47 mmol, 1.2 equiv), PPh₃ (12.26 g, 46.733 mmol, 1.2 equiv), and DEAD (8.1 g, 47 mmol, 1.2 equiv). The resulting mixture was stirred for 3 h at room temperature under N₂ atmosphere. Water was added and the reaction mixture was extracted with EtOAc. The combined organic layers were washed with water and brine, dried over anhydrous Na₂SO₄ and concentrated. The residue was purified by silica gel column chromatography, eluted with EA/PE (0˜20%) to afford the title compound (13.5 g).

### Step 2: 1-(tert-butyl) 2-methyl (2R,4S)-2-(2-(chloromethyl)allyl)-4-hydroxypyrrolidine-1,2-dicarboxylate

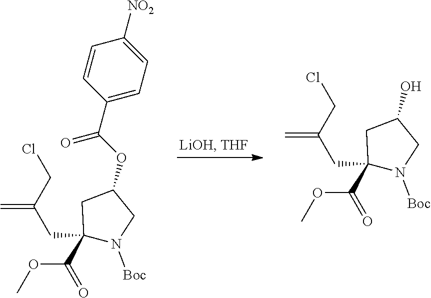 [SMILES: C=C(CCl)C[C@]1(C(=O)OC)C[C@H](OC(=O)c2ccc([N+](=O)[O-])cc2)CN1C(=O)OC(C)(C)C ; C=C(CCl)C[C@]1(C(=O)OC)C[C@H](O)CN1C(=O)OC(C)(C)C]

To a solution of 1-(tert-butyl) 2-methyl (2R,4S)-2-(2-(chloromethyl)allyl)-4-((4-nitrobenzoyl)oxy)pyrrolidine-1,2-dicarboxylate (6.5 g, 13.5 mmol, 1.0 equiv.) in THF (65 mL) was added LiOH (6.5 g, 270 mmol, 20 equiv.) and the resulting mixture was stirred for 6 h at room temperature. Water was added and the mixture was extracted with EtOAc. The combined organic layers were washed with water and brine, dried over anhydrous Na₂SO₄ and concentrated. The crude material was used in the next step without further purification.

### Step 3: ((2S,7aR)-2-methoxy-6-methylenetetrahydro-1H-pyrrolizin-7a(5H)-yl)methanol

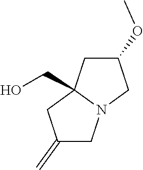

The title compound was prepared by proceeding analogously as described in Intermediate 19, Steps 2-6, using 1-(tert-butyl) 2-methyl (2R,4S)-2-(2-(chloromethyl)allyl)-4-hydroxypyrrolidine-1,2-dicarboxylate instead of 1-(tert-butyl) 2-methyl (2S,4S)-2-(2-(chloromethyl)allyl)-4-hydroxypyrrolidine-1,2-dicarboxylate in Step 2. MS (ES, m/z): [M+H]⁺=184.1.

### Intermediate 23

### Synthesis of ((2R,7aR)-2-(difluoromethoxy)-6-methylenetetrahydro-1H-pyrrolizin-7a(5H)-yl)methanol

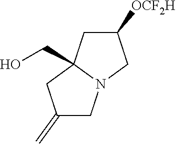

### Step 1. 1-(tert-butyl) 2-methyl (2R,4R)-4-(difluoromethoxy)pyrrolidine-1,2-dicarboxylate

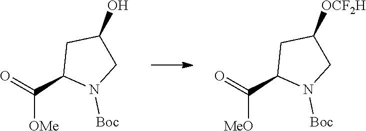

To a solution of 1-(tert-butyl) 2-methyl (2R,4R)-4-hydroxypyrrolidine-1,2-dicarboxylate (25.0 g, 102 mmol, 1.0 equiv) and CuI (3.9 g, 20 mmol, 0.2 equiv) in MeCN (270 mL) was added a solution of 2,2-difluoro-2-(fluorosulfonyl)acetic acid (21.8 g, 122 mmol, 1.2 equiv) in MeCN (100 mL) dropwise at 50° C. under nitrogen atmosphere and the resulting mixture was stirred at 50° C. for 1 h. The reaction mixture was then cooled at 0° C. and quenched with saturated sodium hyposulfite aq. solution. The mixture was extracted with EtOAc and the combined organic layers were washed with water and brine, dried over anhydrous Na₂SO₄ and concentrated. The residue was purified by silica gel column chromatography, eluted with EA/PE (0-50%) to afford the title compound (18 g).

### Step 2: ((2R,7aR)-2-(difluoromethoxy)-6-methylenetetrahydro-1H-pyrrolizin-7a(5H)-yl)methanol

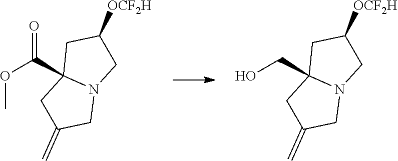

The title compound was prepared analogously by proceeding as described in Intermediate 12, Steps 1-3 using 1-(tert-butyl) 2-methyl (2R,4R)-4-(difluoromethoxy)pyrrolidine-1,2-dicarboxylate instead of 1-tert-butyl 2-methyl (2S,4R)-4-fluoropyrrolidine-1,2-dicarboxylate in Step 1. MS (ES, m/z): [M+H]=184.2.

### Intermediate 24

### Synthesis of ((2R,7aR)-2-(cyclopropylmethoxy)-6-methylenetetrahydro-1H-pyrrolizin-7a(5H)-yl)-methanol

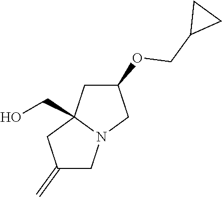 [SMILES: C=C1CN2C[C@H](OCC3CC3)C[C@@]2(CO)C1]

### Step 1: 1-(tert-butyl) 2-methyl (2R,4R)-2-(2-(chloromethyl)allyl)-4-(cyclopropylmethoxy)-pyrrolidine-1,2-dicarboxylate

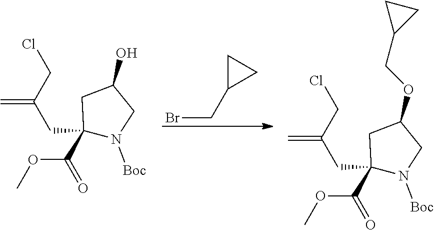

A mixture of 1-(tert-butyl) 2-methyl (2R,4R)-2-(2-(chloromethyl)allyl)-4-hydroxypyrrolidine-1,2-dicarboxylate (10.0 g, 30 mmol, 1.0 equiv), Cs₂CO₃ (29.28 g, 90 mmol, 3.0 equiv) and (bromomethyl)cyclopropane (4.85 g, 36 mmol, 1.2 equiv) in DMF (100 mL) was stirred for 12 h at room temperature. Water was added and the mixture was extracted with EtOAc. The combined organic layers were washed with water and brine, dried over anhydrous Na₂SO₄ and concentrated. The residue was purified by silica gel column chromatography, eluted with EA/PE (0-50%) to afford the title compound (1.0 g).

### Step 3: ((2R,7aR)-2-(cyclopropylmethoxy)-6-methylenetetrahydro-1H-pyrrolizin-7a(5H)-yl)methanol

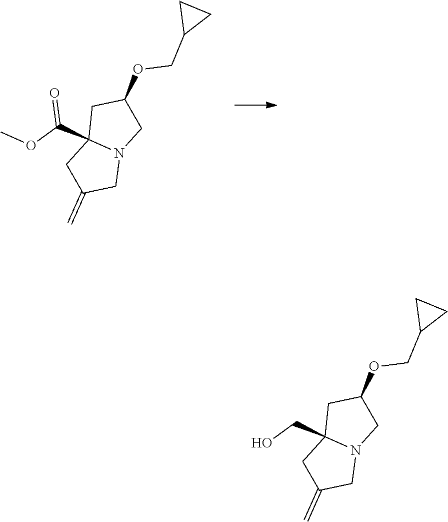 [SMILES: C=C1CN2C[C@H](OCC3CC3)C[C@@]2(C(=O)OC)C1 ; C=C1CN2C[C@H](OCC3CC3)C[C@@]2(CO)C1]

The title compound was prepared by proceeding analogously as described in Intermediate 12, Steps 2-3, using 1-(tert-butyl) 2-methyl (2R,4R)-2-(2-(chloromethyl)allyl)-4-(cyclopropyl-methoxy)pyrrolidine-1,2-dicarboxylate instead of 1-(tert-butyl) 2-methyl (2R,4R)-2-(2-(chloromethyl)allyl)-4-fluoropyrrolidine-1,2-dicarboxylate in Step 2. MS (ES, m/z): [M+H]⁺=224.1.

### Intermediate 25

### Synthesis of ((5S,7aS)-5-(methoxymethyl)-2-methylenetetrahydro-1H-pyrrolizin-7a(5H)-yl)-methanol and ((5S,7aR)-5-(methoxymethyl)-2-methylenetetrahydro-1H-pyrrolizin-7a(5H)-yl)-methanol

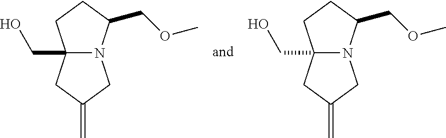

### Step 1: tert-butyl (2S)-2-(hydroxymethyl)-5-methoxypyrrolidine-1-carboxylate

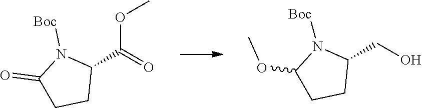 [SMILES: COC(=O)[C@@H]1CCC(=O)N1C(=O)OC(C)(C)C ; COC1CC[C@@H](CO)N1C(=O)OC(C)(C)C]

To a stirred solution of 1-(tert-butyl) 2-methyl (S)-5-oxopyrrolidine-1,2-dicarboxylate (100 g, 411 mmol, 1.0 equiv) in DCM (1500 mL) was added 1.0 M DIBAl-H in toluene (1644 mL, 1644 mmol, 4.0 equiv.) dropwise at −78° C. under nitrogen atmosphere and the resulting mixture was stirred for 30 min. at −78° C. The mixture was then allowed to warm to room temperature and stirred for 2 h at room temperature. After cooling to 0° C., the mixture was quenched with MeOH and 2 M HCl dropwise at 0° C. The mixture was stirred for 2 h at room temperature and then extracted with EtOAc. The combined organic layers were washed with brine, dried over anhydrous Na₂SO₄ and concentrated. The residue was purified by silica gel column chromatography, eluted with EA/PE (20-80%) to afford the title compound (45 g).

### Step 2: tert-butyl (5S)-2-methoxy-5-(methoxymethyl)pyrrolidine-1-carboxylate

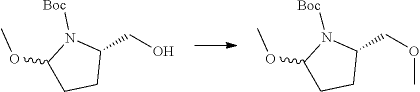

To a solution of tert-butyl (2S)-2-(hydroxymethyl)-5-methoxypyrrolidine-1-carboxylate (36.0 g, 15.6 mmol, 1.0 equiv.) in DMF (120 mL) was added NaH (12.5 g, 31.2 mmol, 2.0 equiv., 60%) in portions for 15 min. at 0° C., followed by the addition of CH₃I (44.3 g, 31.2 mmol, 2.0 equiv.) dropwise at 0° C. The resulting mixture was stirred for 2 h and then quenched with water. The mixture was extracted with EtOAc and the combined organic layers were washed with water and brine, dried over anhydrous Na₂SO₄ and concentrated. The residue was purified by silica gel column chromatography, eluted with EAc/PE (0-50%) to afford the title compound (22.5 g).

### Step 3: tert-butyl (5S)-2-cyano-5-(methoxymethyl)pyrrolidine-1-carboxylate

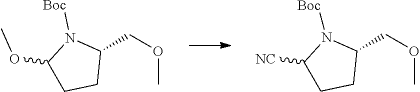

A solution of tert-butyl (5S)-2-methoxy-5-(methoxymethyl)pyrrolidine-1-carboxylate (22.5 g, 91.7 mmol, 1.0 equiv) in DCM (220 mL) was treated with TMSOTf (30.6 g, 137.5 mmol, 1.5 equiv) for 15 min. at 0° C., followed by the addition of trimethylsilyl cyanide (13.6 g, 137.5 mmol, 1.5 equiv.) dropwise at 0° C. The mixture was stirred for 2 h at 0° C. and then quenched with water and extracted with EtOAc. The combined organic layers were washed with water and brine, dried over anhydrous Na₂SO₄ and concentrated. The residue was purified by silica gel column chromatography, eluted with EA/PE (10-30%), to afford the title compound (19 g).

### Step 4: 1-(tert-butyl) 2-methyl (5S)-5-(methoxymethyl)pyrrolidine-1,2-dicarboxylate

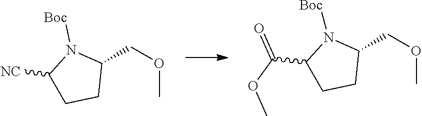 [SMILES: COC[C@@H]1CCC(C#N)N1C(=O)OC(C)(C)C ; COC[C@@H]1CCC(C(=O)OC)N1C(=O)OC(C)(C)C]

A solution of tert-butyl (5S)-2-cyano-5-(methoxymethyl)pyrrolidine-1-carboxylate (13.5 g, 56.2 mmol, 1.0 equiv) and K₂CO₃ (7.8 g, 56.2 mmol, 1.0 equiv) in CH₃OH (135 mL) was stirred for 2 h at room temperature. The mixture was acidified to pH 4 with 10% HCl (70 mL) and then extracted with EtOAc. The combined organic layers were washed with water and brine, dried over anhydrous Na₂SO₄ and concentrated. The residue was purified by silica gel column chromatography, eluted with EA/PE (10-50%), to afford the title compound (10.8 g).

### Step 5: 1-(tert-butyl) 2-methyl (5S)-2-(2-(chloromethyl)allyl)-5-(methoxymethyl)pyrrolidine-1,2-dicarboxylate

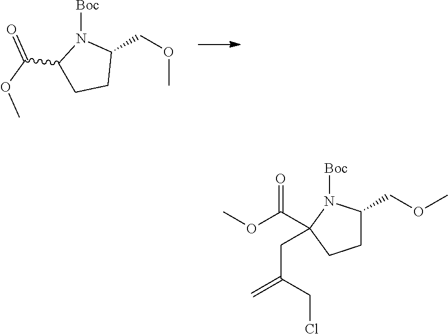

To a solution of 1-(tert-butyl) 2-methyl (5S)-5-(methoxymethyl)pyrrolidine-1,2-dicarboxylate (10.8 g, 39.5 mmol, 1.0 equiv) in THF (108 mL) was added LiHMDS (13.2 g, 79.0 mmol, 2.0 equiv) dropwise for 30 min at −80° C. under nitrogen atmosphere, followed by addition of 3-chloro-2-(chloromethyl)prop-1-ene (19.8 g, 158.0 mmol, 4.0 equiv) dropwise. The resulting mixture was stirred for additional 12 h at room temperature and then quenched with water at 0° C. The resulting mixture was extracted with EA and the combined organic layers were washed with water and brine, dried over anhydrous Na₂SO₄. After filtration, the filtrate was concentrated under reduced pressure and the residue was purified by silica gel column chromatography, eluted with EA/PE (0-25%), to afford the title compound (11.3 g).

### Step 6: methyl (5S,7aS)-5-(methoxymethyl)-2-methylenetetrahydro-1H-pyrrolizine-7a(5H)-carboxylate and methyl (5S,7aR)-5-(methoxymethyl)-2-methylenetetrahydro-1H-pyrrolizine-7a(5H)-carboxy late

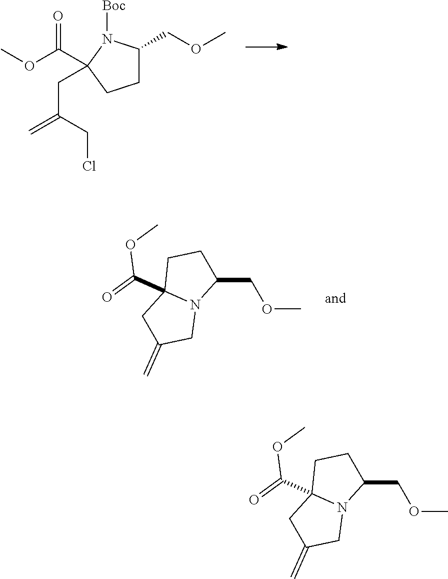

To a stirred solution of 1-(tert-butyl) 2-methyl (5S)-2-(2-(chloromethyl)allyl)-5-(methoxymethyl)pyrrolidine-1,2-dicarboxylate (11.3 g, 31.2 mmol, 1.0 equiv) in DCM (113 mL) was added TFA (40 mL) dropwise at 5° C. The resulting mixture was stirred for 2 h at room temperature and then concentrated under reduced pressure. To the resulting residue was added K₂CO₃ (8.6 g, 62.4 mmol, 2.0 equiv.) and CH₃OH (113 mL) and the mixture was stirred for 2 h at room temperature. EtOAc was added and the mixture was washed with water and brine, and dried over anhydrous Na₂SO₄. After filtration, the filtrate was concentrated under reduced pressure. The residue was purified by silica gel column chromatography, eluted with PE/EA (30-50%), to afford the title compounds (4.5 g) and (400 mg). MS (ES, m/z): [M+H]⁺=226.2.

### Step 7: ((5S,7aS)-5-(methoxymethyl)-2-methylenetetrahydro-1H-pyrrolizin-7a(5H)-yl)methanol and ((5S,7aR)-5-(methoxymethyl)-2-methylenetetrahydro-1H-pyrrolizin-7a(5H)-yl)methanol

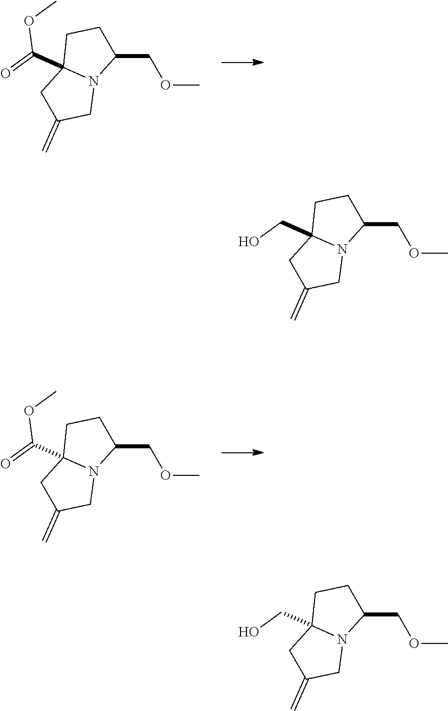 [SMILES: C=C1CN2[C@H](COC)CC[C@@]2(C(=O)OC)C1 ; C=C1CN2[C@H](COC)CC[C@@]2(CO)C1 ; C=C1CN2[C@H](COC)CC[C@]2(C(=O)OC)C1 ; C=C1CN2[C@H](COC)CC[C@]2(CO)C1]

The title compounds were prepared by proceeding analogously as described in Intermediate 11, Step 5, by reducing the ester group with LiAlH₄. MS (ES, m/z): [M+H]⁺=198.2

### Intermediate 26

### Synthesis of: ((5R,7aR)-5-(methoxymethyl)-2-methylenetetrahydro-1H-pyrrolizin-7a(5H)-yl)-methanol and ((5R,7aS)-5-(methoxymethyl)-2-methylenetetrahydro-1H-pyrrolizin-7a(5H)-yl)-methanol

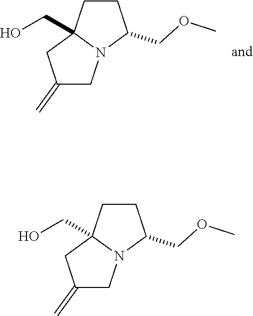

The title compounds were prepared by proceeding analogously as described in Intermediate 23, Steps 1-7, using 1-(tert-butyl) 2-methyl (R)-5-oxopyrrolidine-1,2-dicarboxylate instead of 1-(tert-butyl) 2-methyl (S)-5-oxopyrrolidine-1,2-dicarboxylate in Step 1. MS (ES, m/z): [M+H]⁺=198.2.

### Intermediate 27

### Synthesis of [(2S,7aR)-2-(methoxymethyl)-6-methylidene-tetrahydro-1H-pyrrolizin-7a-yl]-methanol and ((2S,7aS)-2-(methoxymethyl)-6-methylenetetrahydro-1H-pyrrolizin-7a(5H)-yl)-methanol

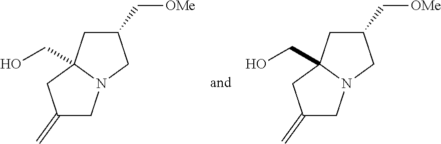

### Step 1: 1-(tert-butyl) 2-methyl (2R,4S)-2-(2-(chloromethyl)allyl)-4-(methoxymethyl)pyrrolidine-1,2-dicarboxylate and 1-(tert-butyl) 2-methyl (2S,4S)-2-(2-(chloromethyl)allyl)-4-(methoxy-methyl)pyrrolidine-1,2-dicarboxylate

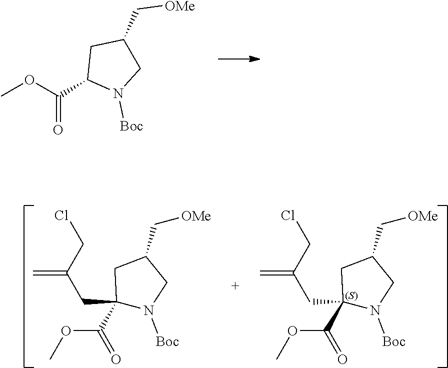

To a stirred solution of HMDS (12.2 g, 75.6 mmol, 1.15 equiv) in THF (360 mL) was added n-BuLi (30.3 mL, 75.6 mmol, 1.15 equiv., 2.5 M in hexane) dropwise at −78° C. under nitrogen atmosphere and the resulting mixture was stirred for 30 min. 1-tert-butyl 2-Methyl (2S,4S)-4-(methoxymethyl)pyrrolidine-1,2-dicarboxylate (18 g, 65.9 mmol, 1 equiv) was added dropwise at −78° C. and the resulting mixture was stirred for 15 min. 3-Chloro-2-(chloromethyl)prop-1-ene (20.5 g, 164 mmol, 2.5 equiv) was added dropwise at −78° C. and the mixture was stirred for 16 h at room temperature under nitrogen atmosphere. The mixture was quenched by the addition of saturated NH₄Cl (aq.) at room temperature and extracted with EA. The combined organic layers were washed with brine, and dried over anhydrous Na₂SO₄. After filtration, the filtrate was concentrated under reduced pressure and the residue was purified by silica gel column chromatography, eluted with EA/PE (0-10%), to afford the title compound (19.9 g).

### Step 2: methyl (2S,7aR)-2-(methoxymethyl)-6-methylidene-tetrahydro-1H-pyrrolizine-7a-carboxylate [A] and methyl (2S,7aS)-2-(methoxymethyl)-6-methylidene-tetrahydro-1H-pyrrolizine-7a-carboxylate [B]

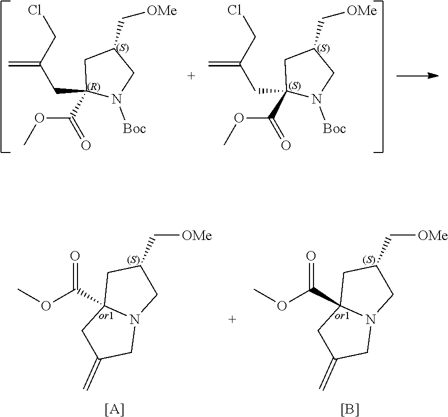

To a stirred solution of 1-tert-butyl 2-methyl (2R,4S)-2-[2-(chloromethyl)prop-2-en-1-yl]-4-(methoxymethyl)pyrrolidine-1,2-dicarboxylate and 1-tert-butyl 2-methyl (2S,4S)-2-[2-(chloromethyl)prop-2-en-1-yl]-4-(methoxymethyl)pyrrolidine-1,2-dicarboxylate (10 g, 13.8 mmol, 1 equiv.) in DCM (75 mL) was added TFA (25 mL) dropwise at 5° C. and the resulting mixture was stirred for 2 h at room temperature. The mixture was concentrated under vacuum. Methanol (100 mL) and K₂CO₃ (19 g, 138 mmol, 10 equiv) were added to the residue, and the mixture was stirred for 2 h at room temperature. After the volatile solvents were removed under vacuum, the mixture was extracted with EtOAc. The combined organic layers were washed with brine, dried over anhydrous Na₂SO₄ and concentrated. The residue was purified by silica gel column chromatography, eluted with EA/PE (0-100%). The material was further purified by prep-HPLC to afford the title compounds.

### Step 4: [(2S,7aR)-2-(methoxymethyl)-6-methylidene-tetrahydro-1H-pyrrolizin-7a-yl]methanol

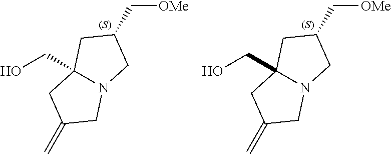

The title compounds were prepared by proceeding analogously as described in Intermediate 11, Step 5, by reducing methyl (2S,7aR)-2-(methoxymethyl)-6-methylidene-tetrahydro-1H-pyrrolizine-7a-carboxylate and methyl (2S,7aS)-2-(methoxymethyl)-6-methylidene-tetrahydro-1H-pyrrolizine-7a-carboxylate with LiAlH₄. MS (ES, m/z): [M+H]⁺=198.2.

### Intermediate 28

### Synthesis of methyl (5S,7aS)-5-(hydroxymethyl)-2-methylenetetrahydro-1H-pyrrolizine-7a(5H)-carboxylate

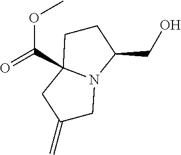

### Step 1: tert-butyl (2S)-2-(((tert-butyldimethylsilyl)oxy)methyl)-5-methoxypyrrolidine-1-carboxylate

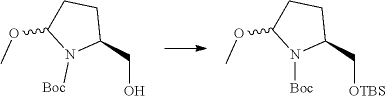 [SMILES: COC1CC[C@@H](CO)N1C(=O)OC(C)(C)C ; COC1CC[C@@H](CO[Si](C)(C)C(C)(C)C)N1C(=O)OC(C)(C)C]

To a stirred solution of tert-butyl (2S)-2-(hydroxymethyl)-5-methoxypyrrolidine-1-carboxylate (50.0 g, 216 mmol, 1.0 eq.) and imidazole (22.0 g, 324 mmol, 1.5 eq.) in DCM (500 mL) was added TBSCl (39.0 g, 259 mmol, 1.2 eq.) in portions at 0° C. under nitrogen atmosphere and the resulting mixture was stirred for 3 h at room temperature. After adding water, and the mixture was extracted with DCM. The combined organic layers were washed with brine, dried over anhydrous Na₂SO₄ and concentrated. The residue was purified by silica gel column chromatography, eluted with PE/EA (0-15%), to afford the title compound (61.0 g).

### Step 2: tert-butyl (2S)-2-(((tert-butyldimethylsilyl)oxy)methyl)-5-cyanopyrrolidine-1-carboxylate

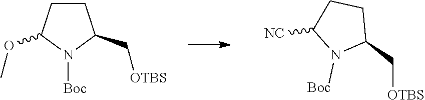 [SMILES: COC1CC[C@@H](CO[Si](C)(C)C(C)(C)C)N1C(=O)OC(C)(C)C ; CC(C)(C)OC(=O)N1C(C#N)CC[C@H]1CO[Si](C)(C)C(C)(C)C]

To a stirred solution of tert-butyl (2S)-2-(((tert-butyldimethylsilyl)oxy)methyl)-5-methoxypyrrolidine-1-carboxylate (65.0 g, 188 mmol, 1.00 eq.) in DCM (650 mL) were added TMSOTf (2.0 g, 9.4 mmol, 0.05 eq.) and TMSCN (28.0 g, 282 mmol, 1.50 eq.) dropwise at −78° C. under nitrogen atmosphere and the resulting mixture was stirred for 1 h at −78° C. The mixture was quenched with sat. NaHCO₃ (aq.) at −78° C. and the resulting mixture was extracted with EtOAc. The combined organic layers were washed with brine, dried over anhydrous Na₂SO₄ and concentrated. The residue was purified by silica gel column chromatography, eluted with EA/PE (0-50%) to afford the title compound (32.0 g).

### Step 3: 1-(tert-butyl) 2-methyl (5S)-5-(hydroxymethyl)pyrrolidine-1,2-dicarboxylate

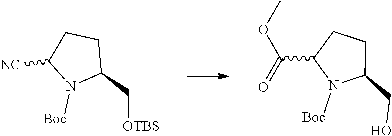 [SMILES: CC(C)(C)OC(=O)N1C(C#N)CC[C@H]1CO[Si](C)(C)C(C)(C)C ; COC(=O)C1CC[C@@H](CO)N1C(=O)OC(C)(C)C]

A mixture of tert-butyl (2S)-2-(((tert-butyldimethylsilyl)oxy)methyl)-5-cyanopyrrolidine-1-carboxylate (32.0 g, 94 mmol, 1.00 eq.) and K₂CO₃ (26.0 g, 188 mmol, 2.00 eq.) in MeOH (340 mL) was stirred for 3 h at room temperature. The mixture was acidified to pH=2 with 10% HCl aq. solution and the resulting mixture was stirred for 16 h at room temperature. The mixture was then extracted with EtOAc and the combined organic layers were washed with brine, dried over anhydrous Na₂SO₄ and concentrated. The residue was purified by silica gel column chromatography, eluted with EA/PE (0-50%) to afford the title compound (17.0 g).

### Step 4: 1-(tert-butyl) 2-methyl (5S)-5-(((tert-butyldiphenylsilyl)oxy)methyl)pyrrolidine-1,2-dicarboxylate

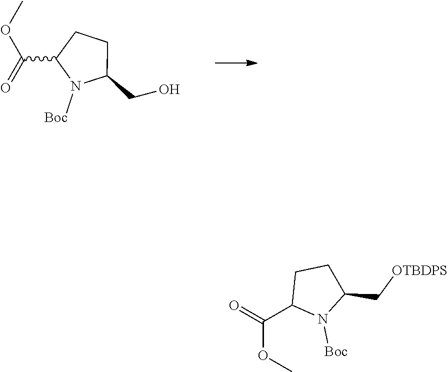

To a stirred solution of 1-(tert-butyl) 2-methyl (5S)-5-(hydroxymethyl)pyrrolidine-1,2-dicarboxylate (17.0 g, 65.5 mmol, 1.00 eq.) and imidazole (6.69 g, 98.3 mmol, 1.50 eq.) in DCM (170 mL) was added TBDPSCl (21.62 g, 78.6 mmol, 1.20 eq.) dropwise at 0° C. under nitrogen atmosphere and the resulting mixture was stirred for 2 h at room temperature. The reaction was quenched with water at 0° C. and the resulting mixture was extracted with DCM. The combined organic layers were washed with brine, dried over anhydrous Na₂SO₄ and concentrated. The residue was purified by silica gel column chromatography, eluted with EA/PE (0-50%), to afford the title compound (24.0 g).

### Step 5: 1-(tert-butyl) 2-methyl (2S,5S)-5-(((tert-butyldiphenylsilyl)oxy)methyl)-2-(2-(chloromethyl) allyl)pyrrolidine-1,2-dicarboxylate

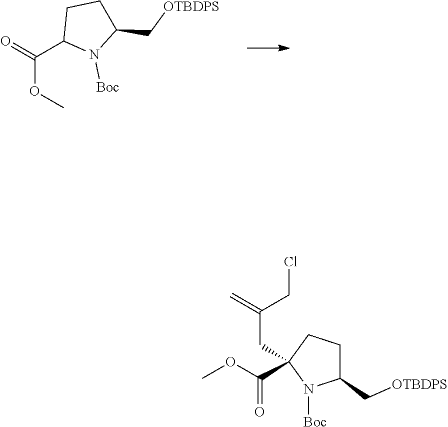

To a stirred solution of 1-(tert-butyl) 2-methyl (5S)-5-(((tert-butyldiphenylsilyl)oxy)methyl)pyrrolidine-1,2-dicarboxylate (23.0 g, 46.2 mmol, 1.00 eq.) in THF (230 mL) was added LiHMDS in THF (92.42 mL, 1.0 M, 92.4 mmol, 2.00 eq.) dropwise at −78° C. under nitrogen atmosphere. The resulting mixture was stirred for 10 min at −78° C. and then 3-chloro-2-(chloromethyl)prop-1-ene (11.55 g, 92.4 mmol, 2.00 eq.) was added dropwise at −78° C. under nitrogen atmosphere. The resulting mixture was stirred for 16 h at room temperature under nitrogen atmosphere. The mixture was quenched with water at 0° C. and the resulting mixture was extracted with EA. The combined organic layers were washed with brine, dried over anhydrous Na₂SO₄ and concentrated. The residue was purified by silica gel column chromatography, eluted with EA/PE (0-10%), to afford the title compound (21.0 g).

### Step 6: methyl (5S,7aS)-5-(((tert-butyldiphenylsilyl)oxy)methyl)-2-methylenetetrahydro-1H-pyrrolizine-7a(5H)-carboxylate

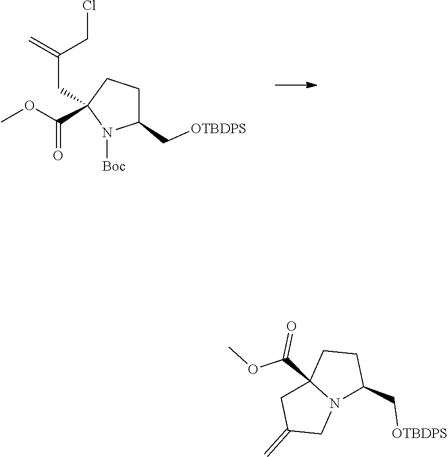

To a solution of 1-(tert-butyl) 2-methyl (2S,5S)-5-(((tert-butyldiphenylsilyl)oxy) methyl)-2-(2-(chloromethyl)allyl)pyrrolidine-1,2-dicarboxylate (21.0 g, 35.82 mmol, 1.00 eq) in DCM (210 mL) was added TFA (100 mL) dropwise at room temperature and the resulting mixture was stirred for 2 h. The reaction mixture was concentrated under reduced pressure. The residue was dissolved in MeOH (210 mL) and K₂CO₃ was added. The mixture was stirred for 40 min. at room temperature and then quenched with water at room temperature. The mixture was extracted with EtOAc and the combined organic layers were washed with brine, dried over anhydrous Na₂SO₄ and concentrated. The residue was purified by silica gel column chromatography, eluted with EA/PE (0-15%), to afford the title compound (8.5 g).

### Step 7: methyl (5S,7aS)-5-(hydroxymethyl)-2-methylenetetrahydro-1H-pyrrolizine-7a(5H)-carboxylate

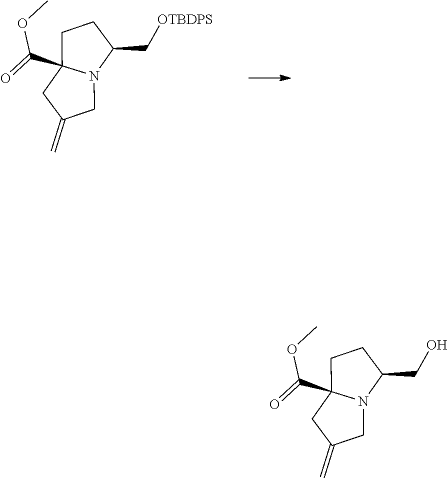

To a stirred solution of methyl (5S,7aS)-5-(((tert-butyldiphenylsilyl)oxy)methyl)-2-methylenetetrahydro-1H-pyrrolizine-7a(5H)-carboxylate (8.5 g, 18.9 mmol, 1.00 eq.) in THF (85 mL) was added TBAF in THF (28.3 mL, 1 M, 28.3 mmol, 1.50 eq.) dropwise at room temperature. The resulting mixture was stirred for 3 h at room temperature and then quenched with water and extracted with EtOAc. The combined organic layers were washed with brine, dried over anhydrous Na₂SO₄ and concentrated. The residue was purified by silica gel column chromatography, eluted with EA/PE (0-100%), to afford the title compound (2.8 g).

### Intermediate 29

### Synthesis of ((5S,7aS)-5-(fluoromethyl)-2-methylenetetrahydro-1H-pyrrolizin-7a(5H)-yl)-methanol

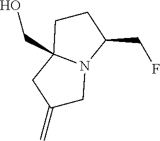

### Step 1: methyl (5S,7aS)-5-(fluoromethyl)-2-methylenetetrahydro-1H-pyrrolizine-7a(5H)-carboxylate

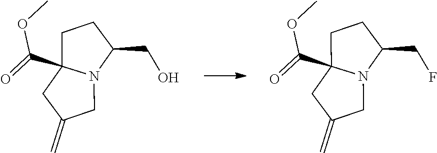

To a stirred solution of methyl (5S,7aS)-5-(hydroxymethyl)-2-methylenetetrahydro-1H-pyrrolizine-7a(5H)-carboxylate (100 mg, 0.51 mmol, 1.00 eq.) in DCM (1 mL) was added BAST (160 mg, 7.12 mmol, 1.50 eq.) dropwise at −78° C. under nitrogen atmosphere and the resulting mixture was stirred for 12 h at room temperature. After cooling to 0° C., the reaction was quenched with brine. The resulting mixture was diluted with NaHCO₃ (aq.) and extracted with EtOAc. The combined organic layers were washed with brine, dried over anhydrous Na₂SO₄ and concentrated. The residue was purified by silica gel column chromatography, eluted with EA/PE (0-70%), to afford the title compound (30 mg).

### Step 2: ((5S,7aS)-5-(fluoromethyl)-2-methylenetetrahydro-1H-pyrrolizin-7a(5H)-yl)methanol

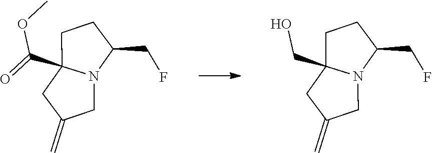

The title compound was prepared by proceeding analogously as described in Intermediate 11, Step 5, by reducing the ester group with LiAlH₄.

### Intermediate 30

### Synthesis of ((5S,7aS)-5-((2-methoxyethoxy)methyl)-2-methylenetetrahydro-1H-pyrrolizin-7a(5H)-yl)methanol

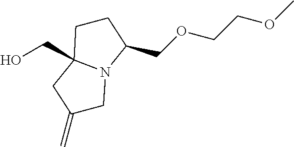 [SMILES: C=C1CN2[C@H](COCCOC)CC[C@@]2(CO)C1]

### Step 1: methyl (5S,7aS)-5-((2-methoxyethoxy)methyl)-2-methylenetetrahydro-1H-pyrrolizine-7a(5H)-carboxylate

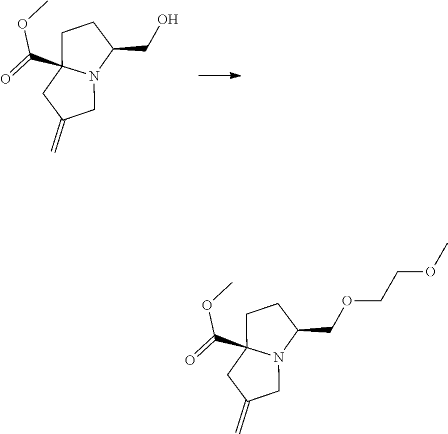 [SMILES: C=C1CN2[C@H](CO)CC[C@@]2(C(=O)OC)C1 ; C=C1CN2[C@H](COCCOC)CC[C@@]2(C(=O)OC)C1]

To a stirred solution of methyl (5S,7aS)-5-(hydroxymethyl)-2-methylenetetrahydro-1H-pyrrolizine-7a(5H)-carboxylate (400 mg, 1.89 mmol, 1.0 eq.) in THF (4 mL) was added NaH (113 mg, 2.84 mmol, 60%,1.5 eq.) in portions at 0° C. and the resulting mixture was stirred for 5 min. at 0° C. 2-Bromoethyl methyl ether (395 mg, 2.84 mmol, 1.5 eq.) was added dropwise at 0° C. and the resulting mixture was stirred for 1 h at room temperature. The reaction was quenched with water at 0° C. and the resulting mixture was extracted with EtOAc. The combined organic layers were washed with brine, dried over anhydrous Na₂SO₄ and concentrated. The residue was purified by silica gel column chromatography, eluted with EA/PE (0-50%), to afford the title compound (80 mg) as a colorless oil.

### Step 2: ((5S,7aS)-5-((2-methoxyethoxy)methyl)-2-methylenetetrahydro-1H-pyrrolizin-7a(5H)-yl)methanol

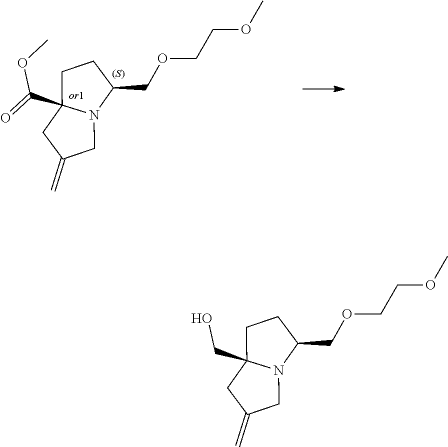

The title compound was prepared by proceeding analogously as described in Intermediate 11, Step 5, by reducing the ester group with LiAlH₄. MS (ES, m/z): [M+H]⁺=242.2.

### Intermediate 31

### Synthesis of ((5S,7aS)-5-((difluoromethoxy)methyl)-2-methylenetetrahydro-1H-pyrrolizin-7a(5H)-yl)methanol

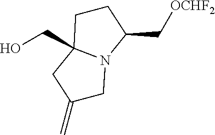 [SMILES: C=C1CN2[C@H](COC(F)F)CC[C@@]2(CO)C1]

### Step 1: methyl (5S,7aS)-5-((difluoromethoxy)methyl)-2-methylenetetrahydro-1H-pyrrolizine-7a(5H)-carboxylate

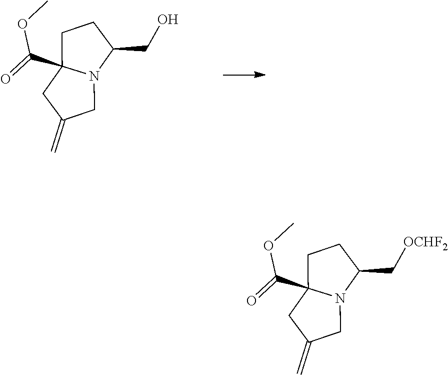 [SMILES: C=C1CN2[C@H](CO)CC[C@@]2(C(=O)OC)C1 ; C=C1CN2[C@H](COC(F)F)CC[C@@]2(C(=O)OC)C1]

To a stirred mixture of methyl (5S,7aS)-5-(hydroxymethyl)-2-methylenetetrahydro-1H-pyrrolizine-7a(5H)-carboxylate (1 g, 4.7 mmol, 1.00 eq) and CuI (0.18 g, 0.9 mmol, 0.20 eq.) in CH₃CN (15 mL) was added 2,2-difluoro-2-(fluorosulfonyl)acetic acid (2.53 g, 14.2 mmol, 3.00 eq.) dropwise at 50° C. under nitrogen atmosphere and the resulting mixture was stirred for 2 h at 50° C. After cooling to room temperature, the reaction mixture was quenched with sat. NaHCO₃ aq. solution at room temperature. The mixture was then extracted with EtOAc and the combined organic layers were washed with brine, dried over anhydrous Na₂SO₄ and concentrated. The residue was purified by silica gel column chromatography, eluted with EA/PE (0-50%), to afford the title compound (100 mg).

### Step 2: ((5S,7aS)-5-((difluoromethoxy)methyl)-2-methylenetetrahydro-1H-pyrrolizin-7a(5H)-yl)methanol

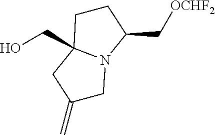 [SMILES: C=C1CN2[C@H](COC(F)F)CC[C@@]2(CO)C1]

The title compound was prepared by proceeding analogously as described in Intermediate 11, Step 5, by reducing the ester group with LiAlH₄.

### Intermediate 32

### Synthesis of ((5S,7aS)-5-(cyclopropoxymethyl)-2-methylenetetrahydro-1H-pyrrolizin-7a(5H)-yl)-methanol

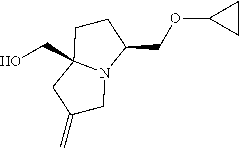 [SMILES: C=C1CN2[C@H](COC3CC3)CC[C@@]2(CO)C1]

### Step 1: tert-butyl (5S)-2-methoxy-5-((vinyloxy)methyl)pyrrolidine-1-carboxylate

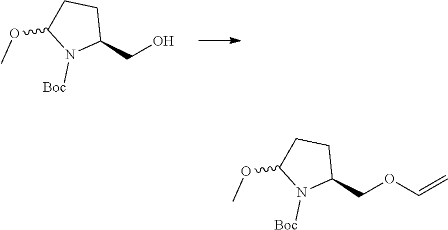 [SMILES: COC1CC[C@@H](CO)N1C(=O)OC(C)(C)C ; C=COC[C@@H]1CCC(OC)N1C(=O)OC(C)(C)C]

A mixture of Pd(OAc)₂ (194 mg, 0.86 mmol, 0.02 eq.) and 1,10-phenanthroline (155 mg, 0.86 mmol, 0.02 eq.) in ethyl vinyl ether (100 mL) was stirred for 20 min at room temperature under nitrogen atmosphere. tert-butyl (2S)-2-(Hydroxymethyl)-5-methoxypyrrolidine-1-carboxylate (10.0 g, 43.2 mmol, 1.00 eq.) was added and the resulting mixture was stirred for 16 h at 45° C. The reaction mixture was concentrated under reduced pressure and water and EtOAc were added to the residue. The phases were separated, and the aqueous layer was extracted with EtOAc. The combined organic layers were washed with water and brine, dried over anhydrous Na₂SO₄ and concentrated. The residue was purified by silica gel column chromatography, eluted with EA/PE (0-5%), to afford the title compound (4.3 g).

### Step 2: tert-butyl (2S)-2-(cyclopropoxymethyl)-5-methoxypyrrolidine-1-carboxylate

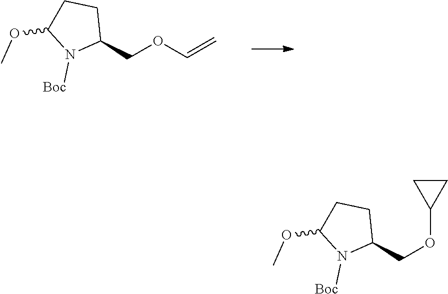 [SMILES: C=COC[C@@H]1CCC(OC)N1C(=O)OC(C)(C)C ; COC1CC[C@@H](COC2CC2)N1C(=O)OC(C)(C)C]

To a stirred mixture of 40% KOH (240 mL) and Et₂O (600 mL) was added 1-methyl-1-nitrosourea (77.5 g, 752 mmol, 45.00 eq.) in portions at 0° C. and the resulting mixture was stirred for 10 min. at 0° C. The aqueous layer was extracted with Et₂O. The ether solution was added to a mixture of tert-butyl (5S)-2-methoxy-5-((vinyloxy)methyl)pyrrolidine-1-carboxylate (4.3 g, 16.7 mmol, 1.00 eq.) and Pd(acac)₂ (510 mg, 1.67 mmol, 0.10 eq.) in DCM (50 mL) dropwise at 0° C. under nitrogen atmosphere. The resulting mixture was stirred for 20 min. at 0° C. under nitrogen atmosphere. The mixture was quenched with water and the aqueous layer was extracted with DCM. The combined organic layers were washed with water and brine, dried over anhydrous Na₂SO₄ and concentrated. The residue was purified by silica gel column chromatography, eluted with EA/PE (0-5%), to afford the title compound (2.5 g).

### Step 3: ((5S,7aS)-5-(cyclopropoxymethyl)-2-methylenetetrahydro-1H-pyrrolizin-7a(5H)-yl)methanol

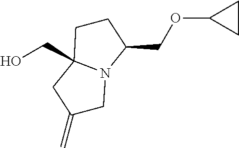 [SMILES: C=C1CN2[C@H](COC3CC3)CC[C@@]2(CO)C1]

The title compound was prepared by proceeding analogously as described in Intermediate 25, Step 3-7, using tert-butyl (2S)-2-(cyclopropoxymethyl)-5-methoxypyrrolidine-1-carboxylate instead of tert-butyl (5S)-2-methoxy-5-(methoxymethyl)pyrrolidine-1-carboxylate In Step 3.

### Intermediate 33

### Synthesis of ((5S,7aS)-5-((cyclopropylmethoxy)methyl)-2-methylenetetrahydro-1H-pyrrolizin-7a(5H)-yl)methanol

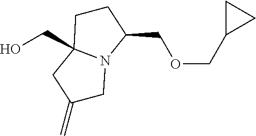 [SMILES: C=C1CN2[C@H](COCC3CC3)CC[C@@]2(CO)C1]

### Step 1: tert-butyl (2S)-2-((cyclopropylmethoxy)methyl)-5-methoxypyrrolidine-1-carboxylate

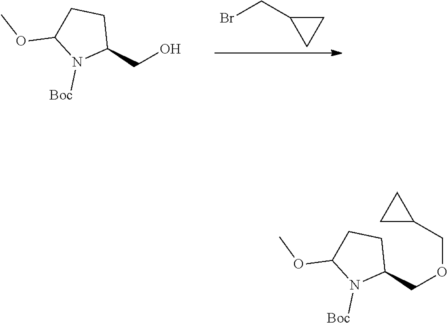 [SMILES: COC1CC[C@@H](CO)N1C(=O)OC(C)(C)C ; BrCC1CC1 ; COC1CC[C@@H](COCC2CC2)N1C(=O)OC(C)(C)C]

To a solution of tert-butyl (2S)-2-(hydroxymethyl)-5-methoxypyrrolidine-1-carboxylate (10.0 g, 43.2 mmol, 1.00 eq.) and (bromomethyl)cyclopropane (17.5 g, 130 mmol, 3.00 eq.) in THF (100 mL) was added t-BuOK in THF (108 mL, 1 M, 108 mmol, 2.50 eq.) dropwise at 0° C. under nitrogen atmosphere and the resulting mixture was stirred overnight at room temperature. The reaction was quenched with sat. NH₄Cl (aq.) at room temperature and the resulting mixture was extracted with EtOAc. The combined organic layers were washed with brine, dried over anhydrous Na₂SO₄ and concentrated. The residue was purified by silica gel column chromatography, eluted with EA/PE (0-10%), to afford the title compound (6.5 g).

### Step 2: ((5S,7aS)-5-((cyclopropylmethoxy)methyl)-2-methylenetetrahydro-TH-pyrrolizin-7a(5H)-yl)methanol

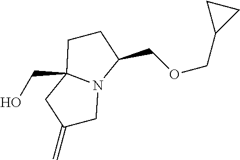 [SMILES: C=C1CN2[C@H](COCC3CC3)CC[C@@]2(CO)C1]

The title compound was prepared by proceeding analogously as described in Intermediate 25, Steps 3-7, using tert-butyl (2S)-2-((cyclopropylmethoxy)methyl)-5-methoxy-pyrrolidine-1-carboxylate instead of tert-butyl (5S)-2-methoxy-5-(methoxymethyl)pyrrolidine-1-carboxylate

### Intermediate 34

### Synthesis of ((5S,7aS)-5-(2-fluoroethyl)-2-methylenetetrahydro-TH-pyrrolizin-7a(5H)-yl)-methanol

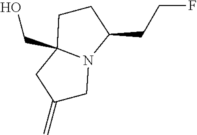

### Step 1: 1-(tert-butyl) 2-methyl (2R,5S)-5-(2-oxoethyl)pyrrolidine-1,2-dicarboxylate

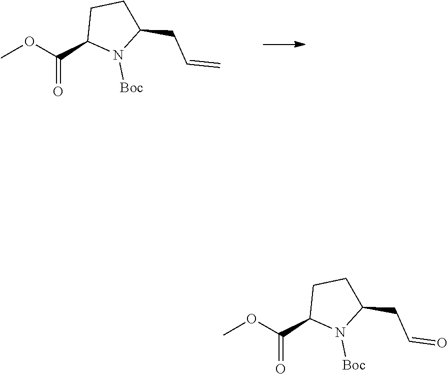 [SMILES: C=CC[C@@H]1CC[C@H](C(=O)OC)N1C(=O)OC(C)(C)C ; COC(=O)[C@H]1CC[C@@H](CC=O)N1C(=O)OC(C)(C)C]

To a solution of 1-tert-butyl 2-methyl (2R,5S)-5-(prop-2-en-1-yl)pyrrolidine-1,2-dicarboxylate (7 g, 25.989 mmol, 1.0 eq.) (prepared by the method described in Synlett 1999, 1660-1662) in THF (90 mL) and H₂O (30 mL) were added K₂OsO₄·2H₂O (0.96 g, 2.6 mmol, 0.1 eq.) and NaIO₄ (16.68 g, 78.0 mmol, 3.0 eq.) at 0° C. The resulting mixture was then stirred for 16 h at room temperature under air. The reaction mixture was filtered, the filter cake was washed with EtOAc. The filtrate was extracted with EtOAc and the combined organic layers were washed with NaHSO₃ (aq.) and brine, dried over anhydrous Na₂SO₄ and concentrated. The residue was purified by silica gel column chromatography, eluted with EA/PE (0-50%), to afford the title compound (3.3 g). MS (ES, m/z): [M-100+H]⁺=172.2.

### Step 2: 1-(tert-butyl) 2-methyl (2R,5S)-5-(2-hydroxyethyl)pyrrolidine-1,2-dicarboxylate

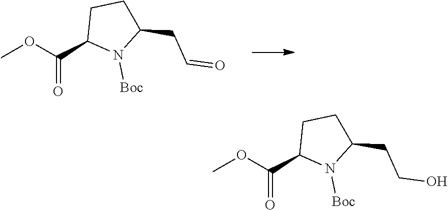 [SMILES: COC(=O)[C@H]1CC[C@@H](CC=O)N1C(=O)OC(C)(C)C ; COC(=O)[C@H]1CC[C@@H](CCO)N1C(=O)OC(C)(C)C]

To a solution of 1-(tert-butyl) 2-methyl (2R,5S)-5-(2-oxoethyl)pyrrolidine-1,2-dicarboxylate (2 g, 7.8 mmol, 1 eq.) in MeOH (15 mL) was added NaBH₄ (557 mg, 14.75 mmol, 2.0 eq. in portions at 0° C. and the resulting mixture was stirred for 30 min. at 0° C. The mixture was quenched with HCl aq. solution to pH=7. The mixture was concentrated under reduced pressure and the remaining material was extracted with EtOAc. The combined organic layers were washed with water and brine, dried over anhydrous Na₂SO₄ and concentrated. The residue was purified by silica gel column chromatography, eluted with EA/PE (0-50%), to afford the title compound (1.7 g).

### Step 3: 1-(tert-butyl) 2-methyl (2R,5S)-5-(2-((methylsulfonyl)oxy)ethyl)pyrrolidine-1,2-dicarboxylate

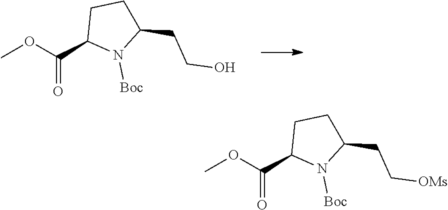 [SMILES: COC(=O)[C@H]1CC[C@@H](CCO)N1C(=O)OC(C)(C)C ; COC(=O)[C@H]1CC[C@@H](CCOS(C)(=O)=O)N1C(=O)OC(C)(C)C]

To a solution of 1-(tert-butyl) 2-methyl (2R,5S)-5-(2-hydroxyethyl)pyrrolidine-1,2-dicarboxylate (1.4 g, 5.1 mmol, 1.0 eq.), DABCO (115 mg, 1.0 mmol, 0.2 eq.) and TEA (622 mg, 6.1 mmol, 1.2 eq.) in toluene (20 mL) was added a solution of MsCl (645 mg, 5.6 mmol, 1.1 eq.) in toluene (1 mL) at 0° C. under nitrogen atmosphere, and the resulting mixture was stirred for 1 h at room temperature. The reaction mixture was diluted with water and extracted with EA. The combined organic layers were washed with water and brine, dried over anhydrous Na₂SO₄ and concentrated. The residue was purified by silica gel column chromatography, eluted with EA/EA (0-50%), to afford the title compound (1.5 g).

### Step 4: 1-(tert-butyl) 2-methyl (2R,5S)-5-(2-fluoroethyl)pyrrolidine-1,2-dicarboxylate

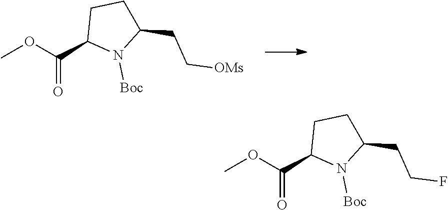 [SMILES: COC(=O)[C@H]1CC[C@@H](CCOS(C)(=O)=O)N1C(=O)OC(C)(C)C ; COC(=O)[C@H]1CC[C@@H](CCF)N1C(=O)OC(C)(C)C]

To a solution of 1-tert-butyl 2-methyl (2R,5S)-5-[2-(methanesulfonyloxy)ethyl]-pyrrolidine-1,2-dicarboxylate (1.4 g, 4.0 mmol, 1 eq.) and TEA (565 mg, 5.6 mmol, 1.4 eq.) in THF (14 mL) was added TBAF (5.18 mL, 5.179 mmol, 1.3 eq.) at room temperature and the resulting mixture was stirred for 3 h at 60° C. The reaction mixture was diluted with water and extracted with EtOAc. The combined organic layers were washed with water and brine, dried over anhydrous Na₂SO₄ and concentrated. The residue was purified by silica gel column chromatography, eluted with EA/PE (0-20%), to afford the title compound (710 mg). MS (ES, m/z): [M-Boc+H]⁺=176.1.

### Step 5: ((5S,7aS)-5-(2-fluoroethyl)-2-methylenetetrahydro-1H-pyrrolizin-7a(5H)-yl)methanol

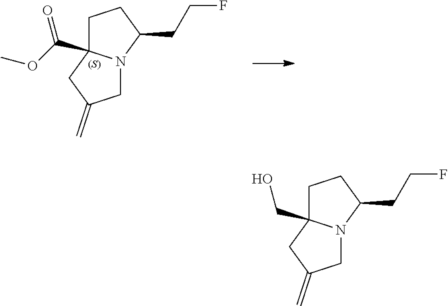

The title compound was prepared by proceeding analogously as described in Intermediate 25, Steps 4-7 using 1-(tert-butyl) 2-methyl (2R,5S)-5-(2-fluoroethyl)pyrrolidine-1,2-dicarboxylate instead of 1-(tert-butyl) 2-methyl (5S)-5-(methoxymethyl)pyrrolidine-1,2-dicarboxylate in Step 4. MS (ES, m/z): [M+H]⁺=200.2.

### Intermediate 35

### Synthesis of ((5S,7aS)-5-(3,3-difluoropropyl)-2-methylenetetrahydro-1H-pyrrolizin-7a(5H)-yl)methanol

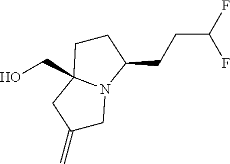 [SMILES: C=C1CN2[C@H](CCC(F)F)CC[C@@]2(CO)C1]

### Step 1: 1-(tert-butyl) 2-methyl (2R,5S)-5-(3-hydroxypropyl)pyrrolidine-1,2-dicarboxylate

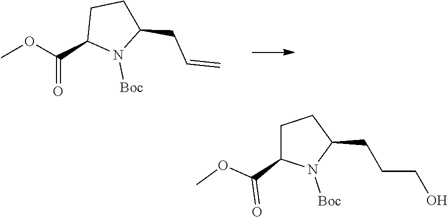 [SMILES: C=CC[C@@H]1CC[C@H](C(=O)OC)N1C(=O)OC(C)(C)C ; COC(=O)[C@H]1CC[C@@H](CCCO)N1C(=O)OC(C)(C)C]

To a solution of 1-(tert-butyl) 2-methyl (2R,5S)-5-allylpyrrolidine-1,2-dicarboxylate (6.5 g, 24.1 mmol, 1.0 eq.) in THF (65 mL) was added 9-BBN (1.0 M solution in THF, 72.4 mL, 72.4 mmol, 3.0 eq.) dropwise at room temperature and the mixture was stirred overnight at room temperature. To the above mixture H₂O₂ (30% aq. solution) (65 mL) was added, followed by sodium tetraborate decahydrate (27.6 g, 72.4 mmol, 3.0 eq.) in portions and the mixture was stirred for 3 h at room temperature. The reaction mixture was then diluted with water and extracted with EtOAc. The combined organic layers were washed with brine, dried over anhydrous Na₂SO₄ and concentrated. The residue was purified by silica gel column chromatography, eluted with EA/PE (0-50%), to afford the title compound (4.6 g).

### Step 2: 1-(tert-butyl) 2-methyl (2R,5S)-5-(3-oxopropyl)pyrrolidine-1,2-dicarboxylate

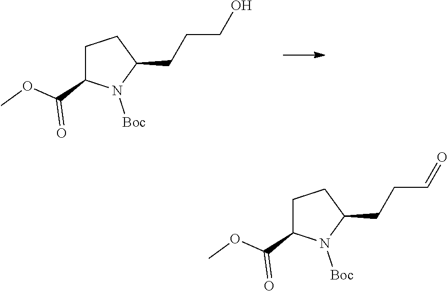 [SMILES: COC(=O)[C@H]1CC[C@@H](CCCO)N1C(=O)OC(C)(C)C ; COC(=O)[C@H]1CC[C@@H](CCC=O)N1C(=O)OC(C)(C)C]

A solution of oxalyl chloride (2.12 g, 16.7 mmol, 2 equiv) in DCM (25 mL) was added DMSO (27 mL). After stirring for 5 min at −78° C., 1-(tert-butyl) 2-methyl (2R,5S)-5-(3-hydroxypropyl)pyrrolidine-1,2-dicarboxylate (2.4 g, 8.4 mmol, 1 eq.) was added dropwise at -78° C., followed by Et₃N (3.4 g, 33.4 mmol, 4 eq.). After stirring for 2 h at −78° C., the mixture was diluted with NH₄Cl aq. solution and extracted with DCM. The combined organic layers were washed with brine, dried over anhydrous Na₂SO₄ and concentrated. The residue was purified by silica gel column chromatography, eluted with EA/PE (0-20%), to afford the title compound (2 g).

### Step 3: 1-(tert-butyl) 2-methyl (2R,5S)-5-(3,3-difluoropropyl)pyrrolidine-1,2-dicarboxylate

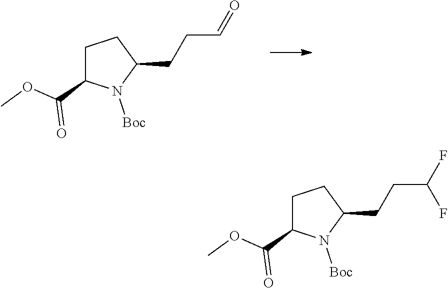 [SMILES: COC(=O)[C@H]1CC[C@@H](CCC=O)N1C(=O)OC(C)(C)C ; COC(=O)[C@H]1CC[C@@H](CCC(F)F)N1C(=O)OC(C)(C)C]

To a solution of 1-(tert-butyl) 2-methyl (2R,5S)-5-(3-oxopropyl)pyrrolidine-1,2-dicarboxylate (2 g, 7 mmol, 1 equiv) in DCM (20 mL) was added BAST (2.33 g, 10.5 mmol, 1.5 eq.) dropwise at −10° C. and the resulting mixture was stirred for 2 h at room temperature. The mixture was diluted with water and then basified to pH=7 with saturated NaHCO₃ aq. solution. The mixture was extracted with DCM, and the combined organic layers were washed with brine, dried over anhydrous Na₂SO₄ and concentrated. The residue was purified by silica gel column chromatography, eluted with EA/PE (0-30%), to afford the title compound (350 mg).

### Step 6: ((5S,7aS)-5-(3,3-difluoropropyl)-2-methylenetetrahydro-1H-pyrrolizin-7a(5H)-yl)-methanol

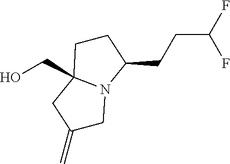 [SMILES: C=C1CN2[C@H](CCC(F)F)CC[C@@]2(CO)C1]

The title compound was prepared by proceeding analogously as described in Intermediate 25, Steps 4-7, using 1-(tert-butyl) 2-methyl (2R,5S)-5-(3,3-difluoropropyl)-pyrrolidine-1,2-dicarboxylate instead of 1-(tert-butyl) 2-methyl (5S)-5-(methoxymethyl)-pyrrolidine-1,2-dicarboxylate in Step 4. MS (ES, m/z): [M+H]⁺=232.1.

### Intermediate 36

### Synthesis of ((5S,7aS)-2-methylene-5-(3,3,3-trifluoropropyl)tetrahydro-1H-pyrrolizin-7a(5H)-yl)-methanol

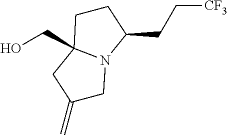 [SMILES: C=C1CN2[C@H](CCC(F)(F)F)CC[C@@]2(CO)C1]

### Step 1: 1-(tert-butyl) 2-methyl (2R,5S)-5-hydroxy-5-(3,3,3-trifluoropropyl)pyrrolidine-1,2-dicarboxylate

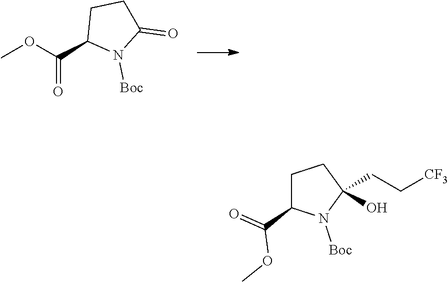 [SMILES: COC(=O)[C@H]1CCC(=O)N1C(=O)OC(C)(C)C ; COC(=O)[C@H]1CC[C@](O)(CCC(F)(F)F)N1C(=O)OC(C)(C)C]

To a solution of 1-tert-butyl 2-methyl (2R)-5-oxopyrrolidine-1,2-dicarboxylate (15 g, 61.7 mmol, 1.0 eq.) in THF (150 mL) was added (3,3,3-trifluoropropyl)magnesium chloride (2.0 M in THF solution, 185 mL, 92.5 mmol, 1.5 eq.) dropwise at −40° C. under nitrogen atmosphere and the resulting mixture was stirred overnight at room temperature. The reaction was quenched by adding sat. NH₄Cl aq. solution at 0° C. and the mixture was extracted with EtOAc. The combined organic layer was washed with brine, dried over anhydrous Na₂SO₄ and concentrated. The residue was purified by silica gel column chromatography, eluted with EA/PE (0-20%), to afford the title compound (8 g).

### Step 2: 1-(tert-butyl) 2-methyl (2R,5S)-5-(3,3,3-trifluoropropyl)pyrrolidine-1,2-dicarboxylate

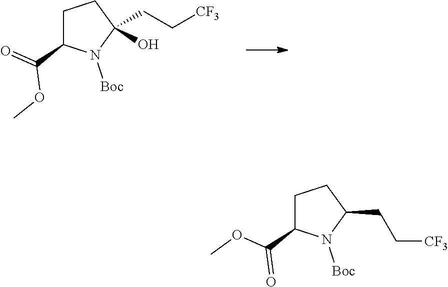 [SMILES: COC(=O)[C@H]1CC[C@](O)(CCC(F)(F)F)N1C(=O)OC(C)(C)C ; COC(=O)[C@H]1CC[C@@H](CCC(F)(F)F)N1C(=O)OC(C)(C)C]

To a solution of 1-tert-butyl 2-methyl (2R,5S)-5-hydroxy-5-(3,3,3-trifluoropropyl)-pyrrolidine-1,2-dicarboxylate (7 g, 20.5 mmol, 1.0 eq.) in EtOAc (70 mL) was added NaBH(OAc)₃ (5.65 g, 26.7 mmol, 1.3 eq.) at 0° C. under nitrogen atmosphere and the reaction mixture was then stirred for 30 min. at 0° C. TFA (7.7 g, 67.7 mmol, 3.3 eq.) was added dropwise at 0° C. and the resulting mixture was stirred overnight at room temperature. The reaction was quenched with sat. NaHCO₃ aq. solution at 0° C. and the mixture was extracted with EtOAc. The combined organic layer was washed with water and brine, dried over anhydrous Na₂SO₄ and concentrated. The residue was purified by silica gel column chromatography, eluted with EA/PE (0-20%), to afford the title compound (5 g).

### Step 3: ((5S,7aS)-2-methylene-5-(3,3,3-trifluoropropyl)tetrahydro-1H-pyrrolizin-7a(5H)-yl)-methanol

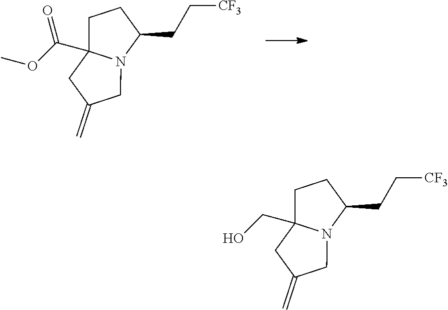 [SMILES: C=C1CN2[C@H](CCC(F)(F)F)CCC2(C(=O)OC)C1 ; C=C1CN2[C@H](CCC(F)(F)F)CCC2(CO)C1]

The title compound was prepared by proceeding analogously as described in Intermediate 25, Steps 4-7, using 1-(tert-butyl) 2-methyl (2R,5S)-5-(3,3,3-trifluoropropyl)pyrrolidine-1,2-dicarboxylate instead of 1-(tert-butyl) 2-methyl (5S)-5-(methoxymethyl)pyrrolidine-1,2-dicarboxylate in Step 4. MS (ES, m/z): [M+H]⁺=250.2

### Intermediate 37

### Synthesis of ((5S,7aS)-5-isobutyl-2-methylenetetrahydro-1H-pyrrolizin-7a(5H)-yl)methanol

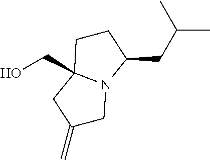

The title compound was prepared by proceeding analogously as described in Intermediate 36, Steps 1-3, using isobutylmagnesium bromide instead of (3,3,3-trifluoropropyl)-magnesium chloride in Step 1. MS (ES, m/z): [M+H]⁺=210.3.

### Intermediate 38

### Synthesis of ((5S,7aS)-5-cyclopropyl-2-methylenetetrahydro-1H-pyrrolizin-7a(5H)-yl)methanol

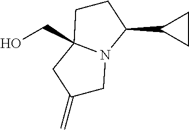

The title compound was prepared by proceeding analogously as described in Intermediate 36, Steps 1-3, using cyclopropylmagnesium bromide instead of (3,3,3-trifluoro-propyl)magnesium chloride in Step 1. MS (ES, m/z): [M+H]⁺=194.4.

### Intermediate 39

### Synthesis of ((5S,7aS)-5-cyclopropyl-2-methylenetetrahydro-1H-pyrrolizin-7a(5H)-yl)methanol

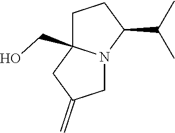

The title compound was prepared by proceeding analogously as described in Intermediate 36, Steps 1-3, using bromo(isopropyl)magnesium instead of (3,3,3-trifluoro-propyl)magnesium chloride in Step 1. MS (ES, m/z): [M+H]⁺=196.2.

### Intermediate 40

### Synthesis of ((2S,7aR)-2-(difluoromethoxy)-6-methylenetetrahydro-1H-pyrrolizin-7a(5H)-yl) methanol

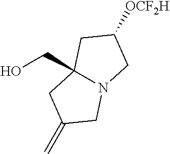

### Step 1: methyl (2S,7aR)-2-(difluoromethoxy)-6-methylenetetrahydro-1H-pyrrolizine-7a(5H)-carboxylate

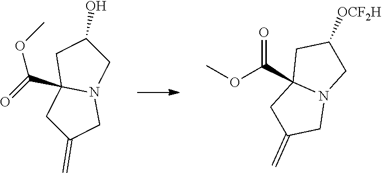

To a mixture of methyl (2S,7aR)-2-hydroxy-6-methylidene-tetrahydro-1H-pyrrolizine-7a-carboxylate (1.0 g, 5.1 mmol, 1.0 eq.) and CuI (386 mg, 2 mmol, 0.40 eq.) in CH₃CN (10 mL) was added 2,2-difluoro-2-(fluorosulfonyl)acetic acid (5.4 g, 30.6 mmol, 6.0 eq.) dropwise at 50° C. under nitrogen atmosphere and the resulting mixture was stirred for 1.5 h at 50° C. After cooling to rt, the mixture was diluted with water and extracted with EtOAc. The combined organic layers were washed with brine, dried over anhydrous Na₂SO₄ and concentrated. The residue was purified by silica gel column chromatography, eluted with EA/PE (0-30%). The material was further purified by reverse flash chromatography to afford the title compound (140 mg).

### Step 2: ((2S,7aR)-2-(difluoromethoxy)-6-methylenetetrahydro-1H-pyrrolizin-7a(5H)-yl) methanol

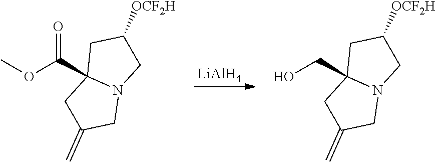

The title compound was prepared by proceeding analogously as described in Intermediate 12, Step 5, by reducing the ester group with LiAlH₄.

### Intermediate 41

### Synthesis of ((2R,7aR)-2-cyclopropoxy-6-methylenetetrahydro-1H-pyrrolizin-7a(5H)-yl)methanol

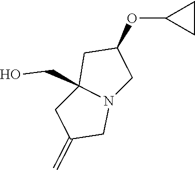

### Step 1: 1-(tert-butyl) 2-methyl (2R,4R)-4-(vinyloxy)pyrrolidine-1,2-dicarboxylate

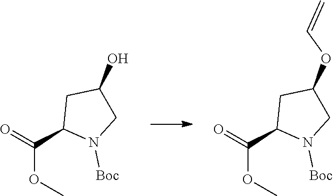 [SMILES: COC(=O)[C@H]1C[C@@H](O)CN1C(=O)OC(C)(C)C ; C=CO[C@@H]1C[C@H](C(=O)OC)N(C(=O)OC(C)(C)C)C1]

A mixture of Pd(OAc)₂ (366 mg, 1.63 mmol, 0.02 eq.) and 1,10-phenanthroline (294 mg, 1.63 mmol, 0.02 eq.) in ethyl vinyl ether (200 mL) was stirred for 20 min. at room temperature under nitrogen atmosphere. To the above mixture was added 1-tert-butyl 2-methyl (2R,4R)-4-hydroxypyrrolidine-1,2-dicarboxylate (20.0 g, 81.5 mmol, 1.00 eq.) dropwise over 5 min. at room temperature and the resulting mixture was stirred for 16 h at 45° C. After cooling to rt, the reaction mixture was concentrated under reduced pressure and the remaining material was added EtOAc. The mixture was then washed with water and brine, dried over anhydrous Na₂SO₄ and concentrated. The residue was purified by silica gel column chromatography, eluted with EA/PE (0-5%) to afford the title compound (6.2 g).

### Step 2: 1-(tert-butyl) 2-methyl (2R,4R)-4-cyclopropoxypyrrolidine-1,2-dicarboxylate

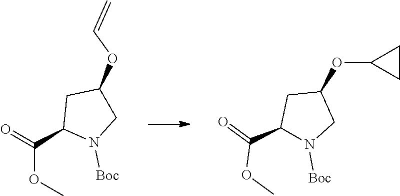 [SMILES: C=CO[C@@H]1C[C@H](C(=O)OC)N(C(=O)OC(C)(C)C)C1 ; COC(=O)[C@H]1C[C@@H](OC2CC2)CN1C(=O)OC(C)(C)C]

To a stirred mixture of 40% KOH (aq. 300 mL) and Et₂O (780 mL) was added 1-methyl-1-nitrosourea (106 g, 1028 mmol, 45.00 eq) in portions at 0° C. and the resulting mixture was stirred for 10 min. The aqueous layer was extracted with Et₂O and the organic layers were combined. This ether solution was added dropwise to a stirred solution of 1-tert-butyl 2-methyl (2R,4R)-4-(ethenyloxy)pyrrolidine-1,2-dicarboxylate (6.2 g, 22.8 mmol, 1.00 eq.) and Pd(acac)₂ (696.2 mg, 2.285 mmol, 0.10 eq.) in DCM (50.0 mL). The resulting mixture was stirred for 1 h, then quenched with water, extracted with DCM. The combined organic layer was washed with water and brine, dried over anhydrous Na₂SO₄ and concentrated. The residue was purified by silica gel column chromatography, eluted with PE/EA (10:1), to afford the title compound (6.2 g).

### Step 3: ((2R,7aR)-2-cyclopropoxy-6-methylenetetrahydro-1H-pyrrolizin-7a(5H)-yl)methanol

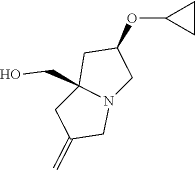

The title compound was prepared by proceeding analogously as described in Intermediate 12, Steps 2-3, using 1-(tert-butyl) 2-methyl (2R,4R)-4-cyclopropoxypyrrolidine-1,2-dicarboxylate instead of 1-(tert-butyl) 2-methyl (2R,4R)-2-(2-(chloromethyl)allyl)-4-fluoropyrrolidine-1,2-dicarboxylate in Step 2.

### Intermediate 42

### Synthesis of ((6R,7aS)-6-fluoro-1-methyl-2-methylenetetrahydro-1H-pyrrolizin-7a(5H)-yl)-methanol

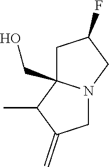

### Step 1: 3-((tert-butyldimethylsilyl)oxy)-2-methylenebutyl 4-methylbenzenesulfonate

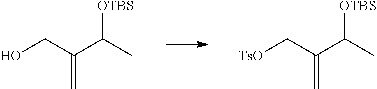 [SMILES: C=C(CO)C(C)O[Si](C)(C)C(C)(C)C ; C=C(COS(=O)(=O)c1ccc(C)cc1)C(C)O[Si](C)(C)C(C)(C)C]

To a stirred solution of 3-((tert-butyldimethylsilyl)oxy)-2-methylenebutan-1-ol (16 g, 74 mmol, 1 eq.) (J. Org. Chem. 1996, 61, 6936-6940]) and TEA (22 g, 222 mmol, 3.0 eq.) in DCM (150 mL) was added p-toluenesulfonyl chloride (17 g, 89 mmol, 1.2 eq.) at room temperature, and the resulting mixture was stirred overnight at room temperature. The mixture was then concentrated under vacuum and the residue was purified by silica gel column chromatography, eluted with EA/PE (0-10%), to afford the title compound (12 g).

### Step 2: methyl (2S,4R)-1-(3-((tert-butyldimethylsilyl)oxy)-2-methylenebutyl)-4-fluoropyrrolidine-2-carboxylate

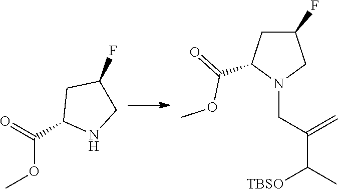

To a stirred solution of methyl (2S,4R)-4-fluoropyrrolidine-2-carboxylate hydrochloride (3.5 g, 19.0 mmol, 1.0 eq.) and TEA (5.8 g, 57 mmol, 3.0 eq.) in DMF (100 mL) was added 3-[(tert-butyldimethylsilyl)oxy]-2-methylidenebutyl 4-methylbenzenesulfonate (7 g, 19 mmol, 1.0 eq.) dropwise at 0° C. under nitrogen atmosphere and the resulting mixture was stirred overnight at room temperature. The mixture was diluted with diethyl ether and the organic layer washed with water, dried over anhydrous Na₂SO₄ and concentrated. The residue was purified by silica gel column chromatography, eluted with EA/PE (0-10%), to afford the title compound (4.7 g).

### Step 3: methyl (2S,4R)-4-fluoro-1-(3-hydroxy-2-methylenebutyl)pyrrolidine-2-carboxylate

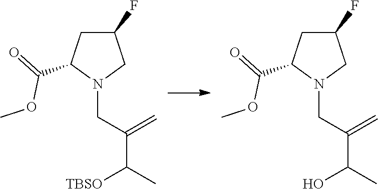 [SMILES: C=C(CN1C[C@H](F)C[C@H]1C(=O)OC)C(C)O[Si](C)(C)C(C)(C)C ; C=C(CN1C[C@H](F)C[C@H]1C(=O)OC)C(C)O]

To a stirred solution of methyl (2S,4R)-1-{3-[(tert-butyldimethylsilyl)oxy]-2-methylidenebutyl}-4-fluoropyrrolidine-2-carboxylate (4.7 g, 13.6 mmol, 1.0 eq.) in THF (50 mL) was added TBAF (1.0 M solution in THF, 27.2 mL, 27.2 mmol, 2.0 eq.) at room temperature and the resulting mixture was stirred overnight at room temperature. The mixture was concentrated under reduced pressure and the residue was purified by silica gel column chromatography, eluted with THF/PE (0-30%), to afford the title compound (2.0 g).

### Step 4: methyl (2S,4R)-1-(3-chloro-2-methylenebutyl)-4-fluoropyrrolidine-2-carboxylate

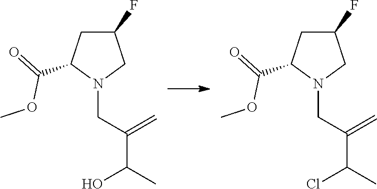 [SMILES: C=C(CN1C[C@H](F)C[C@H]1C(=O)OC)C(C)O ; C=C(CN1C[C@H](F)C[C@H]1C(=O)OC)C(C)Cl]

A solution of methyl (2S,4R)-4-fluoro-1-(3-hydroxy-2-methylenebutyl) pyrrolidine-2-carboxylate (2 g, 8.6 mmol, 1 eq.) in phosphorus oxychloride (10 mL) was stirred for 30. min at 50° C. After cooling to rt, the mixture was concentrated under reduced pressure. DCM as added and the organic layer was washed with brine, dried over anhydrous Na₂SO₄ and concentrated. The residue was purified by silica gel column chromatography, eluted with EA/PE (0-20%), to afford the title compound (240 mg).

### Step 5: methyl (6R,7aS)-6-fluoro-1-methyl-2-methylenetetrahydro-1H-pyrrolizine-7a(5H)-carboxylate

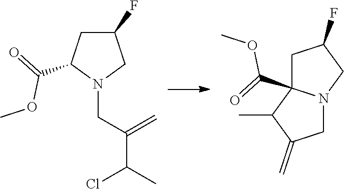 [SMILES: C=C(CN1C[C@H](F)C[C@H]1C(=O)OC)C(C)Cl ; C=C1CN2C[C@H](F)C[C@@]2(C(=O)OC)C1C]

To a stirred solution of methyl (2S,4R)-1-(3-chloro-2-methylenebutyl)-4-fluoro-pyrrolidine-2-carboxylate (700 mg, 2.8 mmol, 1 eq.) in THF (20 mL) were added 1M LiHMDS in THF (11 mL, 11 mmol, 4 eq.) dropwise at 0° C. The resulting mixture was stirred for 1 h at 0° C. and then diluted with water. The resulting mixture was extracted with EA. The combined organic layers were washed with brine, dried over anhydrous Na₂SO₄, and filtered. The filtrate was concentrated under reduced pressure and the residue was purified by silica gel column chromatography, eluted with EA/PE (0-30%), to afford the title compound (100 mg, 16.7%) as a yellow oil.

### Step 6: ((6R,7aS)-6-fluoro-1-methyl-2-methylenetetrahydro-1H-pyrrolizin-7a(5H)-yl)methanol

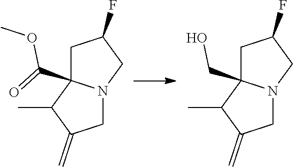

The title compound was prepared by proceeding analogously as described in Intermediate 12, Step 5, by reducing the ester group with LiAlH₄. [M+H]⁺=186.2.

### Intermediate 43

### Synthesis of ((2R,7aS)-2-((tert-butyldiphenylsilyl)oxy)-6-methylenetetrahydro-1H-pyrrolizin-7a(5H)-yl)methanol

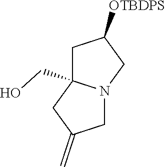

### Step 1: methyl (2R,7aS)-2-((tert-butyldiphenylsilyl)oxy)-6-methylenetetrahydro-1H-pyrrolizine-7a(5H)-carboxylate

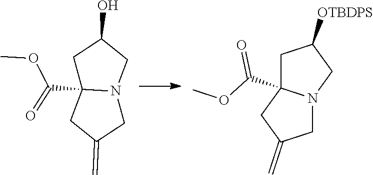

To a solution of methyl (2R,7aS)-2-hydroxy-6-methylenetetrahydro-1H-pyrrolizine-7a(5H)-carboxylate (500 mg, 2.5 mmol, 1.0 equiv.) and imidazole (258 mg, 3.8 mmol, 1.5 equiv.) in CH₂Cl₂ was added TBDPSCl (1.05 g, 3.8 mmol, 1.5 equiv.) dropwise at 0° C. under air atmosphere and the resulting mixture was stirred at room temperature for 2 h. The mixture was diluted with water and extracted with DCM. The combined organic layers were washed with water, brine, dried over anhydrous Na₂SO₄ and concentrated. The residue was purified by silica gel column chromatography, eluted with EA/PE (0-20%), to afford the title compound (834 mg).

### Step 2: ((2R,7aS)-2-((tert-butyldiphenylsilyl)oxy)-6-methylenetetrahydro-1H-pyrrolizin-7a(5H)-yl)methanol

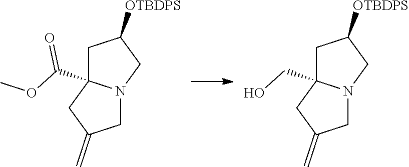

The title compound was prepared by proceeding analogously as described in Intermediate 12, Step 3, by reducing the ester with using LiAlH₄. MS (ES, m/z): [M+H]⁺=408.3;

### Intermediate 44

### Synthesis of ((2S,7aR)-2-((tert-butyldiphenylsilyl)oxy)-6-methylenetetrahydro-1H-pyrrolizin-7a(5H)-yl)methanol

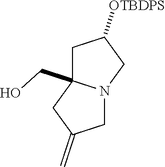

The title compound was prepared by proceeding analogously as described in synthesis of Intermediate 43, Steps 1-2, by using methyl (2S,7aR)-2-hydroxy-6-methylenetetrahydro-1H-pyrrolizine-7a(5H)-carboxylate in step 1 instead of methyl (2R,7aS)-2-hydroxy-6-methylene-tetrahydro-1H-pyrrolizine-7a(5H)-carboxylate in Step 1. MS (ES, m/z): [M+H]⁺=408.3;

### Intermediate 45

### Synthesis of ((2R,7aR)-2-((tert-butyldiphenylsilyl)oxy)-6-methylenetetrahydro-1H-pyrrolizin-7a(5H)-yl)methanol

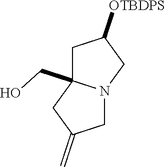

The title compound was prepared by proceeding analogously as described in synthesis of Intermediate 43, Steps 1-2, by using methyl (2R,7aR)-2-((tert-butyldiphenylsilyl)oxy)-6-methylenetetrahydro-1H-pyrrolizine-7a(5H)-carboxylate instead of methyl (2R,7aS)-2-hydroxy-6-methylenetetrahydro-1H-pyrrolizine-7a(5H)-carboxylate in Step 1. MS (ES, m/z): [M+H]⁺=408.3;

### Intermediate 46

### Synthesis of ((2S,3R,7aR)-2-fluoro-3-methyl-6-methylenetetrahydro-1H-pyrrolizin-7a(5H)-yl)methanol

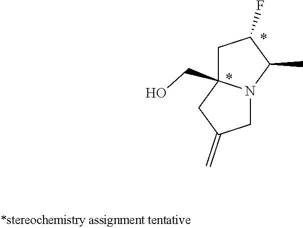

### Step 1: 1-(tert-butyl) 2-methyl (2R,4R,5R)-4-((tert-butyldimethylsilyl)oxy)-2-(2-(chloromethyl)-allyl)-5-methylpyrrolidine-1,2-dicarboxylate

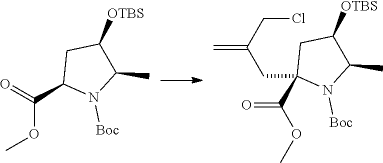 [SMILES: COC(=O)[C@H]1C[C@@H](O[Si](C)(C)C(C)(C)C)[C@@H](C)N1C(=O)OC(C)(C)C ; C=C(CCl)C[C@]1(C(=O)OC)C[C@@H](O[Si](C)(C)C(C)(C)C)[C@@H](C)N1C(=O)OC(C)(C)C]

To a solution of 1-(tert-butyl) 2-methyl (2R,4R,5R)-4-((tert-butyldimethylsilyl)oxy)-5-methylpyrrolidine-1,2-dicarboxylate (22.0 g, 58.8 mmol, 1 equiv.) in THF (250 mL) was dropwise added LiHMDS (1 M, 117 mL, 2 equiv.) and the mixture was stirred at −60° C. for 1 h. 3-Chloro-2-(chloromethyl prop-1-ene (11.0 g, 88.3 mmol, 1.5 eq) was added and the resulting solution was stirred at 20° C. for 2 h. The mixture was quenched with water at 5° C., and extracted with EtOAc. The combined organic layers were concentrated and the residue was purified by column chromatography, eluted with (EA/PE=0-20%), to afford the title compound (22.0 g).

### Step 2: 1-(tert-butyl) 2-methyl (2R,4R,5R)-2-(2-(chloromethyl)allyl)-4-hydroxy-5-methyl-pyrrolidine-1,2-dicarboxylate

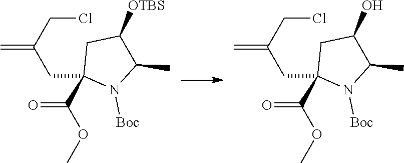 [SMILES: C=C(CCl)C[C@]1(C(=O)OC)C[C@@H](O[Si](C)(C)C(C)(C)C)[C@@H](C)N1C(=O)OC(C)(C)C ; C=C(CCl)C[C@]1(C(=O)OC)C[C@@H](O)[C@@H](C)N1C(=O)OC(C)(C)C]

To a solution of 1-(tert-butyl) 2-methyl (2R,4R,5R)-4-((tert-butyldimethylsilyl)oxy)-2-(2-(chloromethyl)allyl)-5-methylpyrrolidine-1,2-dicarboxylate (21.0 g, 45.4 mmol, 1 eq) in DMF (120 mL) was added CsF (40.0 g, 263 mmol, 9.72 mL, 5.8 eq) and H₂O (2.46 g, 136 mmol, 2.46 mL, 3 eq) and the mixture was stirred at 60° C. for 12 h under N₂. After cooling to rt, the mixture was filtered and concentrated. The residue was purified by column chromatography, eluted with EA/PE (0-50%), to afford the title compound (14.0 g).

### Step 3: 1-(tert-butyl) 2-methyl (2R,4S,5R)-2-(2-(chloromethyl)allyl)-4-fluoro-5-methyl-pyrrolidine-1,2-dicarboxylate

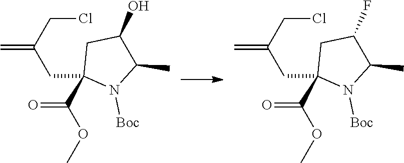 [SMILES: C=C(CCl)C[C@]1(C(=O)OC)C[C@@H](O)[C@@H](C)N1C(=O)OC(C)(C)C ; C=C(CCl)C[C@]1(C(=O)OC)C[C@H](F)[C@@H](C)N1C(=O)OC(C)(C)C]

To a solution of 1-(tert-butyl) 2-methyl (2R,4R,5R)-2-(2-(chloromethyl)allyl)-4-hydroxy-5-methylpyrrolidine-1,2-dicarboxylate (1.00 g, 2.87 mmol, 1 eq) in DCM (20 mL) was added BAST (1.91 g, 8.62 mmol, 1.89 mL, 3 eq) at −60° C. The mixture was slowly warmed to 20° C. and stirred at 20° C. for 12 h. The mixture was quenched with sat. NaHCO₃ aq. solution at 5° C., and then extracted with dichloromethane. The combined organic layers were concentrated and the residue was purified by column chromatography, eluted with EA/PE (0-10%), to afford the title compound (1.50 g).

### Step 4. ((2S,3R,7aR)-2-fluoro-3-methyl-6-methylenetetrahydro-1H-pyrrolizin-7a(5H)-yl)methanol

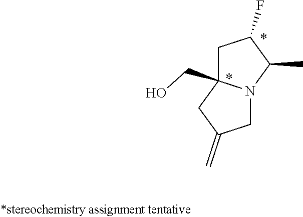

The title compound was prepared by proceeding analogously as described in Intermediate 11, Steps 4-5 using 1-(tert-butyl) 2-methyl (2R,4S,5R)-2-(2-(chloromethyl)allyl)-4-fluoro-5-methylpyrrolidine-1,2-dicarboxylate instead of 1-tert-butyl 2-methyl (2R,4S)-2-[2-(chloromethyl)prop-2-en-1-yl]-4-fluoropyrrolidine-1,2-dicarboxylate in Step 4.

### Example 1

### Synthesis of 4-(4-((1R,5S)-3,8-diazabicyclo[3.2.1]octan-3-yl)-8-fluoro-2-((2-methylenetetrahydro-1H-pyrrolizin-7a(5H)-yl)methoxy)pyrido[4,3-d]pyrimidin-7-yl)-5-ethynylnaphthalen-2-ol

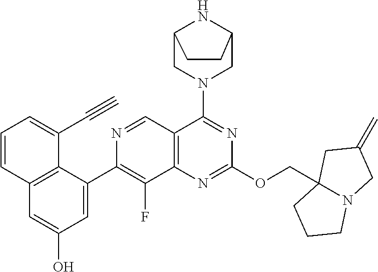 [SMILES: C#Cc1cccc2cc(O)cc(-c3ncc4c(N5CC6CCC(C5)N6)nc(OCC56CCCN5CC(=C)C6)nc4c3F)c12]

### Step 1: ethyl 2-methylene-5-oxotetrahydro-1H-pyrrolizine-7a(5H)-carboxylate

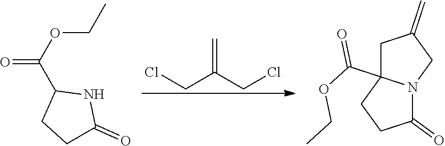

To a stirred mixture of ethyl 5-oxopyrrolidine-2-carboxylate (50.0 g, 318.13 mmol, 1.00 eq.) and 3-chloro-2-(chloromethyl)prop-1-ene (159.0 g, 1272.10 mmol, 4.00 eq.) in THF (300 mL) was added LiHMDS (668 mL, 1.0 M, 668 mmol, 2.10 eq.) dropwise at −40° C. under nitrogen atmosphere. The resulting mixture was stirred overnight at RT, quenched with sat. NH₄Cl (aq.) at 0-5° C., and then neutralized to pH=7 with 1.0 M aqueous HCl. The resulting mixture was extracted with EtOAc. The combined organic layers were washed with water, brine, and then dried over anhydrous Na₂SO₄. After filtration, the filtrate was concentrated and the residue was purified by silica gel column chromatography, eluted with EA/PE (0-70%), to afford the title compound (35.0 g, 52.6%) as a yellow oil.

### Step 2: ethyl 2-methylene-5-oxotetrahydro-1H-pyrrolizine-7a(5H)-carboxylate

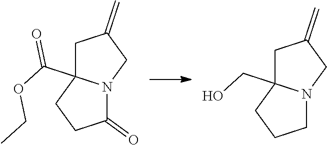

To a stirred solution of ethyl 2-methylidene-5-oxo-tetrahydropyrrolizine-7a-carboxylate (1.0 g, 4.78 mmol, 1.00 eq.) in THF (10 mL) was added 1.0 M LiAlH₄ in THF (14.4 mL, 14.4 mmol, 3.01 eq.) dropwise at RT under nitrogen atmosphere. The resulting mixture was refluxed for 3 h, cooled and then quenched with MeOH and Na₂SO₄·10H₂O. The resulting mixture was filtered through a Celite pad and concentrated. The crude product was purified by Prep-HPLC to afford the title compound (200 mg, 27.3%) as a colorless oil.

### Step 3: tert-butyl (1R,5S)-3-(2,7-dichloro-8-fluoropyrido[4,3-d]pyrimidin-4-yl)-3,8-diazabicyclo[3.2.1]octane-8-carboxylate

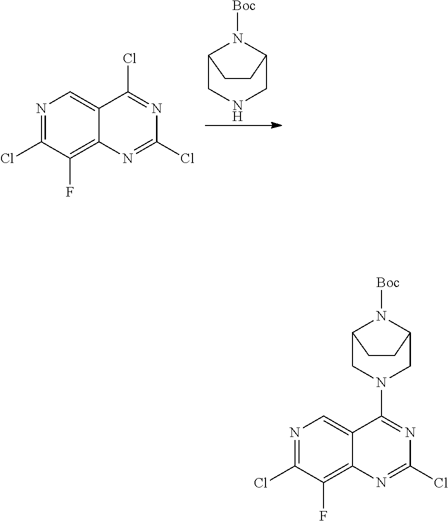 [SMILES: Fc1c(Cl)ncc2c(Cl)nc(Cl)nc12 ; CC(C)(C)OC(=O)N1C2CCC1CNC2 ; CC(C)(C)OC(=O)N1C2CCC1CN(c1nc(Cl)nc3c(F)c(Cl)ncc13)C2]

To a stirred solution of 2,4,7-trichloro-8-fluoropyrido[4,3-d]pyrimidine (10.0 g, 39.61 mmol, 1.00 eq.) in DCM (160 mL) was added DIPEA (12.8 g, 99.03 mmol, 2.50 eq.) slowly at −40° C. The resulting mixture was stirred at −40° C. for 15 min, a solution of tert-butyl (1R,5S)-3,8-diazabicyclo[3.2.1]octane-8-carboxylate (8.4 g, 39.57 mmol, 1.00 eq.) in DCM (35 mL) then was added dropwise at −40° C. The resulting mixture was stirred for additional 15 min at −40° C., diluted with water, and then extracted with EtOAc. The combined organic layers were washed with brine, dried over anhydrous Na₂SO₄, filtered, and then concentrated. The residue was purified by silica gel column chromatography, eluted with EA/PE (0-50%), to afford the title compound (13.6 g, 80.2%) as a yellow solid.

### Step 4: tert-butyl (1R,5S)-3-(7-chloro-8-fluoro-2-((2-methylenetetrahydro-1H-pyrrolizin-7a(5H)-yl)methoxy) pyrido[4,3-d]pyrimidin-4-yl)-3,8-diazabicyclo[3.2.1]octane-8-carboxylate

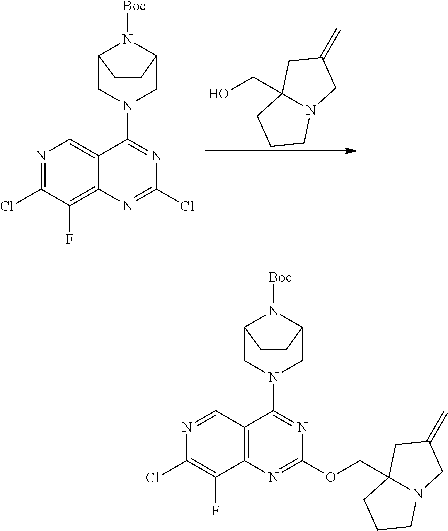 [SMILES: CC(C)(C)OC(=O)N1C2CCC1CN(c1nc(Cl)nc3c(F)c(Cl)ncc13)C2 ; C=C1CN2CCCC2(CO)C1 ; C=C1CN2CCCC2(COc2nc(N3CC4CCC(C3)N4C(=O)OC(C)(C)C)c3cnc(Cl)c(F)c3n2)C1]

A mixture of tert-butyl (1R,5S)-3-(2,7-dichloro-8-fluoropyrido[4,3-d]pyrimidin-4-yl)-3,8-diazabicyclo[3.2.1]octane-8-carboxylate (200 mg, 0.47 mmol, 1.00 eq.), (2-methylidene-tetrahydro-1H-pyrrolizin-7a-yl)methanol (107 mg, 0.70 mmol, 1.49 eq.) and DIPEA (181 mg, 1.40 mmol, 2.98 eq.) in 1,4-dioxane (2 mL) was stirred for 3 h at 80° C. The resulting mixture was cooled and diluted with water. The resulting mixture was extracted with EtOAc. The combined organic layers were washed with water, brine, dried over anhydrous Na₂SO₄, filtered and then concentrated. The residue was purified by silica gel column chromatography, eluted with MeOH/CH₂Cl₂ (0-10%), to afford the title compound (135 mg, 53.2%) as a yellow solid.

### Step 5: tert-butyl (1R,5S)-3-(8-fluoro-7-(3-(methoxymethoxy)-8-((triisopropylsilyl)ethynyl) naphthalen-1-yl)-2-((2-methylenetetrahydro-1H-pyrrolizin-7a(5H)-yl)methoxy)pyrido[4,3-d]pyrimidin-4-yl)-3,8-diazabicyclo[3.2.1] octane-8-carboxylate

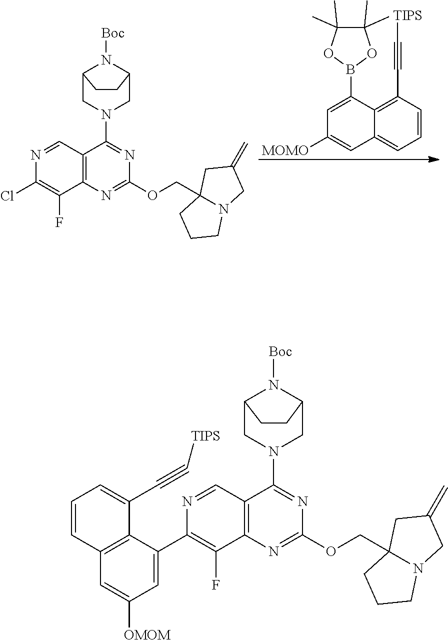

A mixture of tert-butyl (1R,5S)-3-(7-chloro-8-fluoro-2-((2-methylenetetrahydro-1H-pyrrolizin-7a(5H)-yl)methoxy)pyrido [4,3-d]pyrimidin-4-yl)-3,8-diazabicyclo[3.2.1]octane-8-carboxylate (120 mg, 0.22 mmol, 1.00 eq.), triisopropyl((6-(methoxymethoxy)-8-(4,4,5,5-tetramethyl-1,3,2-dioxaborolan-2-yl)naphthalen-1-yl)ethynyl)silane (130 mg, 0.26 mmol, 1.18 eq.), PdCl₂(dtbpf) (14 mg, 0.021 mmol, 0.095 eq.) and K₂CO₃ (60 mg, 0.43 mmol, 1.95 eq.) in 1,4-dioxane (1.0 mL) and H₂O (0.1 mL) was stirred overnight at 85° C. under nitrogen atmosphere. The resulting mixture was cooled, diluted with water and then extracted with EtOAc. The combined organic layers were washed with water, brine, dried over anhydrous Na₂SO₄, filtered and then concentrated. The residue was purified by silica gel column chromatography, eluted with MeOH/CH₂Cl₂ (0-10%), to afford the title compound (30 mg, 15.5%) as a yellow solid.

### Step 6: tert-butyl (1R,5S)-3-(7-(8-ethynyl-3-(methoxymethoxy)naphthalen-1-yl)-8-fluoro-2-((2-methylene tetrahydro-1H-pyrrolizin-7a(5H)-yl)methoxy)pyrido[4,3-d]pyrimidin-4-yl)-3,8-diazabicyclo[3.2.1]octane-8-carboxylate

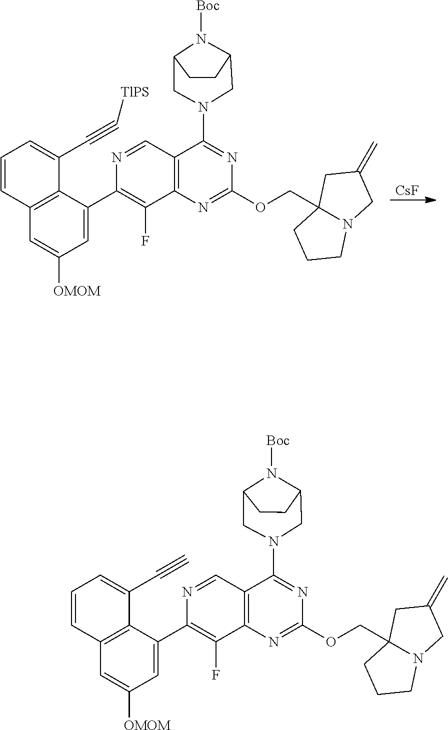

To a stirred solution of tert-butyl (1R,5S)-3-(8-fluoro-7-(3-(methoxymethoxy)-8-((triisopropylsilyl)ethynyl)naphthalen-1-yl)-2-((2-methylenetetrahydro-1H-pyrrolizin-7a(5H)-yl)methoxy)pyrido[4,3-d]pyrimidin-4-yl)-3,8-diazabicyclo[3.2.1]octane-8-carboxylate (33 mg, 0.038 mmol, 1.00 eq.) in DMF (0.6 mL) was added CsF (29 mg, 0.19 mmol, 5.00 eq.) at RT. The resulting mixture was stirred for 3 h at RT, diluted with water, and then extracted with EtOAc. The combined organic layers were washed with water, brine, dried over anhydrous Na₂SO₄, filtered and then concentrated to give the title compound (24 mg, 86.8%) as a light yellow solid.

### Step 7: 4-(4-((1R,5S)-3,8-diazabicyclo[3.2.1]octan-3-yl)-8-fluoro-2-((2-methylenetetrahydro-1H-pyrrolizin-7a(5H)-yl)methoxy)pyrido[4,3-d]pyrimidin-7-yl)-5-ethynylnaphthalen-2-ol

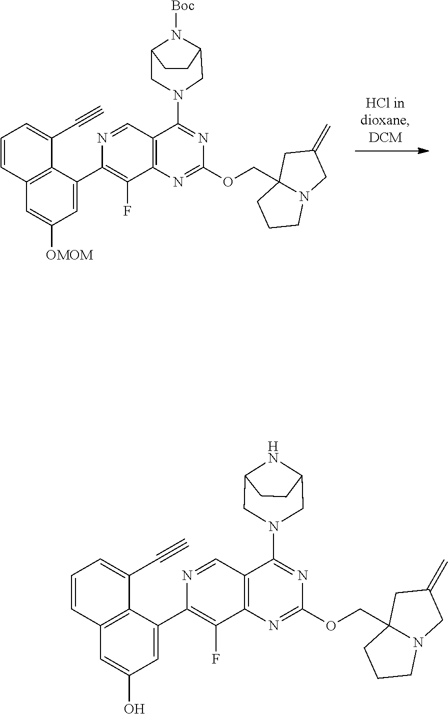

A solution of tert-butyl (1R,5S)-3-(7-(8-ethynyl-3-(methoxymethoxy)naphthalen-1-yl)-8-fluoro-2-((2-methylene tetrahydro-1H-pyrrolizin-7a(5H)-yl)methoxy)pyrido[4,3-d]pyrimidin-4-yl)-3,8-diazabicyclo[3.2.1]octane-8-carboxylate (24 mg, 0.033 mmol, 1.00 eq.) in DCM (0.5 mL) was added HCl in dioxane (0.5 mL, 4.0 M, 2.0 mmol, 60.61 eq.) at 0° C. The reaction mixture was stirred for 30 min at 0° C., basified to pH=8 with NH₃ in MeOH and then concentrated. The crude product was purified by Prep-HPLC to afford the title compound (5.5 mg, 28.8%) as a yellow solid. MS (ES, m/z): [M+1]=577.3.

### Example 2

### Synthesis of 4-(4-((1R,5S)-3,8-diazabicyclo[3.2.1]octan-3-yl)-8-fluoro-2-((2-fluoro-6-methylene-tetrahydro-1H-pyrrolizin-7a(5H)-yl)methoxy)pyrido[4,3-d]pyrimidin-7-yl)-5-ethynyl-naphthalen-2-ol

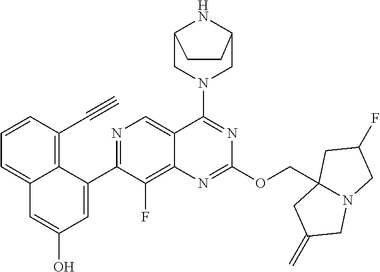 [SMILES: C#Cc1cccc2cc(O)cc(-c3ncc4c(N5CC6CCC(C5)N6)nc(OCC56CC(=C)CN5CC(F)C6)nc4c3F)c12]

### Step 1: mixture of 1-(tert-butyl) 2-methyl (2S,4S)-4-((tert-butyldimethylsilyl)oxy)pyrrolidine-1,2-dicarboxylate and 1-(tert-butyl) 2-methyl (2R,4R)-4-((tert-butyldimethylsilyl)oxy)-pyrrolidine-1,2-dicarboxylate

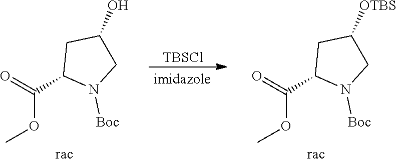

To a stirred solution of 1-(tert-butyl) 2-methyl (2S,4S)-4-hydroxypyrrolidine-1,2-dicarboxylate and 1-(tert-butyl) 2-methyl (2R,4R)-4-hydroxypyrrolidine-1,2-dicarboxylate (30.0 g, 122.31 mmol, 1.00 eq.) and imidazole (16.65 g, 244.58 mmol, 2.00 eq.) in DMF (300 mL) was added TBSCl (22.12 g, 146.76 mmol, 1.20 eq.) in portions at 25° C. The resulting mixture was stirred for 5 h at 25° C., diluted with water and then extracted with EtOAc. The combined organic layers were washed with water, brine, dried over anhydrous Na₂SO₄, filtered and then concentrated. The residue was purified by silica gel column chromatography, eluted with EA/PE (0-10%), to afford the title compound (40.1 g, 91.2%) as a light brown oil.

### Step 2: 1-(tert-butyl) 2-methyl -4-((tert-butyldimethylsilyl)oxy)-2-(2-(chloromethyl)allyl) pyrrolidine-1,2-dicarboxylate

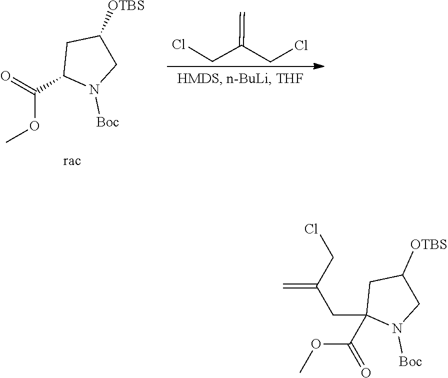

To a stirred solution of HMDS (4.94 g, 30.61 mmol, 1.10 eq.) in anhydrous THF (100 mL) was added n-BuLi (2.5M in hexanes, 12.2 mL, 30.50 mmol, 1.10 eq.) slowly at −78° C. under a nitrogen atmosphere and the resulting mixture was stirred for 30 min at −78° C. A solution of 1-(tert-butyl) 2-methyl (2S,4S)-4-((tert-butyldimethylsilyl)oxy)pyrrolidine-1,2-dicarboxylate and 1-(tert-butyl) 2-methyl (2R,4R)-4-((tert-butyldimethylsilyl)oxy)pyrrolidine-1,2-dicarboxylate (10.0 g, 27.81 mmol, 1.00 eq.) in anhydrous THF (100 mL) slowly at -78° C. and the resulting mixture was stirred for 15 min at −78° C. To the above mixture was added 3-chloro-2-(chloromethyl)prop-1-ene (4.17 g, 33.36 mmol, 1.20 eq.) dropwise in at −78° C. The resulting mixture was stirred for 12h at 25° C., quenched with saturated NH₄Cl aqueous solution at 5° C. and then extracted with EtOAc. The combined organic layers were washed with brine, dried over anhydrous Na₂SO₄, filtered and then concentrated. The residue was purified by silica gel column chromatography, eluted with EA/PE (0-30%), to afford the title compound (10.0 g, 80.3%) as a light-yellow oil.

### Step 3: 1-(tert-butyl) 2-methyl -2-(2-(chloromethyl)allyl)-4-hydroxypyrrolidine-1,2-dicarboxylate

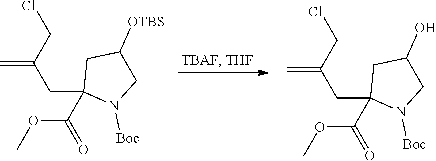 [SMILES: C=C(CCl)CC1(C(=O)OC)CC(O[Si](C)(C)C(C)(C)C)CN1C(=O)OC(C)(C)C ; C=C(CCl)CC1(C(=O)OC)CC(O)CN1C(=O)OC(C)(C)C]

To a stirred solution of 1-(tert-butyl) 2-methyl -4-((tert-butyldimethylsilyl)oxy)-2-(2-(chloromethyl)allyl)pyrrolidine-1,2-dicarboxylate (8.0 g, 17.85 mmol, 1.00 eq.) in THF (80 mL) was added TBAF (6.4 g, 24.48 mmol, 1.37 eq.) in portions at 0° C. The resulting mixture was stirred for 12 h at 25° C., quenched with water at 5° C. and then extracted with EtOAc. The combined organic layers were washed with water, brine, dried over anhydrous Na₂SO₄, filtered and then concentrated. The residue was purified by silica gel column chromatography, eluted with EA/PE (0-50%), to afford the title compound (4.5 g, 75.5%) as a light brown oil.

### Step 4: 1-(tert-butyl) 2-methyl -2-(2-(chloromethyl)allyl)-4-fluoropyrrolidine-1,2-dicarboxylate

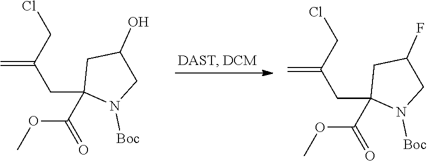 [SMILES: C=C(CCl)CC1(C(=O)OC)CC(O)CN1C(=O)OC(C)(C)C ; C=C(CCl)CC1(C(=O)OC)CC(F)CN1C(=O)OC(C)(C)C]

To a stirred solution of 1-(tert-butyl) 2-methyl -2-(2-(chloromethyl)allyl)-4-hydroxy-pyrrolidine-1,2-dicarboxylate (4.5 g, 13.48 mmol, 1.00 eq.) in DCM (45 mL) was added DAST (3.26 g, 20.22 mmol, 1.50 eq.) dropwise at −78° C. under nitrogen atmosphere. The resulting mixture was stirred for 12 h at 25° C., quenched with water at 5° C. and then extracted with EtOAc. The combined organic layers were washed with water, brine, dried over anhydrous Na₂SO₄, filtered and then concentrated. The residue was purified by silica gel column chromatography, eluted with EA/PE (0-30%), to afford the title compound (3.0 g, 66.2%) as a light brown oil.

### Step 5: methyl 2-fluoro-6-methylenetetrahydro-1H-pyrrolizine-7a(5H)-carboxylate

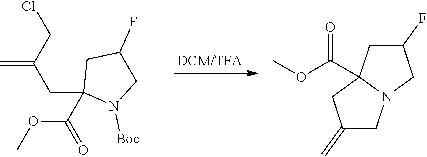 [SMILES: C=C(CCl)CC1(C(=O)OC)CC(F)CN1C(=O)OC(C)(C)C ; C=C1CN2CC(F)CC2(C(=O)OC)C1]

To a stirred solution of 1-(tert-butyl) 2-methyl -2-(2-(chloromethyl)allyl)-4-fluoropyrrolidine-1,2-dicarboxylate (2.5 g, 7.44 mmol, 1.00 eq.) in DCM (25 mL) was added TFA (8.0 mL) dropwise at 5° C. The resulting mixture was stirred for 2 h at 25° C. and then concentrated. The residue was treated with aqueous NaHCO₃ and then extracted with EtOAc. The combined organic layers were washed with water, brine, dried over anhydrous Na₂SO₄, filtered and then concentrated. The residue was purified by silica gel column chromatography, eluted with EA/PE (0-50%), to afford the title compound (1.01 g, 68.1%) as a light brown oil.

### Step 6: 2-fluoro-6-methylenetetrahydro-1H-pyrrolizin-7a(5H)-yl)methanol

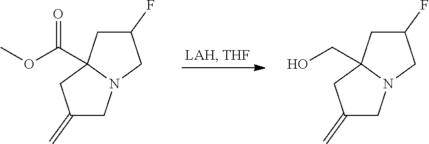

To a stirred solution of methyl 2-fluoro-6-methylidene-tetrahydro-1H-pyrrolizine-7a-carboxylate (600 mg, 3.01 mmol, 1.00 eq.) in anhydrous THF (5 mL) was added LAH (1.0 mol/L, 4.50 mL, 4.50 mmol, 1.50 eq.) dropwise at 0° C. The resulting mixture was stirred for 2 h at 25° C., quenched with water, 15% aqueous NaOH and then water in sequence at 0° C. The resulting mixture was filtered, and the filter cake was washed with EtOAc. The filtrate was concentrated to give the crude title compound (1.1 g) as a light-yellow solid, which was used for next step without further purification.

### Step 7: 4-(4-((1R,5S)-3,8-diazabicyclo[3.2.1]octan-3-yl)-8-fluoro-2-((2-fluoro-6-methylenetetrahydro-1H-pyrrolizin-7a(5H)-yl)methoxy)pyrido[4,3-d]pyrimidin-7-yl)-5-ethynylnaphthalen-2-ol

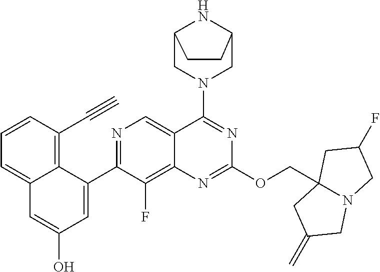 [SMILES: C#Cc1cccc2cc(O)cc(-c3ncc4c(N5CC6CCC(C5)N6)nc(OCC56CC(=C)CN5CC(F)C6)nc4c3F)c12]

Proceeding analogously as described in Example 1, Steps 4-7 above, but using 2-fluoro-6-methylenetetrahydro-1H-pyrrolizin-7a(5H)-yl)methanol instead of (2-methylidene-tetrahydro-1H-pyrrolizin-7a-yl)methanol in Step 4, provided the title compound. MS (ES, m/z): [M+1]=595.3.

### Example 3

### Synthesis of 4-(4-((1R,5S)-3,8-diazabicyclo[3.2.1]octan-3-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol

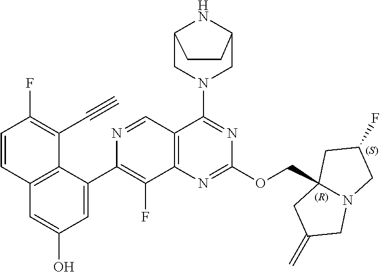

### Step 1: tert-butyl (1R,5S)-3-(2,7-dichloro-8-fluoropyrido[4,3-d]pyrimidin-4-yl)-3,8-diazabicyclo-[3.2.1]octane-8-carboxylate

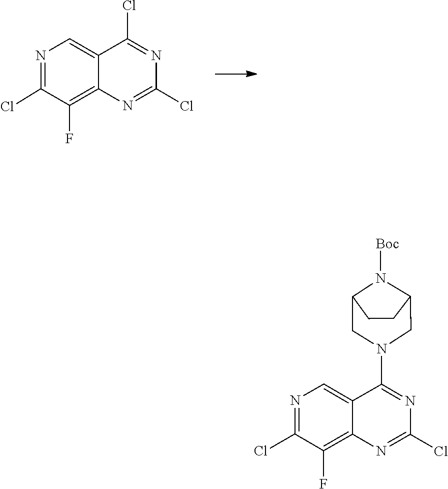 [SMILES: Fc1c(Cl)ncc2c(Cl)nc(Cl)nc12 ; CC(C)(C)OC(=O)N1C2CCC1CN(c1nc(Cl)nc3c(F)c(Cl)ncc13)C2]

To a stirred solution of 2,4,7-trichloro-8-fluoropyrido[4,3-d]pyrimidine (10.0 g, 39.61 mmol, 1.00 eq.) in DCM (160 mL) was added DIPEA (12.8 g, 99.03 mmol, 2.50 eq.) slowly at −40° C. The resulting mixture was stirred at −40° C. for 15 min., and a solution of tert-butyl (1R,5S)-3,8-diazabicyclo[3.2.1]octane-8-carboxylate (8.4 g, 39.57 mmol, 1.00 eq.) in DCM (35 mL) was added dropwise at −40° C. After stirring the resulting mixture for additional 15 min. at −40° C., the mixture was diluted with water, and then extracted with EtOAc. The combined organic layers were washed with brine, dried over anhydrous Na₂SO₄, filtered, and then concentrated. The residue was purified by silica gel column chromatography, eluted with EA/PE (0-50%), to afford the title compound (13.6 g, 80.2%) as a yellow solid.

### Step 2: tert-butyl (1R,5S)-3-(7-chloro-8-fluoro-2-(((2S,7aR)-2-fluoro-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate

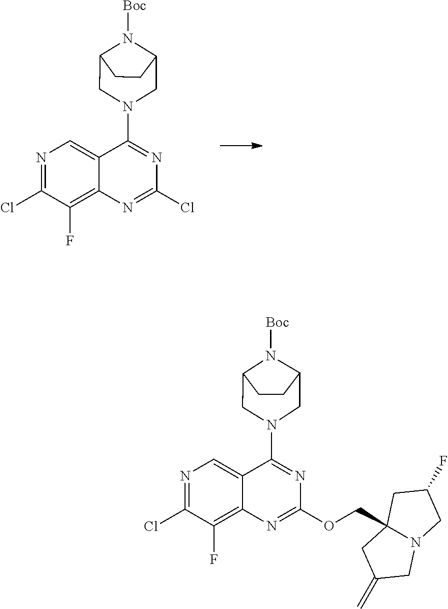 [SMILES: CC(C)(C)OC(=O)N1C2CCC1CN(c1nc(Cl)nc3c(F)c(Cl)ncc13)C2 ; C=C1CN2C[C@@H](F)C[C@@]2(COc2nc(N3CC4CCC(C3)N4C(=O)OC(C)(C)C)c3cnc(Cl)c(F)c3n2)C1]

To a stirred solution of tert-butyl (1R,5S)-3-(2,7-dichloro-8-fluoropyrido[4,3-d]pyrimidin-4-yl)-3,8-diazabicyclo[3.2.1]octane-8-carboxylate (200 mg, 0.47 mmol, 1 equiv.) and ((2S,7aR)-2-fluoro-6-methylenetetrahydro-1H-pyrrolizin-7a(5H)-yl)methanol (79 mg, 0.47 mmol, 1.0 equiv.) in THF (2 mL) at 0° C. was added NaH (22 mg, 0.93 mmol, 2.0 equiv) in portions. The resulting mixture was stirred for 3 h at room temperature under nitrogen atmosphere and then quenched by adding ice/salt (20 mL) at 0° C. The mixture was diluted with water and extracted with EtOAc. The combined organic layers were washed with brine, dried over anhydrous Na₂SO₄ and concentrated. The residue was purified by silica gel column chromatography, eluted with EA/PE (0-50%) to afford the title compound (130 mg) as a light-yellow solid.

### Step 3: tert-butyl (1R,5S)-3-(8-fluoro-7-(7-fluoro-3-(methoxymethoxy)-8-((triisopropylsilyl)-ethynyl)naphthalen-1-yl)-2-(((2S,7aR)-2-fluoro-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate

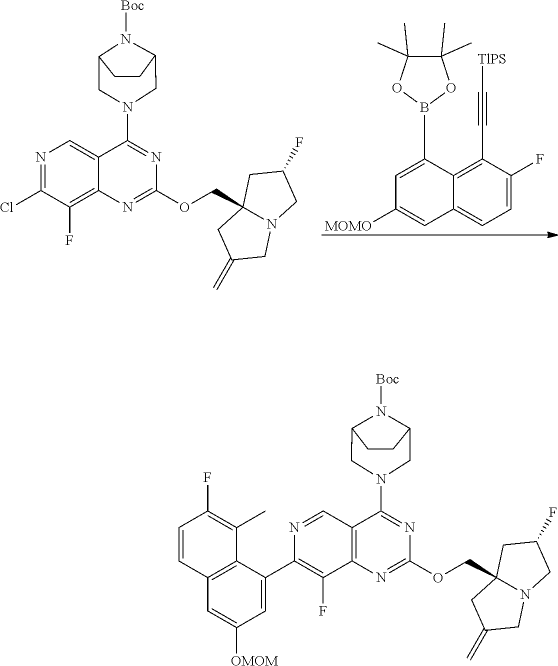

To a stirred mixture of tert-butyl 3-(2-{[(2S,7aR)-2-fluoro-6-methylidene-tetrahydro-1H-pyrrolizin-7a-yl]methoxy}-7-chloro-8-fluoropyrido[4,3-d]pyrimidin-4-yl)-3,8-diazabicyclo[3.2.1]-octane-8-carboxylate (135 mg, 0.24 mmol, 1.0 equiv) and {2-[2-fluoro-6-(methoxymethoxy)-8-(4,4,5,5-tetramethyl-1,3,2-dioxaborolan-2-yl)naphthalen-1-yl]ethynyl}triisopropylsilane (147.47 mg, 0.29 mmol, 1.2 equiv) in DME/H₂O (3 mL) was added K₂CO₃ (66.27 mg, 0.48 mmol, 2.0 equiv), and CATACXIUM A Pd G3 (17.77 mg, 0.024 mmol, 0.1 equiv) in portions at room temperature under nitrogen atmosphere. The resulting mixture was stirred for 3 h at 80° C. under nitrogen atmosphere. After cooling the mixture to rt, the mixture was concentrated under reduced pressure and the residue was purified by silica gel column chromatography, eluted with MeOH/DCM (0-5%) to afford the title compound (85 mg).

### Step 4: tert-butyl (1R,5S)-3-(7-(8-ethynyl-7-fluoro-3-(methoxymethoxy)naphthalen-1-yl)-8-fluoro-2-(((2S,7aR)-2-fluoro-6-methylenetetrahydro-1H-pyrrolizin-7a(5H)-yl)methoxy)-pyrido[4,3-d]pyrimidin-4-yl)-3,8-diazabicyclo[3.2.1]octane-8-carboxylate

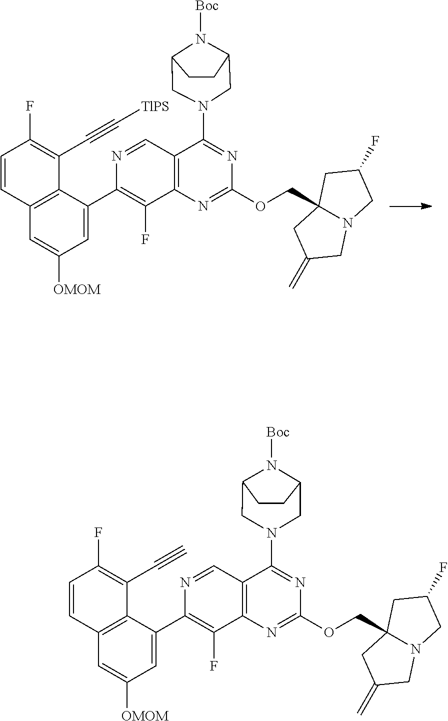

A mixture of tert-butyl 3-(2-{[(2S,7aR)-2-fluoro-6-methylidene-tetrahydro-1H-pyrrolizin-7a-yl]methoxy}-8-fluoro-7-[7-fluoro-3-(methoxymethoxy)-8-[2-(triisopropylsilyl)ethynyl]-naphthalen-1-yl]pyrido[4,3-d]pyrimidin-4-yl)-3,8-diazabicyclo[3.2.1]octane-8-carboxylate (75 mg, 0.08 mmol, 1.0 equiv) and CsF (62.38 mg, 0.41 mmol, 5.0 equiv) in DMF (1.5 mL) was stirred for overnight at room temperature under nitrogen atmosphere. The resulting mixture was diluted with water and then extracted with EtOAc. The organic layers were combined and concentrated under reduced pressure to afford the title compound (50 mg) as a brown crude solid.

### Step 5: 4-(4-((1R,5S)-3,8-diazabicyclo[3.2.1]octan-3-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol

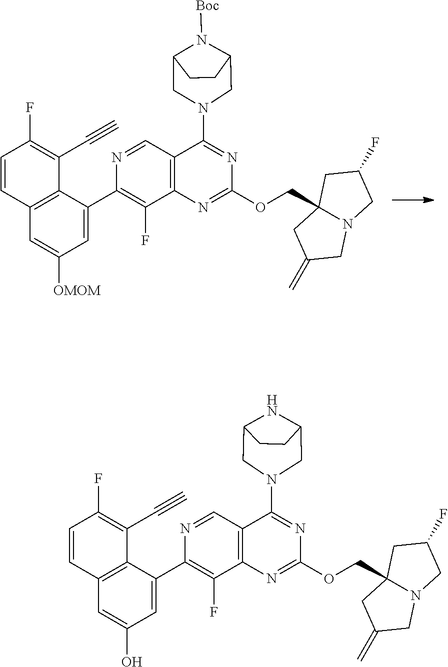

To a stirred mixture of tert-butyl 3-(2-{[(2S,7aR)-2-fluoro-6-methylidene-tetrahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate (40 mg, 0.05 mmol, 1.0 equiv) in DCM (2 mL) was added HCl(gas) in 1,4-dioxane (1 mL) dropwise at 0° C. under nitrogen atmosphere. The resulting mixture was stirred for 1 h at 0° C. under nitrogen atmosphere and then basified to pH 8 with NH₃MeOH. The resulting mixture was concentrated under reduced pressure. The crude product (mg) was purified by Prep-HPLC to afford the title compound (10 mg, 30.88%) as a light yellow solid. MS (ES, m/z): [M+1]⁺=613.4. Proceeding analogously as described in Example 3, Steps 2-5, the following compounds shown in Table A below were prepared by replacing in Step 2, ((2S,7aR)-2-fluoro-6-methylenetetra-hydro-1H-pyrrolizin-7a(5H)-yl)methanol with alcohols indicated therein.

TABLE A

| | Alcohol used in | MS |
|---|---|---|
| Compound | Step 2 | [M + H]⁺ |
| 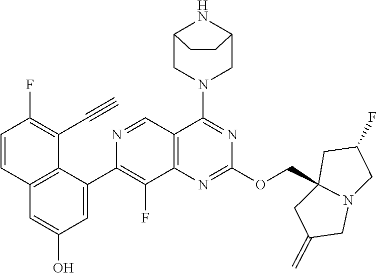 [SMILES: C#Cc1c(F)ccc2cc(O)cc(-c3ncc4c(N5CC6CCC(C5)N6)nc(OC[C@]56CC(=C)CN5C[C@@H](F)C6)nc4c3F)c12] | Intermediate 11 | 613.4 |
| 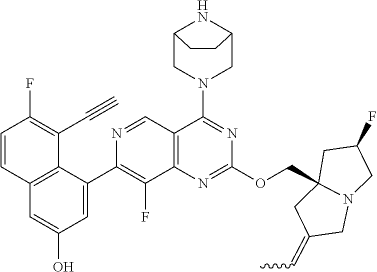 | Intermediate 15 | |
| 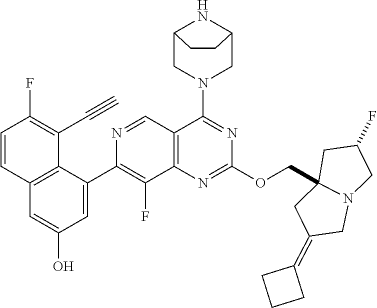 | Intermediate 16 | 653.4 |
| 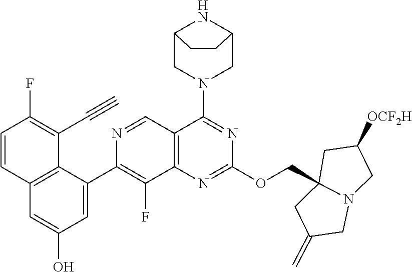 | Intermediate 23 | 661.4 |
| 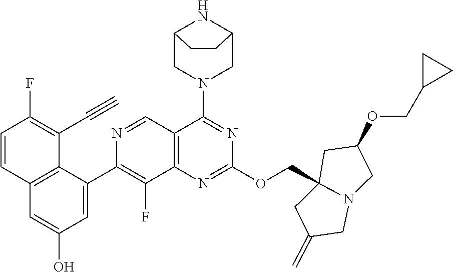 [SMILES: C#Cc1c(F)ccc2cc(O)cc(-c3ncc4c(N5CC6CCC(C5)N6)nc(OC[C@]56CC(=C)CN5C[C@H](OCC5CC5)C6)nc4c3F)c12] | Intermediate 24 | 665.4 |
| 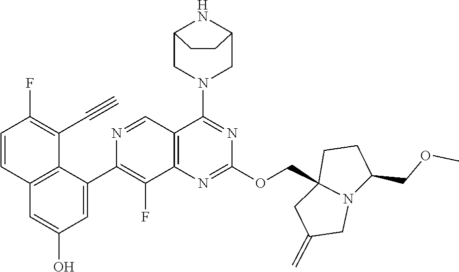 [SMILES: C#Cc1c(F)ccc2cc(O)cc(-c3ncc4c(N5CC6CCC(C5)N6)nc(OC[C@@]56CC[C@@H](COC)N5CC(=C)C6)nc4c3F)c12] | Intermediate 25 | 639.4 |
| and | | |
| 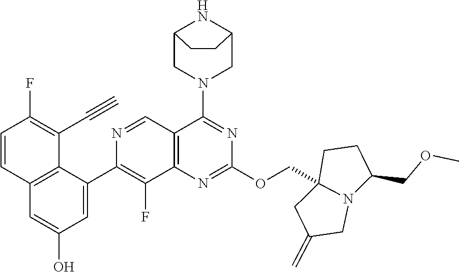 | | |
| 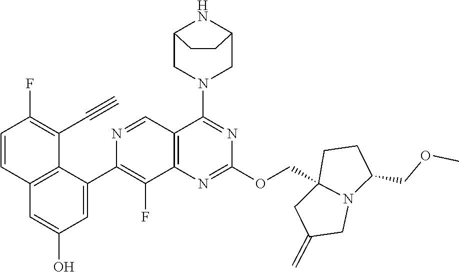 [SMILES: C#Cc1c(F)ccc2cc(O)cc(-c3ncc4c(N5CC6CCC(C5)N6)nc(OC[C@]56CC[C@H](COC)N5CC(=C)C6)nc4c3F)c12] | Intermediate 26 | 639.4 |
| and | | |
| 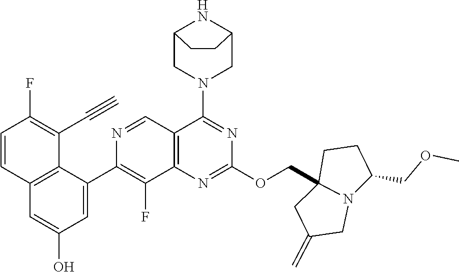 | | |

Proceeding analogously as described in Example 1, Steps 1-5, compounds in Table B below were synthesized by replacing tert-butyl (1R,5S)-3,8-diazabicyclo-[3.2.1]octane-8-carboxylate in Step 1, with amines indicated therein.

TABLE B

| | Amine used | MS |
|---|---|---|
| Compound | in Step 1 | [M + H]+ |
| 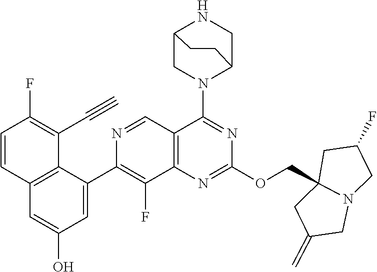 [SMILES: C#Cc1c(F)ccc2cc(O)cc(-c3ncc4c(N5CC6CCC5CN6)nc(OC[C@]56CC(=C)CN5C[C@@H](F)C6)nc4c3F)c12] | 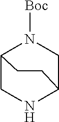 [SMILES: CC(C)(C)OC(=O)N1CC2CCC1CN2] | 631.4 |
| 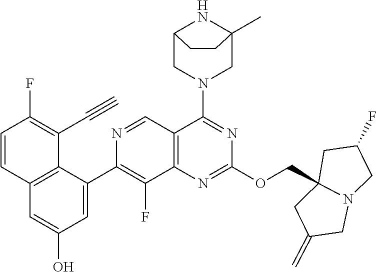 [SMILES: C#Cc1c(F)ccc2cc(O)cc(-c3ncc4c(N5CC6CCC(C)(C5)N6)nc(OC[C@]56CC(=C)CN5C[C@@H](F)C6)nc4c3F)c12] | 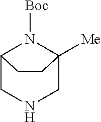 [SMILES: CC(C)(C)OC(=O)N1C2CCC1(C)CNC2] | 627.3 |
| 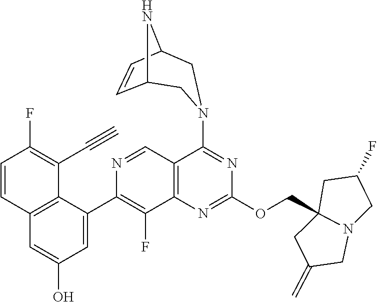 [SMILES: C#Cc1c(F)ccc2cc(O)cc(-c3ncc4c(N5CC6C=CC(C5)N6)nc(OC[C@]56CC(=C)CN5C[C@@H](F)C6)nc4c3F)c12] | 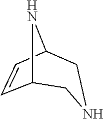 | 611.3 |
| 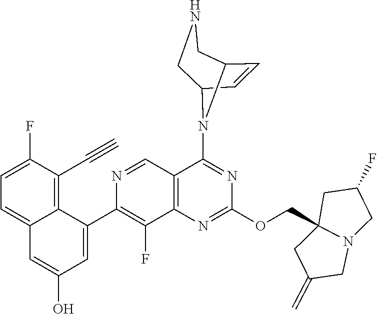 [SMILES: C#Cc1c(F)ccc2cc(O)cc(-c3ncc4c(N5C6C=CC5CNC6)nc(OC[C@]56CC(=C)CN5C[C@@H](F)C6)nc4c3F)c12] | 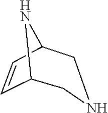 | 611.3 |

Proceeding analogously as described in Example 3, Steps 2-5, compounds in Table C below were prepared using triisopropyl((6-(methoxymethoxy)-8-(4,4,5,5-tetramethyl-1,3,2-dioxaborolan-2-yl)naphthalen-1-yl)ethynyl)silane in Step 3 and replacing in Step 2, ((2S,7aR)-2-fluoro-6-methylenetetra-hydro-1H-pyrrolizin-7a(5H)-yl)methanol with alcohols indicated therein.

TABLE C

| | Alcohol used | MS |
|---|---|---|
| Compound | in Step 2 | [M + H]⁺ |
| 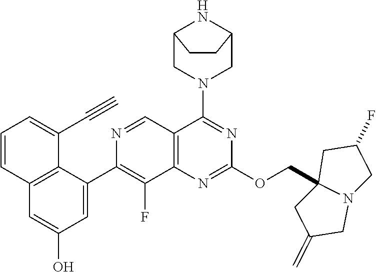 | Intermediate 11 | 595.3 |
| 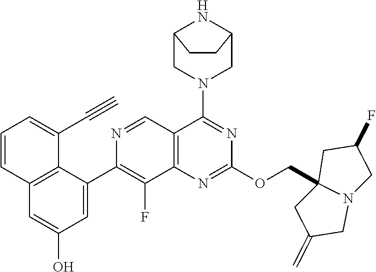 | Intermediate 12 | 595.3 |
| 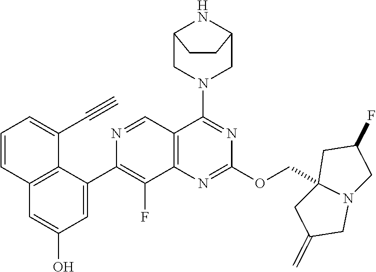 | Intermediate 13 | 595.3 |
| 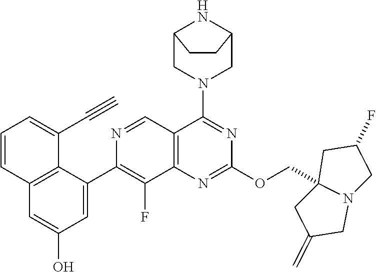 | Intermediate 14 | 595.3 |
| 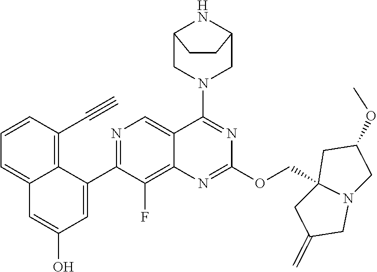 [SMILES: C#Cc1cccc2cc(O)cc(-c3ncc4c(N5CC6CCC(C5)N6)nc(OC[C@@]56CC(=C)CN5C[C@@H](OC)C6)nc4c3F)c12] | Intermediate 19 | 607.4 |
| 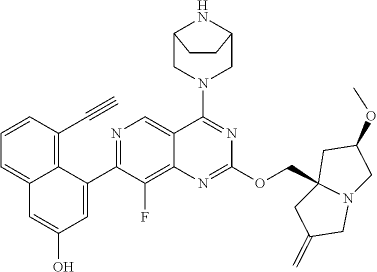 [SMILES: C#Cc1cccc2cc(O)cc(-c3ncc4c(N5CC6CCC(C5)N6)nc(OC[C@]56CC(=C)CN5C[C@H](OC)C6)nc4c3F)c12] | Intermediate 20 | 607.3 |
| 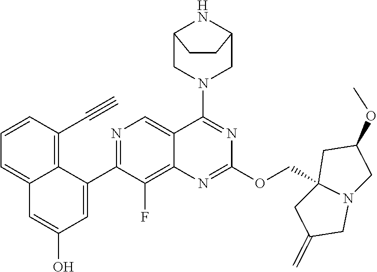 [SMILES: C#Cc1cccc2cc(O)cc(-c3ncc4c(N5CC6CCC(C5)N6)nc(OC[C@@]56CC(=C)CN5C[C@H](OC)C6)nc4c3F)c12] | Intermediate 21 | 607.4 |
| 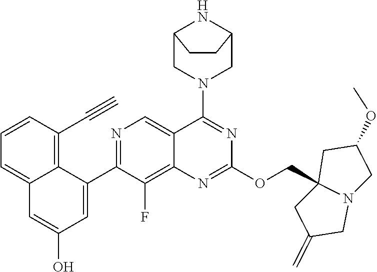 [SMILES: C#Cc1cccc2cc(O)cc(-c3ncc4c(N5CC6CCC(C5)N6)nc(OC[C@]56CC(=C)CN5C[C@@H](OC)C6)nc4c3F)c12] | Intermediate 22 | 607.3 |
| 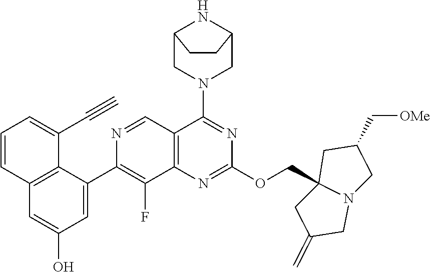 [SMILES: C#Cc1cccc2cc(O)cc(-c3ncc4c(N5CC6CCC(C5)N6)nc(OC[C@]56CC(=C)CN5C[C@@H](COC)C6)nc4c3F)c12] | Intermediate 27 | 621.3 |
| or | | |
| 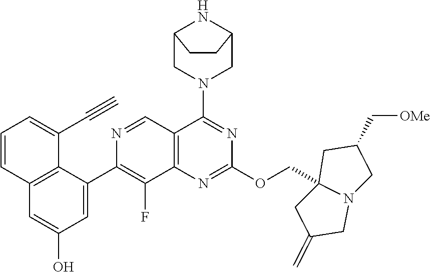 | | |

### Example 4

### Synthesis of 4-(4-((1R,5S)-3,8-diazabicyclo[3.2.1]octan-3-yl)-8-fluoro-2-(((2R,9bS)-2-fluoro-2,3-dihydro-1H-pyrrolo[2,1-a]isoindol-9b(5H)-yl)methoxy)pyrido[4,3-d]pyrimidin-7-yl)-5-ethynylnaphthalen-2-ol

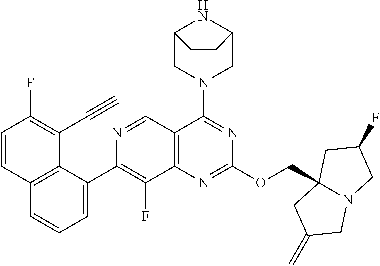 [SMILES: C#Cc1c(F)ccc2cccc(-c3ncc4c(N5CC6CCC(C5)N6)nc(OC[C@]56CC(=C)CN5C[C@H](F)C6)nc4c3F)c12]

### Step 1: tert-butyl (1R,5S)-3-(7-chloro-8-fluoro-2-(((2R,7aR)-2-fluoro-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate

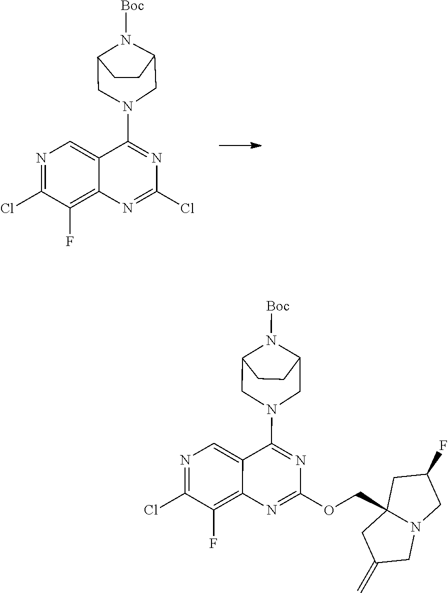 [SMILES: CC(C)(C)OC(=O)N1C2CCC1CN(c1nc(Cl)nc3c(F)c(Cl)ncc13)C2 ; C=C1CN2C[C@H](F)C[C@@]2(COc2nc(N3CC4CCC(C3)N4C(=O)OC(C)(C)C)c3cnc(Cl)c(F)c3n2)C1]

To a stirred solution of tert-butyl (1R,5S)-3-(2,7-dichloro-8-fluoropyrido[4,3-d]pyrimidin-4-yl)-3,8-diazabicyclo[3.2.1]octane-8-carboxylate (3.2 g, 7.47 mmol, 1.0 equiv) and ((2R,7aR)-2-fluoro-6-methylenetetrahydro-1H-pyrrolizin-7a(5H)-yl)methanol (1.28 g, 7.47 mmol, 1.0 equiv) in THF (32 mL) at 5° C. was added NaH (658.81 mg, 14.95 mmol, 2.0 equiv, 40%) in portions and the resulting mixture was stirred for 1 h at room temperature. The mixture was added to water and extracted with EtOAc. The combined organic layers were washed with water and brine, dried over anhydrous Na₂SO₄ and concentrated. The residue was purified by silica gel column chromatography, eluted with EA/PE (50-90%) to afford the title compound (3.4 g).

### Step 2: tert-butyl (1R,5S)-3-(8-fluoro-2-(((2R,7aR)-2-fluoro-6-methylenetetrahydro-1H-pyrrolizin-7a(5H)-yl)methoxy)-7-(7-fluoro-8-((triisopropylsilyl)ethynyl)naphthalen-1-yl)-pyrido[4,3-d]pyrimidin-4-yl)-3,8-diazabicyclo[3.2.1]octane-8-carboxylate

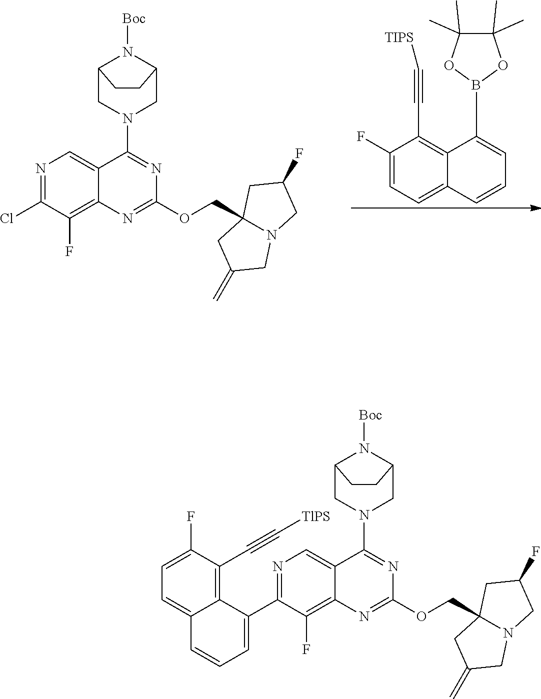

A mixture of tert-butyl (1R,5S)-3-(7-chloro-8-fluoro-2-(((2R,7aR)-2-fluoro-6-methylene-tetrahydro-1H-pyrrolizin-7a(5H)-yl)methoxy)pyrido[4,3-d]pyrimidin-4-yl)-3,8-diazabicyclo-[3.2.1]octane-8-carboxylate (3.4 g, 6.03 mmol, 1.0 equiv.), ((2-fluoro-8-(4,4,5,5-tetramethyl-1,3,2-dioxaborolan-2-yl)naphthalen-1-yl)ethynyl)triisopropylsilane (4.10 g, 9.05 mmol, 1.5 equiv.), CATACXIUM A Pd G3 (0.44 g, 0.60 mmol, 0.1 equiv.) and K₂CO₃ (1.67 g, 12.07 mmol, 2.0 equiv.) in DME (34 mL) and H₂O (3.4 mL) was stirred for 12 h at 85° C. under nitrogen atmosphere. After cooling the mixture to at rt, the mixture was extracted with EtOAc. The combined organic layers were washed with brine, dried over anhydrous Na₂SO₄ and concentrated. The residue was purified by silica gel column chromatography, eluted with EA/PE (0-50%) to afford the title compound (3.5 g).

### Step 3: tert-butyl (1R,5S)-3-(7-(8-ethynyl-7-fluoronaphthalen-1-yl)-8-fluoro-2-(((2R,7aR)-2-fluoro-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate

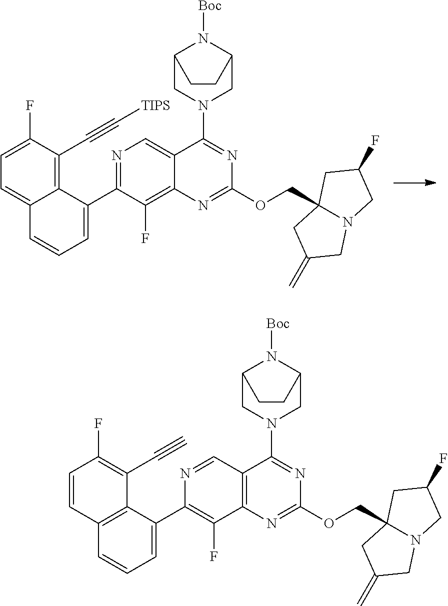

A mixture of tert-butyl (1R,5S)-3-(8-fluoro-2-(((2R,7aR)-2-fluoro-6-methylenetetrahydro-1H-pyrrolizin-7a(5H)-yl)methoxy)-7-(7-fluoro-8-((triisopropylsilyl)ethynyl)naphthalen-1-yl)-pyrido[4,3-d]pyrimidin-4-yl)-3,8-diazabicyclo[3.2.1]octane-8-carboxylate (3.5 g, 4.27 mmol, 1.0 equiv.) and CsF (6499.08 mg, 42.78 mmol, 10.0 equiv.) in DMF (36 mL) was stirred for 2 h at room temperature. The resulting mixture was added EtOAc and then washed with water and brine, and dried over anhydrous Na₂SO₄. After filtration, the filtrate was concentrated under reduced pressure. The residue was purified by silica gel column chromatography, eluted with EA/PE (30-80%) to afford the title compound (2.3 g).

### Step 4: 4-((1R,5S)-3,8-diazabicyclo[3.2.1]octan-3-yl)-7-(8-ethynyl-7-fluoronaphthalen-1-yl)-8-fluoro-2-(((2R,7aR)-2-fluoro-6-methylenetetrahydro-1H-pyrrolizin-7a(5H)-yl)methoxy)pyrido[4,3-d]pyrimidine

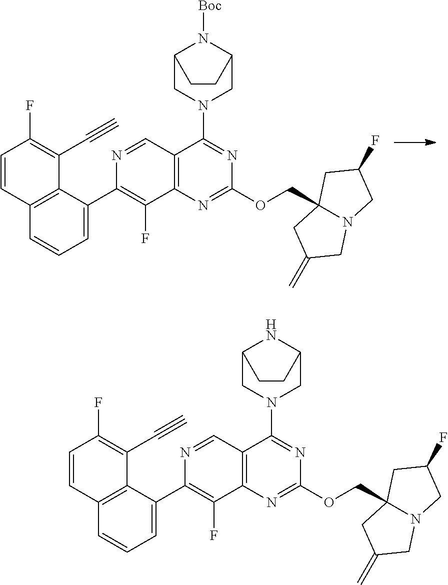 [SMILES: C#Cc1c(F)ccc2cccc(-c3ncc4c(N5CC6CCC(C5)N6C(=O)OC(C)(C)C)nc(OC[C@]56CC(=C)CN5C[C@H](F)C6)nc4c3F)c12 ; C#Cc1c(F)ccc2cccc(-c3ncc4c(N5CC6CCC(C5)N6)nc(OC[C@]56CC(=C)CN5C[C@H](F)C6)nc4c3F)c12]

To a solution of tert-butyl (1R,5S)-3-(7-(8-ethynyl-7-fluoronaphthalen-1-yl)-8-fluoro-2-(((2R,7aR)-2-fluoro-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate (2.3 g, 3.30 mmol, 1.0 equiv.) in DCM (18 ml) was added HCl solution (4.0 M in 1,4-dioxane, 9 mL) dropwise at 0° C. and the resulting mixture was stirred at 5° C. for 1 h. The reaction mixture was then basified to pH 8 with NH₃ in MeOH (1 mol/L) at 5° C. The resulting mixture was concentrated under vacuum and the residue was purified by Pre-HPLC to afford the title compound (900 mg). MS (ES, m/z): [M+H]⁺=597.4.

### Example 5

### Synthesis of 4-((1R,5S)-3,8-diazabicyclo[3.2.1]octan-3-yl)-7-(8-ethynyl-7-fluoronaphthalen-1-yl)-8-fluoro-2-(((2R,7aS)-2-methoxy-6-methylenetetrahydro-1H-pyrrolizin-7a(5H)-yl)methoxy)pyrido[4,3-d]pyrimidine

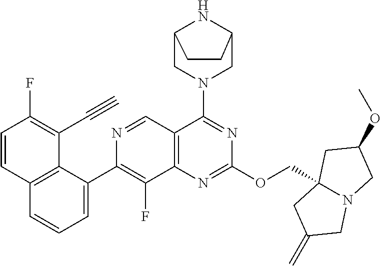 [SMILES: C#Cc1c(F)ccc2cccc(-c3ncc4c(N5CC6CCC(C5)N6)nc(OC[C@@]56CC(=C)CN5C[C@H](OC)C6)nc4c3F)c12]

### Step 1: 8-fluoro-7-(7-fluoro-8-((triisopropylsilyl)ethynyl)naphthalen-1-yl)pyrido[4,3-d]-pyrimidine-2,4-diol

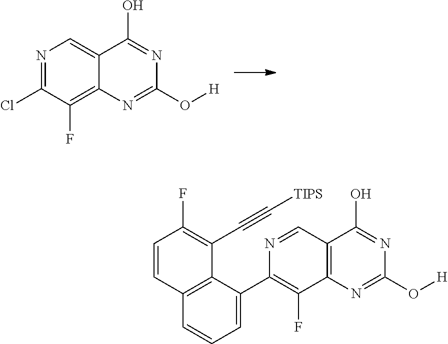

To a mixture of 7-chloro-8-fluoropyrido[4,3-d]pyrimidine-2,4-diol (4.0 g, 18.6 mmol, 1.0 eq.) and ((2-fluoro-8-(4,4,5,5-tetramethyl-1,3,2-dioxaborolan-2-yl)naphthalen-1-yl)ethynyl)-triisopropylsilane (10.9 g, 24.1 mmol, 1.3 eq.) in EtOH (150 mL) and H₂O (50 mL) were added CATACXIUM A Pd G3 (2.4 g, 3.3 mmol, 0.18 eq.), K3PO₄ (11.7 g, 55.1 mmol, 3.0 eq.) at room temperature under nitrogen atmosphere. The resulting mixture was stirred for 16 h at 80° C. under nitrogen atmosphere. After cooling to rt, the mixture was added water, and then extracted with EtOAc. The combined organic layers were washed with water, brine, dried over anhydrous Na₂SO₄, filtered and then concentrated. The residue was purified by silica gel column chromatography, eluted with MeOH/DCM (0-10%), to afford the title compound (5.5 g).

### Step 2: 7-(8-ethynyl-7-fluoronaphthalen-1-yl)-8-fluoropyrido[4,3-d]pyrimidine-2,4-diol

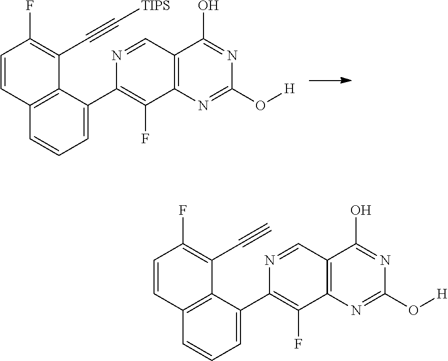

A mixture of 8-fluoro-7-(7-fluoro-8-((triisopropylsilyl)ethynyl)naphthalen-1-yl)pyrido [4,3-d]pyrimidine-2,4-diol (10.0 g, 19.8 mmol, 1.0 eq.) and CsF (12.0 g, 79.0 mmol, 4.0 eq.) in DMF (100 mL) was stirred for 1 h at 50° C. under nitrogen atmosphere. After cooling to rt, the reaction mixture was concentrated and the residue was purified by silica gel column chromatography, eluted with MeOH/DCM (0-10%), to afford the title compound (6.0 g).

### Step 3: 2,4-dichloro-7-(8-ethynyl-7-fluoronaphthalen-1-yl)-8-fluoropyrido[4,3-d]pyrimidine

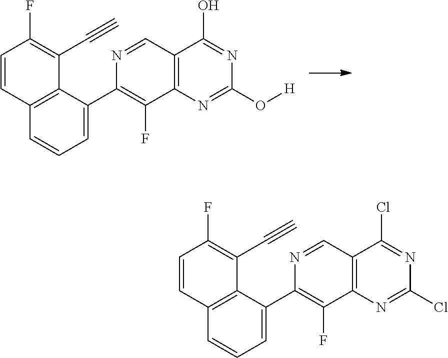 [SMILES: C#Cc1c(F)ccc2cccc(-c3ncc4c(O)nc(O)nc4c3F)c12 ; C#Cc1c(F)ccc2cccc(-c3ncc4c(Cl)nc(Cl)nc4c3F)c12]

To a solution of POCl₃ (15.8 g, 103.0 mmol, 30.3 eq.) and DIPEA (13.4 g, 103.7 mmol, 30.5 eq.) was added 7-(8-ethynyl-7-fluoronaphthalen-1-yl)-8-fluoropyrido[4,3-d]-pyrimidine-2,4-diol (1.2 g, 3.4 mmol, 1.0 eq.) in portions at 0-5° C. and the resulting mixture was stirred for 1 h. The mixture was concentrated and the residue was diluted with ice water, and extracted with EtOAc. The combined organic layers were washed with water, brine, dried over anhydrous Na₂SO₄, filtered and concentrated to afford the title compound (1.5 g, crude) as a brown solid, which was used for next step without further purification.

### Step 4: tert-butyl (1R,5S)-3-(2-chloro-7-(8-ethynyl-7-fluoronaphthalen-1-yl)-8-fluoropyrido[4,3-d]pyrimidin-4-yl)-3,8-diazabicyclo[3.2.1]octane-8-carboxylate

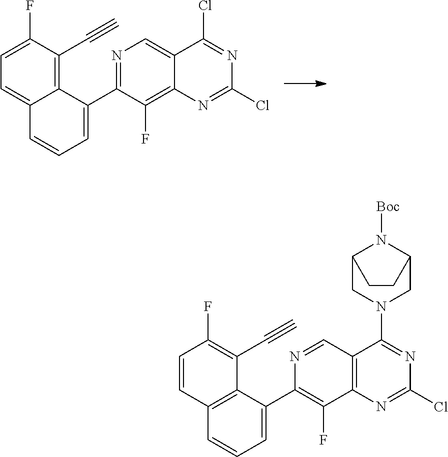 [SMILES: C#Cc1c(F)ccc2cccc(-c3ncc4c(Cl)nc(Cl)nc4c3F)c12 ; C#Cc1c(F)ccc2cccc(-c3ncc4c(N5CC6CCC(C5)N6C(=O)OC(C)(C)C)nc(Cl)nc4c3F)c12]

To a solution of 2,4-dichloro-7-(8-ethynyl-7-fluoronaphthalen-1-yl)-8-fluoropyrido-[4,3-d]pyrimidine (1.5 g, 3.9 mmol, 1.0 eq., crude) in DCM (30 mL) was added DIEA (1.1 g, 8.5 mmol, 2.2 eq.) dropwise at −40° C. After stirring for 5 min at −40° C., a solution of tert-butyl (1R,5S)-3,8-diazabicyclo[3.2.1]octane-8-carboxylate (870 mg, 4.1 mmol, 1.1 eq.) in THF (5 mL) was added dropwise and the resulting mixture was stirred for additional 15 min at −40° C. The reaction mixture was concentrated and the residue was purified by silica gel column chromatography, eluted with EA/PE (0-50%), to afford the title compound (770 mg).

### Step 5: tert-butyl (1R,5S)-3-(7-(8-ethynyl-7-fluoronaphthalen-1-yl)-8-fluoro-2-(((2R,7aS)-2-methoxy-6-methylenetetrahydro-1H-pyrrolizin-7a(5H)-yl)methoxy)pyrido[4,3-d]pyrimidin-4-yl)-3,8-diazabicyclo[3.2.1]octane-8-carboxylate

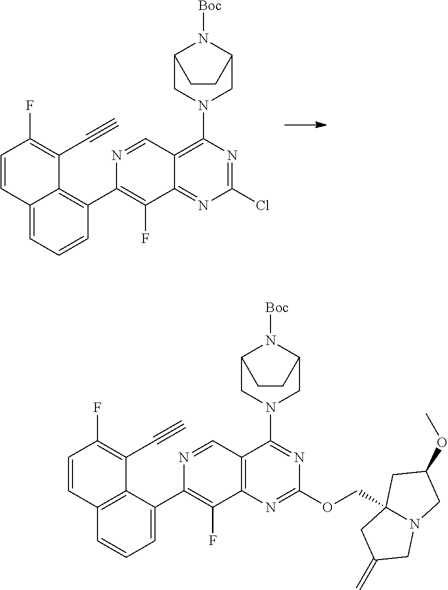 [SMILES: C#Cc1c(F)ccc2cccc(-c3ncc4c(N5CC6CCC(C5)N6C(=O)OC(C)(C)C)nc(Cl)nc4c3F)c12 ; C#Cc1c(F)ccc2cccc(-c3ncc4c(N5CC6CCC(C5)N6C(=O)OC(C)(C)C)nc(OC[C@@]56CC(=C)CN5C[C@H](OC)C6)nc4c3F)c12]

To a solution of tert-butyl (1R,5S)-3-(2-chloro-7-(8-ethynyl-7-fluoronaphthalen-1-yl)-8-fluoropyrido[4,3-d]pyrimidin-4-yl)-3,8-diazabicyclo[3.2.1]octane-8-carboxylate (50.0 mg, 0.90 mmol, 1.0 equiv.) and ((2R,7aS)-2-methoxy-6-methylenetetrahydro-1H-pyrrolizin-7a(5H)-yl)methanol (18.0 mg, 0.10 mmol, 1.1 equiv.) in THF(0.5 mL) was added NaH (7.0 mg, 0.21 mmol, 2.0 equiv.) in portions at 0° C. and the resulting mixture was stirred for 1 h at room temperature. The reaction mixture was added water and extracted with EtOAc. The organic layers were combined and concentrated. The residue was purified by silica gel column chromatography, eluted with EA/PE (0-100%) to afford the title compound (51.0 mg, 83.61%) as white solid.

### Step 6: 4-((1R,5S)-3,8-diazabicyclo[3.2.1]octan-3-yl)-7-(8-ethynyl-7-fluoronaphthalen-1-yl)-8-fluoro-2-(((2R,7aS)-2-methoxy-6-methylenetetrahydro-1H-pyrrolizin-7a(5H)-yl)methoxy)-pyrido[4,3-d]pyrimidine

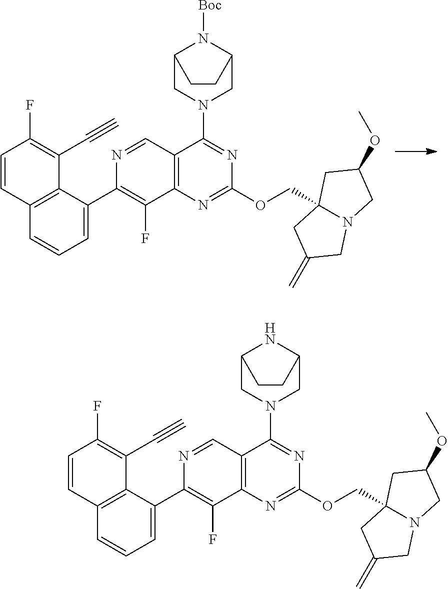 [SMILES: C#Cc1c(F)ccc2cccc(-c3ncc4c(N5CC6CCC(C5)N6C(=O)OC(C)(C)C)nc(OC[C@@]56CC(=C)CN5C[C@H](OC)C6)nc4c3F)c12 ; C#Cc1c(F)ccc2cccc(-c3ncc4c(N5CC6CCC(C5)N6)nc(OC[C@@]56CC(=C)CN5C[C@H](OC)C6)nc4c3F)c12]

To a solution of tert-butyl (1R,5S)-3-(7-(8-ethynyl-7-fluoronaphthalen-1-yl)-8-fluoro-2-(((2R,7aS)-2-methoxy-6-methylenetetrahydro-1H-pyrrolizin-7a(5H)-yl)methoxy)pyrido[4,3-d]-pyrimidin-4-yl)-3,8-diazabicyclo[3.2.1]octane-8-carboxylate (51.0 mg, 0.07 mmol, 1.0 equiv.) in DCM (0.5 mL) was added HCl solution (4.0 M solution in 1,4-dioxane, 156 uL, 0.62 mmol, 8.7 equiv.) dropwise at 0-5° C. and the resulting mixture was stirred for 30 min at 0-5° C. The mixture was then basified to pH 8-9 with NH₃ in MeOH. After removing volatile solvents, the residue was purified by silica gel column chromatography, eluted with EA/PE (0-100%) to afford 30 mg crude material, which was further purified by Prep-HPLC to afford the title compound (7.0 mg). MS (ES, m/z): [M+H]⁺=609.5.

Proceeding analogously as described in Example 5, Steps 5 and 6, compounds in Table D below were prepared by replacing in Step 5, ((2R,7aS)-2-methoxy-6-methylenetetrahydro-1H-pyrrolizin-7a(5H)-yl)methanol with alcohols indicated therein.

TABLE D

| | Alcohol used | MS |
|---|---|---|
| Compound | in Step 5 | [M + H]⁺ |
| 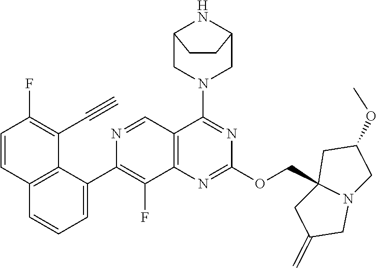 [SMILES: C#Cc1c(F)ccc2cccc(-c3ncc4c(N5CC6CCC(C5)N6)nc(OC[C@]56CC(=C)CN5C[C@@H](OC)C6)nc4c3F)c12] | Intermediate 22 | 609.5 |
| 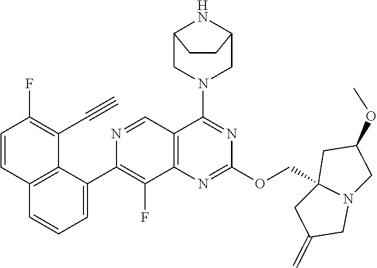 [SMILES: C#Cc1c(F)ccc2cccc(-c3ncc4c(N5CC6CCC(C5)N6)nc(OC[C@@]56CC(=C)CN5C[C@H](OC)C6)nc4c3F)c12] | Intermediate 11 | 609.5 |
| 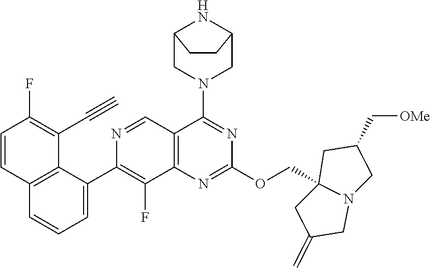 [SMILES: C#Cc1c(F)ccc2cccc(-c3ncc4c(N5CC6CCC(C5)N6)nc(OC[C@@]56CC(=C)CN5C[C@@H](COC)C6)nc4c3F)c12] | Intermediate 27 | 623.4 |
| and | | |
| 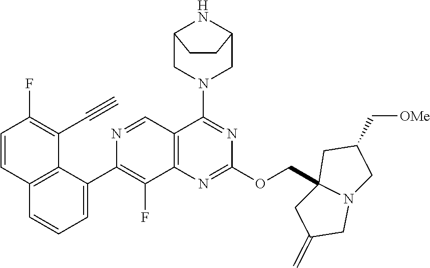 | | |
| 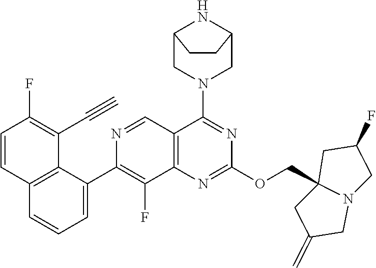 [SMILES: C#Cc1c(F)ccc2cccc(-c3ncc4c(N5CC6CCC(C5)N6)nc(OC[C@]56CC(=C)CN5C[C@H](F)C6)nc4c3F)c12] | Intermediate 12 | 597.4 |
| 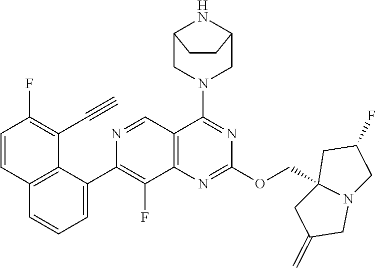 [SMILES: C#Cc1c(F)ccc2cccc(-c3ncc4c(N5CC6CCC(C5)N6)nc(OC[C@@]56CC(=C)CN5C[C@@H](F)C6)nc4c3F)c12] | Intermediate 14 | 597.3 |
| 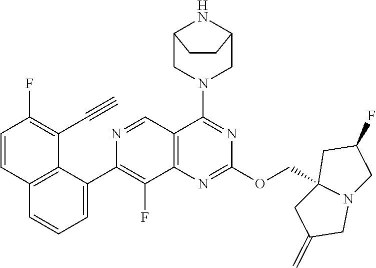 [SMILES: C#Cc1c(F)ccc2cccc(-c3ncc4c(N5CC6CCC(C5)N6)nc(OC[C@@]56CC(=C)CN5C[C@H](F)C6)nc4c3F)c12] | Intermediate 13 | 597.3 |
| 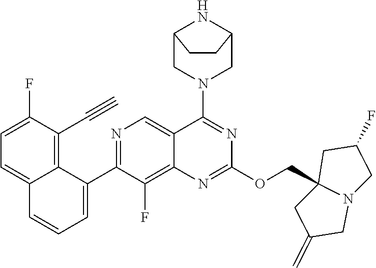 [SMILES: C#Cc1c(F)ccc2cccc(-c3ncc4c(N5CC6CCC(C5)N6)nc(OC[C@]56CC(=C)CN5C[C@@H](F)C6)nc4c3F)c12] | Intermediate 11 | 597.3 |
| 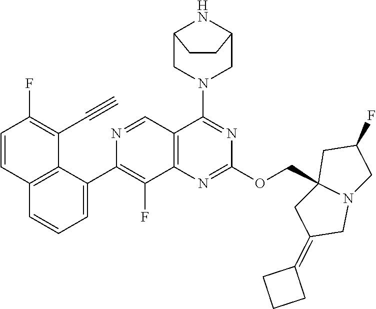 [SMILES: C#Cc1c(F)ccc2cccc(-c3ncc4c(N5CC6CCC(C5)N6)nc(OC[C@]56CC(=C7CCC7)CN5C[C@H](F)C6)nc4c3F)c12] | Intermediate 17 | 637.4 |
| 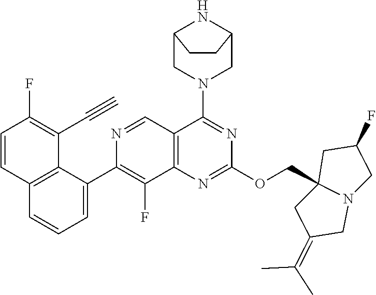 [SMILES: C#Cc1c(F)ccc2cccc(-c3ncc4c(N5CC6CCC(C5)N6)nc(OC[C@]56CC(=C(C)C)CN5C[C@H](F)C6)nc4c3F)c12] | Intermediate 18 | 625.4 |
| 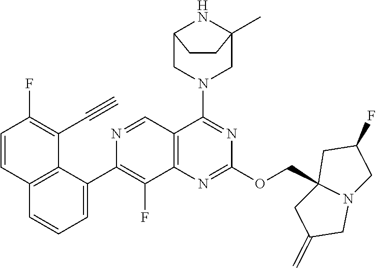 [SMILES: C#Cc1c(F)ccc2cccc(-c3ncc4c(N5CC6CCC(C)(C5)N6)nc(OC[C@]56CC(=C)CN5C[C@H](F)C6)nc4c3F)c12] | Intermediate 11 | 611.3 |
| 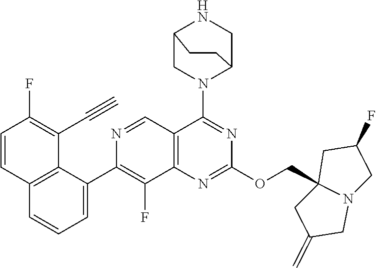 [SMILES: C#Cc1c(F)ccc2cccc(-c3ncc4c(N5CC6CCC5CN6)nc(OC[C@]56CC(=C)CN5C[C@H](F)C6)nc4c3F)c12] | Intermediate 12 | 597.3 |
| 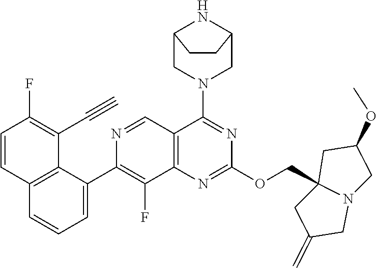 [SMILES: C#Cc1c(F)ccc2cccc(-c3ncc4c(N5CC6CCC(C5)N6)nc(OC[C@]56CC(=C)CN5C[C@H](OC)C6)nc4c3F)c12] | Intermediate 20 | 609.4 |
| 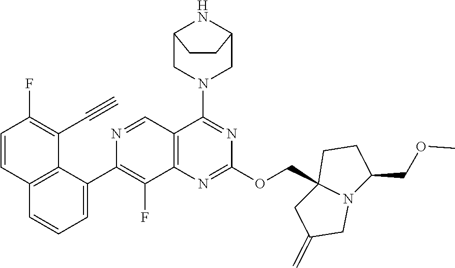 [SMILES: C#Cc1c(F)ccc2cccc(-c3ncc4c(N5CC6CCC(C5)N6)nc(OC[C@@]56CC[C@@H](COC)N5CC(=C)C6)nc4c3F)c12] | Intermediate 25 | 623.4 623.4 |
| and | | |
| 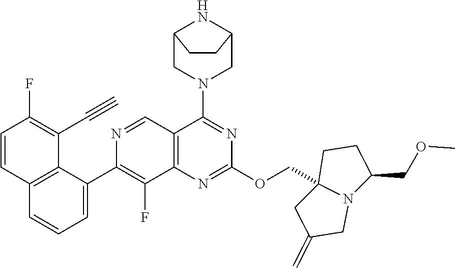 | | |
| 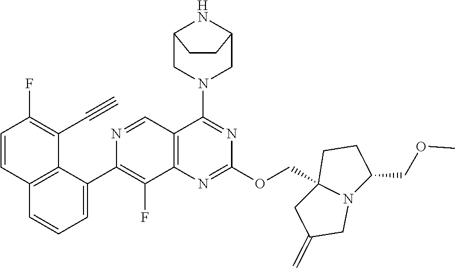 [SMILES: C#Cc1c(F)ccc2cccc(-c3ncc4c(N5CC6CCC(C5)N6)nc(OC[C@]56CC[C@H](COC)N5CC(=C)C6)nc4c3F)c12] | Intermediate 26 | 623.4 623.4 |
| and | | |
| 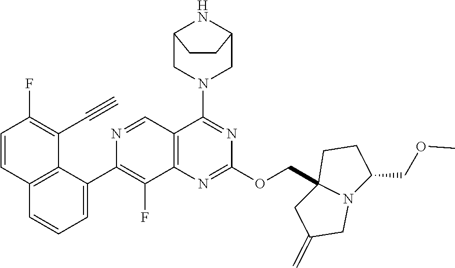 | | |
| 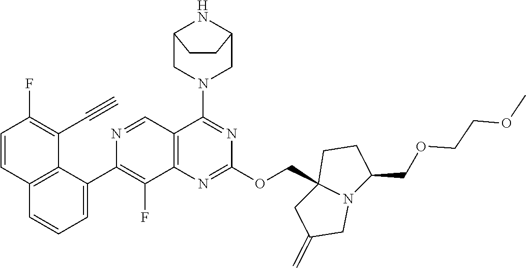 [SMILES: C#Cc1c(F)ccc2cccc(-c3ncc4c(N5CC6CCC(C5)N6)nc(OC[C@@]56CC[C@@H](COCCOC)N5CC(=C)C6)nc4c3F)c12] | Intermediate 30 | 667.4 |
| 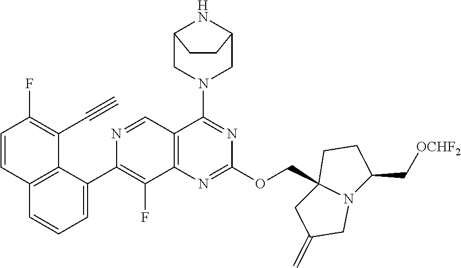 [SMILES: C#Cc1c(F)ccc2cccc(-c3ncc4c(N5CC6CCC(C5)N6)nc(OC[C@@]56CC[C@@H](COC(F)F)N5CC(=C)C6)nc4c3F)c12] | Intermediate 31 | 659.4 |
| 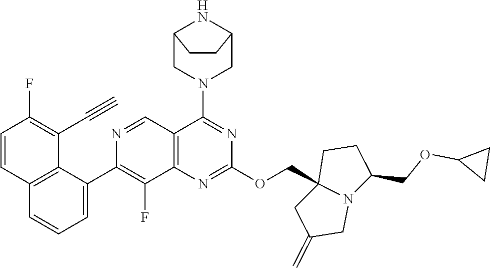 [SMILES: C#Cc1c(F)ccc2cccc(-c3ncc4c(N5CC6CCC(C5)N6)nc(OC[C@@]56CC[C@@H](COC7CC7)N5CC(=C)C6)nc4c3F)c12] | Intermediate 32 | 649.4 |
| 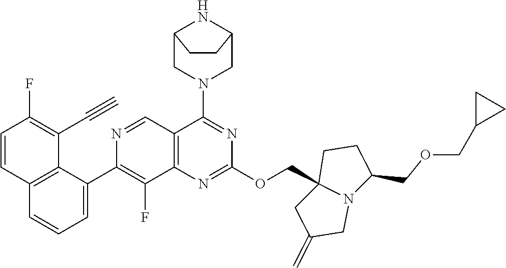 [SMILES: C#Cc1c(F)ccc2cccc(-c3ncc4c(N5CC6CCC(C5)N6)nc(OC[C@@]56CC[C@@H](COCC7CC7)N5CC(=C)C6)nc4c3F)c12] | Intermediate 33 | 663.4 |
| 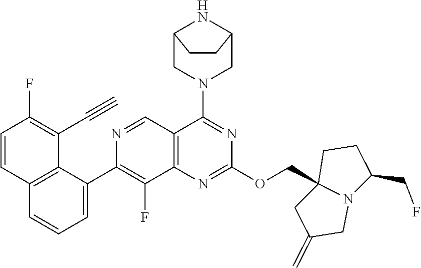 [SMILES: C#Cc1c(F)ccc2cccc(-c3ncc4c(N5CC6CCC(C5)N6)nc(OC[C@@]56CC[C@@H](CF)N5CC(=C)C6)nc4c3F)c12] | Intermediate 29 | 609.4 |
| 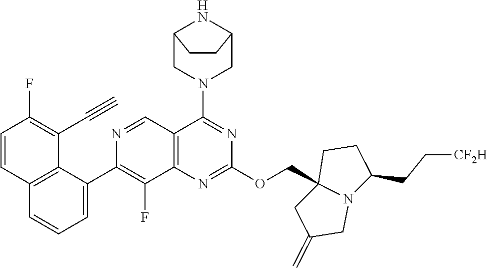 [SMILES: C#Cc1c(F)ccc2cccc(-c3ncc4c(N5CC6CCC(C5)N6)nc(OC[C@@]56CC[C@@H](CCC(F)F)N5CC(=C)C6)nc4c3F)c12] | Intermediate 35 | 657.4 |
| 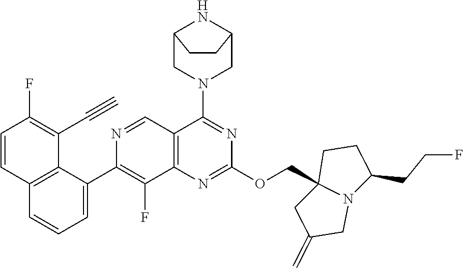 [SMILES: C#Cc1c(F)ccc2cccc(-c3ncc4c(N5CC6CCC(C5)N6)nc(OC[C@@]56CC[C@@H](CCF)N5CC(=C)C6)nc4c3F)c12] | Intermediate 34 | 625.4 |
| 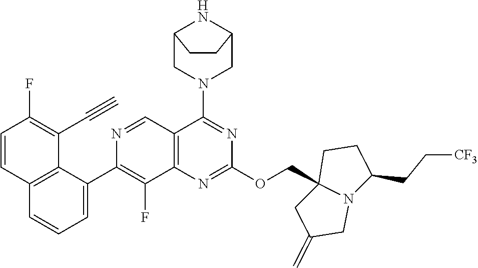 [SMILES: C#Cc1c(F)ccc2cccc(-c3ncc4c(N5CC6CCC(C5)N6)nc(OC[C@@]56CC[C@@H](CCC(F)(F)F)N5CC(=C)C6)nc4c3F)c12] | Intermediate 36 | 675.3 |
| 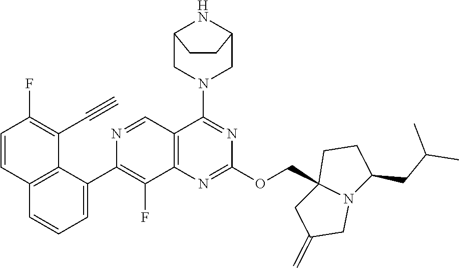 [SMILES: C#Cc1c(F)ccc2cccc(-c3ncc4c(N5CC6CCC(C5)N6)nc(OC[C@@]56CC[C@@H](CC(C)C)N5CC(=C)C6)nc4c3F)c12] | Intermediate 37 | 635.4 |
| 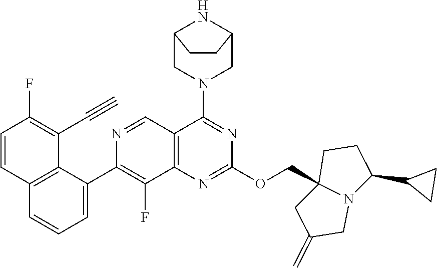 [SMILES: C#Cc1c(F)ccc2cccc(-c3ncc4c(N5CC6CCC(C5)N6)nc(OC[C@@]56CC[C@@H](C7CC7)N5CC(=C)C6)nc4c3F)c12] | Intermediate 38 | 619.4 |
| 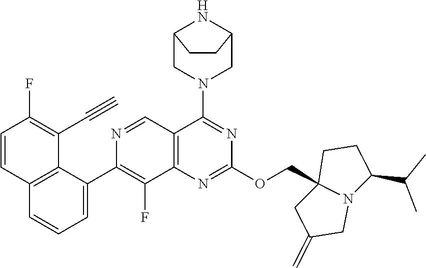 [SMILES: C#Cc1c(F)ccc2cccc(-c3ncc4c(N5CC6CCC(C5)N6)nc(OC[C@@]56CC[C@@H](C(C)C)N5CC(=C)C6)nc4c3F)c12] | Intermediate 39 | 621.4 |
| 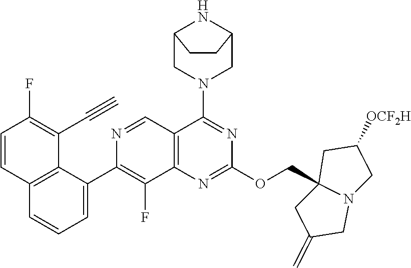 | Intermediate 40 | 645.3 |
| 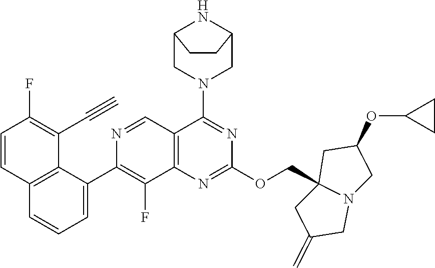 [SMILES: C#Cc1c(F)ccc2cccc(-c3ncc4c(N5CC6CCC(C5)N6)nc(OC[C@]56CC(=C)CN5C[C@H](OC5CC5)C6)nc4c3F)c12] | Intermediate 41 | 635.4 |
| 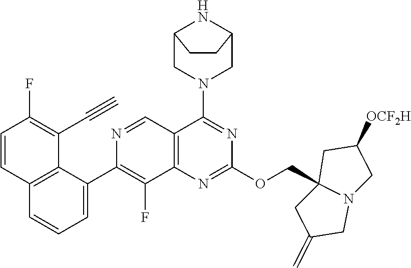 | Intermediate 23 | 645.3 |
| 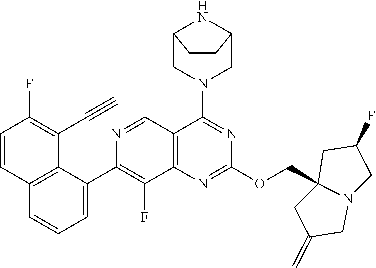 [SMILES: C#Cc1c(F)ccc2cccc(-c3ncc4c(N5CC6CCC(C5)N6)nc(OC[C@]56CC(=C)CN5C[C@H](F)C6)nc4c3F)c12] | Intermediate 42 | 611.4 |
| 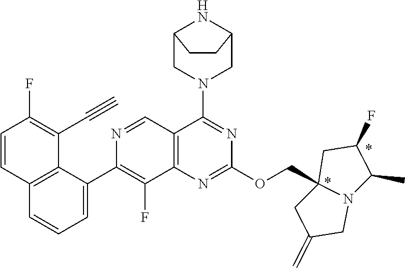 [SMILES: C#Cc1c(F)ccc2cccc(-c3ncc4c(N5CC6CCC(C5)N6)nc(OC[C@]56CC(=C)CN5[C@H](C)[C@H](F)C6)nc4c3F)c12] | Intermediate 60 | 611.3 |
| 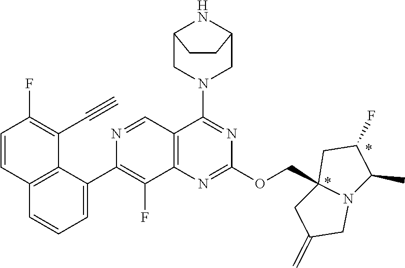 [SMILES: C#Cc1c(F)ccc2cccc(-c3ncc4c(N5CC6CCC(C5)N6)nc(OC[C@]56CC(=C)CN5[C@H](C)[C@@H](F)C6)nc4c3F)c12] | Intermediate 46 | 611.3 |

* stereochemistry assignment tentative

### Example 6

### Synthesis of [8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-6-methylene-2,3,5,7-tetrahydro-1H-pyrrolizin-3-yl]methyl 3,3-difluoropyrrolidine-1-carboxylate

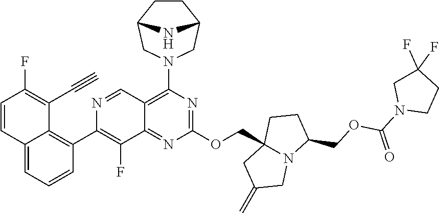 [SMILES: C#Cc1c(F)ccc2cccc(-c3ncc4c(N5C[C@H]6CC[C@@H](C5)N6)nc(OC[C@@]56CC[C@@H](COC(=O)N7CCC(F)(F)C7)N5CC(=C)C6)nc4c3F)c12]

### Step 1: tert-butyl 3-[2-[[(3S,8S)-3-[[tert-butyl(diphenyl)silyl]oxymethyl]-6-methylene-2,3,5,7-tetrahydro-1H pyrrolizin-8-yl]methoxy]-7-(8-ethynyl-7-fluoro-1-naphthyl)-8-fluoro-pyrido[4,3-d]pyrimidin-4-yl]-3,8-diazabicyclo[3.2.1]octane-8-carboxylate

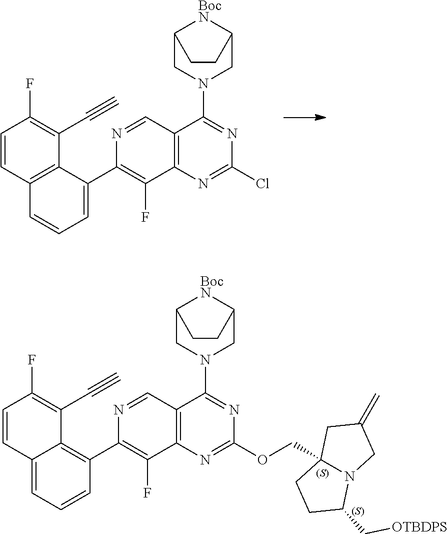

To a solution of tert-butyl 3-[2-chloro-7-(8-ethynyl-7-fluoro-1-naphthyl)-8-fluoro-pyrido[4,3-d]pyrimidin-4-yl]-3,8-diazabicyclo[3.2.1]octane-8-carboxylate (900 mg, 1.60 mmol, 1 eq) and [(3S,8S)-3-[[tert-butyl(diphenyl)silyl]oxymethyl]-6-methylene-2,3,5,7-tetrahydro-1H-pyrrolizin-8-yl]methanol (742 mg, 1.76 mmol, 1.1 eq) in THF (20 mL) was added NaH (160 mg, 4.00 mmol, 60% purity, 2.5 eq) at 0° C. slowly. The mixture was stirred at 20° C. for 2 h under N₂. The mixture was quenched by addition aq. NH₄Cl 10 mL at 0° C., and then extracted with ethyl acetate. The combined organic layers were concentrated under reduced pressure and the residue was purified by column chromatography, eluted with EA/PE (0-30%) to afford the title compound (1.00 g).

### Step 2: tert-butyl 3-[7-(8-ethynyl-7-fluoro-1-naphthyl)-8-fluoro-2-[[(3S,8S)-3-(hydroxymethyl)-6-methylene-2,3,5,7-tetrahydro-1H-pyrrolizin-8-yl]methoxy]pyrido[4,3-d]pyrimidin-4-yl]-3,8-diazabicyclo[3.2.1]octane-8-carboxylate

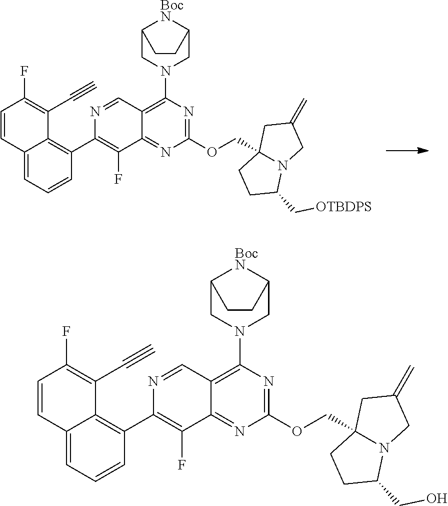

To a solution of tert-butyl 3-[2-[[(3S,8S)-3-[[tert-butyl(diphenyl)silyl]oxymethyl]-6-methylene-2,3,5,7-tetrahydro-1H-pyrrolizin-8-yl]methoxy]-7-(8-ethynyl-7-fluoro-1-naphthyl)-8-fluoro-pyrido[4,3-d]pyrimidin-4-yl]-3,8-diazabicyclo[3.2.1]octane-8-carboxylate (450 mg, 475 umol, 1 eq) in THF (5 mL) was added TBAF (1 M, 2.38 mL, 5 eq) and AcOH (142 mg, 2.38 mmol, 135 uL, 5 eq). The mixture was stirred at 40° C. for 4 h under N₂. The mixture was quenched with water and then extracted with ethyl acetate. The combined organic layers were concentrated under reduced pressure and the residue was purified by column chromatography eluted with EA/PE (0-10%) to afford the title compound (650 mg).

### Step 3: tert-butyl 3-[2-[[3-[(3,3-difluoropyrrolidine-1-carbonyl)oxymethyl]-6-methylene-2,3,5,7-tetrahydro-1H-pyrrolizin-8-yl]methoxy]-7-(8-ethynyl-7-fluoro-1-naphthyl)-8-fluoro-pyrido[4,3-d]pyrimidin-4-yl]-3,8-diazabicyclo[3.2.1]octane-8-carboxylate

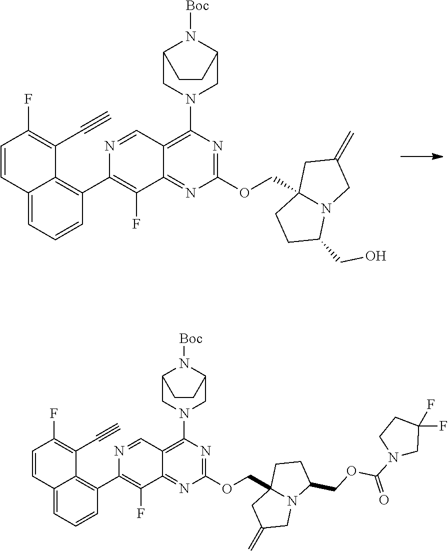

To a solution of tert-butyl 3-[7-(8-ethynyl-7-fluoro-1-naphthyl)-8-fluoro-2-[[(3S,8S)-3-(hydroxymethyl)-6-methylene-2,3,5,7-tetrahydro-1H-pyrrolizin-8-yl]methoxy]pyrido[4,3-d]-pyrimidin-4-yl]-3,8-diazabicyclo[3.2.1]octane-8-carboxylate (100 mg, 141 umol, 1 eq.) in THF (2 mL) was added TEA (85.6 mg, 0.846 mmol, 0.117 mL, 6.0 equiv) and 4-nitrophenylcarbonochloridate (142 mg, 0.705 mmol, 5 equiv.). The mixture was stirred at 25° C. for 2 h. 3,3-Difluoropyrrolidine (90.6 mg, 0.846 mmol, 0.057 mL, 6 equiv) was added and the mixture was stirred at 25° C. for 0.5 h. The mixture was quenched with saturated aqueous solution of NaHCO₃, and extracted with EtOAc. The combined organic layers were dried over anhydrous Na₂SO₄, filtered and concentrated. The residue was purified by column chromatography, eluted with EA/PE (0-70%) to afford the title compound (60.0 mg).

### Step 4: [8-[[4-(3,8-diazabicyclo[3.2.1]octan-3-yl)-7-(8-ethynyl-7-fluoro-1-naphthyl)-8-fluoro-pyrido[4,3-d]pyrimidin-2-yl]oxymethyl]-6-methylene-2,3,5,7-tetrahydro-1H-pyrrolizin-3-yl]-methyl 3,3-difluoropyrrolidine-1-carboxylate

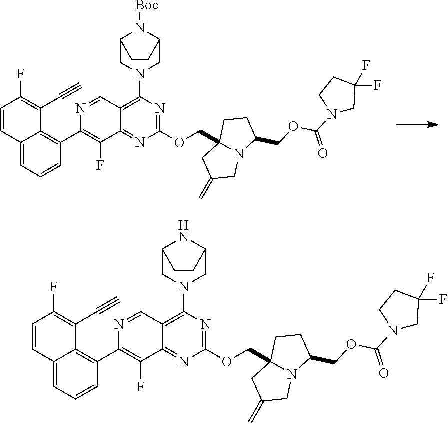

To a solution of tert-butyl 3-[2-[[3-[(3,3-difluoropyrrolidine-1-carbonyl)oxymethyl]-6-methylene-2,3,5,7-tetrahydro-1H-pyrrolizin-8-yl]methoxy]-7-(8-ethynyl-7-fluoro-1-naphthyl)-8-fluoropyrido[4,3-d]pyrimidin-4-yl]-3,8-diazabicyclo[3.2.1]octane-8-carboxylate (60.0 mg, 71.2 umol, 1 eq) in DCM (1 mL) was added TFA (154 mg, 1.35 mmol, 0.1 mL, 18.9 eq) at 0° C. and the mixture was stirred at 0° C. for 0.5 h. The reaction mixture was basified to pH=8 by adding NH₃ solution in MeOH. After removing volatile solvents, the residue was purified by prep-HPLC to afford the title compound (31.0 mg). MS (ES, m/z): [M+1]⁺=742.2.

Proceeding analogously as described in Example 6, Steps 3 and 4, compounds in Table E below were prepared by replacing in Step 3, 3,3-difluoropyrrolidine with alcohols indicated therein.

| | Amino used | MS |
|---|---|---|
| Compound | in Step 3 | [M + H]⁺ |
| 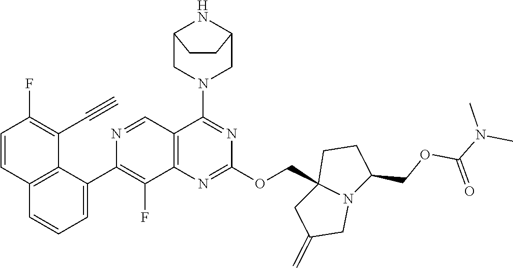 [SMILES: C#Cc1c(F)ccc2cccc(-c3ncc4c(N5CC6CCC(C5)N6)nc(OC[C@@]56CC[C@@H](COC(=O)N(C)C)N5CC(=C)C6)nc4c3F)c12] | 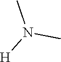 | 680.2 |
| 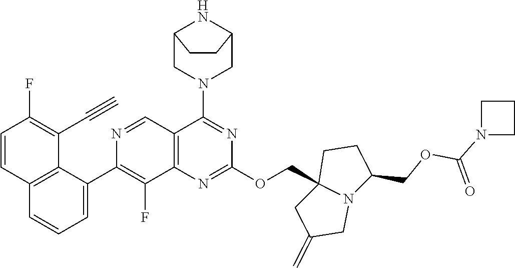 [SMILES: C#Cc1c(F)ccc2cccc(-c3ncc4c(N5CC6CCC(C5)N6)nc(OC[C@@]56CC[C@@H](COC(=O)N7CCC7)N5CC(=C)C6)nc4c3F)c12] | 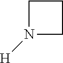 | 692.3 |
| 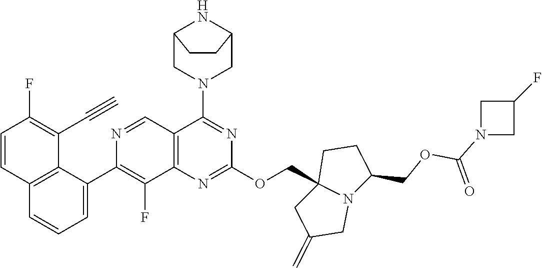 [SMILES: C#Cc1c(F)ccc2cccc(-c3ncc4c(N5CC6CCC(C5)N6)nc(OC[C@@]56CC[C@@H](COC(=O)N7CC(F)C7)N5CC(=C)C6)nc4c3F)c12] | 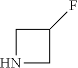 | 710.2 |
| 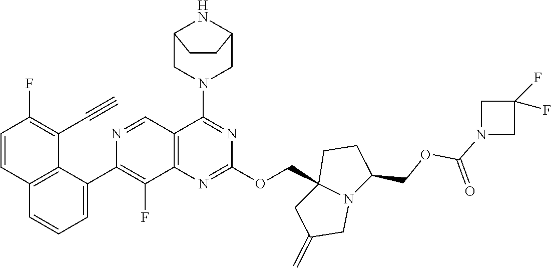 [SMILES: C#Cc1c(F)ccc2cccc(-c3ncc4c(N5CC6CCC(C5)N6)nc(OC[C@@]56CC[C@@H](COC(=O)N7CC(F)(F)C7)N5CC(=C)C6)nc4c3F)c12] | 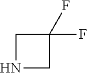 | 728.2 |
| 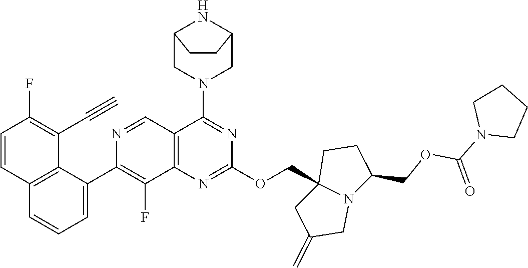 [SMILES: C#Cc1c(F)ccc2cccc(-c3ncc4c(N5CC6CCC(C5)N6)nc(OC[C@@]56CC[C@@H](COC(=O)N7CCCC7)N5CC(=C)C6)nc4c3F)c12] | 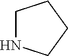 | 706.3 |
| 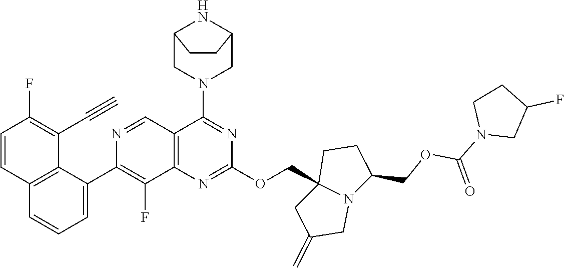 [SMILES: C#Cc1c(F)ccc2cccc(-c3ncc4c(N5CC6CCC(C5)N6)nc(OC[C@@]56CC[C@@H](COC(=O)N7CCC(F)C7)N5CC(=C)C6)nc4c3F)c12] | 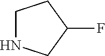 | 724.2 |
| 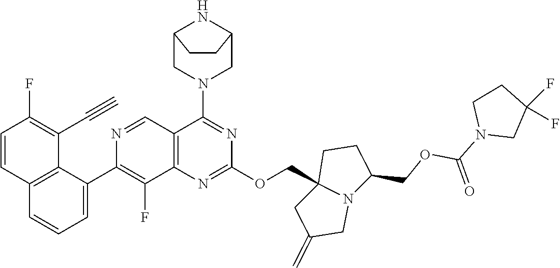 [SMILES: C#Cc1c(F)ccc2cccc(-c3ncc4c(N5CC6CCC(C5)N6)nc(OC[C@@]56CC[C@@H](COC(=O)N7CCC(F)(F)C7)N5CC(=C)C6)nc4c3F)c12] | 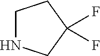 | 742.2 |
| 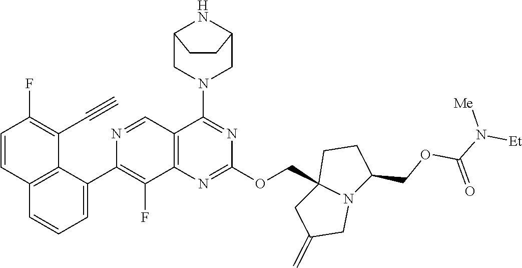 [SMILES: C#Cc1c(F)ccc2cccc(-c3ncc4c(N5CC6CCC(C5)N6)nc(OC[C@@]56CC[C@@H](COC(=O)N(C)CC)N5CC(=C)C6)nc4c3F)c12] | 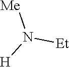 | 694.2 |
| 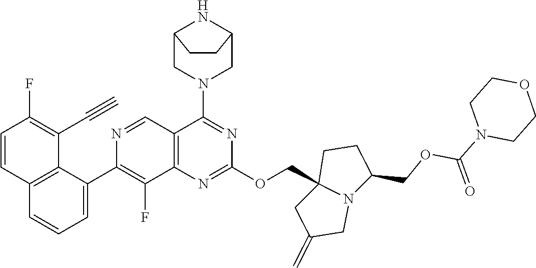 [SMILES: C#Cc1c(F)ccc2cccc(-c3ncc4c(N5CC6CCC(C5)N6)nc(OC[C@@]56CC[C@@H](COC(=O)N7CCOCC7)N5CC(=C)C6)nc4c3F)c12] | 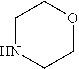 | 722.1 |
| 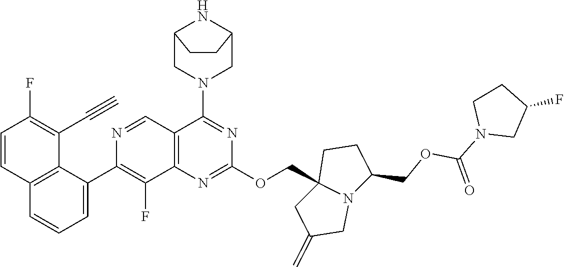 [SMILES: C#Cc1c(F)ccc2cccc(-c3ncc4c(N5CC6CCC(C5)N6)nc(OC[C@@]56CC[C@@H](COC(=O)N7CC[C@H](F)C7)N5CC(=C)C6)nc4c3F)c12] | 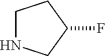 | 724.2 |
| 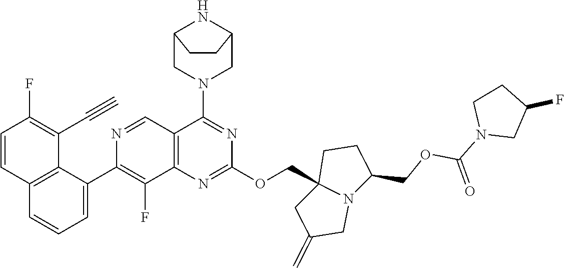 [SMILES: C#Cc1c(F)ccc2cccc(-c3ncc4c(N5CC6CCC(C5)N6)nc(OC[C@@]56CC[C@@H](COC(=O)N7CC[C@@H](F)C7)N5CC(=C)C6)nc4c3F)c12] | 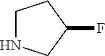 | 724.2 |
| 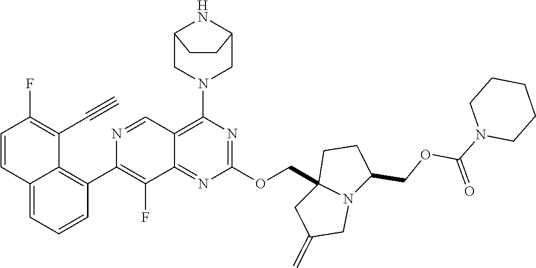 | 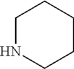 | 720.3 |
| 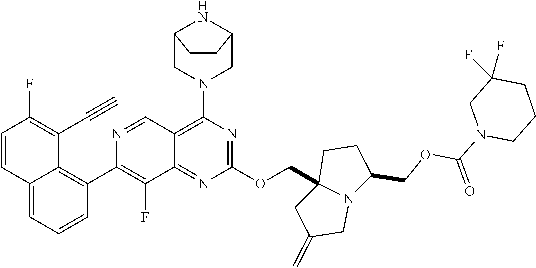 | 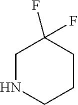 | 756.4 |
| 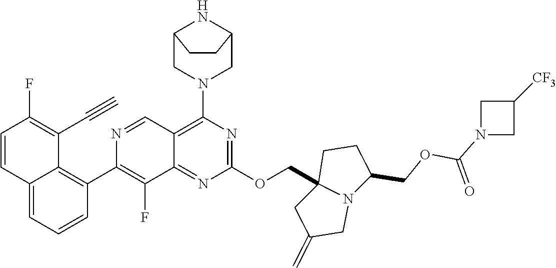 | 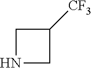 | 760.2 |
| 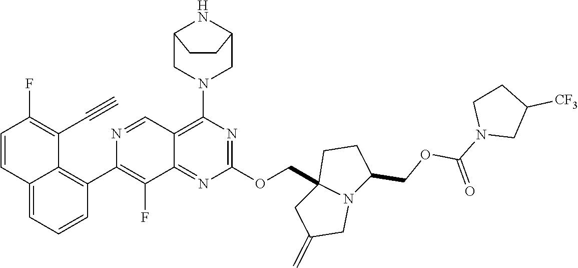 | 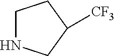 | 774.2 |

### BIOLOGICAL EXAMPLES

### Example 1

### p-ERK Cellular 1-Plate Assay

The ability of the compound of Formula (I) or a pharmaceutically acceptable salt thereof (test compound) to inhibit K-Ras G12D activity was tested using AGS (Cobioer, CBP60476) cell lines which harbor KRAS G12D mutation as described below.

AGS (Cobioer, CBP60476) were seeded in 384-well plates and cultured overnight (5,000 cells per well, 40 μl total volume). The following morning, cells were treated with test compound, with starting concentration at 10 μM and 3-fold dilution down to 0.5 nM for 3 h at 37° C. DMSO treatment served as control, p-ERK was then measured using AlphaLISA SureFire Ultra p-ERK1/2 (Thr202/Tyr204) Assay Kit (Perkin Elmer, cat #ALSU-PERK) following the manufacturer's instruction as follows.

Briefly, the culture medium was removed and 10 μl 1× lysis buffer was added to each well, followed by 10 minutes incubation on a plate shaker at room temperature. Acceptor mixture was prepared according to manufacturer's instruction. 5 μl acceptor mixture was added to the cell lysate and the plate was wrapped with foil, spun at 500 rpm for 10 s and incubated at RT for 60 min. Donor mixture was prepared under subdued light. 5 μl donor mixture was added to the cell lysate and the plate was spun at 500 rpm for 10s and incubated at RT for another 60 min. in the dark. Signal was then measured on a EnVision 2105 multimode plate reader. Percentage inhibition was calculated with DMSO treatment as 100% of signal, and IC₅₀ was calculated by XLfit 5.5.x.

### Example 2

### p-ERK Cellular 2-Plate Assay

The ability of the compound of Formula (I) or a pharmaceutically acceptable salt thereof (test compound) to inhibit K-Ras G12) activity was tested using AGS (Cobioer, CBP60476) cell lines which harbor KRAS G12D mutation as described below.

AGS (Cobioer, CBP60476) were seeded in 384-well plates and cultured overnight (10,000 cells per well, 40 μl total volume) The following morning, cells were treated with test compound, with starting concentration at 10 μM and 3-fold dilution down to 0.5 nM for 3 h at 37° C. DMSO treatment served as control. p-ERK was then measured using AlphaLISA SureFire Ultra p-ERK1/2 (Thr202/Tyr204) Assay Kit (Perkin Elmer, cat #ALSU-PERK) following the manufacturer's instruction as follows,

Briefly, the culture medium was removed and 20 μl 1× lysis buffer was added to each well, followed by 30 minutes incubation on a plate shaker at room temperature. Then 10 μl of lysate was transferred to a white 384-well plate. Acceptor mixture was prepared according to manufacturer's instruction. 5 μl acceptor mixture was added to the cell lysate and the plate was wrapped with foil, spun at 500 rpm for 10 s, and incubated at RT for 60 min. Donor mixture was prepared under subdued light. 5 μl donor mixture was added to the cell lysate and the plate was spun at 500 rpm for 10s and incubated at RT for another 60 min. in the dark. Signal was then measured on a EnVision 2105 multimode plate reader. Percentage inhibition was calculated with DMSO treatment as 100% of signal, and IC50 was calculated by XLfit 5.5.x.

### Example 3

### p-ERK Cellular Assay

The ability of the compound of Formula (I) or a pharmaceutically acceptable salt thereof (test compound) to inhibit K-Ras G12D activity were tested using the PANC-1 (ATCC, CRL-1469) and A-427 (ATCC, HTB-53) cell lines as described below %.

PANC-1 (ATCC, CRL-1469) or A-427 (ATCC, HTB-53) (harbor KRAS G12D mutation) were seeded in 96-well plates and cultured overnight (20,000 cells per well, 200 μl total volume). On the following morning, cells were treated with test compound, with starting concentration at 10 μM and ½ log dilution down to 1 nM for 3 hours at 37° C. DMSO treatment serves as control, p-ERK is then measured using Advanced Phospho-ERK 1/2 (Thr202/Tyr204) Assay Kit (Cisbio, Cat #64AERPET) following the manufacturer's instruction.

Briefly, medium was removed and 40 μl 1× lysis buffer was added to each well, followed by 30 minutes incubation on a plate shaker at room temperature. Then 8 μl of lysate was transferred to a white low volume 384-well plate. Acceptor d2 antibody and Cryptate antibody were diluted (1:20) with detection buffer and gently mixed (1:1) according to manufacturer's instruction. 2 μl antibodies mix was added to the cell lysate and the plate was wrapped with foil, shaken for 1-2 minutes on a plate reader. and incubated for >4 hours at room temperature. Signal was then measured on a CLARIOstar® plate reader. Percentage inhibition is calculated with DMSO treatment as 100% of signal, and IC₅₀ is calculated by Graphpad Prism 7.

The IC₅₀ of the compounds disclosed in Compound Table 1 above in above assays are disclosed in Table below.

| Cpd. # (From | AGS 1 plate | AGS 2 plate | A427 | Cpd. # (From | AGS 1 plate | AGS 2 plate | A427 |
|---|---|---|---|---|---|---|---|
| Cpd. | EC₅₀ | EC₅₀ | EC₅₀ | Cpd. | EC₅₀ | EC₅₀ | EC₅₀ |
| Table 1) | nM | nM | uM | Table 1) | nM | nM | uM |
| 1 | | 0.07 | 0.35 | 30 | 170 | | |
| 2 | 0.36 | | | 31 | >100 | | |
| 3 | 3.1 | | | 32 | | 5.9 | 23 |
| 4 | 0.57 | | | 33 | 21 | 3.8 | 18 |
| | | | | | | 8 | 23 |
| 5 | 1.1 | | | 34a and | | One of 34a | |
| | | | | 34b | | and 34b is | |
| | | | | | | 9.6. | |
| | | | | | | the other is | |
| | | | | | | >100 | |
| 6a and | | one of 6a | | 37 | 22 | | |
| 6b | | and 6b is | | | | | |
| | | 0.03 and | | | | | |
| | | the other is | | | | | |
| | | 0.12 | | | | | |
| 7 | | 0.16 | 1.9 | 38 | 15 | | |
| | | 1.0 | | | | | |
| 8 | 11 | | | 39 | 724 | | |
| 9 | 1.2 | | | 40 | | | |
| 10 | | 0.55 | | 41 | | | |
| 11 | | 28 | | 42 | | | 25 |
| 12 | | 0.04 | 0.6 | 43 | | | 12 |
| 13 | | 0.08 | 0.79 | 44 | 14 | | 44 |
| 14 | | 0.2 | 1.0 | 45 | 14 | | 38 |
| 15 | | 8.3 | | 46 | 62 | | |
| 16 | | 23 | | 47 | 23 | | |
| 17 | | 0.36 | | 48 | 54 | | |
| 18 | | 0.19 | | 49 | | | |
| 19 | | 0.06 | 0.59 | 50 | | | |
| 20a and | | one of 20a | | 51 | | | 42 |
| 20b | | and 20b | | | | | |
| | | 0.07 and | | | | | |
| | | the other is | | | | | |
| | | 1.8 | | | | | |
| 21 | | 7.1 | 22 | 52 | | | 12 |
| 22 | | 32 | | 53 | | | 105 |
| 23 | | 58 | | 54 | | 9.5 | 23 |
| 24 | | 3.0 | 5.5 | 55 | | 5.7 | 15 |
| 25 | | 82 | | 56 | | 3.7 | 12 |
| 26 | | 22 | 48 | 57 | | 21 | 15 |
| 27 | | 13 | 71 | 58 | | 9 | 13 |
| 28 | | | 85 | 59 | 28 | 1.6 | 9 |
| 29 | 15 | | | 60 | 11 | | 13 |
| 30 | 170 | | | 61 | 28 | | |
| 31 | >100 | | | 62 | 19 | | |
| 32 | | 5.9 | 23 | 64 | 41 | | |
| 33a and | | one of 33a | | 65 | 37 | | |
| 33b | | and 33b is | | | | | |
| | | 3.8. | | | | | |
| | | the other is | | | | | |
| | | 8 | | | | | |
| 35 | 4.4 | | | 66 | 47 | | |
| 36 | 10 | | | 67 | 50 | | |

### Example 4

### PK Study

PK parameters and bioavailability of some non-phenolic compounds (i.e., R⁵ is naphthyl ring lacking —OH) disclosed in compound Table 1 (test compounds) were determined in mouse and rat as described below.

### Mouse PK Study

Male CD-1 mice were used in the study. All animals for IV and PO administration were fasted overnight and fed after 4 h collection. In each study, two or three animals were included. Mice were dosed by IV at 1˜5 mg/kg, or orally dosed at 1˜30 mg/kg. The test compounds were formulated in one of the formulations below:
1. 10% DMSO: 40% PEG 400: 50% “10% HP-β-CD in water
2. 10% DMSO/5% Cremophor RH 40/85% (10% HP-β-CD in water);
3. DMSO/PEG400/‘10% HP-3-CD’ in water(v:v): 1:4:5.
4. 10% Captisol in “50 mM citrate buffer pH 5.0”

Blood samples were taken at 5 min., 15 min., 30 min., 1 h, 2 h, 4h, 6 h, 8 h and 24 h post IV dose and 15 min., 30 min., 1 h, 2 h, 4 h, 6 h, 8 h, and 24 h post PO dose. The collected blood of each sample was transferred into plastic micro centrifuge tubes containing anticoagulant of Heparin-Na or EDTA-K₂ and mixed well with anticoagulant. The samples were centrifuged at 4000 g for 5 minutes in a 4° C. ice bath to obtain plasma. The samples were be stored in a freezer at −75±15° C. prior to analysis.

The concentration of each tested compound in the plasma samples determined by LC-MS/MS methods. Oral bioavailability was calculated by following formula

$$\\ F \% = ( Oral AUC / dose ) / ( IV AUC / dose )$$

### Rat PK Study

The Rat PK study was conducted using above protocol except Male SD rats were used in the study.

In general, the compounds tested had oral bioavailabilites (PO F %) of between about 7% and about 22% in mice, and between about 5% and about 18% in rats. In an embodiment, a compound of the disclosure had a bioavailability of about 70% in mice and about 47% in rat.

### Formulation

### Examples

The following are representative pharmaceutical formulations containing a compound of the present disclosure.

### Tablet Formulation

The following ingredients are mixed intimately and pressed into single scored tablets.

| Ingredient | Quantity per tablet (mg) |
|---|---|
| compound Formula (I) | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule (mg) |
|---|---|
| compound Formula (I) | 200 |
| lactose spray dried | 148 |
| magnesium stearate | 2 |

### Injectable Formulation

Compound of the disclosure (e.g., compound 1) in 2% HPMC, 1% Tween 80 in DI water, pH 2.2 with MSA, q.s. to at least 20 mg/mL

### Inhalation Composition

To prepare a pharmaceutical composition for inhalation delivery, 20 mg of a compound disclosed herein is mixed with 50 mg of anhydrous citric acid and 100 mL of 0.9% sodium chloride solution. The mixture is incorporated into an inhalation delivery unit, such as a nebulizer, which is suitable for inhalation administration.

### Topical Gel Composition

To prepare a pharmaceutical topical gel composition, 100 mg of a compound disclosed herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Ophthalmic Solution Composition

To prepare a pharmaceutical ophthalmic solution composition, 100 mg of a compound disclosed herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Nasal Spray Solution

To prepare a pharmaceutical nasal spray solution, 10 g of a compound disclosed herein is mixed with 30 mL of a 0.05M phosphate buffer solution (pH 4.4). The solution is placed in a nasal administrator designed to deliver 100 ul of spray for each application.

1. A compound of Formula (I′c): 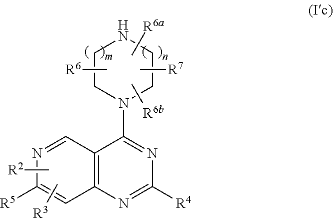
wherein:
one of m and n is 0, 1, or 2, and the other of m and n is 0, 1, 2, or 3;
R⁶ is hydrogen, deuterium, alkyl, alkoxy, halo, haloalkyl, hydroxy, hydroxylalkyl, alkoxyalkyl, cyano, or cyanomethyl, provided R⁶, R¹⁰, and R²⁸ are not attached to the ring —NH—;
R⁷ is hydrogen, deuterium, alkyl, alkoxy, halo, haloalkyl, hydroxy, hydroxylalkyl, or alkoxyalkyl, provided R⁷ is not attached to the ring —NH—; or
when R⁶ and R⁷ are attached to the carbon atoms of the ring that are opposite or diagonal to each other, then R⁶ and R⁷ can combine to form —(CH₂)_{z}— where (z is 1, 2, or 3), or —CH═CH—;
R^{6a} is hydrogen, deuterium, alkyl, alkylidenyl, alkoxy, halo, haloalkyl, hydroxy, hydroxyalkyl, alkoxyalkyl, cyano, cyanomethyl, cyanoethyl, or 2-cyanovinyl, provided R^{6a} is not attached to the ring —NH—;
R^{6b} is hydrogen or alkyl, provided R^{6b} is not attached to the ring —NH—; or
when R^{6a} and R^{6b} are attached to the same carbon of ring (a′), R^{6a} and R^{6b} can combine to form cycloalkylene;
R² is hydrogen, deuterium, alkyl, halo, haloalkyl, alkoxy, hydroxy, or cyano;
R³ is hydrogen, deuterium, alkyl, halo, haloalkyl, alkoxy, cycloalkyl, cycloalkyloxy, hydroxy, or cyano;
R⁴ is —Z—R³⁰ where Z is a bond, O, NH, N(alkyl), or S; and R³⁰ is bicyclic heterocyclylalkyl wherein bicyclic heterocyclyl part of bicyclic heterocyclylalkyl, is substituted with R^{d}, R^{e}, and R^{f}where R^{d} is alkenyl, haloalkenyl, alkylidenyl, haloalkylidenyl, alkoxyalkylidenyl, or R^{d} is ═CR³³R³⁴;
where R³³ is hydrogen, alkyl, or fluoro and R³⁴ is cyano, alkoxyalkyloxyalkyl, cycloalkyl, cycloalkylalkyl, or cycloalkylalkyloxyalkyl (where cycloalkyl, by itself or as part of cycloalkylalkyl and cycloalkylalkyloxyalkyl is optionally substituted with one or two substituents independently selected from alkyl, halo, haloalkoxy, alkoxy, alkoxyalkyl, and hydroxy), heterocyclyl, phenyl, or heteroaryl (where heterocyclyl, phenyl, and heteroaryl are optionally substituted with one, two, or three substituents independently selected from alkyl, halo, haloalkyl, haloalkoxy, alkoxy, cyano, and hydroxy), or R³³ and R³⁴ together with the carbon atom to which they are attached form cycloalkylene optionally substituted with alkyl, halo, alkoxy, or hydroxy;
R^{e} is hydrogen, alkyl, halo, haloalkyl, haloalkoxy, alkoxy, hydroxy, alkoxyalkyl, alkoxyalkoxy, alkoxyalkyloxyalkyl, cycloalkyl, cycloalkylalkyl, cycloalkoxy, cycloalkylalkyloxy, cycloalkoxyalkyl, cycloalkylalkyloxyalkyl, cycloalkylalkyloxyalkyloxy, bridged cycloalkyloxy, bridged cycloalkyloxyalkyl, bridged cycloalkylalkyloxy, bridged cycloalkylalkyloxyalkyl, heterocyclyl, heterocyclylalkyl, heterocyclyloxy, heterocyclyloxyalkyl, heterocyclylalkyloxy, heterocyclylalkyloxyalkyl (where cycloalkyl, by itself or as part of cycloalkylalkyl, cycloalkoxy, cycloalkoxyalkyl, cycloalkylalkyloxy, cycloalkylalkyloxyalkyl, and cycloalkylalkyloxyalkyloxy, bridged cycloalkyl, as part of bridged cycloalkyloxy, bridged cycloalkyloxyalkyl, bridged cycloalkylalkyloxy, and bridged cycloalkylalkyloxyalkyl, and heterocyclyl, by itself or as part of heterocyclylalkyl, heterocyclyloxy, heterocyclyloxyalkyl, heterocyclylalkyloxy, and heterocyclylalkyloxyalkyl are optionally substituted with one, two, or three substituents independently selected from alkyl, halo, haloalkyl, haloalkoxy, alkoxy, alkoxyalkyl, cyano, and hydroxy), or -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy), provided that when R^{d} is alkenyl, haloalkenyl, alkylidenyl, haloalkylidenyl, or alkoxyalkylidenyl, then R^{e} is other than hydrogen; and
R^{f} is hydrogen, alkyl, halo, alkoxy, alkoxyalkyl, or hydroxy; and
R⁵ is -Q-R⁴⁴ where Q is bond, alkylene, or —C(═O)—; and R⁴⁴ is cycloalkyl, fused cycloalkyl, fused spirocycloalkyl, aryl, aralkyl, heteroaryl, fused heteroaryl, or heteroaralkyl wherein aryl, aryl in aralkyl, heteroaryl, fused heteroaryl, and heteroaryl in heteroaralkyl are independently substituted with R^{aa}, R^{bb}, R^{cc} and R^{dd} wherein R^{aa} and R^{bb} are each independently selected from hydrogen, alkyl, cycloalkyl, halo, haloalkyl, haloalkoxy, hydroxy, alkoxy, heteroalkyl, hydroxyalkyl, amino, and cyano, R^{cc} is hydrogen, alkenyl, alkynyl, cyanoalkenyl, cyanoalkynyl, or halo, and R^{dd} is hydrogen, alkyl, alkylthio, cycloalkyl, halo, haloalkyl, haloalkoxy, alkoxy, heteroalkyl, hydroxyalkyl, amino, cyano, optionally substituted aryl, optionally substituted heteroaryl, optionally substituted heterocyclyl, or optionally substituted heterocyclylalkyl; or
a pharmaceutically acceptable salt thereof.

2. The compound of claim 1, or a pharmaceutically acceptable salt thereof, wherein: 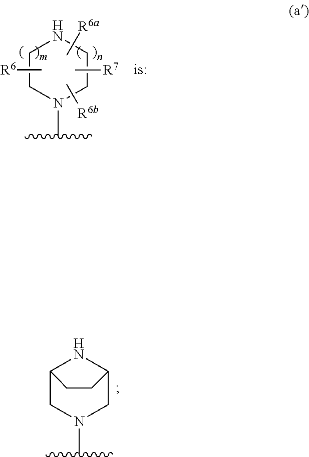
and
the bicyclic heterocyclylalkyl is a ring of formula: 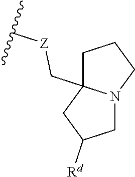
substituted with R^{e} and R^{f} and where R^{d} is alkylidenyl.

3. The compound of claim 2, or a pharmaceutically acceptable salt thereof, wherein the bicyclic heterocyclylalkyl is a ring of formula: 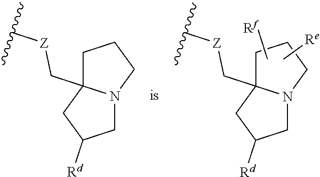
wherein R^{d} is methylidenyl and Z is —O—.

4. The compound of claim 3, or a pharmaceutically acceptable salt thereof, wherein R^{e} is -(alkylene)-OC(O)R³⁹ (where R³⁹ is amino, alkylamino, dialkylamino or heterocyclyl optionally substituted with one or two substituents independently selected from alkyl, halo, haloalkyl, haloalkoxy, cyano, alkoxy, and hydroxy).

5. (canceled)

6. The compound of claim 4, or a pharmaceutically acceptable salt thereof, wherein R^{e} is -(alkylene)-OC(O)R³⁹ where alkylene is methylene or ethylene and R³⁹ is pyrrolidin-1-yl, piperidin-1-yl, or morpholin-4-yl, each ring optionally substituted with one or two substituents independently selected from methyl, fluoro, trifluoromethyl, trifluoromethoxy, and methoxy.

7. The compound of claim 3, or a pharmaceutically acceptable salt thereof, wherein R^{e} is alkyl, halo, haloalkyl, haloalkoxy, alkoxy, hydroxy, cycloalkyl, cycloalkylalkyl, heterocyclyl, or heterocyclylalkyl, (where cycloalkyl, by itself or as part of cycloalkylalkyl, and heterocyclyl, by itself or as part of heterocyclylalkyl, are optionally substituted with one, two, or three substituents independently selected from alkyl, halo, haloalkyl, haloalkoxy, alkoxy, alkoxyalkyl, cyano, and hydroxy).

8. The compound of claim 7, or a pharmaceutically acceptable salt thereof, wherein R^{e} is methyl, fluoro, chloro, difluoromethyl, trifluoromethyl, difluoromethoxy, trifluoromethoxy, methoxy, ethoxy, hydroxy, cyclopropyl, cyclopropylmethyl, pyrrolidinyl, pyrrolidinylmethyl, pyrrolidinylethyl, piperidinyl, piperidinylmethyl, piperidinylethyl, piperizinyl, piperizinylmethyl, or piperizinylethyl (wherein cyclopropyl, by itself or as part of cyclopropylmethyl, pyrrolidinyl, by itself or as part of pyrrolidinylmethyl and pyrrolidinylethyl, piperidinyl, by itself or as part of piperidinylmethyl and piperidinylethyl, and piperazinyl, by itself or as part of piperazinylmethyl and piperazinylethyl are optionally substituted with one or two substituents independently selected from methyl, chloro, fluoro, trifluoromethyl, trifluoromethoxy, methoxy, methoxymethyl, and cyano).

9-11. (canceled)

12. The compound of claim 8, or a pharmaceutically acceptable salt thereof, wherein R^{f} is hydrogen, methyl, ethyl, methoxy, ethoxy, methyloxy, ethyloxy, chloro, or fluoro.

13. The compound of claim 6, or a pharmaceutically acceptable salt thereof, wherein R^{f} is hydrogen.

14. The compound of claim 2, or a pharmaceutically acceptable salt thereof, wherein

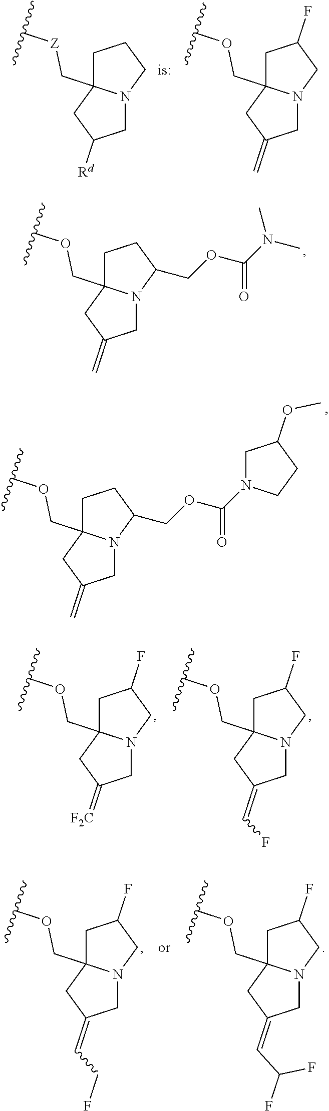

15. The compound of Formula (I′c) of claim 12, or a pharmaceutically acceptable salt thereof, having a structure of formula (I′d) as follows:

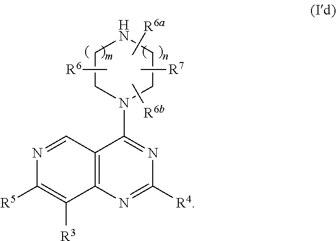

16. The compound of claim 15, or a pharmaceutically acceptable salt thereof, wherein R⁵ is -Q-R⁴⁴ where Q is bond and R⁴⁴ is phenyl or naphthyl substituted with R^{aa}, R^{bb}, R^{cc} and R^{dd}.

17. The compound of claim 16, or a pharmaceutically acceptable salt thereof, wherein R^{aa} and R^{bb} are independently selected from hydrogen, methyl, ethyl, fluoro, chloro, trifluoromethyl, difluoromethyl, trifluoromethoxy, hydroxy, methyl, ethoxy, cyclopropyl, amino, cyano, and hydroxymethyl, R^{cc} is hydrogen, ethynyl, 2-cyanovinyl, 2-cyanoethyn-1-yl, or fluoro, and R^{dd} is hydrogen, methyl, fluoro, amino, or cyclopropyl.

18. The compound of claim 15, or a pharmaceutically acceptable salt thereof, wherein R⁵ is -Q-R⁴⁴ where Q is bond and R⁴⁴ is heteroaryl or fused heteroaryl substituted with R^{aa}, R^{bb}, R^{cc} and R^{dd}.

19. The compound of claim 15, or a pharmaceutically acceptable salt thereof, wherein R⁵ is -Q-R⁴⁴ where Q is bond and R⁴⁴ is:

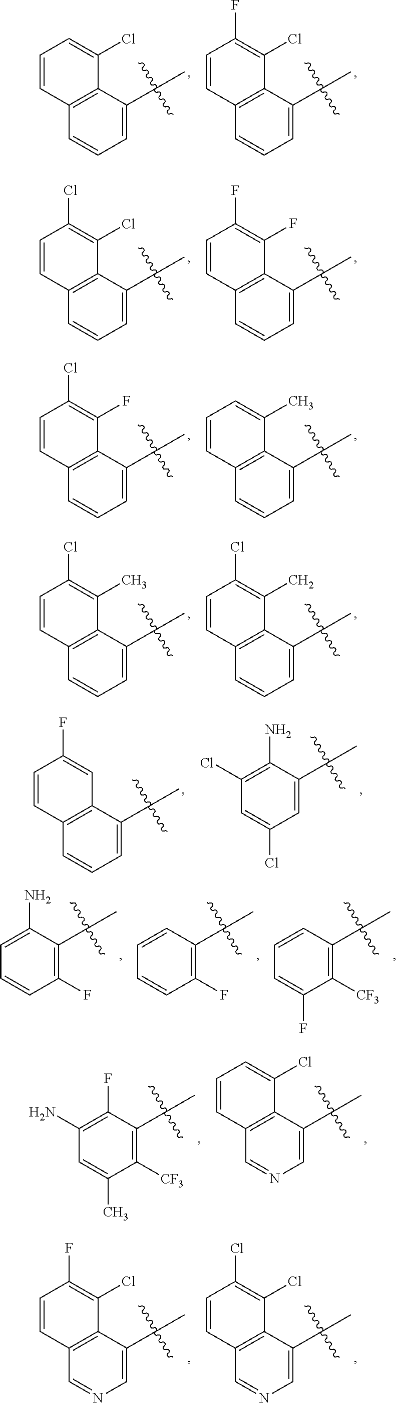

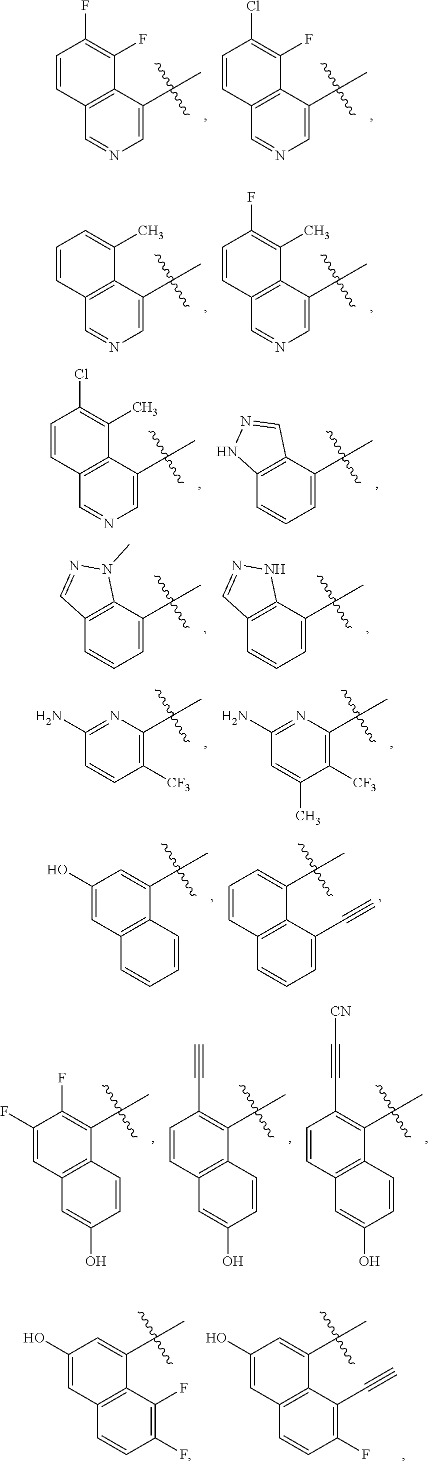

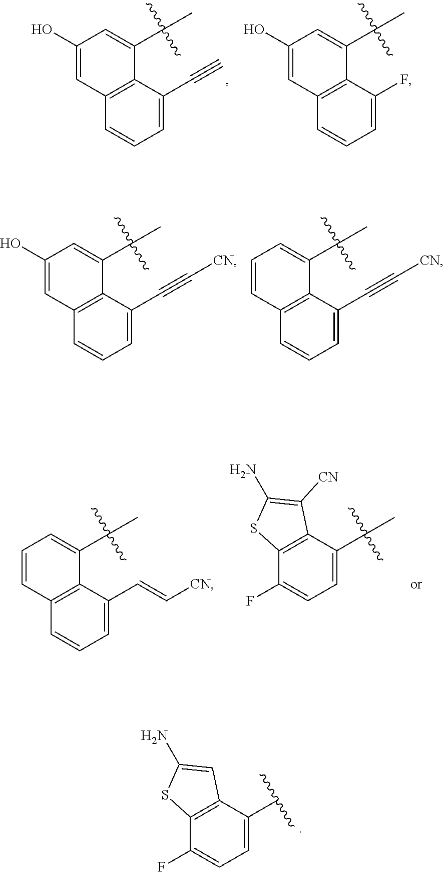

20-21. (canceled)

22. The compound of claim 19, or a pharmaceutically acceptable salt thereof, wherein R² is hydrogen, halo, or alkyl, and R³ hydrogen, halo, cycloalkyloxy, or alkyl.

23. The compound of claim 13, or a pharmaceutically acceptable salt thereof, wherein R⁵ is -Q-R⁴⁴ where Q is bond and R⁴⁴ is phenyl, naphthyl, heteroaryl or fused heteroaryl, each ring substituted with R^{aa}, R^{bb}, R^{cc} and R^{dd}.

24. The compound of claim 23, or a pharmaceutically acceptable salt thereof, wherein R² is hydrogen or chloro and R³ is fluoro.

25. The compound of claim 1 selected from:
4-(4-((1R,5S)-3,8-diazabicyclo[3.2.1]octan-3-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-((1R,5S)-3,8-diazabicyclo[3.2.1]octan-3-yl)-2-(((6R,7aR)-2-ethylidene-6-fluorotetrahydro-1H-pyrrolizin-7a(5H)-yl)methoxy)-8-fluoropyrido[4,3-d]pyrimidin-7-yl)-5-ethynyl-6-fluoronaphthalen-2-ol;
4-(4-((1R,5S)-3,8-diazabicyclo[3.2.1]octan-3-yl)-2-(((6S,7aR)-2-cyclobutylidene-6-fluorotetrahydro-1H-pyrrolizin-7a(5H)-yl)methoxy)-8-fluoropyrido[4,3-d]pyrimidin-7-yl)-5-ethynyl-6-fluoronaphthalen-2-ol;
4-(4-((1R,5S)-3,8-diazabicyclo[3.2.1]octan-3-yl)-2-(((2R,7aR)-2-(difluoromethoxy)-6-methylenetetrahydro-1H-pyrrolizin-7a(5H)-yl)methoxy)-8-fluoropyrido[4,3-d]pyrimidin-7-yl)-5-ethynyl-6-fluoronaphthalen-2-ol;
4-(4-((1R,5S)-3,8-diazabicyclo[3.2.1]octan-3-yl)-2-(((2R,7aR)-2-(cyclopropylmethoxy)-6-methylenetetrahydro-1H-pyrrolizin-7a(5H)-yl)methoxy)-8-fluoropyrido[4,3-d]pyrimidin-7-yl)-5-ethynyl-6-fluoronaphthalen-2-ol;
4-(4-((1R,5S)-3,8-diazabicyclo[3.2.1]octan-3-yl)-8-fluoro-2-(((5S,7aS)-5-(methoxymethyl)-2-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-((1R,5S)-3,8-diazabicyclo[3.2.1]octan-3-yl)-8-fluoro-2-(((5S,7aR)-5-(methoxymethyl)-2-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-((1R,5S)-3,8-diazabicyclo[3.2.1]octan-3-yl)-8-fluoro-2-(((5R,7aR)-5-(methoxymethyl)-2-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-((1R,5S)-3,8-diazabicyclo[3.2.1]octan-3-yl)-8-fluoro-2-(((5R,7aS)-5-(methoxymethyl)-2-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-(2,5-diazabicyclo[2.2.2]octan-2-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol;
5-ethynyl-6-fluoro-4-(8-fluoro-2-(((2S,7aR)-2-fluoro-6-methylenetetrahydro-1H-pyrrolizin-7a(5H)-yl)methoxy)-4-(1-methyl-3,8-diazabicyclo[3.2.1]octan-3-yl)pyrido[4,3-d]pyrimidin-7-yl)naphthalen-2-ol;
4-(4-((1R,5S)-3,8-diazabicyclo[3.2.1]oct-6-en-3-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-(3,8-diazabicyclo[3.2.1]oct-6-en-8-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynyl-6-fluoronaphthalen-2-ol;
4-(4-((1R,5S)-3,8-diazabicyclo[3.2.1]octan-3-yl)-8-fluoro-2-(((2S,7aR)-2-fluoro-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2R,7aR)-2-fluoro-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2R,7aS)-2-fluoro-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2S,7aS)-2-fluoro-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2S,7aS)-2-methoxy-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2R,7aR)-2-methoxy-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2R,7aS)-2-methoxy-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2S,7aR)-2-methoxy-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2S,7aS)-2-(methoxymethyl)-6-methylenetetrahydro-1H-pyrrolizin-7a(5H)-yl)methoxy)pyrido[4,3-d]pyrimidin-7-yl)-5-ethynylnaphthalen-2-ol;
4-(4-((1R,5S)-3,8-diazabicyclo[3.2.1]octan-3-yl)-8-fluoro-2-(((2S,7aR)-2-(methoxymethyl)-6-methylenetetrahydro-1H-pyrrolizin-7a(5H)-yl)methoxy)pyrido[4,3-d]pyrimidin-7-yl)-5-ethynylnaphthalen-2-ol;
4-((1R,5S)-3,8-diazabicyclo[3.2.1]octan-3-yl)-7-(8-ethynyl-7-fluoronaphthalen-1-yl)-8-fluoro-2-(((2S,7aR)-2-methoxy-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2R,7aS)-2-methoxy-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2S,7aR)-2-(methoxymethyl)-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2S,7aS)-2-(methoxymethyl)-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2R,7aR)-2-fluoro-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2S,7aS)-2-fluoro-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2R,7aS)-2-fluoro-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2S,7aR)-2-fluoro-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-2-(((6R,7aR)-2-cyclobutylidene-6-fluorotetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2R,7aR)-2-fluoro-6-(propan-2-ylidene)tetrahydro-1H-pyrrolizin-7a(5H)-yl)methoxy)pyrido[4,3-d]pyrimidine;
7-(8-ethynyl-7-fluoronaphthalen-1-yl)-8-fluoro-2-(((2S,7aR)-2-fluoro-6-methylenetetrahydro-1H-pyrrolizin-7a(5H)-yl)methoxy)-4-(1-methyl-3,8-diazabicyclo[3.2.1]octan-3-yl)pyrido[4,3-d]pyrimidine;
2-(7-(8-ethynyl-7-fluoronaphthalen-1-yl)-8-fluoro-2-(((2R,7aR)-2-fluoro-6-methylenetetrahydro-1H-pyrrolizin-7a(5H)-yl)methoxy)pyrido[4,3-d]pyrimidin-4-yl)-2,5-diazabicyclo[2.2.2]octane;
4-((1R,5S)-3,8-diazabicyclo[3.2.1]octan-3-yl)-7-(8-ethynyl-7-fluoronaphthalen-1-yl)-8-fluoro-2-(((2R,7aR)-2-methoxy-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-5-(methoxymethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aR)-5-(methoxymethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5R,7aR)-5-(methoxymethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5R,7aS)-5-(methoxymethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-5-((2-methoxyethoxy)methyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-2-(((5S,7aS)-5-((difluoromethoxy)methyl)-2-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-2-(((5S,7aS)-5-(cyclopropoxymethyl)-2-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-2-(((5S,7aS)-5-((cyclopropylmethoxy)methyl)-2-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-5-(fluoromethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-2-(((5S,7aS)-5-(3,3-difluoropropyl)-2-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-5-(2-fluoroethyl)-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-2-methylene-5-(3,3,3-trifluoropropyl)tetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((5S,7aS)-5-isobutyl-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-2-(((5S,7aS)-5-cyclopropyl-2-methylenetetrahydro-1H-pyrrolizin-7a(5H)-yl)methoxy)-7-(8-ethynyl-7-fluoronaphthalen-1-yl)-8-fluoropyrido[4,3-d]pyrimidine; 50
4-((1R,5S)-3,8-diazabicyclo[3.2.1]octan-3-yl)-7-(8-ethynyl-7-fluoronaphthalen-1-yl)-8-fluoro-2-(((5S,7aS)-5-isopropyl-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-2-(((2S,7aR)-2-(difluoromethoxy)-6-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-2-(((2R,7aR)-2-cyclopropoxy-6-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-2-(((2R,7aR)-2-(difluoromethoxy)-6-methylenetetrahydro-1H-pyrrolizin-7a(5H)-yl)methoxy)-7-(8-ethynyl-7-fluoronaphthalen-1-yl)-8-fluoropyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((6R,7aS)-6-fluoro-1-methyl-2-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2R,3R,7aR)-2-fluoro-3-methyl-6-methylenetetrahydro-1H-pyrrolizin-7a(5H)-yl)methoxy)pyrido[4,3-d]pyrimidine;
4-((1R,5S)-3,8-diazabicyclo[3.2.1]octan-3-yl)-7-(8-ethynyl-7-fluoronaphthalen-1-yl)-8-fluoro-2-(((2S,3R,7aR)-2-fluoro-3-methyl-6-methylenetetrahydro-1H-pyrrolizin-7a(5H)-yl)methoxy)pyrido[4,3-d]pyrimidine;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl dimethylcarbamate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl azetidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl 3-fluoroazetidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl 3,3-difluoroazetidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl pyrrolidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl 3-fluoropyrrolidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl 3,3-difluoropyrrolidine-1-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl ethyl(methyl)carbamate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl morpholine-4-carboxylate;
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl (S)-3-fluoropyrrolidine-1-carboxylate; and
((3S,7aS)-7a-(((4-((1R,5S)-3,8-diazabicyclo[3.2.1]octan-3-yl)-7-(8-ethynyl-7-fluoronaphthalen-1-yl)-8-fluoropyrido[4,3-d]pyrimidin-2-yl)oxy)methyl)-6-methylenehexahydro-1H-pyrrolizin-3-yl)methyl (R)-3-fluoropyrrolidine-1-carboxylate; or
a pharmaceutically acceptable salt thereof.

26. A pharmaceutical composition comprising a compound of claim 1, or a pharmaceutically acceptable salt thereof and a pharmaceutically acceptable excipient.

27. A method of treating cancer in a patient comprising administering to the patient, a therapeutically effective amount of a compound of claim 1, or a pharmaceutically acceptable salt thereof.

28. The method of claim 27, wherein the cancer is mediated with kras G12D mutant.

29. The method of claim 28, wherein the cancer is non-small cell lung cancer, colorectal cancer, or pancreatic cancer.

30. The method of claim 29, wherein the compound of claim 29 is administered in combination with at least one additional anticancer agent.
